SEQUENCE LISTING (10 sequences)

SEQ ID NO: 1 - DNA, 1317 bp, Pseudomonas aeruginosa
atgaatacttattttgatattccgcatcggctagtgggaaaggctctgtatgaatcatattatgatcattttggccaaatggatatattgtcagatggaagtttgtacctaatatataggcgggcaacagagcatgtaggtggtagtgatgggcgtgttgttttcagtaaactggagggtggtatttggagtgcgcctaccatagttgcccaggcgggaggacaggattttcgagatgtagctggtgggacgatgcctagcggaaggatcgtcgcggcctcgacggtttatgaaacaggagaggtgaaggtctatgtatctgatgattcaggtgtgacatgggtacataaattcacattggctagaggtggggcggattataattttgctcatggaaaaagttttcaagtaggggcacgctatgtgattcctctttacgcggcgacgggagtcaattatgaactgaaatggctagagtcttctgatggcggagaaacttggggcgaaggaagcacgatctacagcgggaacacaccatacaatgagacttcttaccttcctgtgggagatggcgtaattctggctgtagccagagtaggatctggtgccggaggagcgttacgtcagtttataagtctggacgacggcggcacatggactgatcagggtaatgtgactgcacaaaatggtgactctaccgatattctcgtagccccttctctctcctatatttattctgagggaggtacaccacatgtagttcttttgtacacgaacagaacgactcatttttgttattacagaacaatcctgctggcgaaagccgtagcaggctcctccggctggacggagcgtgttcctgtgtatagtgcgccagccgcctccggttatactagtcaagtcgttttgggaggtcgaagaatactgggtaatctattcagggagacgtcttctacgacgtcgggagcttatcagtttgaagtgtatctgggaggagtcccggattttgagtcagactggttttctgtttcttcaaatagtttgtataccctgagtcatggactccagaggtcgccgcgtcgggtggtcgttgagtttgcaaggtcatcgagtccatcaacatggaacattgtcatgcccagttatttcaatgatggagggcataaaggcagtggggctcaggttgaagtgggtagcttgaatattcggcttggaactggagcggcagtatggggcacgggatatttcggaggaatcgacaatagtgccacgactcgattcgctaccgggtattatcgggtcagagcatggatttag

SEQ ID NO: 2 - protein, 438 aa, Pseudomonas aeruginosa
MNTYFDIPHRLVGKALYESYYDHFGQMDILSDGSLYLIYRRATEHVGGSDGRVVFSKLEGGIWSAPTIVAQAGGQDFRDVAGGTMPSGRIVAASTVYETGEVKVYVSDDSGVTWVHKFTLARGGADYNFAHGKSFQVGARYVIPLYAATGVNYELKWLESSDGGETWGEGSTIYSGNTPYNETSYLPVGDGVILAVARVGSGAGGALRQFISLDDGGTWTDQGNVTAQNGDSTDILVAPSLSYIYSEGGTPHVVLLYTNRTTHFCYYRTILLAKAVAGSSGWTERVPVYSAPAASGYTSQVVLGGRRILGNLFRETSSTTSGAYQFEVYLGGVPDFESDWFSVSSNSLYTLSHGLQRSPRRVVVEFARSSSPSTWNIVMPSYFNDGGHKGSGAQVEVGSLNIRLGTGAAVWGTGYFGGIDNSATTRFATGYYRVRAWI

SEQ ID NO: 3 - DNA, 21 bp, Artificial Sequence
  Description of Artificial Sequence Synthetic primer
cgcactatacacaggaacacg

SEQ ID NO: 4 - DNA, 21 bp, Artificial Sequence
  Description of Artificial Sequence Synthetic primer
gcctagcggaaggatcgtcgc

SEQ ID NO: 5 - DNA, 20 bp, Artificial Sequence
  Description of Artificial Sequence Synthetic primer
gattataagtctgccgtcgg

SEQ ID NO: 6 - DNA, 20 bp, Artificial Sequence
  Description of Artificial Sequence Synthetic primer
ctcgggaaacgtgcacatcc

SEQ ID NO: 7 - DNA, 26 bp, Artificial Sequence
  Description of Artificial Sequence Synthetic primer
ccgcgcctatcgccaatgagctgcgc

SEQ ID NO: 8 - DNA, 28 bp, Artificial Sequence
  Description of Artificial Sequence Synthetic primer
cttggggacaccttttagcatcttttgg

SEQ ID NO: 9 - DNA, 20 bp, Artificial Sequence
  Description of Artificial Sequence Synthetic primer
gtggggcgccccaggcacca

SEQ ID NO: 10 - DNA, 25 bp, Artificial Sequence
  Description of Artificial Sequence Synthetic primer
cggttggccttggggttcagggggg

(19) United States
(12) Patent Application Publication
(10) Pub. No.: US 2009/0175805 A1
(43) Pub. Date: Jul. 9, 2009

(54) Neuraminidase Inhibitors and uses thereof

(75) Inventors: Alice Prince, Larchmont, NY (US); Liang Tong, Scarsdale, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 12/282,759

(22) PCT Filed: Mar. 13, 2007
(86) PCT No.: PCT/US07/06337

§ 371 Date: Nov. 24, 2008

Related U.S. Application Data
(60) Provisional application No. 60/781,993, filed on Mar. 13, 2006.
(60) Provisional application No. 60/837,957, filed on Aug. 16, 2006.

(51) Int. Cl. A61K 8/49

(52) U.S. Cl. 424/54

(57) ABSTRACT

The invention is related to various methods for inhibiting or reducing biofilm formation, treating a biofilm production-related disorder, preventing biofilm formation, and screening for neuraminidase inhibitors. The invention also encompasses a mutant bacterial strain with a deleted neuraminidase gene.

The work described herein was supported in whole, or in part, by National Institute of Health Grant No. RO1 DK29693. Thus, the United States Government has certain rights to the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Many respiratory pathogens including Hemophilus influenzae (H. influenzae), Streptococcus pneumoniae (S. pneumoniae), and Pseudomonas aeruginosa (P. aeruginosa) express neuraminidases that can cleave α-2,3 linked sialic acids from glycoconjugates. As mucosal surfaces are heavily sialylated, neuraminidases have been thought to modify epithelial cells by exposing potential bacterial receptors. However, in contrast to neuraminidase produced by the influenza virus, a role for bacterial neuraminidase in pathogenesis has not been clearly established, especially as it pertains to regulating the formation of biofilms.

Neuraminidases (sialidases) are produced by a wide variety of mucosal pathogens, ranging from S. pneumoniae in the airway to Vibrio cholerae in the gut (Vimr et al., (2004) Microbiol. Mol Biol. Rev. 68:132-153). While the central role of viral neuraminidase in pathogenesis of influenza is established (Colman (1994) Protein. Sci. 3:1687-1696) and provides a target for both vaccines and chemotherapy, the contribution of bacterial neuraminidase to the pathogenesis of infection is not as clearly defined. Neuraminidase producing species such as Hemophilus (Vimr et al., (2002) Trends. Microbiol. 10:254-257), Streptococcus pneumoniae (Camara et al., (1994) Infect. Immun. 62:3688-3695; King et al., (2004) Mol. Microbiol. 54:159-171), and P. aeruginosa (Cacalano et al., (1992) J. Clin. Invest. 89:1866-1874) share a common ecological niche, colonizing the heavily sialylated secretions and surfaces of the upper respiratory tract. Although each can bind to asialylated glycolipids exposed by neuraminidase activity (Krivan et al., (1988) Proc. Natl. Acad. Sci. U.S.A. 85:6157-6161), they differ substantially in their ability to either metabolize (Godoy et al., (1993) Infect. Immun. 61:4415-4426) or incorporate sialic acid into surface structures (Bouchet et al., (2003) Proc. Natl. Acad. Sci. U.S.A. 100:8898-8903). Thus, it is likely that bacterial neuraminidases interact with both microbial as well as eukaryotic glycoconjugates (Vimr et al., (2004) Microbiol. Mol. Biol. Rev. 68:132-153).

Viral neuraminidase inhibitors have been very useful in the prevention and treatment of influenza, targeting similar high-risk patient populations, such as those patients afflicted with CF or chronic obstructive pulmonary disease (COPD). The PΔ2794 neuraminidase of P. aeruginosa shares many conserved elements and folds in the manner predicted for other microbial neuraminidases (Roggentin et al., (1989) Glycoconj. J. 6:349-353; Rothe et al., (1991) Mol. Gen. Genet. 226:190-197). P. aeruginosa (a Gram-negative bacterium) is a major opportunistic pathogen, an important cause of nosocomial pneumonia as well as the chief cause of lung infection in cystic fibrosis (CF), and is the most common lethal genetic disease of Caucasians. Over two decades ago, neuraminidase production in isolates of P. aeruginosa from CF patients was described and suggested to contribute to pulmonary infection (Leprat et al., (1980) Ann. Microbiol. (Paris) 131B:209-222).

Many pulmonary pathogens, including P. aeruginosa, bind to the GalNAcβ1-4Gal moiety exposed on asialylated glycolipids (Krivan et al., (1988) Proc. Natl. Acad. Sci. U.S.A. 85:6157-6161). Therefore, the ability to de-sialylate mucosal surfaces could contribute to bacterial colonization of the airways. The P. aeruginosa neuraminidase was shown to be osmo-regulated, and accordingly, to be consistent with expression in the milieu of the CF lung (Cacalano et al., (1992) J. Clin. Invest. 89:1866-1874). This neuraminidase is capable of exposing the receptor asialoganglioside gangliotetraosylceramide (asialoGM 1) (Galβ1,2GalNAcβ1,4Galβ1,4Glcβ1,1Cer) on the surface of CF airway cells in vitro (Saiman et al., (1993) J. Clin. Invest. 92:1875-1880). However, data to document P. aeruginosa adherence to the airway surface in CF patients has been lacking (Baltimore et al., (1989) Am. Rev. Respir. Dis. 140:1650-1661). The current consensus is that organisms are predominantly entrapped in dehydrated secretions of the lung and by shedding proinflammatory products activate airway inflammation, a model that does not require direct attachment of organisms to the epithelial surface (Boucher (2004) Eur. Respir. J 23:146-158). Nonetheless, analyses of P. aeruginosa gene expression in CF patients document that the PΔ2794 neuraminidase locus is one of the most highly expressed genes in this patient population in vivo (Lanotte et al., (2004) J. Med. Microbiol. 53:73-81). Unlike other respiratory pathogens, P. aeruginosa cannot use sialic acid as a carbon source nor does it contain sialic acid as a component of its LPS (Knirel et al., (1988) Acta. Microbiol. Hung. 35:3-24).

### SUMMARY OF THE INVENTION

One aspect of the present invention provides a method for reducing or inhibiting biofilm formation where a surface is contacted with a neuraminidase inhibitor for a sufficient time so as to modulate neuraminidase activity. The neuraminidase inhibitor modulates the activity or the expression of a neuraminidase, thereby resulting in inhibiting or reducing the formation of the biofilm. In one embodiment, the surface comprises a biofilm. A biofilm can be produced by a bacterium, a virus, a protozoan, a fungus, or by any combination of the organisms mentioned. In one embodiment, the biofilm is a bacterial biofilm. In some embodiments, the neuraminidase is a bacterial neuraminidase or a viral neuraminidase. In other embodiments the neuraminidase inhibitor targets bacterial neuraminidases. In some embodiments of the invention, the expression or the activity of the neuraminidase in the biofilm is reduced after the neuraminidase inhibitor is applied to a surface. In one embodiment, the neuraminidase inhibitor is a viral neuraminidase inhibitor. In specific embodiments, the viral neuraminidase inhibitor comprises oseltamivir, peramivir, zanamivir, or a variant thereof. In other embodiments, the neuraminidase inhibitor comprises one or more compounds having a structure depicted in Table 4. In particular embodiments, the neuraminidase inhibitor comprises Formula I:

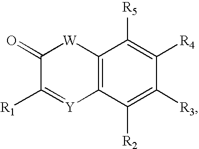

wherein:
W is —O—, or —NH—;
Y is N, or CR6;
R1 is —H, —OH, —R7, or —C1-C6 alkyl;
R2 is —H, or —OH;
R3 is —H, -halogen, or —C(O)—NH—CR8R8R9;
R4 is —H, -methyl, —C(O)—NH-naphthyl, or —OR7;
R5 is —H, —OH, or —CH₂—R10;
R6 is -methyl, -phenyl, or —CH2-R11, or R1 and R6 can combine to form a carbocycle;
R7 is —H, or —CR8R8-C(O)—R10, wherein —CR8R8- can be achiral, an R or S enantiomer or a mixture of both enantiomers;
each R8 is independently —H, or -methyl;
R9 is —H, -phenyl, -2-(imidazol-1-yl)ethyl, or -2,3-dihydrobenzo[b][1,4]dioxin-6-yl;
R10 is —OH, or an amino acid linked through the a-nitrogen of the amino acid;
R11 is -4-phenyl-piperazin-1-yl;
wherein the amino acid can be a natural or unnatural amino acid including alanine, arginine, aspartic acid, cystine, glutamic acid, glycine, histidine, isoleucine, leucine, lysine, methionine, phenylalanine, proline, serine, threonine, tryptophan, tyrosine, valine, phenylglycine, norleucine, homoproline, or norvaline, and the amino acid (except glycine) can be the D- or L-isomer or the compound of the above Formula can be a mixture of both amino acid isomers; and wherein each phenyl or naphthyl group is unsubstituted or substituted with one or more of C1-6 alkoxy including methoxy, a halogen, or any combination thereof, or a pharmaceutically acceptable salt, hydrate, cation, or anion thereof.

In one embodiment, the C1-C6 alkyl is methyl, ethyl, propyl, butyl, pentyl, or hexyl. In another embodiment, R3, R5, R6, R7, and R8 are not all hydrogen.

In one embodiment R3 is not methyl. In another embodiment, R6 is not methyl. In other embodiments, when W is O and Y is CH, R3 and R6 are not methyl. In yet further embodiments, when W is O and Y is CH, R3, R4, R5, R6, R7, and R8 are not all hydrogen. In another embodiment, R7 is hydrogen. In some embodiments R3 is methyl or hexyl. In further embodiments, R3 and R4 can combine to form a cyclohexene ring.

In other embodiments, the neuraminidase inhibitor comprises Formula II:

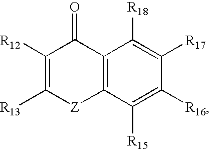

wherein:
Z is —O—, —NEt-, or —CR14-;
   R12 is -phenyl, —CO2H, -3,4-dihydro-2H-benzo[b][1,4]dioxepine-7-yl, or -(1-phenyl-but-2-en-1-one)-4-yl;
R13 is —H or -methyl;
R14 is —CO2H;
R15 is —H or —CH₂—R19
R16 is —H, —OH, —O—CH2CO2H, or —NH-(2-phenyl-thiazolidin-4-one)-3-yl;
R17 is —H, -methyl, —C1-6-alkyl, or -halogen;
R18 is —H or —OH;
R19 is —OH, or an amino acid linked through the a-nitrogen of the amino acid;
wherein the amino acid can be a natural or unnatural amino acid including alanine, arginine, aspartic acid, cystine, glutamic acid, glycine, histidine, isoleucine, leucine, lysine, methionine, phenylalanine, proline, serine, threonine, tryptophan, tyrosine, valine, phenylglycine, norleucine, homoproline, or norvaline, and the amino acid (except glycine) can be the D- or L-isomer or the compound of the above Formula can be a mixture of both amino acid isomers; and wherein each phenyl group is unsubstituted or substituted with one or more of C1-6-alkoxy including methoxy, a halogen, or any combination thereof; or a pharmaceutically acceptable salt, hydrate, cation, or anion thereof.

In one embodiment, the halogen is a fluoride or a chloride. In one embodiment, the —C1-6-alkyl group is methyl.

In further embodiments, the neuraminidase inhibitor comprises Formula III:

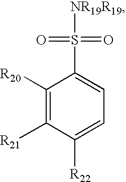

wherein: each R19 is independently —H, -benzyl, -phenyl, -naphthyl, —O-phenyl, or R23;
R20 is —H or —OH;
R21 is —H or —CO2H, or R20 and R21 combine to form a saturated or aromatic carbocyclic ring;
R22 is —H, —CO2H, —C1-6-alkyl, or -halogen;
R23 is:

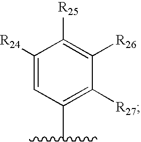

R24 is —H or —S—(CH2)n-furanyl;
R25 is —H, —OH, N-piperidinyl, or pyridinylmethyl;
R26 is —H;
R27 is —H, or R26 and R27 combine to form a saturated or aromatic carbocyclic ring; and
n is 1-6, wherein each phenyl or naphthyl is unsubstituted or substituted with one or more of C1-6-alkyl including methyl, C1-6-alkoxy including methoxy, or halogen, or any combination thereof, or a pharmaceutically acceptable salt, hydrate, cation, or anion thereof.

In some embodiments, the neuraminidase inhibitor comprises Formula IV:

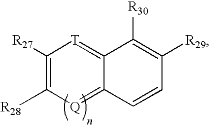

wherein: T is CR31 or N; Q is CR31 or N; n is 0, 1 R27 is —H, phenyl, or benzo-3,4-dioxolane;
R28 is -phenyl, 4-carboxymethyl-piperazin-1-yl, benzo-3,4-dioxolane, 4-([1-carboxyethoxy]-3-methoxy)-phenyl, or 2-(5-(carboxymethyl)-4-oxothiazolidin-2-ylidene)hydrazono-ethyl;
R29 is —H, —OH, or halogen;
R30 is —H, —OH, -halogen, —CO2H, or R31 and R32 combine to form an unsubstituted or substituted aromatic or saturated carbocyclic ring; and
R31 is —H, —CO2H, or (4-(2-(carboxymethoxy)benzylideneamino)-5-mercapto-4H-1,2,4-triazol-3-yl)methyl, wherein each saturated or aromatic carbocyclic ring, including phenyl, are unsubstituted or substituted with one or more of —OH, a -halogen, a C1-6-alkyl group, or a C1-6-alkoxy group; or a pharmaceutically acceptable salt, hydrate, cation, or anion thereof.

In yet other embodiments, the neuraminidase inhibitor comprises Formula V:

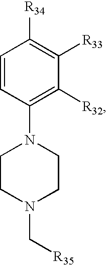

wherein: R32 is —H or -halogen; R33 is —H or -halogen;
R34 is —H, phenyl, or cyclohexyl, or R33 and R34 combine to form an aromatic carbocyclic ring;
R35 is —CO2H, -phenyl, 2-hydroxy-5-nitrophenyl, or 5-(1-carboxypentyloxy)-4-oxo-4H-pyran-2-yl; wherein each phenyl or cyclohexyl group is unsubstituted or substituted with one or more of —OH, a C1-6-alkyl including methyl, a C1-6-alkoxy including methoxy, a halogen, a nitro group, or any combination thereof; or a pharmaceutically acceptable salt, hydrate, cation, or anion thereof.

In one embodiment, R32 is fluoride. In another embodiment, R33 is chloride.

Any biofilm-forming organism can comprise the biofilm mass. In certain embodiments, those organisms are viruses, bacteria, protozoa, and fungi. In various embodiments, the biofilm comprises a Gram-negative bacterium. In some embodiments, the bacterium is Pseudomonas and in particular embodiments these Gram-negative bacterium are Pseudomonas, (e.g., Pseudomonas aeruginosa); Haemophilus (e.g., Haemophilus influenzae); or Vibrio (e.g., Vibrio cholerae). A biofilm can be found on various surfaces and such a surface can be contacted with a neuraminidase inhibitor. In one embodiment, the surface comprises a cellular surface of a subject, an in vitro surface, or an oral surface of a subject. In another embodiment, the surface comprises a cellular surface of a subject, an in vitro surface, or an oral surface of a subject. In particularly useful embodiments, the surface comprises a prosthetic graft, a catheter, a wound dressing, a wound site, a medical device, a contact lens, an implanted device, an oral device, a pipe, or industrial equipment. In further embodiments of the invention, the contacting comprises administering the neuraminidase inhibitor to a subject via subcutaneous, intra-muscular, intra-peritoneal, or intravenous injection; infusion; oral, nasal, or topical delivery; or a combination thereof. In some embodiments, the subject is a human, mouse, rat, bird, dog, cat, cow, horse, or pig. In another embodiment, the neuraminidase inhibitor is applied to the surface of a prosthetic graft to be introduced into a subject. In other embodiments, the neuraminidase inhibitor is applied to the surface of a catheter to be implanted into a subject. In yet further embodiments, the neuraminidase inhibitor is applied to the surface of a wound dressing to be applied on or in a subject. In other embodiments, the neuraminidase inhibitor is applied to the surface of a wound site on a subject. In additional embodiments, the neuraminidase inhibitor is applied to the surface of a medical device to be implanted or inserted into a subject. The subject in many of these instances can harbor the biofilm or has the propensity to form a biofilm. The neuraminidase inhibitor also can be administered to the subject prior to, or during, or after the implantation or insertion of a prosthetic graft, medical device, or a catheter, the application of the wound dressing or to the wound site.

The neuraminidase inhibitor according to the method of the invention can be applied to a surface where a biofilm has formed. In one embodiment, the surface comprises a contact lens, an implanted device, an oral device, a pipe, or industrial equipment. In particular embodiments, industrial equipment is found in a GMP facility. In some embodiments, the industrial equipment comprises a plumbing system. In other embodiments, the surface where a biofilm has formed comprises an oral surface of a subject. In particular embodiments, the biofilm is associated with dental caries while in other embodiments it is associated with periodontal disease. In some embodiments, the neuraminidase inhibitor is in a formulation of a paste, a liquid, a powder, a gel, or a tablet. According to an embodiment of the invention, the neuraminidase inhibitor can be in a paste formulation that can further comprise an abrasive, such as toothpaste. In other embodiments, the neuraminidase inhibitor can be a liquid formulation, such as a mouthwash.

A second therapeutic composition, different than the neuraminidase inhibitor, can also be administered to a subject. In some embodiments of the invention, administration occurs sequentially while in others administration occurs simultaneously. In various embodiments, the therapeutic composition comprises an antibiotic. In yet additional embodiments, the antibiotic comprises a cephalosporin, a macrolide, a penicillin, a quinolone, a sulfonamide, and a tetracycline, or any combination of the listed antibiotics.

Another aspect of the current invention provides for methods of treating a biofilm production-related disorder in a subject in need thereof. The method comprises administering to the subject an effective amount of a neuraminidase inhibitor that reduces biofilm formation in the subject. A reduction or inhibition in the growth of biofilm production-related bacteria in the subject can then be determined. A reduction in bacterial growth is indicative of the reduction in or inhibition of biofilm production in the subject. Thus, the method is useful for treating the biofilm production-related disorder. In one embodiment, the subject being treated is a mammal, whereas in particular embodiments the subject is a human. In some embodiments, the subject can also be a mouse, rat, bird, dog, cat, cow, horse, or pig. A biofilm production-related disorder of the invention can be a disorder or disease that is characterized by a disease-related growth of bacteria, which can result in the establishment of a biofilm. In other embodiments, the disorder affects an epithelial surface, a mucosal surface, or a combination of those surfaces. In particular embodiments of the invention, the surface is a lung surface. In some embodiments, the biofilm production-related disorder is caused by a bacterium, such as a Gram-negative bacterium. In other embodiments, the bacterium comprises Pseudomonas (such as Pseudomonas aeruginosa); Haemophilus (such as Haemophilus influenzae); or Vibrio (such as Vibrio cholerae). In particular embodiments, the bacterium is Pseudomonas aeruginosa. In yet further embodiments, the disorder is cystic fibrosis (CF), otitis media, or chronic obstructive pulmonary disease (COPD). According to the invention, in additional embodiments, the disorder is a medical device-related bacterial infection. The infection arises from the device being implanted or inserted into the subject.

The reduction in bacterial growth can be indicative of the reduction in or inhibition of biofilm production in a subject. In some embodiments, the growth of biofilm production-related bacteria can be determined by measuring the biofilm production-related bacteria in a biological sample. In other embodiments, the presence or growth of biofilm production-related bacteria is measured by detecting the presence of antigens of biofilm production-related bacteria in a biological sample. The biological sample can be blood, serum, sputum, lacrimal secretions, semen, urine, vaginal secretions, or a tissue sample. For example, an antibody to P. aeruginosa components can be used as a test for colonization/infection in a subject afflicted with a biofilm production-related condition or disorder, wherein the presence of Pseudomonas antigens is detected in a biological sample, such as blood. These antibodies can be generated according to methods well established in the art or can be obtained commercially (for example, from Abcam, Cambridge, Mass.; Cell Sciences Canton, Mass.; Novus Biologicals, Littleton, Colo.; or GeneTex, San Antonio, Tex.). The reduction in the growth of biofilm production-related bacteria can also be measured by chest x-rays, or by a pulmonary function test (PFT), such as spirometry or forced expiratory volume (FEV₁) as described below.

Yet, another aspect of the invention provides a method for preventing biofilm formation in the airway of an asymptomatic subject afflicted with cystic fibrosis and who is free of bacterial infection in his/her airway. The method comprises administering to the subject an effective amount of a neuraminidase inhibitor that prevents biofilm disorder-related growth of bacteria in the airways of the subject. The absence of the bacterial growth in the airways of the subject can be determined and could be indicative of the absence of biofilm formation in the airways of the subject. In one embodiment, the subject is a human of about 5 years of age or less. In further embodiments, the bacterium associated with the biofilm-producing disorder, cystic fibrosis, is a Gram-negative bacterium, for example, Pseudomonas aeruginosa. The neuraminidase inhibitor can be administered to the subject by subcutaneous, intra-muscular, intra-peritoneal, or intravenous injection; infusion; by oral, nasal, or topical delivery; or by a combination of the administration modes.

Additional aspects of the current invention provide methods for inhibiting biofilm formation on an industrial surface. The method comprises applying a neuraminidase inhibitor to the biofilm on the industrial surface. The neuraminidase inhibitor-modulated activity or expression of a neuraminidase on the surface can subsequently be determined. The reduction in the neuraminidase inhibitor-modulated activity or expression indicates that biofilm formation has been inhibited. In one embodiment, the neuraminidase is a bacterial neuraminidase. Any biofilm-forming organism, such as viruses, bacteria, protozoa, and fungi, can comprise the biofilm. In various embodiments of the invention, the biofilm comprises a viruses, protozoa, fungi, or bacteria, such as a Gram-negative bacterium. In some embodiments, the bacterium is Pseudomonas (such as Pseudomonas aeruginosa); Haemophilus (such as Haemophilus influenzae); or Vibrio (such as Vibrio cholerae). In particular embodiments, the bacterium is Pseudomonas aeruginosa. According to the invention, a neuraminidase inhibitor that is applied to a surface likely to develop a biofilm modulates the activity or expression of a targeted neuraminidase, such as a bacterial neuraminidase. In particular embodiments, the expression of the neuraminidase is reduced, while in other embodiments, the activity of the neuraminidase is reduced. In various embodiments, the neuraminidase inhibitor is applied as a formulation comprising a paste, liquid, powder, gel, or tablet. In certain embodiments, the industrial surface to which the neuraminidase inhibitor is applied is part of a plumbing system.

A useful neuraminidase inhibitor according to the invention can be any compound, small molecule, peptide, protein, aptamer, ribozyme, RNAi, or antisense oligonucleotide, and the like. In one embodiment, the neuraminidase inhibitor is a viral neuraminidase inhibitor. In particular embodiments, the viral neuraminidase inhibitor comprises oseltamivir, peramivir, zanamivir, or a variant thereof.

Other aspects of the invention provide screening methods for identifying a compound that modulates neuraminidase activity. The method comprises providing an electronic library of test compounds stored on a computer, then providing atomic coordinates for at least twenty amino acid residues of Pseudomonas neuraminidase listed in Table 2, or coordinates having a root mean square deviation therefrom, with respect to at least 50% of the Cα atoms, not more than about 2 Å, in a computer readable format. The atomic coordinates are then converted into electrical signals readable by a computer processor to generate a three-dimensional model of the neuraminidase. A data processing method is then performed, wherein electronic test compounds from the library are superimposed upon the three-dimensional model of the neuraminidase. Whether a test compound fits into the binding pocket of the three-dimensional model of the neuraminidase is subsequently determined, enabling the identification of which compound would modulate the activity of the neuraminidase.

In another aspect of the invention, the method for identifying a compound that modulates neuraminidase activity comprises providing an electronic library of test compounds stored on a computer, then providing atomic coordinates listed in Table 2 in a computer readable format for at least 10, 15, 20, 25, 30, 35, or 40 amino acid residues located within about 10 Å of the neuraminidase active site, wherein the residues comprise 10 or more of the following residues: Tyr21, His23, Phe24, Glu44, His45, Val46, Gly47, Asp76, Arg78, Asp79, Val80, Thr95, Tyr97, Tyr127, Phe129, Ala130, His131, Tyr146, Tyr153, Pro179, Tyr180, Asn181, Glu182, Arg198, Val199, Gly200, Ser201, Gly202, Ile235, Leu236, Val237, Ala238, Thr258, Arg260, Ala294, Ser295, Gly296, Tyr297, Phe313, or Glu315. The atomic coordinates are then converted into electrical signals readable by a computer processor to generate a three-dimensional model of the neuraminidase active site. A data processing method is then performed, wherein electronic test compounds from the library are superimposed upon the three-dimensional model of the neuraminidase active site. Whether a test compound fits into the binding pocket of the three-dimensional model of the neuraminidase is subsequently determined, enabling the identification of which compound would modulate the activity of the neuraminidase.

The methods described above can further comprise obtaining or synthesizing the compound determined to be a potential modulator of the neuraminidase activity; contacting a bacterium with the compound in vitro; and determining whether the compound modulates neuraminidase activity using a biological assay. In one embodiment, the bacterium is a Gram-negative bacterium. In another embodiment, the bacterium is Pseudomonas (i.e., Pseudomonas aeruginosa), Haemophilus, (i.e Haemophilus influenzae), or Vibrio (such as Vibrio cholerae). In further embodiments, the biological assay comprises a biofilm assay, an adherence assay, or a combination of the two mentioned assays. In one embodiment, the biological assay may entail contacting a surface harboring a biofilm (for example, produced by a pathogenic organism, such as a bacterium) in vitro with a test neuraminidase inhibitor, and then determining whether the test neuraminidase inhibitor inhibits biofilm formation at the surface. Inhibition of biofilm formation is indicative of the ability of the test neuraminidase inhibitor to inhibit the pathogenic infection, such as a bacterial infection. In one embodiment, the pathogen is a Gram-negative bacterium, such as Pseudomonas aeruginosa. Thus, the method may be used for identifying neuraminidase inhibitors that can inhibit a pathogenic infection.

In a further aspect, the invention provides a compound identified by the screening methods above, wherein the compound binds to the neuraminidase active site, and comes within 10 Å of amino acid residues listed in Table 3. In one embodiment, the compound inhibits ore reduces biofilm formation. In another embodiment, the compound is a peptide that binds to a neuraminidase, such as an anti-neuraminidase antibody or a binding fragment thereof. In a further embodiment, the peptide interacts with a protein having the amino acid sequence of SEQ ID NO: 2. In some embodiments, the compound interacts with a protein having the amino acid sequence of SEQ ID NO: 2.

According to the methods of the present invention, a candidate or test neuraminidase inhibitor can be any compound, small molecule, peptide, protein, aptamer, ribozyme, RNAi, or antisense oligonucleotide, and the like. In one embodiment, the test inhibitor is a peptide that binds to a neuraminidase. In particular embodiments, the neuraminidase can be a bacterial neuraminidase. In other embodiments, the test inhibitor is an anti-neuraminidase antibody or a binding fragment thereof. In specific embodiments of the invention, the test inhibitor is a peptide that interacts with a protein comprising the amino acid sequence of SEQ ID NO: 2. In various embodiments, the test inhibitor is a viral neuraminidase inhibitor while in other particular embodiments the viral neuraminidase inhibitor comprises oseltamlvir, peramivir, zanamivir, or a variant thereof. In further embodiments of the invention, the test inhibitor is a peptide that interacts with a protein having the amino acid sequence of SEQ ID NO: 2.

One aspect of the invention provides for a mutant P. aeruginosa strain having a deletion in a gene encoding a neuraminidase protein. In one embodiment, the deletion is in the PΔ2794 gene having a nucleic acid sequence of SEQ ID NO:1.

### BRIEF DESCRIPTION OF THE FIGURES

FIGS. 1A-1C are schematic representations that depict the properties of the PΔ2794 locus. The gene PΔ2794 is depicted 3 times (FIGS. 1A-C) and shaded regions demonstrate distinct functional predictions. Regions are indicated using amino acid positions. Predicted transmembrane (TM) regions are shown first (FIG. 1A). The second gene depiction (FIG. 1B) shows the region most similar to other sialidase genes. Lighter gray shading indicates the region predicted by BLAST analysis and the darker shading corresponds to the sialidase region predicted by the SMART program (FIG. 1B). The third gene depiction (FIG. 1C) shows the region similar to autotransporter genes.

FIG. 2 is a schematic representation of the predicted PAO1 neuraminidase amino acid sequence, where ASP boxes are shaded gray. As bacterial and viral neuraminidases can share common ASP boxes that interact with sialic acid, neuraminidase inhibitors designed to block the influenza enzyme might have sufficient avidity for the active site of the bacterial neuraminidase to inhibit biofilm formation (Roggentin et al., (1989) Glycoconj. J. 6:349-353; Hayden et al., (1999) JAMA 282:1240-1246).

FIG. 3A is a photographic representation of a DNA gel image that represents colony PCR for expression of nanA in the Δ2794 mutant. lane 1, molecular weight marker. lanes 2-5 show the nanA PCR product using primers from within the nanA gene: lane 2, PAO1; lane 3, PAO1Δ2794; lane 4, PAOΔ2794+nana; lane 5, PAO1Δ2794 with vector alone. lanes 6-9 show the nanA PCR product using primers flanking the nanA gene: lane 6, PAO1; lane 7, PAK; lane 8, PAO1Δ2794; lane 9, PAKΔ2794. lane 10, molecular weight marker.

FIG. 3B is a graphic representation of the amount of superficial asialoGM1 quantified by flow cytometry on 16HBE cells following exposure to bacterial supernatant from strains indicated: Unstimulated PAO1; PAO1; PAO1Δ2794.

FIG. 3C is a graphic representation showing the growth of strains in M9 media as determined by OD₆₀₀.

FIG. 4A is a graphic representation of PAO1 and Δ2794 virulence in mouse model of pneumonia where the percentage of the total mice intranasally inoculated with each strain that developed pneumonia, bacteremia or died, is indicated on the y axis.

FIG. 4B is a graphic representation of KC mRNA expression in PAO1 and Δ2794 determined by Real Time PCR and standardized to actin. The short horizontal lines indicate the median values of each group.

FIG. 4C is a graphic representation depicting lung cell suspensions from mice inoculated with P. aeruginosa PAO1 or Δ2794 that were stained for CD45 and Ly-6G (PMN). Individual mouse values are shown and the short horizontal lines indicate the median values of each group.

FIG. 5A is a photographic representation of micrographs depicting PAO1 and Δ2794 virulence in a mouse model of pneumonia, where hematoxylin-eosin stained sections of murine lungs are shown 16 h following inoculation with P. aeruginosa PAO1 (left-hand side) or Δ2794 (right-hand side). Black arrows indicate airways, which are full of PMNs in a PAO1 infected lung, but clear in Δ2794, infected lung.

FIG. 5B is graphic representation of the comparison of PAO1 and Δ2794 virulence when introduced intraperitoneally into the mouse model. The percentage of the total number of mice inoculated with each strain that developed pneumonia, bacteremia or died is indicated on they axis.

FIG. 6A is a graphic representation demonstrating bacterial adherence of PAO1, Δ2794, Δ2794+nanA, and Δ2794+vector, alone, following a 1 hour exposure to 16HBE airway epithelial cells as determined by flow cytometry and quantitated via mean fluorescence intensity.

FIG. 6B is a graphic representation showing the induction of IL-8 production by confluent monolayers of 1HAEo—airway epithelial cells following exposure to a media control, PAO1, or Δ2794 and was quantified by ELISA (Ratner et al., (2001) J. Biol. Chem. 276:19267-19275).

FIG. 7A is a graphic representation depicting binding and phagocytosis of PAO1 and Δ2794 by RAW cells as determined by flow cytometry.

FIG. 7B is a graphic representation of TNFα production by RAW cells treated with media alone (Unstimulated), bacterial culture supernatant, or LPS harvested from cultures of PAO1 and Δ2794 that was quantified by ELISA. Purified LPS (Sigma) was used as a positive control.

FIG. 8A is a graphic representation of biofilm production by PAO1 and Δ2794 bacterial strains transformed with a control vector or cloned nanA detected via crystal violet staining.

FIG. 8B is a graphic representation of biofilm production detected via crystal violet staining corresponding to the following bacterial strains: PAK, Δ2794 PAK, Δ2794 PAK+neuraminidase locus (Δ2794+nanA), and Δ2794 PAK+empty control vector (Δ2794+CV). (*P<0.0001, **P<0.001, ***P<0.01 as compared with PAO1+control vector or PAK).

FIG. 9A is a micrographic representation that depicts a fluorescence microscope image (top row) and a profile (bottom row) of biofilm production by PAO1 in a flow cell.

FIG. 9B is a micrographic representation that depicts a fluorescence microscope image (top row) and a profile (bottom row) of biofilm production by PAO1 Δ2794 in a flow cell.

FIG. 9C is a micrographic representation that depicts a fluorescence microscope image (top row) and a profile (bottom row) of biofilm production by PAO1Δ2794+nanA in a flow cell.

FIG. 9D is a micrographic representation that depicts a fluorescence microscope image (top row) and a profile (bottom row) of biofilm production by PAO1Δ2794+control vector (CV) in a flow cell.

FIG. 10A is a micrographic representation depicting light microscope images of biofilms produced using a rotating disc reactor under high shear forced to determine matrix integrity and attachment ability of a biofilm. White, fluffy material is biofilm produced by PAO1 (left-hand side). The lack of biofilm formation by the Δ2794 mutant (right-hand side) could be caused by defective matrix synthesis.

FIG. 10B is a micrographic representation of bacterial biofilm production by PAO1 (left-hand side) and Δ2794 (right-hand side). Bacteria (˜10⁶ cells) expressing GFP were incubated with confluent monolayers of 16HBE airway epithelial cells for 5 hours and visualized by confocal microscopy to determine clustering and agglutination phenotypes.

FIG. 11A is a graphic representation of the dose dependent inhibition of biofilm production by PAO1 in response to various concentrations of viral neuraminidase inhibitor, oseltamivir, where biofilm production was detected by crystal violet staining method.

FIG. 11B is a graphic representation of the dose dependent inhibition of biofilm production by PAO1 in response to various concentrations of viral neuraminidase inhibitor, peramivir, where biofilm production was detected by crystal violet staining method.

FIG. 11C is a graphic representation of the inhibition of bacterial neuraminidases (N'ase), PAO1 N'ase and Vibrio Cholera (V. cholera) N'ase, by different concentrations of peramivir. The neuraminidase activity was measured using the fluorescent substrate 2′-(4-methylumbelliferyl)-α-D-N-acetylneuraminic acid. (*P<0.001, **P<0.01, ***P<0.05).

FIG. 12 is a graphic representation of the dose dependent inhibition of biofilm production by PAO1 in response to various concentrations of viral neuraminidase inhibitor, zanamivir, where biofilm production was detected by crystal violet staining method.

FIG. 13A depicts representative models of neuraminidase active sites for Influenza virus (left panel) and P. aeruginosa (PAO1, right panel). The inhibitor shown in the PAO1 structure was modeled into the structure to show where the binding site is located.

FIG. 13B depicts ribbon models of neuraminidase active sites for Influenza virus (left panel) and P. aeruginosa (PAO1, right panel). The inhibitor shown in the PAO1 structure was modeled into the structure to show where the binding site is located.

### DETAILED DESCRIPTION OF THE INVENTION

The invention is related to various methods for inhibiting biofilm formation, treating a biofilm production-related disorder, preventing biofilm formation, and screening for neuraminidase inhibitors. The invention also encompasses a mutant bacterial strain with a deletion in a neuraminidase gene.

### Definitions

All scientific and technical terms used in this application have meanings commonly used in the art unless otherwise specified. As used in this application, the following words or phrases have the meanings specified.

As used herein, the term “inhibitor of biofilm formation,” or “biofilm synthesis inhibitor” (such as a neuraminidase inhibitor) encompasses an agent that inhibits (e.g., disrupts) the attachment of microorganisms onto a surface, to the biofilm matrix itself, to other cells comprising the biofilm, or a combination thereof, and/or inhibits the ability of such microorganisms to produce, synthesize and/or accumulate biofilm on a surface.

A “derivative” refers to a molecule that shares substantial structural similarity to its parent molecule. A protein derivative encompasses a protein, which includes a change to its amino acid sequence and/or chemical quality of the amino acid e.g., amino acid analogs, when compared to its parent protein. For example, in the context of a protein molecule (e.g., proteins, polypeptides, and peptides, such as antibodies), “derivative” refers to a protein molecule that comprises an amino acid sequence that has been altered by the introduction of amino acid residue substitutions, deletions, and/or additions. The term “derivative” as used herein also refers to a protein molecule that has been modified, for example, by the covalent attachment of any type of molecule to the protein molecule. A derivative of a protein molecule may be produced by chemical modifications using techniques known to those of skill in the art.

The terms “disorder” and “disease” are used herein interchangeably for a condition in a subject. A disorder is a disturbance or derangement that affects the normal function of the body of a subject. A disease is a pathological condition of an organ, a body part, or a system resulting from various causes, such as infection, genetic defect, or environmental stress that is characterized by an identifiable group of symptoms. A disorder or disease can refer to a biofilm production-related disorder of the invention that is characterized by a disease-related growth of bacteria in that a biofilm is established.

“Effective amount” means the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The terms “prevent,” “preventing,” and “prevention” refer herein to the inhibition of the development or onset of a disorder or the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, to “block” or “inhibit” a molecule, signal, or a receptor means to interfere with the binding of, or activation of the molecule, signal, or a receptor as detected by a test recognized in the art (such as binding assays). Blockage or inhibition may be partial or total, resulting in a reduction, increase, or modulation in the activation of the molecule, signal, or a receptor as detected by a test recognized in the art.

The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target of the present invention is neuraminidase, and hence the term neuraminidase aptamer or nucleic acid ligand or neuraminidase aptamer or nucleic acid ligand is used. Inhibition of the target by the aptamer may occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers may be comprised of multiple ribonucleotide units, deoxyribonucleotide units, or a mixture of both types of nucleotide residues. Aptamers may further comprise one or more modified bases, sugars or phosphate backbone units as described in further detail herein.

“Binding” refers to the interaction or association of a molecule with another entity, such as its target. This interaction may be covalent or noncovalent. The interaction of a molecule and its target site can be regulated by compositions of the invention. For example, administration of a neuraminidase inhibitor or a derivative thereof can block the action of its target, a neuraminidase.

As used herein, a “fragment” or “portion” is any part or segment of a molecule. For example, a fragment of a molecule includes that part that recognizes and binds its natural target. In the case of an antibody, the fragment is a binding portion of the whole antibody; in the case of a neuraminidase inhibitor, the fragment is that smaller portion of the entire inhibitor.

The terms “subject” and “patient” are used interchangeably throughout this disclosure. The terms refer to an animal, or a human. For example, the terms can refer to a mammal including, but not limited to, a non-primate (e.g., a cow, pig, bird, sheep, goat, horse, cat, dog, rat, and mouse) and a primate (e.g., a monkey, such as a cynomolgous monkey, a chimpanzee, and a human). For example, the subject can be a non-human animal such as a bird (e.g., a quail, chicken, or turkey), a farm animal (e.g., a cow, horse, pig, or sheep), a pet (e.g., a cat, dog, or guinea pig), or laboratory animal (e.g., an animal model for a disorder). In particular, the subject according to the invention is a human (e.g., an infant, child, adult, or senior citizen).

A “plumbing system” encompasses the faucets, valves, plumbing fixtures, piping (metal, plastic, and the like), water storage tanks, water recycles, coils, bilges, hoses, tubing, and backflow preventers as well as their respective interior and exterior surfaces.

Aspects of the invention are related to methods of inhibiting biofilm formation. The method entails applying a neuraminidase inhibitor to the biofilm and measuring a reduction in the formation of a biofilm. The neuraminidase inhibitor modulates the activity or the expression of the neuraminidase (for example, a bacterial neuraminidase), thereby inhibiting biofilm formation.

Gram-negative bacteria and other unicellular organisms can produce biofilms. Bacterial biofilms are surface-attached communities of cells that are encased within an extracellular polysaccharide matrix produced by the colonizing cells. Biofilm development occurs via a series of programmed steps, which include an initial attachment to a surface, formation of three-dimensional microcolonies, and the subsequent development of a mature biofilm. Biofilms can be composed of various microorganisms (such as viruses, bacteria, protozoa, and fungi) co-existing within the community and a particular cellular type may predominate. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and may include, but are not limited to carbohydrates (simple and complex), lipids, proteins (including polypeptides), and lipid complexes of sugars and proteins (lipopolysaccharides and lipoproteins).

Bacterial biofilms exist in nature as well as in medical and industrial environments, such as a GMP facility. The biofilm may allow bacteria to exist in a dormant state for a certain amount of time until suitable growth conditions arise thus offering the microorganism a selective advantage to ensure its survival. However, this selection could pose serious threats to human health in that biofilms have been observed to be involved in about 65% of human bacterial infections (Smith (2005) Adv. Drug Deliv. Rev. 57:1539-1550; Hall-Stoodley et al., (2004) Nat. Rev. Microbiol. 2: 95-108). In fact, the majority of infections that occur in animals are biofilm-based. In particular, biofilms are problematic with respect to respiratory conditions and diseases. Cystic Fibrosis (CF), one of the most common fatal genetic disorders in the United States, is most prevalent in Caucasians. It occurs on an average of one in every 3,300 live births, and causes the death of patients inflicted with CF by the age of 30. A mutation in a gene that encodes a chloride transport channel produces partially functional or completely dysfunctional transport channels. Typically, CF patients develop thick mucus secretions, which result from disruption of physiological salt/water balance due to the defective transport channel. The secretions clog bronchial tubes in the lungs and can additionally block exit passages of the pancreas and intestines, which lead to loss of function of these organs.

The mucus secretions are depleted of oxygen due to the metabolic activity of neutrophils, aerobic bacteria, and even epithelial cells. Within this mucus, P. aeruginosa is found to thrive. P. aeruginosa also is an important cause of nosocomial pneumonia. It infects the elderly, cancer chemotherapy patients, and immuno-compromised individuals.

Other medical conditions and treatments resulting in the development of undesirable biofilms include, but are not limited to, medical device-related infections, catheter-related infection (kidney, vascular, peritoneal), chronic otitis media, prostatitis, dental caries, wounds, acne, chronic obstructive pulmonary disease, infectious kidney stones, orthopedic implant infection, cystitis, bronchiectasis, bacterial endocarditis, Legionnaire's disease, osteomyelitis, and biliary stents (see US Appln. Pub. No. 20050158253). Thus, there is a need in the art for improved therapeutic approaches for the inhibition of biofilm formation and/or the reduction or elimination of biofilms.

Harsh treatments (such as chemicals and abrasives) have been used to reduce, prevent, or control biofilm formation. However, biological environments (for example, airways, the urinary tract, wound sites, etc) are particularly sensitive to such harsh treatments. Thus, better methods are needed to control biofilm formation.

In industrial settings, biofilms (comprised of viruses, bacteria, protozoa, fungi, and the like) can adhere to surfaces, such as pipes and filters. Biofilms are problematic in industrial settings because they cause biocorrosion and biofouling in industrial systems, such as heat exchangers, oil pipelines, water systems, filters, and the like (Coetser et al., (2005) Crit. Rev. Micro. 31: 212-32). Thus, biofilms can inhibit fluid flow-through in pipes, clog water and other fluid systems, as well as serve as reservoirs for pathogenic bacteria, protozoa, and fungi. As such, industrial biofilms are an important cause of economic inefficiency in industrial processing systems.

Biofilms (also referred to as “slime residues”) can affect a wide variety of commercial, industrial, and processing operations (such as Good Manufacturing Practices (GMP) facilities). Since biofilms are ubiquitous in water handling systems, P. aeruginosa a gram-negative, rod bacterium (and/or other bacteria, protozoa, fungi and some viruses) is also likely to be associated with these biofilms. In many instances, P. aeruginosa is the major microbial component. Thus, there is a need for compositions and methods for controlling biofilms in commercial settings as well as biological environments.

The biofilm to be inhibited can be harbored by a subject, can be in vitro, or can be on the surface of an implantable/insertable device to be inserted into a subject. For example, the subject according to the invention can be an animal, such as a mammal. The mammal can be a non-primate (for example, a cow, pig, bird, sheep, goat, horse, cat, dog, rat, rabbit, mouse, and the like) or a primate (for example, a monkey, such as a cynomolgous monkey, a chimpanzee, a human). Non-limiting representative subjects according to the invention may be a human infant, a pre-adolescent child, an adolescent, an adult, or a senior/elderly adult.

A neuraminidase is an enzyme protein (for example, bacterial, viral, and the like) that cleaves terminal sialic acid residues from carbohydrate moieties on the surfaces of cells infected with such pathogens (for example, bacteria or viruses). This cleavage can result in the release of progeny pathogens from infected cells. Thus, administration of neuraminidase inhibitors can serve as a treatment that limits the severity and spread of pathogenic infections. The neuraminidase inhibitor can also modulate the expression of a neuraminidase via reducing the expression of the neuraminidase. The modulation of neuraminidase activity and/or expression (for example, its reduction) can be due to decreased transcription and/or translation of the neuraminidase molecule, which results in reduced amounts of neuraminidase synthesized by the cell.

Initial studies of the P. aeruginosa neuraminidase performed with purified enzyme, and in vitro analyses were consistent with a role for the enzyme in modifying airway epithelial cell surfaces to facilitate bacterial attachment (Cacalano et al., (1992) J. Clin. Invest. 89:1866-1874). Moreover, as CF airways were more readily modified than were normal airway cells, the Pseudomonas enzyme seemed likely to be important in that disease (Saiman et al., (1993) J. Clin. Invest. 92:1875-1880). However, it has been determined with tests performed under more physiological conditions in vivo using isogenic mutants that the P. aeruginosa neuraminidase has a different function. The neuraminidase is important for biofilm production, the cell-cell interactions that were critical even in the initial colonization process. Although the components of the PAO1 biofilm have not been fully defined, those of another P. aeruginosa, PA14, do not include sialic acid, indicating that the neuraminidase activity may be directed against a different sugar linkage on the bacterial surface (Wozniak et al., (2003) Proc. Natl. Acad. Sci. U.S.A. 100:7907-7912). Pseudaminic acid, or a structure containing pseudaminic acid, a nine carbon acidic sugar with structural similarity to the neuraminic acids is a potential substrate and modifies several surface structures including LPS, pili, and flagella in P. aeruginosa (Rocchetta et al., (1999) Microbiol. Mol. Biol. Rev. 63:523-553; Corner et al., (2002) Infect. Immun. 70:2837-2845; Schirm et al., (2004) J. Bacteriol. 186:2523-2531). Recent studies indicate that there are significant homologies between the genes involved in sialic acid O-acetylation in many bacterial species, including the P. aeruginosa strain 012, which produces pseudaminic acid but not sialic acid (Lewis et al., (2006) J. Biol. Chem. 281:11186-11192). Just as autolysins are necessary for cell wall biosynthesis, enzymes capable of cleaving carbohydrate linkages are necessary for the growth and modification of extracellular polysaccharides during biofilm biosynthesis (Vuong et al., (2004) J. Biol. Chem. 279:54881-54886). The invention provides for methods for inhibiting or reducing biofilm formation using neuraminidase inhibitors.

A neuraminidase inhibitor according to the invention can be used to inhibit the formation of a biofilm by any biofilm-forming organism, such as viruses, bacteria, protozoa, and fungi. Biofilms are comprised of various microorganisms, such as viruses, bacteria, protozoa, and fungi, (e.g., Borrelia sp., Neisseria sp., Pseudomonas sp., Haemophilus sp., Vibrio sp., Bacillus sp., Klebsiella sp., Burkholderia sp., Salmonella sp., Legionella sp., P. aeruginosa, H. influenzae, V. cholerae, Yersinia pestis, Escherichia coli, Proteus mirablis, and Francisella tularensis) and can be found in a live subject, in vitro, or on a surface, such as on or in the pipes of a plumbing system or industrial equipment.

The neuraminidase inhibitor to be used to inhibit biofilm formation in the method of the invention can be any compound, small molecule, peptide, protein, aptamer, ribozyme, RNAi, or antisense oligonucleotide and the like.

For example, a neuraminidase inhibitor according to the invention can be a protein, such as an antibody (monoclonal, polyclonal, humanized, and the like), or a binding fragment thereof, directed against a neuraminidase protein, such as a viral, protozoan, fungal, or bacterial neuraminidase (such as P. aeruginosa, H. influenzae, or V. cholerae). An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

Additionally, a neuraminidase inhibitor can be a non-antibody peptide or polypeptide that binds to a bacterial neuraminidase. A peptide or polypeptide can be a portion of a protein molecule of interest other than the full-length form, and includes peptides that are smaller constituents that exist within the full-length amino acid sequence of a protein molecule of interest. These peptides can be obtained commercially or synthesized via liquid phase or solid phase synthesis methods (Atherton et al., (1989) Solid Phase Peptide Synthesis: a Practical Approach. IRL Press, Oxford, England). For example, the neuraminidase inhibitor can be a peptide that interacts with a Pseudomonas neuraminidase, such as the protein encoded by the PΔ2794 gene (e.g., a protein comprising the amino acid sequence of SEQ ID NO:2). The peptide or protein-related neuraminidase inhibitors can be isolated from a natural source, genetically engineered or chemically prepared. These methods are well known in the art.

A neuraminidase inhibitor can also be a small molecule that binds to a neuraminidase and disrupts its function. Small molecules are a diverse group of synthetic and natural substances generally having low molecular weights. They are isolated from natural sources (for example, plants, fungi, microbes and the like), are obtained commercially and/or available as libraries or collections, or synthesized. Candidate neuramindase inhibitor small molecules can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6).

According to this invention, the neuraminidase inhibitor can also be an FDA approved viral neuraminidase inhibitor, such as the viral neuraminidase inhibitor oseltamivir (Tamiflu), zanamivir (Relenza; Glaxo Smith Kline, Research Triangle Park, N.C.), peramivir (BioCryst, Birmingham, Ala.), or a variant thereof. For example, the viral neuraminidase inhibitor, oseltamivir is an ethyl ester prodrug that can be purchased from Roche Laboratories (Nutley, N.J.). Amino acid sequences of FDA approved viral neuraminidase inhibitors may also be derivatized, for example, bearing modifications other than insertion, deletion, or substitution of amino acid residues, thus resulting in a variation of the original product (a variant). These modifications can be covalent in nature, and include for example, chemical bonding with lipids, other organic moieties, inorganic moieties, and polymers. For reviews on viral neuraminidase inhibitors, please see Klumpp et al., (2006) Curr. Top. Med. Chem. 6(5):423-34; Zhang et al., (2006) Mini Rev. Med. Chem. 6(4):429-48; Jefferson et al., (2006) Lancet 367(9507):303-13; Alymova et al., (2005) Curr Drug Targets Infect. Disord. 5(4):401-9; Moscona (2005) N. Engl. J. Med. 353(13):1363-73; De Clercq (2004) J. Clin. Virol. 30(2):115-33; Stiver (2003) CMAJ 168(1):49-56; Oxford et al., (2003) Expert Rev. Anti. Infect. Ther. 1(2):337-42; Cheer et al., (2002) Am. J. Respir. Med. 1(2):147-52; Sidewell et al., (2002) Expert Opin. Investig. Drugs. 11(6):859-69; Doucette et al., (2001) Expert Opin. Pharmacother. 2(10):1671-83; Young et al., (2001) Philos. Trans. R. Soc. Lond. B. Biol. Sci. 356(1416):1905-13; Lew et al., (2000) Curr. Med. Chem. 7(6):663-72); Taylor et al., (1996) Curr. Opin. Struct. Biol. 1996 6(6):830-7; and U.S. Patent Appln. Nos. 20060057658 and 20040062801.

Inhibition of RNA can effectively inhibit expression of a gene from which the RNA is transcribed. Inhibitors are selected from the group comprising: siRNA, interfering RNA or RNAi; dsRNA; RNA Polymerase III transcribed DNAs; ribozymes; and antisense nucleic acid, which may be RNA, DNA, or artificial nucleic acid. Also within the scope of the present invention are oligonucleotide sequences that include antisense oligonucleotides and ribozymes that function to bind to, degrade and/or inhibit the translation of an mRNA encoding a neuraminidase, such as a bacterial neuraminidase.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a neuraminidase polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59).

siRNA comprises a double stranded structure typically containing 15 to 50 base pairs and preferably 21 to 25 base pairs and having a nucleotide sequence identical or nearly identical to an expressed target gene or RNA within the cell. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The inhibitor may be polymerized in vitro, recombinant RNA, contain chimeric sequences, or derivatives of these groups. The inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

Ribozymes are enzymatic RNA molecules capable of catalyzing the specific cleavage of RNA. The mechanism of ribozyme action involves sequence specific hybridization of the ribozyme molecule to complementary target RNA encoding the neuraminidase, followed by endonucleolytic cleavage. Engineered hammerhead motif ribozyme molecules that specifically and efficiently catalyze endonucleolytic cleavage of mRNA sequences encoding a neuraminidase inhibitor, such as a bacterial neuraminidase inhibitor, are also within the scope of the present invention. Scanning the target molecule for ribozyme cleavage sites that include the following sequences, GUA, GUU, and GUC initially identifies specific ribozyme cleavage sites within any potential RNA target. Once identified, short RNA sequences of between about 15 and 20 ribonucleotides corresponding to the region of the target gene containing the cleavage site can be evaluated for predicted structural features such as secondary structure that may render the oligonucleotide sequence unsuitable. The suitability of candidate targets can also be evaluated by testing their accessibility to hybridization with complementary oligonucleotides using, e.g., ribonuclease protection assays.

Both the antisense oligonucleotides and ribozymes of the present invention can be prepared by known methods. These include techniques for chemical synthesis such as, e.g., by solid phase phosphoamite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters.

Various modifications to the oligonucleotides of the present invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Aptamers nucleic acid sequences are readily made that bind to a wide variety of target molecules. The aptamer nucleic acid sequences of the invention can be comprised entirely of RNA or partially of RNA, or entirely or partially of DNA and/or other nucleotide analogs. Aptamers are typically developed to bind particular ligands by employing known in vivo or in vitro (most typically, in vitro) selection techniques known as SELEX (Systematic Evolution of Ligands by Exponential Enrichment). Methods of making aptamers are described in, for example, Ellington and Szostak (1990) Nature 346:818, Tuerk and Gold (1990) Science 249:505, U.S. Pat. No. 5,582,981; PCT Publication No. WO 00/20040; U.S. Pat. No. 5,270,163; Lorsch and Szostak (1994) Biochem. 33:973; Mannironi et al., (1997) Biochem. 36:9726; Blind (1999) Proc. Nat'l. Acad. Sci. USA 96:3606-3610; Huizenga and Szostak (1995) Biochem. 34:656-665; PCT Publication Nos. WO 99/54506, WO 99/27133, and WO 97/42317; and U.S. Pat. No. 5,756,291.

Generally, in their most basic form, in vitro selection techniques for identifying RNA aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques. The DNA pool is then transcribed in vitro. The RNA transcripts are then subjected to affinity chromatography. The transcripts are most typically passed through a column or contacted with magnetic beads or the like on which the target ligand has been immobilized. RNA molecules in the pool, which bind to the ligand, are retained on the column or bead, while nonbinding sequences are washed away. The RNA molecules, which bind the ligand, are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules that are capable of acting as aptamers for the target ligand.

One can generally choose a suitable ligand without reference to whether an aptamer is yet available. In most cases, an aptamer can be obtained which binds the small, organic molecule of choice by someone of ordinary skill in the art. The unique nature of the in vitro selection process allows for the isolation of a suitable aptamer that binds a desired ligand despite a complete dearth of prior knowledge as to what type of structure might bind the desired ligand.

The association constant for the aptamer and associated ligand is, for example, such that the ligand functions to bind to the aptamer and have the desired effect at the concentration of ligand obtained upon administration of the ligand. For in vivo use, for example, the association constant should be such that binding occurs below the concentration of ligand that can be achieved in the serum or other tissue (such as ocular vitreous fluid). For example, the required ligand concentration for in vivo use is also below that which could have undesired effects on the organism.

The aptamer nucleic acid sequences, in addition to including RNA, DNA and mixed compositions, may be modified. For example, certain modified nucleotides can confer improved characteristic on high-affinity nucleic acid ligands containing them, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, entitled “High Affinity Nucleic Acid Ligands Containing Modified Nucleotides,” that describes oligonucleotides containing nucleotide derivatives chemically modified at the 5- and 2′-positions of pyrimidines. U.S. Pat. No. 5,637,459, supra, describes highly specific nucleic acid ligands containing one or more nucleotides modified with 2′-amino (2′—NH.sub.2), 2′-fluoro (2′-F), and/or 2′-O-methyl (2′-OMe). U.S. application Ser. No. 08/264,029, filed Jun. 22, 1994, entitled “Novel Method of Preparation of Known and Novel 2′ Modified Nucleosides by Intramolecular Nucleophilic Displacement,” describes oligonucleotides containing various 2′-modified pyrimidines.

The aptamer nucleic acid sequences of the invention further may be combined with other selected oligonucleotides and/or non-oligonucleotide functional units as described in U.S. Pat. No. 5,637,459, entitled “Systematic Evolution of Ligands by Exponential Enrichment: Chimeric SELEX,” and U.S. Pat. No. 5,683,867, entitled “Systematic Evolution of Ligands by Exponential Enrichment: Blended SELEX,” respectively.

Diversity libraries, such as random or combinatorial peptide or non-peptide libraries can be screened for small molecules and compounds that specifically bind to a bacterial, viral, yeast, or protozoan neuraminidase. Many libraries are known in the art that can be used such as, e.g., chemically synthesized libraries, recombinant (e.g., phage display) libraries, and in vitro translation-based libraries.

Any screening technique known in the art can be used to screen for agonist or antagonist molecules (such as neuraminidase inhibitors) directed at a target of interest (e.g. a neuraminidase, such as a bacterial neuraminidase). The present invention contemplates screens for small molecule ligands or ligand analogs and mimics, as well as screens for natural ligands that bind to and antagonize neuraminidase inhibitors, such as via examining the degree of biofilm inhibition utilizing previously described biofilm assays. For example, natural products libraries can be screened using assays of the invention for molecules that agonize or antagonize the activity of a molecule of interest, such as a neuraminidase.

Knowledge of the primary sequence of a molecule of interest, such as a neuraminidase inhibitor, and the similarity of that sequence with proteins of known function (e.g., a viral neuraminidase inhibitor such as Tamiflu), can provide an initial clue as the inhibitors or antagonists of the protein. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as test neuraminidase inhibitors, are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups. Screening compound libraries listed above [also see EXAMPLE 14 and U.S. Patent Application Publication No. 2005/0009163, which is hereby incorporated by reference], in combination with biofilm assays described below (such as the one depicted in EXAMPLE 4) can be used to identify neuraminidase inhibitors capable of disrupting the formation of a biofilm (Lew et al., (2000) Curr. Med. Chem. 7(6):663-72; Werner et al., (2006) Brief Funct. Genomic Proteomic 5(1):32-6).

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Screening methods of the invention allowed for the identification of potential neuraminidase inhibitors. In some embodiments of the invention, the neuraminidase inhibitor comprises one or more compounds having a structure depicted in Table 4. In particular embodiments, the neuraminidase inhibitor comprises Formula I:

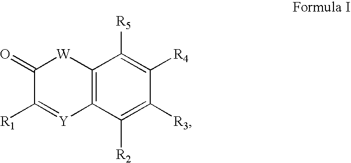

wherein:
W is —O—, or —NH—;
Y is N, or CR6;
R1 is —H, —OH, —R7, or —C1-C6 alkyl;
R2 is —H, or —OH;
R3 is —H, -halogen, or —C(O)—NH—CR8R8R9;
R4 is —H, -methyl, —C(O)—NH-naphthyl, or —OR7;
R5 is —H, —OH, or —CH2-R10;
R6 is -methyl, -phenyl, or —CH2-R11, or R1 and R6 can combine to form a carbocycle;
R7 is —H, or —CR8R8-C(O)—R10, wherein —CR8R8- can be achiral, an R or S enantiomer or a mixture of both enantiomers;
each R8 is independently —H, or -methyl;
R9 is —H, -phenyl, -2-(imidazol-1-yl)ethyl, or -2,3-dihydrobenzo[b][1,4]dioxin-6-yl;
R10 is —OH, or an amino acid linked through the a-nitrogen of the amino acid;
R11 is -4-phenyl-piperazin-1-yl;
wherein the amino acid can be a natural or unnatural amino acid including alanine, arginine, aspartic acid, cystine, glutamic acid, glycine, histidine, isoleucine, leucine, lysine, methionine, phenylalanine, proline, serine, threonine, tryptophan, tyrosine, valine, phenylglycine, norleucine, homoproline, or norvaline, and the amino acid (except glycine) can be the D- or L-isomer or the compound of the above Formula can be a mixture of both amino acid isomers; and wherein each phenyl or naphthyl group is unsubstituted or substituted with one or more of C1-6 alkoxy including methoxy, a halogen, or any combination thereof, or a pharmaceutically acceptable salt, hydrate, cation, or anion thereof.

In one embodiment, the C1-C6 alkyl is methyl, ethyl, propyl, butyl, pentyl, or hexyl. In another embodiment, R3, R5, R6, R7, and R8 are not all hydrogen.

In one embodiment R3 is not methyl. In another embodiment, R6 is not methyl. In other embodiments, when W is O and Y is CH, R3 and R6 are not methyl. In yet further embodiments, when W is O and Y is CH, R3, R4, R5, R6, R7, and R8 are not all hydrogen. In another embodiment, R7 is hydrogen. In some embodiments R3 is methyl or hexyl. In further embodiments, R3 and R4 can combine to form a cyclohexene ring.

In other embodiments, the neuraminidase inhibitor comprises Formula II:

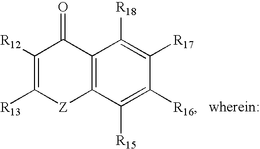

wherein:
Z is —O—, —NEt-, or —CR14-;
R12 is -phenyl, —CO2H, -3,4-dihydro-2H-benzo[b][1,4]dioxepine-7-yl, or -(1-phenyl-but-2-en-1-one)-4-yl;
R13 is —H or -methyl;
R14 is —CO2H;
R15 is —H or —CH₂—R19
R16 is —H, —OH, —O—CH2CO2H, or —NH-(2-phenyl-thiazolidin-4-one)-3-yl;
R17 is —H, -methyl, —C1-6-alkyl, or -halogen;
R18 is —H or —OH;
R19 is —OH, or an amino acid linked through the a-nitrogen of the amino acid;
wherein the amino acid can be a natural or unnatural amino acid including alanine, arginine, aspartic acid, cystine, glutamic acid, glycine, histidine, isoleucine, leucine, lysine, methionine, phenylalanine, proline, serine, threonine, tryptophan, tyrosine, valine, phenylglycine, norleucine, homoproline, or norvaline, and the amino acid (except glycine) can be the D- or L-isomer or the compound of the above Formula can be a mixture of both amino acid isomers; and wherein each phenyl group is unsubstituted or substituted with one or more of C1-6-alkoxy including methoxy, a halogen, or any combination thereof; or a pharmaceutically acceptable salt, hydrate, cation, or anion thereof.

In one embodiment, the halogen is a flouride or a chloride. In one embodiment, the —C1-6-alkyl group is methyl.

In further embodiments, the neuraminidase inhibitor comprises Formula III:

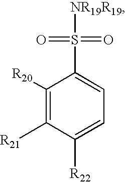

wherein: each R19 is independently —H, -benzyl, -phenyl, -naphthyl, —O-phenyl, or R23;
R20 is —H or —OH;
R21 is —H or —CO2H, or R20 and R21 combine to form a saturated or aromatic carbocyclic ring;
R22 is —H, —CO2H, —C1-6-alkyl, or -halogen;
R23 is:

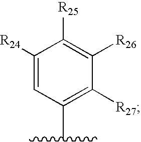

R24 is —H or —S—(CH2)n-furanyl;
R25 is —H, —OH, N-piperidinyl, or pyridinylmethyl;
R26 is —H;
R27 is —H, or R26 and R27 combine to form a saturated or aromatic carbocyclic ring; and
n is 1-6, wherein each phenyl or naphthyl is unsubstituted or substituted with one or more of C1-6-alkyl including methyl, C1-6-alkoxy including methoxy, or halogen, or any combination thereof; or a pharmaceutically acceptable salt, hydrate, cation, or anion thereof.

In some embodiments, the neuraminidase inhibitor comprises Formula IV:

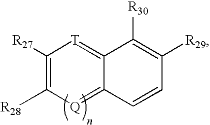

wherein: T is CR31 or N; Q is CR31 or N; n is 0, 1
R27 is —H, phenyl, or benzo-3,4-dioxolane;
R28 is -phenyl, 4-carboxymethyl-piperazin-1-yl, benzo-3,4-dioxolane, 4-([1-carboxyethoxy]-3-methoxy)-phenyl, or 2-(5-(carboxymethyl)-4-oxothiazolidin-2-ylidene)hydrazono-ethyl;
R29 is —H, —OH, or halogen;
R30 is —H, —OH, -halogen, —CO2H, or R31 and R32 combine to form an unsubstituted or substituted aromatic or saturated carbocyclic ring; and
R31 is —H, —CO2H, or (4-(2-(carboxymethoxy)benzylideneamino)-5-mercapto-4H-1,2,4-triazol-3-yl)methyl, wherein each saturated or aromatic carbocyclic ring, including phenyl, are unsubstituted or substituted with one or more of —OH, a -halogen, a C1-6-alkyl group, or a C1-6-alkoxy group; or a pharmaceutically acceptable salt, hydrate, cation, or anion thereof.

In yet other embodiments, the neuraminidase inhibitor comprises Formula V:

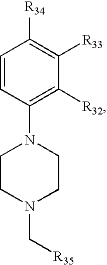

wherein: R32 is —H or -halogen; R33 is —H or -halogen;
R34 is —H, phenyl, or cyclohexyl, or R33 and R34 combine to form an aromatic carbocyclic ring;
R35 is —CO2H, -phenyl, 2-hydroxy-5-nitrophenyl, or 5-(1-carboxypentyloxy)-4-oxo-4H-pyran-2-yl; wherein each phenyl or cyclohexyl group is unsubstituted or substituted with one or more of —OH, a C1-6-alkyl including methyl, a C1-6-alkoxy including methoxy, a halogen, a nitro group, or any combination thereof; or a pharmaceutically acceptable salt, hydrate, cation, or anion thereof.

In one embodiment, R32 is fluoride. In another embodiment, R33 is chloride.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318.

In vitro translation-based libraries include but are not limited to those described in PCT Publication No. WO 91/05058; and Mattheakis et al., (1994) Proc. Natl. Acad. Sci. USA 91:9022-9026.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been penmethylated to generate a chemically transformed combinatorial library, is described by Ostresh et al. (1994), Proc. Natl. Acad. Sci. USA 91:11138-11142.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

One of skill in the art will be familiar with methods for predicting the effect on protein conformation of a change in protein sequence, and can thus “design” a variant which functions as an antagonist according to known methods. One example of such a method is described by Dahiyat and Mayo in Science (1997) 278:82 87, which describes the design of proteins de novo. The method can be applied to a known protein to vary only a portion of the polypeptide sequence. By applying the computational methods of Dahiyat and Mayo, specific variants of neuraminidase inhibitors confined to regions which bind the active site of a neuraminidase (such as bacterial neuraminidase) can be proposed and tested to determine whether the variant retains a desired conformation. Similarly, Blake (U.S. Pat. No. 5,565,325) teaches the use of known ligand structures to predict and synthesize variants with similar or modified function.

Other methods for preparing or identifying peptides that bind to a particular target are known in the art. Molecular imprinting, for instance, may be used for the de novo construction of macromolecular structures such as peptides that bind to a particular molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of neuraminidase inhibitors involves the steps of: (i) polymerization of functional monomers around a known substrate (the template or in this case, the neuraminidase active domain) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

A neuraminidase inhibitor according to the method of the invention modulates the activity of a neuraminidase via either reducing the activity of the neuraminidase in the biofilm after the neuraminidase inhibitor is applied, thus inhibiting formation of the biofilm. For example, a reduction in the formation of the biofilm can be measured by looking at a decrease in the surface area covered by the biofilm, thickness, or consistency (such as the integrity of the biofilm).

An inhibition or reduction in a biofilm via treatment with a neuraminidase inhibitor composition (such as a bacterial neuraminidase inhibitor) can be measured via techniques established in the art. These techniques enable one to assess bacterial attachment via measuring the staining of the adherent biomass, to view microbes in vivo via microscopy methods; or to monitor cell death in the biomass in response to toxic agents. The biofilm can be reduced with respect to the surface area covered by the biofilm, thickness, and consistency (for example, the integrity of the biofilm). Non-limiting examples of biofilm assays include microtiter plate biofilm assays, fluorescence-based biofilm assays, static biofilm assays according to Walker et al., ((2005) Infect. Immun. 73(6): 3693-3701), Air-liquid interface assays, colony biofilm assays, and Kadouri Drip-Fed Biofilm assays (Merritt et al., (2005) Current Protocols in Microbiology 1.B.1.1-1.B.1.17). Biofilms (such as their morphology, thickness, and the like) also can be analyzed via confocal microscopy methods (Walker et al., (2005) Infect. Immun. 73(6): 3693-3701). Thus, these biofilm assays (such as the one depicted in EXAMPLE 4) in combination with screening compound libraries as described above can be used to identify neuraminidase inhibitors capable of disrupting the formation of a biofilm (Lew et al., (2000) Curr. Med. Chem. 7(6):663-72; Werner et al., (2006) Brief Funct. Genomic Proteomic 5(1):32-6).

A reduction in a biofilm indicates that the neuraminidase inhibitor, inhibited formation of the biofilm as determined by observing that the inhibitor modulated the activity or the expression of the neuraminidase protein, because biofilms are comprised of various microorganisms, thus a neuraminidase inhibitor according to the method of the present invention can inhibit such microorganisms from producing a biofilm. Thus, the formation of biofilm by, e.g., of Gram-negative bacteria, can be inhibited.

Application of a neuraminidase inhibitor to a biofilm can be accomplished by any means such as spraying it onto the biofilm, infusing it into the biofilming, or pipetting into the depth of the biofilm, and the like (e.g., as shown in EXAMPLE 4).

If the neuraminidase inhibitor is to be administered to a subject, it will be in the form of a pharmaceutically acceptable composition or formulation as described below, wherein the composition or formulation is free of toxicity, which satisfies FDA requirements (see Remington: The Science and Practice of Pharmacy, 20^{th} ed., Lippincott Williams & Wilkins, 2000; U.S. Pat. No. 6,030,604). Such a neuraminidase inhibitor composition, comprising compounds or pharmaceutically acceptable salts, can be administered to a subject harboring a biofilm or is at risk of developing a biofilm (for example patient has undergone surgery, implantation, and the like) or is afflicted with a biofilm production-related disorder (discussed below). Administration can occur alone or with other therapeutically effective composition(s) (e.g., antibiotics) either simultaneously or at different times.

Formulations can include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form, will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The neuraminidase inhibitor composition can optionally comprise a suitable amount of a physiologically acceptable excipient. Non-limiting examples of physiologically acceptable excipients can be liquids, such as water and oils, including those of petroleum, animal, vegetable, or synthetic origin, such as peanut oil, soybean oil, mineral oil, sesame oil and the like; saline; gum acacia; gelatin; starch paste; talc; keratin; colloidal silica; urea and the like. In addition, auxiliary, stabilizing, thickening, lubricating, and coloring agents can be used. For example, the neuraminidase inhibitor composition and physiologically acceptable excipient are sterile when administered to a subject (such as an animal; for example a human). The physiologically acceptable excipient should be stable under the conditions of manufacture and storage and should be preserved against the contaminating action of microorganisms.

Water is a useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The neuraminidase inhibitor composition can be administered to the subject by any effective route, for example, orally, by infusion or bolus injection, by absorption through epithelial or mucocutaneous linings (e.g., oral, rectal, vaginal, and intestinal mucosa, etc.), intradermal, intramuscular, intraperitoneal, intravenous, subcutaneous, infusion, intranasal, epidural, oral, sublingual, intracerebral, intravaginal, transdermal, rectal, by inhalation, or topical, particularly to the ears, nose, eyes, or skin.

Pulmonary administration can also be employed, e.g., by use of an inhaler or nebulizer, and formulation with an aerosolizing agent, or via perfusion in a fluorocarbon or synthetic pulmonary surfactant. For example, the neuraminidase inhibitor composition can be formulated as a suppository, with traditional binders and excipients such as triglycerides. Various known delivery systems, including encapsulation in liposomes, microparticles, microcapsules, and capsules, can be used. Thus, the neuraminidase inhibitor composition can be delivered in a vesicle, in particular a liposome (see, e.g., Langer (1990) Science 249:1527-1533; Treat et al., Liposomes in the Therapy of Infectious Disease and Cancer 317-327 and 353-365 (1989)).

The neuraminidase inhibitor composition also can be delivered in a controlled-release system or sustained-release system (see, e.g., Goodson, in Medical Applications of Controlled Release, vol. 2, pp. 115-138 (1984)). Other controlled or sustained-release systems previously discussed can be used as well (Langer (1990) Science 249:1527-1533). For example, a pump can be used (Langer (1990) Science 249:1527-1533; Sefton (1987) CRC Crit. Ref Biomed. Eng. 14:201; Buchwald et al., (1980) Surgery 88:507; and Saudek et al., (1989) N. Engl. J. Med. 321:574); or polymeric materials can be used (see Langer and Wise (1985) Medical Applications of Controlled Release; CRC Press Inc., U.S.; Smolen and Ball (1984) Controlled Drug Bioavailability, Drug Product Design and Performance; Ranger and Peppas, (1983) J. Macromol. Sci. Rev. Macromol. Chem. 2:61; Levy et al., (1935) Science 228:190; During et al., (1989) Ann. Neural. 25:351; and Howard et al., (1989) J. Neurosurg. 71:105). The controlled- or sustained-release systems can be placed in proximity of a target of the compound or a pharmaceutically acceptable salt of the compound, e.g., the respiratory tract, thus requiring only a fraction of the systemic dose.

Modulation of neuraminidase activity can also result in the reduction or prevention of the formation of a biofilm on semi-solid and solid surfaces. For example, these surfaces can be the surface of implanted and/or inserted devices (a medical device, a catheter, an infusion set of an insulin pump, a stent, a prosthetic graft); a wound dressing; the oral cavity; the alimentary or vaginal tracts; the ears or eyes; a contact lens, in addition to the cases or containers that hold the lenses when not in use; industrial equipment, or plumbing systems.

Additionally, a neuraminidase inhibitor according to the method of the invention can be applied to a surface of a contact lens or an implantable/insertable device and other surgical or medical devices (such as a medical device, a catheter, the infusion set of an insulin pump, a stent, a prosthetic graft, a wound dressing) or a wound site via covering, coating, contacting, associating with, filling, or loading the device with a therapeutic amount of a neuraminidase inhibitor in any known manner including, but not limited to the following: (1) directly affixing to the implant, device, or wound site a therapeutic agent or composition of the neuraminidase inhibitor (for example, by either spraying the implant or device with a polymer/neuraminidase inhibitor film, or by dipping the implant or device into a polymer/neuraminidase inhibitor solution, or by other covalent or noncovalent means); (2) coating the implant, wound site, or device with a substance, (such as a hydrogel) that will in turn absorb the therapeutic neuraminidase inhibitor composition; (3) interweaving a therapeutic neuraminidase inhibitor composition coated thread (or the polymer itself formed into a thread) into the implant or device or wound site; (4) inserting the implant or device into a sleeve or mesh which is comprised of or coated with a therapeutic neuraminidase inhibitor composition; (5) constructing the implant or device itself with a therapeutic neuraminidase inhibitor composition (or with respect to a wound site, constructing the wound dressing with a therapeutic neuraminidase inhibitor composition; or (6) adapting the implant or device or wound dressing to release the therapeutic neuraminidase inhibitor composition. Specific disease conditions (for example, cystic fibrosis, pneumonia, and the like as described below) that are bacteria-based can also benefit from a treatment that modulates the activity of an enzyme involved in biofilm formation (for example, treatment with a neuraminidase inhibitor).

For example, application of a neuraminidase inhibitor onto the surface of implanted and/or inserted devices (as described above) in order to reduce or prevent bacterial biofilm formation thus allows for long-term implantation and can diminish the resultant likelihood of premature failure of the device due to encrustation and occlusion by such biofilm. The amount of the neuraminidase inhibitor present in a coating, spray, film, and the like (as described above) applied to the surfaces in order to prevent the formation of a bacterial biofilm is an amount effective to inhibit the attachment of microbes onto the surface and/or the synthesis and/or accumulation of biofilm by attached microbes on such a surface.

Methods of the invention can further protect a subject from premature failure of an insertable or implantable device due to encrustation and occlusion by a bacterial biofilm. According to this method, the subject is administered a therapeutically effective amount of the neuraminidase inhibitor of the invention prior to, at the same time, or after an insertable or implantable device is introduced. The subject is administered the neuraminidase inhibitor that prevents formation of a bacterial biofilm prior to, at the same time, or after the introduction of the implantable/insertable device. Treatment before or after implantation can take place immediately before or after the implantation or several hours before or after implantation, or at a time or times that the skilled physician deems appropriate. According to the present invention, a subject containing a wound site in addition to those subjects receiving implants can harbor a biofilm. For example, a neuraminidase inhibitor can be administered to the subject prior to, during, or after implantation/insertion of a medical device, catheter, stent, prosthesis, and the like or application of a wound dressing. The neuraminidase inhibitor can be administered to the subject according to routes previously described and can further aid in inhibiting biofilm formation on a surface an/or within a subject.

In the case of the oral cavity, the alimentary or vaginal tracts, the ears or eyes, or a contact lens, a therapeutic amount of a neuraminidase inhibitor can be applied via coating, contacting, associating with, filling, or loading the region with a formulation comprising a paste, gel, liquid, powder, tablet, and the like. With respect to the cases or containers that hold the lenses when not in use, industrial equipment, or plumbing systems, an effective amount of a neuraminidase inhibitor can be applied in the same manners as described above. These applications would thus aid in the inhibition of biofilm formation on such surfaces.

In a subject, a biofilm can form on an oral surface (such as teeth, tongue, back of throat, and the like). These biofilms can be associated with day-to-day bacterial activity of natural flora located in such environments, but can also be associated with oral-related disease(s), such as periodontal disease (for example, gingivitis or periodontitis) or dental carries. Application of the neuraminidase inhibitor (according to methods previously described) onto such oral surfaces can inhibit or prevent bacterial biofilm formation. The amount of the neuraminidase inhibitor that can be applied to the surfaces in order to prevent the formation of a bacterial biofilm is an amount effective to inhibit the attachment of microbes onto the surface and/or the synthesis and/or accumulation of biofilm by attached microbes on such a surface.

The neuraminidase inhibitor for use on oral surfaces can comprise a paste formulation (such as toothpaste), which can then be directly applied to the biofilm of such a surface in a subject. The paste formulation can further comprise an abrasive. The neuraminidase inhibitor can also exist as a gel formulation or in liquid formulation. For example, the neuraminidase inhibitor in a liquid formulation (such as a mouthwash) can directly come in contact with the biofilm on the oral surface of a subject.

Other aspects of the invention are directed at methods of treating biofilm production-related disorders in subjects in need thereof. The method entails administering to the subject an effective amount of a neuraminidase inhibitor that reduces biofilm formation in the subject, and then measuring a reduction or inhibition in the growth of biofilm production-related bacteria in the subject. The reduction in bacterial growth is indicative of the reduction in, or inhibition of, biofilm production in the subject, thereby treating the biofilm production-related disorder. For example, the administered neuraminidase inhibitor can reduce the activity of the neuraminidase or alter the expression of the neuraminidase, thereby inhibiting or preventing the formation of a bacterial biofilm.

According to the present invention, modulation of the neuraminidase enzyme (for example, via reducing enzymatic activity or protein expression as described above) can inhibit or reduce biofilm formation due to diminished adherence of microorganisms to a surface or to increased microorganism death. This therapeutic approach thus can be useful for the treatment of biofilm-production-related disorders/conditions and medical-device related infections associated with the formation of microbial biofilms.

Non-limiting examples of biofilm production-related disorders include chronic otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and infections from implanted/inserted devices. In one specific example, subjects with CF display an accumulation of biofilm in the lungs and digestive tract. In subjects afflicted with COPD, such as emphysema and chronic bronchitis, patients display a characteristic inflammation of the airways wherein airflow through such airways, and subsequently out of the lungs, is chronically obstructed. The methods of treatment according to the invention can also benefit a subject having chronic otitis media. Otitis media refers to an infection or inflammation in the middle ear area. The inflammation begins when infections (for example, those caused by bacterial or viral infections) that cause sore throats, colds, or other respiratory/breathing problems spread to the middle ear. Acute otitis media is the presence of fluid, typically pus, in the middle ear with symptoms of pain, redness of the eardrum, and possible fever. However the biofilm production-related disorder can be further classified as chronic if fluid is present in the middle ear for six or more weeks.

Biofilm production-related disorders can also encompass infections derived from implanted/inserted devices (such as those described previously), medical device-related infections, such as infections from biliary stents, orthopedic implant infections, and catheter-related infections (kidney, vascular, peritoneal). An infection can also originate from sites where the integrity of the skin and/or soft tissue has been compromised. Non-limiting examples include dermatitis, ulcers from peripheral vascular disease, a burn injury, and trauma. For example, a Gram-negative bacterium, such as P. aeruginosa, can cause opportunistic infections in such tissues. The ability of P. aeruginosa to infect burn wound sites, e.g., is enhanced due to the breakdown of the skin, burn-related immune defects, and antibiotic selection.

A subject in need of treatment (for example those previously described, such as an animal or human) can be one afflicted with the infections or disorders described above. As such, the subject is at risk of developing a biofilm on or in a biologically relevant surface, or already has developed such a biofilm. Such a subject at risk could be a candidate for treatment with a neuraminidase inhibitor in order to inhibit the development or onset of a biofilm-production-related disorder/condition or prevent the recurrence, onset, or development of one or more symptoms of a biofilm-production-related disorder/condition.

The subject in need can be administered a neuraminidase inhibitor as described above. It can be administered alone or in combination with a second therapeutic, e.g., such as an antibiotic, in order to prevent or inhibit the formation of bacterial biofilms. An antibiotic can be co-administered with the bacterial neuraminidase inhibitor, either sequentially or simultaneously. Upon contacting the cell, the bacterial neuraminidase inhibitor modulates the activity or the expression of the bacterial neuraminidase wherein the inhibitor reduces the activity or the expression of the bacterial neuraminidase, as described above.

An antibiotic refers to any compound known to one of ordinary skill in the art that will inhibit the growth of, or kill, bacteria. Useful, non-limiting examples of an antibiotic include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Administration of neuraminidase inhibitors to a subject can serve as a treatment that limits the severity and spread of pathogenic infections, such as bacterial infections. Neuraminidase inhibitors intended for human use must be efficacious and function in inhibiting the formation of biofilms, but must also not be toxic. The skilled physician via clinical trials can determine efficacy and toxicity.

An effective amount of a neuraminidase inhibitor refers to the amount of a therapy sufficient to reduce or ameliorate the severity and/or duration of a disorder, such as a biofilm production-related disorder (for example, CF, COPD, otitis media, and others described above). An effective amount of a neuraminidase inhibitor can also be sufficient to reduce the degree and time-span of one or more symptoms associated with a biofilm production-related disorder. Additionally, this amount can prevent the advancement of a biofilm production-related disorder, cause regression of such a disorder, prevent the recurrence, development, onset or progression of one or more symptoms associated with a biofilm production-related disorder. The skilled physician can determine a therapeutic dose of a neuraminidase inhibitor that inhibits biofilm formation and/or reduces the duration of a disorder or symptoms thereof. Methods of administration of a neuraminidase inhibitor composition have been described above.

A neuraminidase inhibitor according to the methods of the invention can reduce biofilms associated with a biofilm production-related disorder with respect to the surface area the biofilm covers, thickness, and/or consistency (for example, the integrity of the biofilm). This reduction can be assessed via measuring the growth of bacteria associated with biofilm-production-related disorders, conditions, or diseases. For example, the growth of bacteria of a biofilm-production-related disease can be quantified via measuring the density of bacteria of a biofilm-production-related-disease in a biological sample. Non-limiting examples of biological samples include blood, serum, sputum, lacrimal secretions, semen, urine, vaginal secretions, and tissue samples. The reduction in the growth of bacteria of a biofilm-production-related disease can also be measured by chest x-rays or by a pulmonary function test (PFT) (for example, spirometry or forced expiratory volume (FEV₁)).

In another non-limiting example, the presence or growth of biofilm production-related bacteria can be measured by detecting the presence of antigens of biofilm production-related bacteria in a biological sample, such as those described above. For example, an antibody to P. aeruginosa components can be used as a test for colonization/infection in a subject afflicted with a biofilm production-related condition or disorder, wherein the presence of Pseudomonas antigens is detected in a biological sample, such as blood. These antibodies can be generated according to methods well established in the art or can be obtained commercially (for example, from Abcam, Cambridge, Mass.; Cell Sciences Canton, Mass.; Novus Biologicals, Littleton, Colo.; or GeneTex, San Antonio, Tex.).

Spirometry measures lung function, for example, the volume and/or flow of air that can be inhaled and exhaled. The FEV₁ is a measurement of the volume exhaled during the first second of a forced expiratory maneuver started from the level of total lung capacity. FEV₁ is the most frequently used index for evaluating bronchoconstriction, airway obstruction, or bronchodilatation. These methods are important for assessing biofilm production-related conditions such as cystic fibrosis and COPD. A reduction in the growth of bacteria associated with biofilm production-related disorders and/or conditions is indicative of a reduction in or inhibition of biofilm production.

Methods of the invention are provided that can prevent or reduce biofilm formation (such as a bacterial biofilm) on a biologically relevant surface, wherein a neuraminidase inhibitor is administered to a subject (such as a mammal, for example a human) in order to prevent or reduce the formation of bacterial biofilms. These surfaces include, but are not limited to, an epithelial or mucosal surface of the respiratory tract, lungs, the oral cavity, the alimentary and vaginal tracts, in the ear or the surface of the eye, and the urinary tract. For example, a biofilm can affect the surface of a lung (such as the lung of a subject with CF or COPD), which is comprised of epithelial cells.

Epithelial cells are named on the basis of their cell type: simple squamous, simple cuboidal, simple columnar, stratified squamous, stratified cuboidal, or stratified columnar epithelia. Such epithelial cells can be obtained from any tissue organ having such cells, for example from the lining of cavities such as the mouth, blood vessels, heart and lungs; from the outer layers of the skin; from the lining of the air passages, stomach, and intestines; in the nose, ears and the taste buds of the tongue; from the lining of the vaginal and urinary tracts, rectum, uterus, and oviducts, and from the larger ducts of certain glands and the papillary ducts of the kidneys. Epithelial cells can also be obtained from in vitro epithelial cell culture systems well known in the art (see, e.g., Harris, A. (ed.), (1996) Epithelial Cell Culture, Cambridge University Press). Such cell lines may be available commercially or can be generated via standard cell culturing techniques (see e.g. Harris, supra).

Other aspects of the current invention are directed to methods that are useful for treating a subject (such as an animal or human) that has, is developing, or is at risk of developing a biofilm-production-related disorder/condition. A subject who is developing a biofilm-production-related disorder/condition is an individual harboring an immature biofilm clinically evident or detectable to the skilled artisan, but that has not yet fully formed. A subject at risk of developing a biofilm can be one in which the introduction of a medical device, a graft implantation, and the like is scheduled. The risk of developing a biofilm can also be due to a biofilm production-related disease (such as the channel transporter mutation associated with CF) that is in its earlier stages, e.g., no bacterial infection and/or biofilm formation is yet detected.

In a specific example, methods are provided for preventing biofilm formation in the airways of cystic fibrosis patients who are free of bacterial infection of the airways. Such patients are at risk of developing a biofilm, and as such, are “in need thereof.” The method entails administering to the subject an effective amount of a neuraminidase inhibitor, which prevents growth of bacteria associated with a biofilm production-related disorder in the airways of a subject, and detecting the absence of such bacterial growth in the airways of the subject. The absence of bacterial growth is indicative of the lack of biofilm formation in the airways of the subject. For example, the subject may be one afflicted with CF and is a human (such as an individual of 5 years of age or less) that has not yet developed a bacterial infection of the airways indicating that P. aeruginosa has not yet colonized the epithelial cells of the lung airways. Airways of the lung include bronchii, bronchioles, aleveolar ducts, alveolar sacs, and alveoili.

The growth of bacteria associated with CF can be quantified by detecting the presence of P. aeruginosa (e.g. by measuring the density of the bacteria) in a biological sample according to methods practiced in the art. Non-limiting examples of biological samples include blood, serum, sputum, lacrimal secretions, sweat, semen, urine, vaginal secretions, and tissue samples. For example, the presence or absence of bacteria can be measured via detecting the presence of bacterial in a biological sample, such as those described above. An antibody to P. aeruginosa components can be used as a test for colonization/infection in a subject afflicted with a biofilm production-related condition or disorder (such as CF), wherein the presence of Pseudomonas antigens is detected in a biological sample, such as blood. These antibodies can be generated according to methods well established in the art or can be obtained commercially (for example, from Abcam, Cambridge, Mass.; Cell Sciences Canton, Mass.; Novus Biologicals, Littleton, Colo.; or GeneTex, San Antonio, Tex.). The absence of bacterial growth and its associated biofilm can also be measured, e.g., by chest x-rays or by a pulmonary function test (PFT) (for example, spirometry or FEV₁, methods described above).

According to the invention, administration of neuraminidase inhibitors to a subject (for example, one afflicted with CF who is free of bacterial infection in the airways) can serve as a preventive means by which to deter the development of pathogenic infections, such as bacterial infections (eg. P. aeruginosa).

An effective amount of a neuraminidase inhibitor to be administered can be the amount sufficient to prevent the onset or development of a pathogenic infection associated with a biofilm production-related disease or disorder (for example, COPD or CF). The skilled physician can determine a therapeutic dose of a neuraminidase inhibitor that prevents pathogenic infection in addition to biofilm formation. An effective amount of a neuraminidase inhibitor, for example, one directed at the Pseuidomonas enzyme, can be administered according to methods of this invention. Methods of administration of a neuraminidase inhibitor composition have been described above.

Aspects of the present invention also provide methods of preventing or reducing biofilm formation associated with a wide variety of commercial, industrial, and processing operations, such as those found in water handling/processing industries. The method for inhibiting biofilm formation on an industrial/commercial surface entails applying a neuraminidase inhibitor to the biofilm found on such surfaces. The neuraminidase inhibitor modulated activity or expression of the neuraminidase protein can then be measured. A reduction in the neuraminidase inhibitor modulated activity or expression of the neuraminidase protein is indicative of the inhibition of biofilm formation. The neuraminidase inhibitor can be directed at any neuraminidase produced by organisms in the biofilm. These have been described above.

The neuraminidase inhibitors useful in the invention that prevent or reduce the formation of bacterial biofilms can be utilized in order to prevent microorganisms from adhering to surfaces. These surfaces may be hard, semi-hard, porous, soft, semi-soft, regenerating, or non-regenerating; and can include, but are not limited to, metal, alloy, polyurethane, water, polymeric surfaces of implantable/insertable devices (such as medical devices or catheters), the enamel of teeth, and surfaces of mammalian cellular membranes.

For example, some surfaces can be the surfaces of industrial equipment (such as, equipment located in Good Manufacturing Practice (GMP) facilities, food processing plants, photo processing venues, and the like), the surfaces of plumbing systems, or the surfaces bodies of water (such as lakes, swimming pools, oceans, and the like). Embodiments of the invention further provide methods for inhibiting and/or reducing biofilm formation within a plumbing system.

The surfaces may be coated, sprayed, or impregnated with a neuraminidase inhibitor prior to use to prevent the formation of bacterial biofilms. Surfaces also may be treated with a neuraminidase inhibitor to reduce, control, or eradicate microorganisms (such as those described above) adhering to such surfaces. In a specific example, the method can be used in an open re-circulating water system used for cooling to control the temperature of fermentation tanks. In such a system, the water circulates through coils and jackets in the tank, over an induced draft-cooling tower, and then is pumped back from the sump. Biofilm-producing microorganisms can flourish in the cooling water system due to contamination and highly nutritive substances from the surrounding environment (Coetser et al., (2005) Crit. Rev. Micro. 31: 212-32). This biofilm can form on the cooling tower water distribution elements, its support components, and on the heat transfer surfaces of the system resulting in poor cooling efficiency. Thus, to prevent formation of the biofilm, a neuraminidase inhibitor is applied to treat the water-cooling system. Not only is the treatment suitable for the water-cooling system of a fermentation tank, but can also be applicable to air conditioning condensers, (such as those found in hospitals or industrial plants), that are served by a rooftop open-deck cooling tower (described in U.S. Pat. No. 6,395,189 and U.S. Appln. Pub. No. 2005/0158253).

The neuraminidase inhibitor can be added directly to a water handling or collection system (such as the systems described above). Alternatively, the bacterial neuraminidase inhibitor can be applied to the biofilm, itself, or to the bacteria within, or the producers of the biofilm or which can produce the biofilm. It can be applied as a formulation comprising a paste, liquid, powder, gel, or tablet. The neuraminidase inhibitor functions via modulating the activity or the expression of a bacterial neuraminidase protein. Upon the neuraminidase inhibitor contacting the bacterial cell, the activity or expression of the bacterial neuraminidase is reduced, thereby preventing or reducing the formation of a bacterial biofilm. For example, the biofilm formed on the surfaces of systems (which include but are not limited to plumbing, tubing, and support components) involved with water condensate collections, sewerage discharges, paper pulping operations, re-circulating water systems (such as air conditioning systems, a cooling tower, and the like), and, in water bearing, handling, processing, collection systems of an industrial setting can be formed by a Gram-negative bacterium (as described above).

Addition of the neuraminidase inhibitor prevents or reduces the formation of biofilms on the surface of the water or on the surfaces of the pipes or plumbing of water-handling systems, or other surfaces of the collection and/or operation systems that the water contacts.

Also provided are methods for identifying or screening for inhibitors of a neuraminidase protein useful in preventing or inhibiting the formation of biofilms. The method entails contacting a cell infected with a biofilm-producing microbe, such as a protozoa, yeast, virus, or bacterium, (e.g., Pseudomonas) with a test (or candidate) neuraminidase inhibitor, and then determining whether the test neuraminidase inhibitor inhibits biofilm formation. Inhibition of biofilm formation thus is indicative of the ability of the test neuraminidase inhibitor to prevent or inhibit microbial infection.

Inhibition of biofilm formation can be determined by any known method, such as a visual method performed with the aid of a microscope, colorimterically via densitometry, and the like. Neuraminidase inhibitors that reduce or prevent the formation of a biofilm on surfaces are described or can be identified via biofilm assays as described above (see, e.g., EXAMPLE 4). Thus, one skilled in the art can carry out any known biofilm assay, such as those previously described.

Additionally, neuraminidase gene products, including polynucleotides, oligonucleotides and polypeptides, can be used in screening assays to identify compounds that specifically bind to bacterial, viral, yeast, or protozoan neuraminidase gene products and thus have potential use as agonists, or antagonists of such neuraminidases. In a particular use, the bacterial, viral, yeast, or protozoan neuraminidase polynucleotides and polypeptides of the invention are useful to screen for compounds that affect the sialidase or biofilm formation activities of bacterial, viral, yeast, or protozoan neuraminidase gene products.

The invention thus provides assays to detect molecules that specifically bind to bacterial, viral, yeast, or protozoan neuraminidases. For example, recombinant cells expressing a gene encoding bacterial, viral, yeast, or protozoan neuraminidase can be used to recombinantly produce a bacterial, viral, yeast, or protozoan neuraminidases polypeptide, respectively, and to screen for molecules that bind to a bacterial, viral, yeast, or protozoan neuraminidases polypeptide, respectively. Methods that can be used to carry out the foregoing are commonly known in the art.

A neuraminidase inhibitor that can be used according to the invention has been described above.

Non-limiting examples of cells to be contacted with the neuraminidase inhibitor include bacterial cells, yeast cells, protozoan cells, and cells infected with a viral or other pathogen. Representative bacteria include but are not limited to Legionella sp., P. aeruginosa, H. influenzae, V. cholerae, Yersinia pestis, Escherichia coli. Alternatively, the cell to be contacted is an animal cell, such as a mammalian cell, or more specifically, a human cell. The cell may be from a particular tissue or cell line, such as an epithelial cell.

Another aspect of the invention is directed to a mutant P. aeruginosa strain having a deletion in the gene encoding a neuraminidase protein. Deleting a portion of the gene so that the gene cannot function may be accomplished by mutation or insertion of another DNA in the base sequence of the gene (also referred to as a gene disruption). As a result, the gene cannot be transcribed into mRNA, the structural gene is not translated, and the transcription product mRNA becomes incomplete. A mutation or deletion occurs in the amino acid sequence of the translation product or structural protein, rendering the protein incapable of performing its original function.

Any method known in the art may be used for constructing a gene-disrupted strain, such as a strain wherein the gene encodes a neuraminidase protein. For example, the gene disruption can occur via homologous recombination or other methods described in Nickoloff (ed.), (1995) Methods in Molecular Biology 47: 291-302, Humana Press Inc., Totowa, N.J.; or in Sambrook et al. (eds.), Molecular Cloning: A Laboratory Manual, Second Edition (1989) Cold Spring Harbor Laboratory Press. For example, the deletion may be in the PΔ2794 gene of P. aeruginosa. This gene may have the nucleic acid sequence set forth as SEQ ID NO:1. The resultant mutant protein has an amino acid sequence such as SEQ ID NO:2. A non-limiting representative deleted strain according to the invention may be the P. aeruginosa mutant strain Δ2794. The P. aeruginosa mutant strain with a deletion mutation in its neuraminidase fails to form a biofilm. Accordingly, this mutant can be used to identify other genes that can rescue the biofilm formation defect, such as, for example other genes that encode neuraminidase-like proteins.

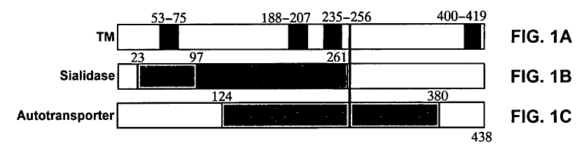

In one non-limiting representative embodiment, a mutant of P. aeruginosa PAO1 was constructed via deleting the PΔ2794 neuraminidase locus and testing its virulence and immunostimulatory capabilities in a mouse model of infection. FIGS. 1A-1C are schematic representations that depict various properties of the PΔ2794 locus. The gene PΔ2794 is depicted 3 times and shaded regions demonstrate distinct functional predictions. Regions are indicated using amino acid positions. Predicted transmembrane (TM) regions are shown first. The second gene depiction shows the region most similar to other sialidase genes. Lighter gray shading indicates the region predicted by BLAST analysis. For an inclusive definition of the boundaries of this region we used the most extreme amino and carboxy terminal positions of BLAST alignments for the first 50 hits that were also labeled as sialidase or neuraminidase genes. The darker shading corresponds to the sialidase region predicted by the SMART program (http://smart.embl-heidelberg.de/). The third gene depiction shows the region similar to autotransporter genes. This region had an e-value score of 6×10⁻⁴ in our BLAST search.

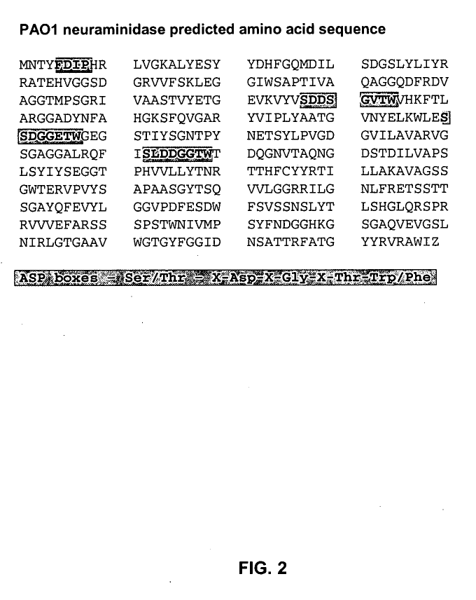

In the PΔ2794 locus, the sialidase region (in black) and a domain expected to have autotransporter function (FIG. 1B and FIG. 1C, respectively) were identified (Henderson et al., (2004) Microbiol. Mol. Biol. Rev. 68:692-744; Corfield et al., (1981) Biochem. J. 197:293-299). FIG. 2 is the predicted PAO1 neuraminidase amino acid sequence. This sequence includes the ASP boxes, which could interact with sialic acid (FIG. 2) (Roggentin et al., (1989) Glycoconj. J. 6:349-353).

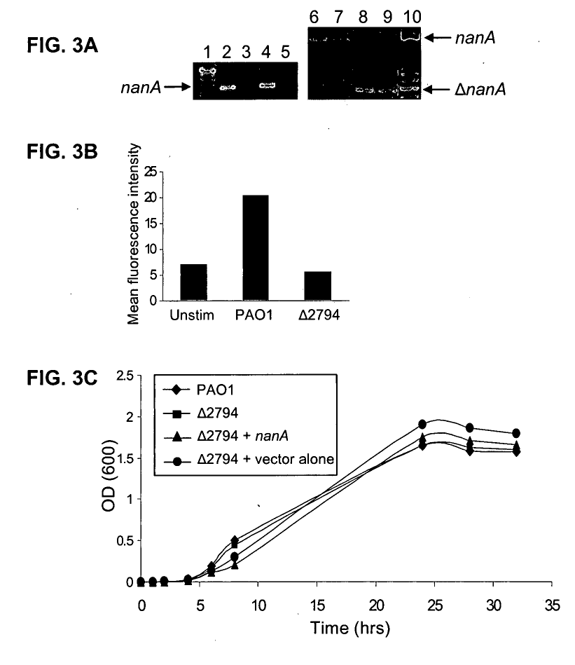

An inframe non-polar deletion allele of the predicted neuraminidase open reading frame (PΔ2794, nanA) was constructed and used to replace the wild type gene in PAO1 (FIG. 3A). Loss of neuraminidase activity was documented by an assay monitoring the ability of culture supernatants to expose asialoGM1 from human airway epithelial cells (FIG. 3B) (Cacalano et al., (1992) J. Clin. Invest. 89:1866-1874). The deletion was shown not to impose any metabolic consequences on the fitness of the mutant as growth curves of the wild type, mutant and complemented strains were comparable (FIG. 3C).

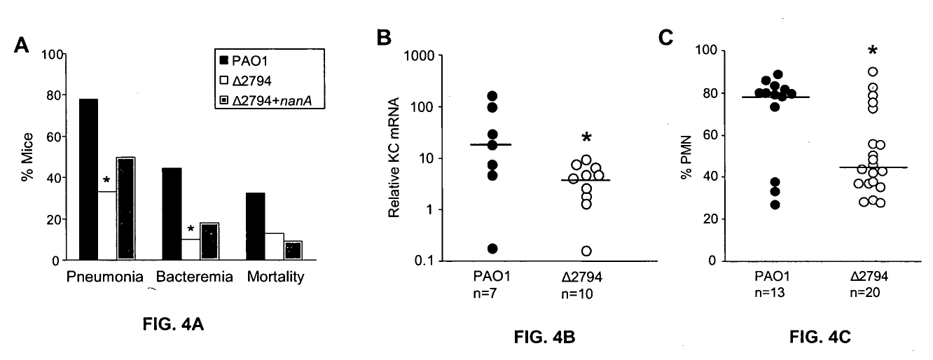

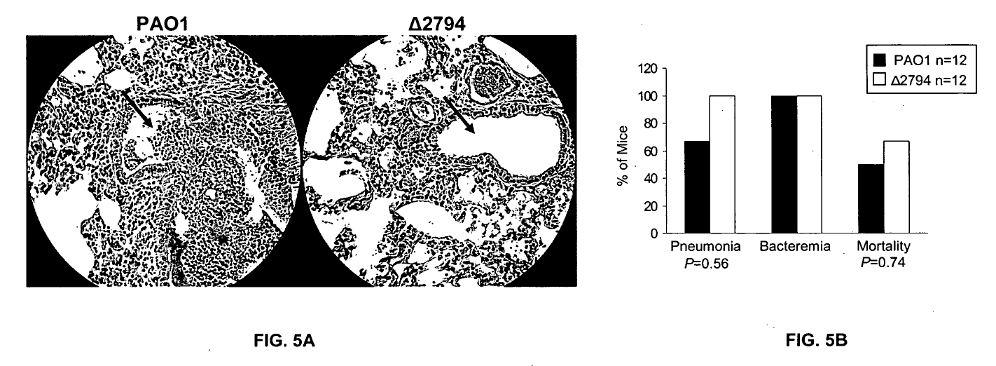

Virulence properties of Δ2794 neuraminidase mutant were then characterized. To evaluate the role of the neuraminidase in respiratory tract infection, 2×10⁸ cfu inocula of the wild type PAO1, Δ2794 and Δ2794+nanA strains were used to infect 7-10 day old BALB/c mice by the intranasal route, and morbidity and mortality assessed at 18 hours post infection (FIGS. 4A-D). In contrast to PAO1, the Δ2794 mutant was readily cleared from the respiratory tract; 33% of the mice infected with Δ2794 developed pneumonia versus 78% of the mice infected with PAO1 (P<0.05). Significantly fewer mice infected with the Δ2794 mutant became bacteremic; 10% versus 44% (P<0.05). Mortality rates were not statistically different (FIG. 4A). Leukocytes were gated based on CD45 expression and the percentage of Ly-6G+cells was determined. Quantification of lung mRNA indicated less chemokine KC expression that correlated with fewer PMNs recruited to the lungs inoculated with Δ2794 as compared with PAO1 (P<0.05) (FIGS. 4B-C). Histopathology similarly demonstrated substantially decreased inflammation in Δ2794-infected lungs (FIG. 5A). This shows that a functional neuraminidase contributes to increased and virulence and is required to establish a biofilm.

The virulence of wild type and Δ2794 strains was compared when introduced into mice by the intraperitoneal (IP) route. Mice were inoculated intraperitoneally with PAO1 or Δ2794 (n=12 for each). 16 hours later, mice were euthanized and lungs and spleens harvested for plating of single cells suspensions. Pneumonia was defined as the recovery of more than 1000 CFU per lung and bacteremia was defined as the presence of bacteria in the spleen. There were no differences in virulence when equal inocula of the wild type and mutant strains were injected by the IP route (FIG. 5B).

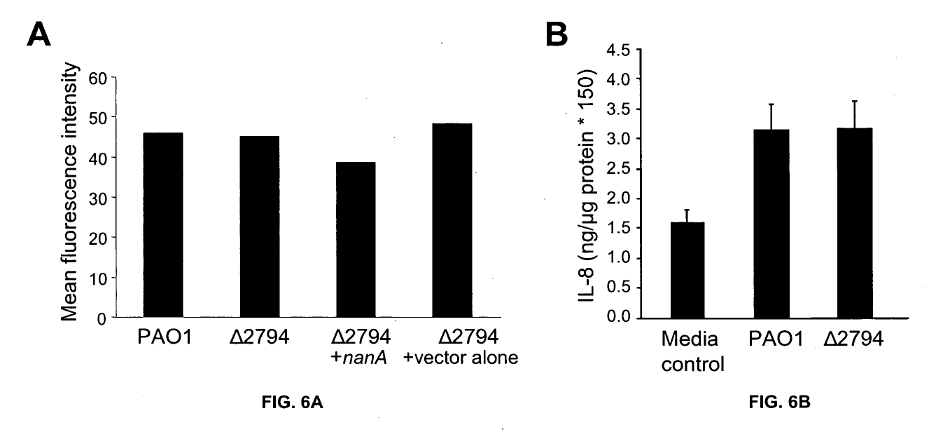

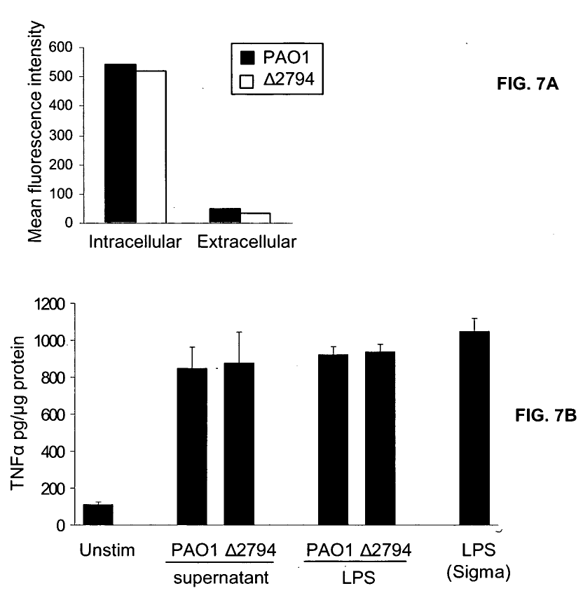

Immunostimulatory properties of the PΔ2794 mutant were also examined. To account for the attenuated phenotype of the Δ2794 mutant when introduced into the airways, its ability to attach to and stimulate chemokine and cytokine expression was characterized in both airway epithelial cells and macrophages (FIGS. 6 and 7). Although the PAO1 culture supernatant exposed more asialoGM1 on the surface of human airway cells than did Δ2794 (FIG. 3B), there appeared to be no significant differences in either bacterial adherence or the induction of IL-8 expression by the wild type and Δ2794 mutant strains (FIGS. 6A-B), in contrast to published reports that used concentrated purified enzyme (Cacalano et al., (1992) J. Clin. Invest. 89:1866-1874). As bacterial modification of surface glycoconjugates could affect interactions with phagocytic cells, the uptake and killing by RAW cells was compared. Adherent organisms were quantified by staining with anti-OMP and adherent plus internalized bacteria quantified in RAW cells that were permeabilized prior to staining to determine total RAW cell associated bacteria. Both wild type and mutant bacteria were efficiently phagocytosed by RAW cells and both induced equivalent amounts of TNFα production (FIGS. 7A-B). In addition, outer membrane proteins and secreted exoproducts were compared from the wild type and mutant strains and detected no differences.

The Effects of the PΔ2794 neuraminidase locus on LPS were investigated. A biochemical analysis of P. aeruginosa LPS had previously failed to identify sialic acid in contrast to other neuraminidase producing organisms, such as Haemophilus (Knirel et al., (1988) Acta. Microbiol. Hung. 35:3-24; Swords et al., (2004) Infect. Immun. 72:106-113). Although the enzyme could target a different amino sugar involved in LPS biosynthesis, GC-Mass spectroscopic analysis performed did not reveal any differences in LPS lipid A structures between the wild type, mutant, or complemented strains (Ernst et al., (1999) Science 286:1561-1565). Having found no biologically important alteration in eukaryotic surface glycosylation that could be attributed to effects of the neuraminidase, possible effects on surface structures, including LPS, were examined. As LPS structure is responsible for the resistance of P. aeruginosa to the lytic affects of normal human complement, PAO1 and Δ2794 sensitivity was compared to 10% human serum and was found to have no differences. These negative results along with the observation that the wild type and mutant strains were equally virulent when injected intraperitoneally, indicate that the Δ2794 mutation does not appear to have a major effect on LPS or its immunogenicity.

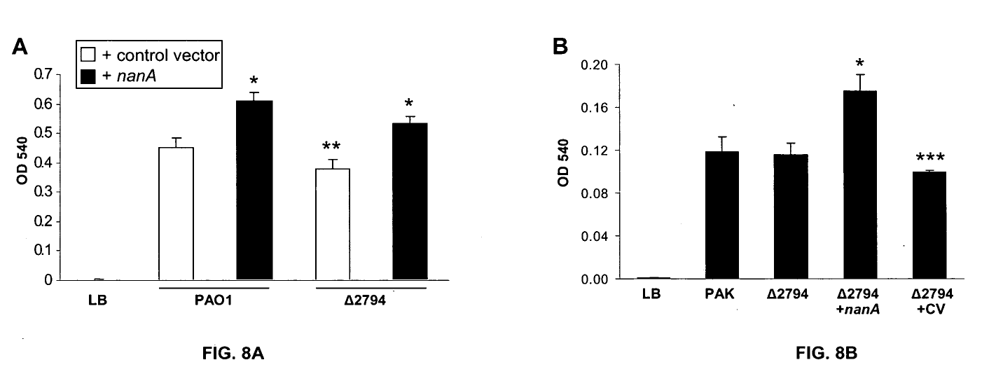

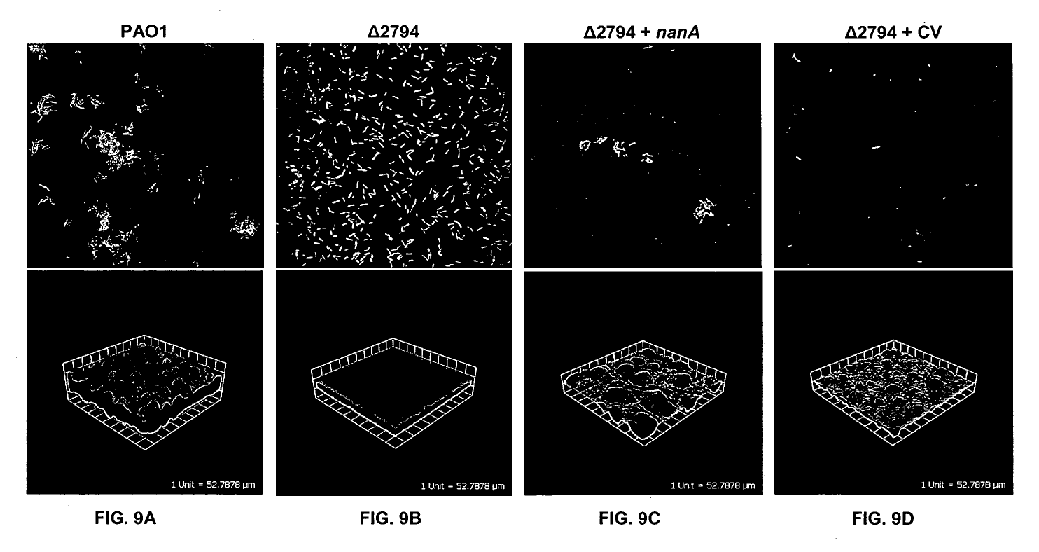

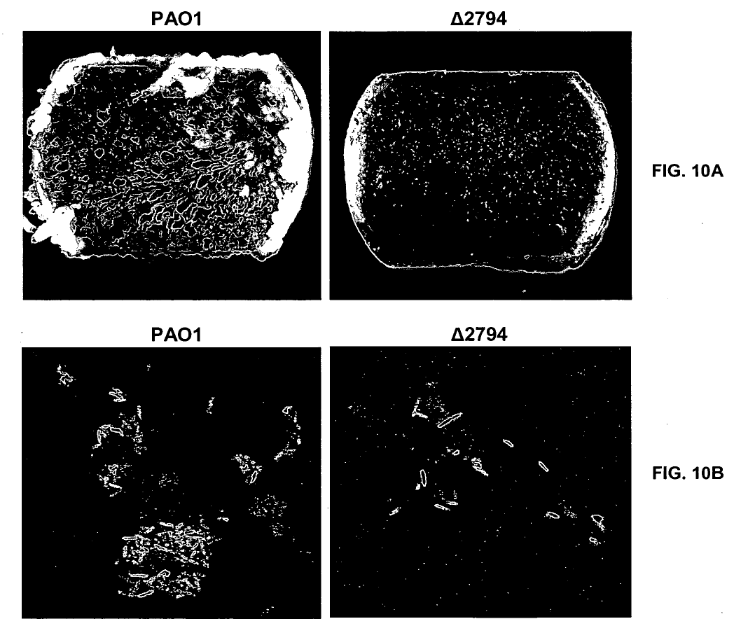

The PΔ2794 locus affects biofilm formation. Since the Δ2794 mutant appeared to be deficient solely in its ability to initiate infection by the mucosal route, the neuraminidase could target other bacterial exopolysaccharides, such as those involved in biofilm formation. Bacteria-bacteria interactions were examined using crystal violet staining to quantify biofilm production (FIG. 8A). The wild type strain PAO1 containing an empty vector was used as a control and all strains were grown under identical conditions in gentamicin selection. The Δ2794 mutant with the empty vector produced significantly less biofilm (P<0.001) than PAO1 also containing the control vector. Biofilm production by the Δ2794 mutant expressing the cloned nanA locus was significantly greater than that of the parental strain (P<0.0001) consistent with the constitutive expression of the cloned gene. Similarly, overexpression of nanA by expressing the cloned gene in PAO1 also resulted in significantly greater biofilm production (P<0.0001). In addition, an independently derived mutation in strain PAK and a Δ2794 PAK mutant was tested (FIG. 8B). Although PAK did not produce significant amounts of biofilm under control conditions and deletion of the 2794 locus had no apparent effect, overexpression of the cloned 2794 locus in Δ2794 PAK resulted in a significant increase in the production of biofilm (P<0.0001) as compared to PAK parental strain. Biofilm assays were then performed in a flow cell as described below (FIG. 9). Compared to PAO1, Δ2794 formed less differentiated and flattened layers (FIG. 9A vs. FIG. 9B, respectively). Δ2794+nanA complemented mutant showed restored, differentiated clumps of cells (FIG. 9C) while the Δ2794+control vector mutant vector did not (FIG. 9D). Biofilm assays were also performed on a rotating disc reactor as described below (FIG. 10A). These methods allow one to formally evaluate the ability of the bacteria to form structured communities. Biofilms were produced under more dynamic conditions using a rotating disc reactor, which uses removable discs attached to a rotor in a chemostat. The disc rotation produces high shear forces. High shear conditions can accentuate deficiencies in attachment and matrix integrity. In each assay system, the biofilm produced by Δ2794 exhibited dramatically changed architecture and complementation of the phenotype by the cloned gene was observed. To establish that the mutant Δ2794 exhibits similarly impaired biofilm formation on epithelial cells, GFP expressing bacteria was incubated with monolayers of airway epithelial cells and observed by fluorescence imaging that the Δ2794 organisms did not cluster or auto-agglutinate, in contrast to PAO1 (FIG. 10B).

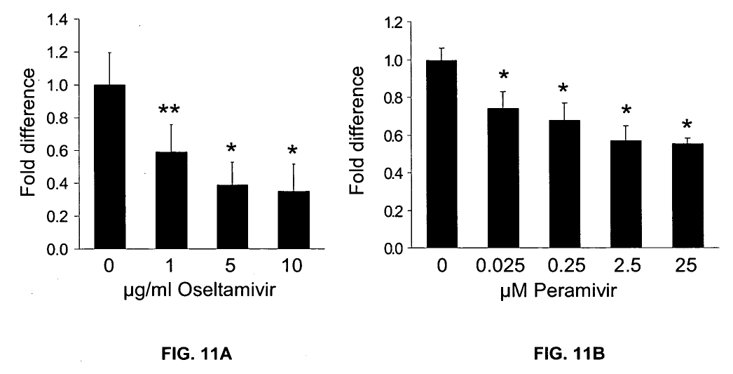

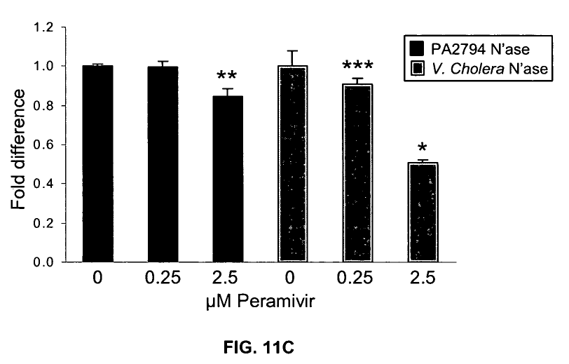

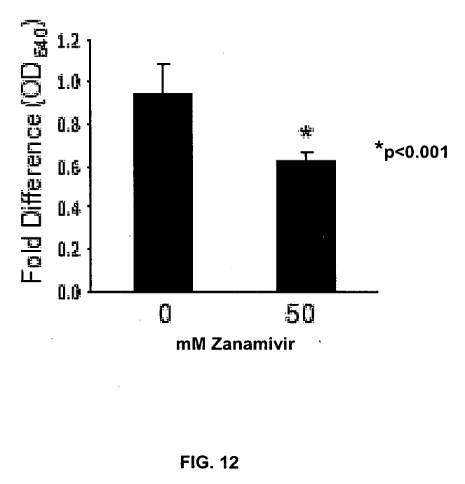

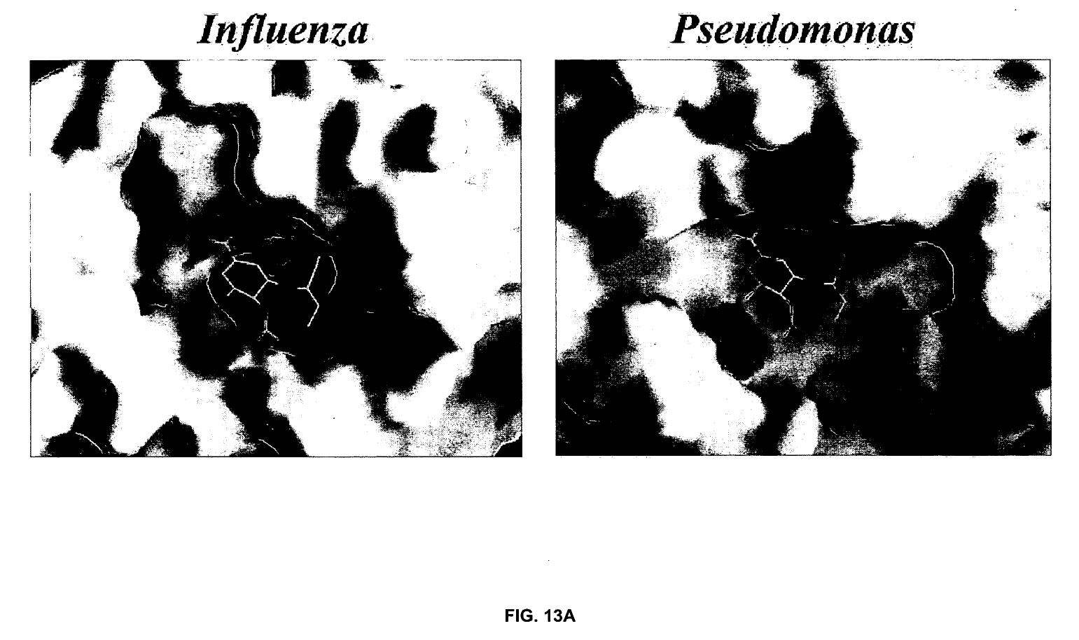

The ability of neuraminidase inhibitors to block biofilm production was examined. Drugs that target the neuraminidase produced by influenza viruses are an important component of anti-viral chemotherapy used for both prophylaxis and treatment. The effects of the influenza virus neuraminidase inhibitors oseltamivir, peramivir, and zanamivir were tested on PAO1 biofilm formation using the crystal violet assay (FIGS. 11A-B and FIG. 12). A dose response effect was observed with each of the two drugs, suggesting that P. aeruginosa biofilm formation may be a target to prevent infections in patients at risk, by using neuraminidase inhibitors.

To document that these viral neuraminidase inhibitors are specifically interacting with the bacterial neuraminidase, their effect in blocking neuraminidase activity was also tested using the fluorescent substrate 2′-(4-methylumbelliferyl)-α-D-N-acetylneuraminic acid (FIG. 11C). The Vibrio cholerae enzyme as well as the P. aeruginosa PAO1 enzyme were inhibited by peramivir, suggesting that the active sites of both types of neuraminidases have some affinity for the available viral neuraminidase inhibitors.

In contrast to current paradigms regarding P. aeruginosa gene expression in the CF lung, this data shows that cell-cell communication, as manifested by biofilm production, is critical even in the initial colonization process. Although the presence of “mature” biofilms with complex secondary structures may be more typical of long standing infection, the data presented below suggest that neuraminidase production is involved in cell-cell interactions necessary for colonization and persistence in the airway. The involvement of the neuraminidase locus, for example in Pseudomonas, appears to contribute to the initial stages of biofilm development. The Δ2794 mutants trapped in a planktonic form of growth were fully virulent in a model of intraperitoneal sepsis in which replication and induction of a host immune response are sufficient to cause mortality, but could not efficiently colonize the lung. Additional in vitro assays assessing immuno-stimulatory interactions with both macrophages and epithelial cells revealed no significant differences between the wild type and mutant strain. The biologically significant consequence of the loss of neuraminidase appears to be limited to its defect in cell-cell aggregation.

Although fully virulent when introduced by the intraperitoneal route, the Δ2794 mutant is unable to establish respiratory infection by intranasal inoculation. The inability to colonize the respiratory tract correlated with diminished production of biofilm, as assessed by scanning electron microscopy and in vitro assays. The importance of neuraminidase in biofilm production was further demonstrated by showing that viral neuraminidase inhibitors in clinical use block P. aeruginosa biofilm production in vitro as well. Thus, P. aeruginosa neuraminidase has a key role in the initial stages of pulmonary infection by targeting bacterial glycoconjugates and contributing to the formation of biofilm.

SEQUENCE LISTINGS

SEQ ID NO: 1 -

atgaatactt attttgatat tccgcatcgg ctagtgggaa
aggctctgta tgaatcatat tatgatcatt ttggccaaat
ggatatattg tcagatggaa gtttgtacct aatatatagg
cgggcaacag agcatgtagg tggtagtgat gggcgtgttg
ttttcagtaa actggagggt ggtatttgga gtgcgcctac
catagttgcc caggcgggag gacaggattt tcgagatgta
gctggtggga cgatgcctag cggaaggatc gtcgcggcct
cgacggttta tgaaacagga gaggtgaagg tctatgtatc
tgatgattca ggtgtgacat gggtacataa attcacattg
gctagaggtg gggcggatta taattttgct catggaaaaa
gttttcaagt aggggcacgc tatgtgattc ctctttacgc
ggcgacggga gtcaattatg aactgaaatg gctagagtct
tctgatggcg gagaaacttg gggcgaagga agcacgatct
acagcgggaa cacaccatac aatgagactt cttaccttcc
tgtgggagat ggcgtaattc tggctgtagc cagagtagga
tctggtgccg gaggagcgtt acgtcagttt ataagtctgg
acgacggcgg cacatggact gatcagggta atgtgactgc
acaaaatggt gactctaccg atattctcgt agccccttct
ctctcctata tttattctga gggaggtaca ccacatgtag
ttcttttgta cacgaacaga acgactcatt tttgttatta
cagaacaatc ctgctggcga aagccgtagc aggctcctcc
ggctggacgg agcgtgttcc tgtgtatagt gcgccagccg
cctccggtta tactagtcaa gtcgttttgg gaggtcgaag
aatactgggt aatctattca gggagacgtc ttctacgacg
tcgggagctt atcagtttga agtgtatctg ggaggagtcc
cggattttga gtcagactgg ttttctgttt cttcaaatag
tttgtatacc ctgagtcatg gactccagag gtcgccgcgt
cgggtggtcg ttgagtttgc aaggtcatcg agtccatcaa
catggaacat tgtcatgccc agttatttca atgatggagg
gcataaaggc agtggggctc aggttgaagt gggtagcttg
aatattcggc ttggaactgg agcggcagta tggggcacgg
gatatttcgg aggaatcgac aatagtgcca cgactcgatt
cgctaccggg tattatcggg tcagagcatg gatttag

SEQ ID NO:2 -

MNTYFDIPHRLVGKALYESYYDHFGQMDILSDGSLYLIYRRATEHVGGSD
GRVVFSKLEGGIWSAPTIVAQAGGQDFRDVAGGTMPSGRIVAASTVYETG
EVKVYVSDDSGVTWVHKFTLARGGADYNFAHGKSFQVGARYVIPLYAATG
VNYELKWLESSDGGETWGEGSTIYSGNTPYNETSYLPVGDGVILAVARVG
SGAGGALRQFISLDDGGTWTDQGNVTAQNGDSTDILVAPSLSYIYSEGGT
PHVVLLYTNRTTHFCYYRTILLAKAVAGSSGWTERVPVYSAPAASGYTSQ
VVLGGRRILGNLFRETSSTTSGAYQFEVYLGGVPDFESDWFSVSSNSLYT
LSHGLQRSPRRVVVEFARSSSPSTWNIVMPSYFNDGGHKGSGAQVEVGSL
NIRLGTGAAVWGTGYFGGIDNSATTRFATGYYRVRAWIZ
(Z represents a stop-codon)

SEQ ID NO: 3 -

CGC ACT ATA CAC AGG AAC ACG

SEQ ID NO: 4 -

GCC TAG CGG AAG GAT CGT CGC

SEQ ID NO: 5 -

GAT TAT AAG TCT GCC GTC GG

SEQ ID NO: 6 -

CTC GGG AAA CGT GCA CAT CC

SEQ ID NO: 7 -

CCGCGCCTATCGCCAATGAGCTGCGC

SEQ ID NO: 8 -

CTTGGGGACACCTTTTAGCATCTTTTGG

SEQ ID NO: 9 -

GTGGGGCGCCCCAGGCACCA

SEQ ID NO: 10 -

CGGTTGG CCTTGGGGTTCAGGGGGG

### Examples

A number of Examples are provided below to facilitate a more complete understanding of the present invention. The following examples illustrate the exemplary modes of making and practicing the present invention. However, the scope of the invention is not limited to specific embodiments disclosed in these Examples, which are for purposes of illustration only, since alternative methods may be utilized to obtain similar results.

### P. Aeruginosa PAO1 Mutant

A mutant of P. aeruginosa PAO1 was constructed via deleting the PΔ2794 neuraminidase locus and testing its virulence and immunostimulatory capabilities in a mouse model of infection.

### Example 1

### Constriction of a P. aeruginosa PA1 Neurotraminidase Null Mutant

A nanA null mutant (Δ2794) was constructed by allelic replacement. An inframe non-polar deletion allele was constructed by removing the nanA coding sequence corresponding to amino acids 5-435 of the predicted 438-residue polypeptide and used to replace the full-length gene (1317 base pairs) by a method previously described (Wolfgang et al., (2003) Dev. Cell. 4:253-263). Primers were designed using the published DNA sequence for the neuraminidase gene (designated PΔ2794) from P. aeruginosa strain PAO1 (GenBank Accession # AF236853). A nanA complementation plasmid was constructed by cloning a PCR product corresponding to the full-length neuraminidase open reading frame into plasmid pMMBGW with either a gentamicin or penicillinase resistance marker (Wolfgang et al., (2003) Dev. Cell. 4:253-263). The complementation clone or an empty vector control was introduced into the Δ2794 mutant by conjugation and selection on gentamicin (40 μg/ml) (Invitrogen, Carlsbad, Calif.) or piperacillin (100 μg/ml) (Sigma-Aldrich, St. Louis, Mo.). The same procedure was carried out to generate a Δ2794 mutation in the P. aeruginosa strain PAK. The following primers were used for genotyping: SEQ ID NO: 3 is the forward primer, internal to PΔ2794; SEQ ID NO: 4 is the reverse primer, internal to PΔ2794; SEQ ID NO: 5 is the forward primer, external to PΔ2794; and SEQ ID NO: 6 is the reverse primer, external to PΔ2794.

The transmembrane (TM) region predictions depicted in FIGS. 1A-1C were made using the program TMPred (http://www.ch.embnet.org/software/TMPRED_form.html) and (TMAPhttp://bioweb.pasteur.fr/seqanal/interfaces/tmap.html). The autotransporter-like region shown in FIG. 1C was determined by the BLAST alignment with a putative autotransporter (MagnO3008734, GenBank Accession Number: ZP_{—}00054112) from the Magnetospirillum magnetotacticum genome.

Sequence predictions for the PΔ2794 locus were analyzed using ORFcurator (Rosenfeld et al., (2004) Bioinformatics 20:3462-3465).

### Example 2

### Bacterial Strains and Culture Conditions

The standard laboratory strain of P. aeruginosa PAO1 (Dr. Stephen Lory, Harvard University, Cambridge, Mass.) was used as a prototype, grown in Luria broth (LB) or M9 media with Mg-glu as indicated. For complementation studies, the PΔ2794 locus was overexpressed in E. coli using pMMB67EH.gm (ATCC, Manassas, Va.) and pMMB67EH.amp (ATCC, Manassas, Va.). Growth curves were obtained by growing bacteria in M9 media, with 40 μg/ml gentamicin or 100 μg/ml of piperacillin selection for strains containing plasmid, overnight to stationary phase then diluted 1:1000 in fresh media and incubated at 37° C. with shaking and OD₆₀₀ readings taken over time.

### Example 3

### Epithelial Cell Culture

1HAEo- and 16HBE cells originally obtained from Dieter Gruenert (California Pacific Medical Center Research Institute, San Francisco) were grown in Minimum Essential Medium (MEM) with Earle's salts supplemented with 10% fetal calf serum (Invitrogen, Carlsbad, Calif.) as has been previously described (Ratner et al., (2001) J. Biol. Chem. 276:19267-19275). RAW cells (Sigma-Aldrich, St. Louis, Mo.) were grown in RPMI Medium 1640 with 10% fetal calf serum.

### Example 4

### Biofilm Assays

An overnight culture of bacteria grown in LB with shaking was diluted 1:100, and 100 μl aliquots added to 96-well microtiter plates incubated for 24-48 hours at 37° C. (O'Toole et al., (2000) J. Bacteriol. 182:425-431; O'Toole et al., (2000) Annu. Rev. Microbiol. 54:49-79). 0.1% crystal violet was added to each well for 15 minutes, rinsed three times with water then released with the addition of 200 μl of 95% ethanol. Absorbance was determined at 540 nm. For experiments with inhibitors, the following modifications in the assay were used: for oseltamivir (Tamiflu) (Roche, Nutley, N.J.), an overnight culture of bacteria was diluted 1:100 in different doses of inhibitor in LB and 100 μl aliquots plated and assayed as above; for peramivir (BioCryst, Birmingham, Ala.), an overnight culture of bacteria was diluted 1:100 in different doses of inhibitor in LB and incubated at room temperature for 48 hours then diluted 1:2 in LB and 100 μl aliquots plated and assayed as above (McKimm-Breschkin et al., (2003) Antimicrob. Agents. Chemother. 47:2264-2272; Sidwell et al., (2002) Expert Opin. Investig. Drugs 11:859-869). Each sample was tested in sextuplicate and the assay was repeated on three separate occasions and representative data shown. A mean and standard deviation were calculated and statistical significance determined using a two-tailed unpaired t test (Graph Pad Instat version 3.0, Graphpad Software, San Diego, Calif.) to test the null hypothesis that there was no difference in the amount of biofilm production under each test condition, as compared to the media alone or untreated control.

For the complementation studies, all of the PAO1 strains were transformed with either the control vector or the vector expressing the 2794 locus. Flow cell experiments and confocal microscopy were performed as previously described (Boles et al., (2004) Proc. Natl. Acad. Sci. U.S.A. 101:16630-16635). For visualization by confocal microscopy, pMRP9-1 (which expresses green fluorescent protein) was transformed into appropriate strains (Davies et al., (1998) Science 280:295-298). The rotating disk reactor was used for generating biofilms for microscopy and quantitative counts (Singh et al., (2002) Nature 417:552-555). 1/100 strength TSB medium was used for these experiments.

### Example 5

### Neuraminidase Assays

The PAO1 enzyme (10 μg/ml) was overexpressed and purified from E. coli using the pET28a vector (Novagen, San Diego, Calif.) and a control V. cholera neuraminidase (0.1 U/ml) (Calbiochem, San Diego, Calif.) were incubated with the fluorescent substrate 2′-(4-methylumbelliferyl)-α-D-N-acetylneuraminic acid (Sigma-Aldrich, St. Louis, Mo.) (25 μM in 0.9% NaCl) with or without peramivir (0.25 and 2.5 μM) for 24-48 hours and fluorescence read at excitation 360 nm and emission 465 nm. Each data point was performed in sextuplicate. A mean and standard deviation were calculated and statistical significance determined using a one-way analysis of variance with Bonferroni's post test (Graph Pad Instat version 3.0, Graphpad Software, San Diego, Calif.) to test the null hypothesis that there was no difference in the amount of fluorescence under each test condition, as compared to the untreated control. A representative experiment is shown in FIG. 11C.

### Example 6

### IL-8 Assays

Confluent monolayers of 1HAEo-cells, weaned from serum overnight, were washed and stimulated with bacteria (1×10⁸ cfu/ml) for 30 minutes. Fresh media+gentamicin (100 μg/ml) (Invitrogen, Carlsbad, Calif.) was added and after 3 hours removed for chemokine analysis. ELISA for IL-8 (R&D Systems, Minneapolis, Minn.) was performed as previously described (Ratner et al., (2001) J. Biol. Chem. 276:19267-19275). Each data point was performed in quintuplicate and standardized by protein. A mean and standard deviation were calculated and statistical significance determined using a one-way analysis of variance with Bonferroni's post-test (Graph Pad Instat version 3.0, Graphpad Software, San Diego, Calif.) to test the null hypothesis that there was no difference in the amount of IL-8 under each test condition, as compared to the untreated control. Each experiment was done at least 3 times and a representative study is shown in FIG. 6B.

### Example 7

### Quantification of Epithelial Sialylation by Flow Cytometry

An assay that monitors the ability of culture supernatants to expose asialoGM1 from human airway epithelial cells was performed as previously described (Cacalano et al., (1992) J. Clin. Invest. 89:1866-1874). Briefly, 16HBE cells obtained from Dieter Gruenert (California Pacific Medical Center Research Institute, San Francisco) were grown in 24-well plates to confluence and exposed to bacterial supernatant concentrated 30-fold for 3-5 hours followed by 3 PBS washes. Cells were stained with rabbit polyclonal anti-asialoGM1 antibody (WAKO, Richmond, Va.) followed by donkey anti-rabbit IgG Alexa Fluor 488 (Molecular Probes, Carlsbad, Calif.). Cells detached from the plastic using 0.02% EGTA in HBSS were then fixed with 1% paraformaldehyde and analyzed on a FACSCalibur using Cell Quest software (Becton Dickinson, Franklin Lakes, N.J.).

### Example 8

### RAW Cell Binding and Phagocytosis as Determined by Flow Cytometry

RAW cells (Sigma-Aldrich, St. Louis, Mo.) were grown in 10 cm dishes and exposed to 1×10⁸ bacteria for 30 minutes at 37° C. After 4 washes with PBS, 2 ml HBSS+0.02% EGTA was added and cells harvested. Cells were counted in a hemacytometer and 1×10⁶ cells aliquoted per microfuge tube. 1 ml PBS was added to each tube and cells pelleted at 2,000 rpm×5 minutes. For extracellular binding determination, cells were incubated in 5% normal serum in PBS. For determination of total external and internalized bacteria, cells were incubated with Perm/Wash buffer (BD Biosciences Pharmingen, San Jose, Calif.). In both cases, cells were then stained with rabbit anti-OMP (outer membrane protein) antibody followed by donkey anti-rabbit Alexa Fluor 488 secondary (Molecular Probes, Carlsbad, Calif.), fixed with 1% paraformaldehyde, and analyzed on a FACSCalibur using Cell Quest software (Becton Dickinson, Franklin Lakes, N.J.).

### Example 9

### Mouse Models of Infection

7 day old BALB/c mice (The Jackson Laboratory, Bar Harbor, Me.) were inoculated intranasally with 2×10⁸ CFU of PAO1 or Δ2794 in 10 μl of PBS; or intraperitoneally with 5×10⁵ cfu of PAO1 or Δ2794 and euthanized 16 hours later with pentobarbital (Tang et al., (1996) Infect. Immun. 64:37-43). Pneumonia was defined as the recovery of more than 1000 CFU per lung, and bacteremia was defined as the recovery of bacteria from the spleen. The inflammatory response in vivo was assayed by flow cytometry as previously described (Gomez et al., (2004) Nat. Med. 10:842-848). Single cell suspensions of the lung were screened for the percentage of PMNs in the total leukocyte population by double staining with PE labeled anti-CD45 and FITC labeled anti-Ly6G antibodies (BD Biosciences Pharmingen, San Jose, Calif.). Irrelevant, isotype-matched antibodies were used as a control. Cells were gated on the basis of their forward and side scatter profiles and analyzed for the expression of both CD45 and Ly6G.

### Example 10

### Immunohistochemistry

Paraffin lung sections from mice infected with PAO1 and Δ2794 were hematoxylin-eosin stained (Molnar (1975) Histologic 5(1): 59; Molnar (1976) Histologic 6(4): 87).

### Example 11

### Real Time PCR

Lungs from PAO1 and Δ2794 inoculated mice were obtained at 16-18 hours post-inoculation and stored in RNAlater (Qiagen, Valencia, Calif.). RNA was isolated using the Qiagen RNeasy Mini Kit (Qiagen, Valencia, Calif.). cDNA was made from 1 μg of RNA using iScript synthesis kit (Bio-Rad, Hercules, Calif.). For quantitative real time PCR, amplification was done in a Light Cycler using the DNA Master SYBR Green I kit (Roche, Roche, Nutley, N.J.). The forward primer used for KC amplification was SEQ ID NO: 7. The reverse primer used for KC amplification was SEQ ID NO: 8. 35 cycles were run with denaturation at 95° C. for 8 seconds, amplification at 56° C. for 10 seconds and extension at 72° C. for 12 seconds. Actin was amplified on each individual sample and used as control for standardization. The forward primer used for actin amplification was SEQ ID NO: 9. The reverse primer used for actin amplification was SEQ ID NO: 10. 35 cycles were run with denaturation at 95° C. for 8 sec, amplification at 63° C. for 10 sec and extension at 72° C. for 12 sec.

### Example 12

### Adherence Assay

16HBE airway epithelial cells obtained from Dieter Gruenert (California Pacific Medical Center Research Institute, San Francisco) were stimulated with bacteria for 1 hour. After washing with PBS to remove unbound organisms, cells were stained with polyclonal anti-OMP (Biodesign, Saco, Me.) followed by Alexa Fluor 488-conjugated anti-rabbit IgG (Molecular Probes, Carlsbad, Calif.). Fixed cells were analyzed by flow cytometry to quantitate the number of bacteria bound to the surface.

### Example 13

### Crystallization of Pseudomonas Neuraminidase—Materials and Methods

Protein expression and purification. Residues 1-438 of wild-type Pseudomonas aeruginosa sialidase gene was sub-cloned into the pET28a vector (Novagen) and over-expressed in E. coli at 20° C. The expression construct contains an N-terminal hexa-histidine tag. The soluble protein was purified by nickel-agarose affinity chromatography, anion exchange and gel filtration chromatography. The protein was concentrated to 37 mg/ml in a solution containing 20 mM Tris (pH 8.5), 200 mM NaCl, flash-frozen in liquid nitrogen in the presence of 5% (v/v) glycerol, and stored at −80° C. The N-terminal His-tag was not removed for crystallization.

For the production of selenomethionyl proteins, the expression construct was transformed into B834 (DE3) cells (Novagen). The bacterial growth was carried out in defined LeMaster media supplemented with selenomethionine (Hendrickson et al., (1990) EMBO J. 9: 1665-1672), and the protein was purified following the same protocol as that for the native protein.

Protein Crystallization. Crystals of Pseudomonas aeruginosa sialidase free enzyme were obtained at 21° C. by the sitting-drop vapor diffusion method. The reservoir solution contained 100 mM Hepes (pH 7.0), 5% Tacsimate, 7% (w/v) PEG 5000MME, and the protein was at 10 mg/ml concentration. Selenomethionine-labeled protein was cross-microseeded with crystals from native protein to obtain adequate crystals. For data collection, the crystals were cryoprotected with the introduction of 20% (v/v) ethylene glycol, and flash-frozen in liquid nitrogen.

Data collection and processing. X-ray diffraction data were collected at the National Synchrotron Light Source (NSLS). A selenomethionyl single-wavelength anomalous diffraction (SAD) data set to 1.9 Å resolution was collected on an ADSC CCD at the X4A beamline, and a native data set to 1.6 Å resolution was collected on a Mar imaging plate at the X4C beamline. The diffraction images were processed and scaled with the HKL package (Otwinowski and Minor, (1997) Methods Enzymol. 276: 307-326). The crystals belong to space group P2₁3, with cell dimensions of a=b=c=125.6 Å. There is one molecule in the crystallographic asymmetric unit. A summary of the data is presented in Table 1.

Structure determination and refinement. The positions of 3 Se atoms were determined with the program SnB (Weeks and Miller, (1999) J. Appl. Crystallogr. 32: 120-124). Reflection phases to 1.9 Å resolution were calculated based on the SAD data and improved with the program SOLVE/RESOLVE (Terwilliger and Berendzen, (1999) Acta Crystallogr. D55: 849-861), which also automatically located 80% of the residues in the molecule. The atomic model was fit into the electron density with the program 0 (Jones et al., (1991) Acta Crystallogr. A47: 110-119). Further structure refinement to extend atomic model to 1.6 Å was carried out with the program CNS (Brunger et al., (1998) Acta Crystallogr. D54: 905-921).

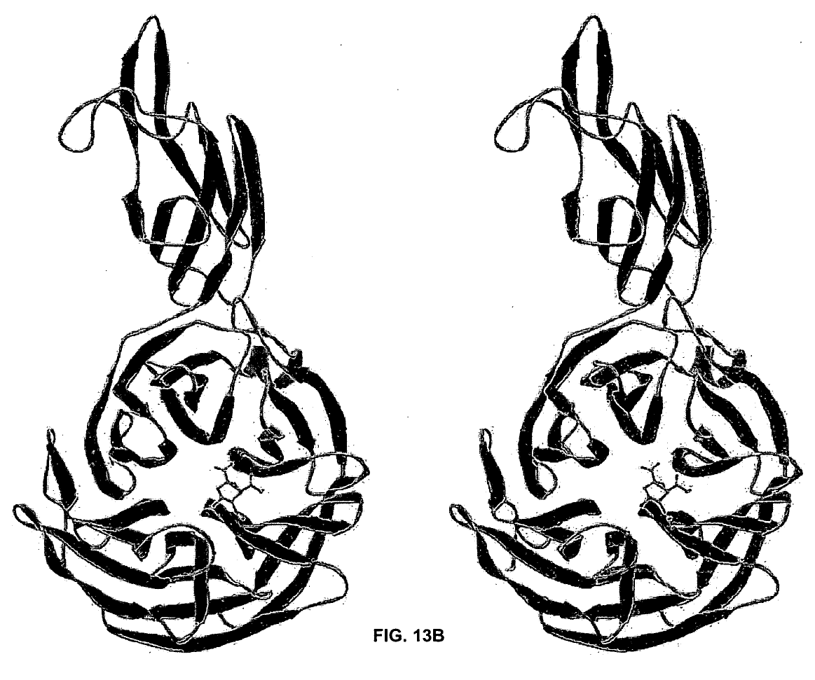

Representative models generated using the X-ray diffraction data are depicted in FIGS. 13A-B. An inhibitor shown in the PAO structure (right panel, FIG. 13A and FIG. 13B) was modeled into the structure to show where the binding site is located.

TABLE 1
Summary of Information from Crystallography Study

| Structure | Free enzyme (see Table 2) |
|---|---|
| Maximum resolution (Å) | 1.6 |
| Resolution range for refinement | 500-1.6 |
| Number of reflections | 87,941 |
| Completeness (%) | 99.5 |
| R factor² (%) | 18 |
| Free R factor² (%) | 20 |
| rms deviation in bond lengths (Å) | 0.005 |
| rms deviation in bond angles (°) | 1.4 |

TABLE 2
Atomic Coordinates for Residues of a Pseudomonas Neuraminidase Crystal

CRYST1 126.340 126.340 126.340 90.00 90.00 90.00 P 21 3

| | | | | | | | | | | | |
|---|---|---|---|---|---|---|---|---|---|---|---|
| ATOM | 1 | CB | MET | A | 1 | 102.189 | 32.906 | 25.057 | 1.00 | 39.36 | A |
| ATOM | 2 | CG | MET | A | 1 | 102.977 | 34.197 | 24.864 | 1.00 | 43.07 | A |
| ATOM | 3 | SD | MET | A | 1 | 102.117 | 35.409 | 23.832 | 1.00 | 48.81 | A |
| ATOM | 4 | CE | MET | A | 1 | 102.993 | 35.232 | 22.271 | 1.00 | 47.81 | A |
| ATOM | 5 | C | MET | A | 1 | 101.914 | 30.708 | 26.219 | 1.00 | 32.56 | A |
| ATOM | 6 | O | MET | A | 1 | 100.817 | 30.662 | 25.671 | 1.00 | 30.96 | A |
| ATOM | 7 | N | MET | A | 1 | 104.169 | 31.514 | 25.574 | 1.00 | 36.98 | A |
| ATOM | 8 | CA | MET | A | 1 | 102.819 | 31.921 | 26.051 | 1.00 | 35.19 | A |
| ATOM | 9 | N | ASN | A | 2 | 102.382 | 29.729 | 26.985 | 1.00 | 29.15 | A |
| ATOM | 10 | CA | ASN | A | 2 | 101.603 | 28.527 | 27.234 | 1.00 | 26.19 | A |
| ATOM | 11 | CB | ASN | A | 2 | 102.534 | 27.325 | 27.413 | 1.00 | 25.04 | A |
| ATOM | 12 | CG | ASN | A | 2 | 101.789 | 26.056 | 27.753 | 1.00 | 25.75 | A |
| ATOM | 13 | OD1 | ASN | A | 2 | 100.589 | 25.942 | 27.513 | 1.00 | 23.58 | A |
| ATOM | 14 | ND2 | ASN | A | 2 | 102.505 | 25.083 | 28.307 | 1.00 | 25.75 | A |
| ATOM | 15 | C | ASN | A | 2 | 100.752 | 28.737 | 28.479 | 1.00 | 24.33 | A |
| ATOM | 16 | O | ASN | A | 2 | 101.217 | 28.531 | 29.599 | 1.00 | 22.66 | A |
| ATOM | 17 | N | THR | A | 3 | 99.510 | 29.165 | 28.275 | 1.00 | 21.62 | A |
| ATOM | 18 | CA | THR | A | 3 | 98.602 | 29.418 | 29.385 | 1.00 | 19.44 | A |
| ATOM | 19 | CB | THR | A | 3 | 97.347 | 30.200 | 28.920 | 1.00 | 19.99 | A |
| ATOM | 20 | OG1 | THR | A | 3 | 96.618 | 29.421 | 27.964 | 1.00 | 20.10 | A |
| ATOM | 21 | CG2 | THR | A | 3 | 97.752 | 31.526 | 28.280 | 1.00 | 20.15 | A |
| ATOM | 22 | C | THR | A | 3 | 98.158 | 28.125 | 30.061 | 1.00 | 18.28 | A |
| ATOM | 23 | O | THR | A | 3 | 97.513 | 28.156 | 31.111 | 1.00 | 17.31 | A |
| ATOM | 24 | N | TYR | A | 4 | 98.503 | 26.988 | 29.464 | 1.00 | 17.70 | A |
| ATOM | 25 | CA | TYR | A | 4 | 98.135 | 25.698 | 30.040 | 1.00 | 17.22 | A |
| ATOM | 26 | CB | TYR | A | 4 | 98.002 | 24.630 | 28.949 | 1.00 | 17.29 | A |
| ATOM | 27 | CG | TYR | A | 4 | 96.886 | 24.900 | 27.973 | 1.00 | 15.54 | A |
| ATOM | 28 | CD1 | TYR | A | 4 | 97.123 | 25.592 | 26.787 | 1.00 | 17.33 | A |
| ATOM | 29 | CE1 | TYR | A | 4 | 96.090 | 25.864 | 25.895 | 1.00 | 16.95 | A |
| ATOM | 30 | CD2 | TYR | A | 4 | 95.585 | 24.485 | 28.246 | 1.00 | 15.64 | A |
| ATOM | 31 | CE2 | TYR | A | 4 | 94.546 | 24.752 | 27.364 | 1.00 | 15.85 | A |
| ATOM | 32 | CZ | TYR | A | 4 | 94.804 | 25.440 | 26.193 | 1.00 | 16.23 | A |
| ATOM | 33 | OH | TYR | A | 4 | 93.778 | 25.708 | 25.319 | 1.00 | 16.16 | A |
| ATOM | 34 | C | TYR | A | 4 | 99.151 | 25.228 | 31.070 | 1.00 | 18.11 | A |
| ATOM | 35 | O | TYR | A | 4 | 98.851 | 24.362 | 31.893 | 1.00 | 18.63 | A |
| ATOM | 36 | N | PHE | A | 5 | 100.350 | 25.801 | 31.026 | 1.00 | 18.15 | A |
| ATOM | 37 | CA | PHE | A | 5 | 101.394 | 25.415 | 31.965 | 1.00 | 19.00 | A |
| ATOM | 38 | CB | PHE | A | 5 | 102.736 | 26.032 | 31.567 | 1.00 | 18.32 | A |
| ATOM | 39 | CG | PHE | A | 5 | 103.919 | 25.304 | 32.137 | 1.00 | 17.83 | A |
| ATOM | 40 | CD1 | PHE | A | 5 | 104.250 | 25.429 | 33.483 | 1.00 | 17.98 | A |
| ATOM | 41 | CD2 | PHE | A | 5 | 104.668 | 24.442 | 31.340 | 1.00 | 16.90 | A |
| ATOM | 42 | CE1 | PHE | A | 5 | 105.306 | 24.704 | 34.028 | 1.00 | 17.01 | A |
| ATOM | 43 | CE2 | PHE | A | 5 | 105.725 | 23.713 | 31.876 | 1.00 | 17.16 | A |
| ATOM | 44 | CZ | PHE | A | 5 | 106.045 | 23.842 | 33.223 | 1.00 | 18.50 | A |
| ATOM | 45 | C | PHE | A | 5 | 101.005 | 25.865 | 33.364 | 1.00 | 20.19 | A |
| ATOM | 46 | O | PHE | A | 5 | 100.812 | 27.053 | 33.615 | 1.00 | 21.90 | A |
| ATOM | 47 | N | ASP | A | 6 | 100.897 | 24.907 | 34.274 | 1.00 | 19.85 | A |
| ATOM | 48 | CA | ASP | A | 6 | 100.503 | 25.202 | 35.641 | 1.00 | 19.34 | A |
| ATOM | 49 | CB | ASP | A | 6 | 99.446 | 24.186 | 36.094 | 1.00 | 21.28 | A |
| ATOM | 50 | CG | ASP | A | 6 | 98.907 | 24.478 | 37.478 | 1.00 | 23.38 | A |
| ATOM | 51 | OD1 | ASP | A | 6 | 99.583 | 24.136 | 38.474 | 1.00 | 22.67 | A |
| ATOM | 52 | OD2 | ASP | A | 6 | 97.803 | 25.054 | 37.569 | 1.00 | 25.33 | A |
| ATOM | 53 | C | ASP | A | 6 | 101.667 | 25.216 | 36.624 | 1.00 | 18.33 | A |
| ATOM | 54 | O | ASP | A | 6 | 102.524 | 24.334 | 36.605 | 1.00 | 18.94 | A |
| ATOM | 55 | N | ILE | A | 7 | 101.698 | 26.244 | 37.465 | 1.00 | 15.46 | A |
| ATOM | 56 | CA | ILE | A | 7 | 102.720 | 26.369 | 38.496 | 1.00 | 14.12 | A |
| ATOM | 57 | CB | ILE | A | 7 | 103.398 | 27.757 | 38.488 | 1.00 | 14.23 | A |
| ATOM | 58 | CG2 | ILE | A | 7 | 104.370 | 27.862 | 39.661 | 1.00 | 15.94 | A |
| ATOM | 59 | CG1 | ILE | A | 7 | 104.128 | 27.986 | 37.158 | 1.00 | 16.79 | A |
| ATOM | 60 | CD1 | ILE | A | 7 | 105.294 | 27.046 | 36.917 | 1.00 | 18.08 | A |
| ATOM | 61 | C | ILE | A | 7 | 101.963 | 26.212 | 39.808 | 1.00 | 11.97 | A |
| ATOM | 62 | O | ILE | A | 7 | 101.233 | 27.116 | 40.226 | 1.00 | 13.17 | A |
| ATOM | 63 | N | PRO | A | 8 | 102.112 | 25.056 | 40.469 | 1.00 | 10.85 | A |
| ATOM | 64 | CD | PRO | A | 8 | 102.896 | 23.871 | 40.074 | 1.00 | 11.90 | A |
| ATOM | 65 | CA | PRO | A | 8 | 101.418 | 24.822 | 41.736 | 1.00 | 10.56 | A |
| ATOM | 66 | CB | PRO | A | 8 | 101.491 | 23.310 | 41.880 | 1.00 | 11.45 | A |
| ATOM | 67 | CG | PRO | A | 8 | 102.855 | 23.016 | 41.330 | 1.00 | 11.97 | A |
| ATOM | 68 | C | PRO | A | 8 | 102.094 | 25.538 | 42.895 | 1.00 | 10.61 | A |
| ATOM | 69 | O | PRO | A | 8 | 103.199 | 26.059 | 42.757 | 1.00 | 10.25 | A |
| ATOM | 70 | N | HIS | A | 9 | 101.417 | 25.565 | 44.036 | 1.00 | 8.51 | A |
| ATOM | 71 | CA | HIS | A | 9 | 101.968 | 26.184 | 45.231 | 1.00 | 8.65 | A |
| ATOM | 72 | CB | HIS | A | 9 | 101.404 | 27.595 | 45.414 | 1.00 | 9.00 | A |
| ATOM | 73 | CG | HIS | A | 9 | 101.988 | 28.586 | 44.464 | 1.00 | 9.28 | A |
| ATOM | 74 | CD2 | HIS | A | 9 | 101.471 | 29.177 | 43.362 | 1.00 | 10.02 | A |
| ATOM | 75 | ND1 | HIS | A | 9 | 103.310 | 28.969 | 44.523 | 1.00 | 9.46 | A |
| ATOM | 76 | CE1 | HIS | A | 9 | 103.585 | 29.745 | 43.491 | 1.00 | 9.70 | A |
| ATOM | 77 | NE2 | HIS | A | 9 | 102.487 | 29.886 | 42.771 | 1.00 | 11.01 | A |
| ATOM | 78 | C | HIS | A | 9 | 101.669 | 25.343 | 46.446 | 1.00 | 9.39 | A |
| ATOM | 79 | O | HIS | A | 9 | 100.676 | 24.617 | 46.484 | 1.00 | 8.54 | A |
| ATOM | 80 | N | ARG | A | 10 | 102.564 | 25.411 | 47.420 | 1.00 | 8.41 | A |
| ATOM | 81 | CA | ARG | A | 10 | 102.398 | 24.686 | 48.668 | 1.00 | 8.76 | A |
| ATOM | 82 | CB | ARG | A | 10 | 103.185 | 23.377 | 48.660 | 1.00 | 9.99 | A |
| ATOM | 83 | CG | ARG | A | 10 | 103.062 | 22.609 | 49.961 | 1.00 | 13.28 | A |
| ATOM | 84 | CD | ARG | A | 10 | 103.738 | 21.256 | 49.875 | 1.00 | 16.34 | A |
| ATOM | 85 | NE | ARG | A | 10 | 104.150 | 20.769 | 51.183 | 1.00 | 23.26 | A |
| ATOM | 86 | CZ | ARG | A | 10 | 103.329 | 20.287 | 52.107 | 1.00 | 24.84 | A |
| ATOM | 87 | NH1 | ARG | A | 10 | 102.024 | 20.212 | 51.875 | 1.00 | 26.98 | A |
| ATOM | 88 | NH2 | ARG | A | 10 | 103.819 | 19.882 | 53.275 | 1.00 | 27.52 | A |
| ATOM | 89 | C | ARG | A | 10 | 102.920 | 25.602 | 49.754 | 1.00 | 10.25 | A |
| ATOM | 90 | O | ARG | A | 10 | 104.128 | 25.800 | 49.889 | 1.00 | 11.14 | A |
| ATOM | 91 | N | LEU | A | 11 | 101.997 | 26.169 | 50.521 | 1.00 | 9.65 | A |
| ATOM | 92 | CA | LEU | A | 11 | 102.359 | 27.097 | 51.578 | 1.00 | 8.95 | A |
| ATOM | 93 | CB | LEU | A | 11 | 101.469 | 28.337 | 51.489 | 1.00 | 8.16 | A |
| ATOM | 94 | CG | LEU | A | 11 | 101.700 | 29.186 | 50.232 | 1.00 | 8.68 | A |
| ATOM | 95 | CD1 | LEU | A | 11 | 100.800 | 30.420 | 50.266 | 1.00 | 8.97 | A |
| ATOM | 96 | CD2 | LEU | A | 11 | 103.165 | 29.611 | 50.176 | 1.00 | 9.66 | A |
| ATOM | 97 | C | LEU | A | 11 | 102.279 | 26.501 | 52.973 | 1.00 | 9.88 | A |
| ATOM | 98 | O | LEU | A | 11 | 102.551 | 27.183 | 53.961 | 1.00 | 9.80 | A |
| ATOM | 99 | N | VAL | A | 12 | 101.911 | 25.228 | 53.054 | 1.00 | 9.93 | A |
| ATOM | 100 | CA | VAL | A | 12 | 101.804 | 24.543 | 54.335 | 1.00 | 10.32 | A |
| ATOM | 101 | CB | VAL | A | 12 | 101.405 | 23.065 | 54.132 | 1.00 | 10.86 | A |
| ATOM | 102 | CG1 | VAL | A | 12 | 101.251 | 22.369 | 55.483 | 1.00 | 11.76 | A |
| ATOM | 103 | CG2 | VAL | A | 12 | 100.099 | 22.998 | 53.352 | 1.00 | 12.57 | A |
| ATOM | 104 | C | VAL | A | 12 | 103.119 | 24.602 | 55.111 | 1.00 | 10.97 | A |
| ATOM | 105 | O | VAL | A | 12 | 104.151 | 24.128 | 54.636 | 1.00 | 12.31 | A |
| ATOM | 106 | N | GLY | A | 13 | 103.069 | 25.204 | 56.297 | 1.00 | 10.64 | A |
| ATOM | 107 | CA | GLY | A | 13 | 104.244 | 25.310 | 57.145 | 1.00 | 10.33 | A |
| ATOM | 108 | C | GLY | A | 13 | 105.311 | 26.305 | 56.722 | 1.00 | 11.26 | A |
| ATOM | 109 | O | GLY | A | 13 | 106.355 | 26.398 | 57.371 | 1.00 | 12.73 | A |
| ATOM | 110 | N | LYS | A | 14 | 105.066 | 27.057 | 55.653 | 1.00 | 10.46 | A |
| ATOM | 111 | CA | LYS | A | 14 | 106.049 | 28.024 | 55.173 | 1.00 | 9.67 | A |
| ATOM | 112 | CB | LYS | A | 14 | 105.798 | 28.338 | 53.696 | 1.00 | 10.49 | A |
| ATOM | 113 | CG | LYS | A | 14 | 106.156 | 27.192 | 52.776 | 1.00 | 12.09 | A |
| ATOM | 114 | CD | LYS | A | 14 | 107.641 | 26.885 | 52.877 | 1.00 | 14.65 | A |
| ATOM | 115 | CE | LYS | A | 14 | 108.039 | 25.720 | 51.996 | 1.00 | 15.56 | A |
| ATOM | 116 | NZ | LYS | A | 14 | 109.482 | 25.402 | 52.188 | 1.00 | 16.18 | A |
| ATOM | 117 | C | LYS | A | 14 | 106.049 | 29.311 | 55.985 | 1.00 | 9.01 | A |
| ATOM | 118 | O | LYS | A | 14 | 105.017 | 29.724 | 56.512 | 1.00 | 9.86 | A |
| ATOM | 119 | N | ALA | A | 15 | 107.217 | 29.939 | 56.093 | 1.00 | 9.11 | A |
| ATOM | 120 | CA | ALA | A | 15 | 107.337 | 31.181 | 56.844 | 1.00 | 8.97 | A |
| ATOM | 121 | CB | ALA | A | 15 | 107.380 | 30.883 | 58.341 | 1.00 | 10.44 | A |
| ATOM | 122 | C | ALA | A | 15 | 108.562 | 31.988 | 56.457 | 1.00 | 9.34 | A |
| ATOM | 123 | O | ALA | A | 15 | 109.564 | 31.451 | 55.978 | 1.00 | 9.71 | A |
| ATOM | 124 | N | LEU | A | 16 | 108.454 | 33.294 | 56.666 | 1.00 | 9.80 | A |
| ATOM | 125 | CA | LEU | A | 16 | 109.545 | 34.219 | 56.411 | 1.00 | 9.76 | A |
| ATOM | 126 | CB | LEU | A | 16 | 109.116 | 35.337 | 55.452 | 1.00 | 10.12 | A |
| ATOM | 127 | CG | LEU | A | 16 | 110.255 | 36.221 | 54.914 | 1.00 | 10.37 | A |
| ATOM | 128 | CD1 | LEU | A | 16 | 109.751 | 37.105 | 53.779 | 1.00 | 9.46 | A |
| ATOM | 129 | CD2 | LEU | A | 16 | 110.824 | 37.076 | 56.043 | 1.00 | 12.55 | A |
| ATOM | 130 | C | LEU | A | 16 | 109.830 | 34.804 | 57.787 | 1.00 | 10.12 | A |
| ATOM | 131 | O | LEU | A | 16 | 109.036 | 35.582 | 58.310 | 1.00 | 10.92 | A |
| ATOM | 132 | N | TYR | A | 17 | 110.945 | 34.405 | 58.387 | 1.00 | 10.86 | A |
| ATOM | 133 | CA | TYR | A | 17 | 111.310 | 34.913 | 59.703 | 1.00 | 10.91 | A |
| ATOM | 134 | CB | TYR | A | 17 | 110.834 | 33.967 | 60.807 | 1.00 | 11.86 | A |
| ATOM | 135 | CG | TYR | A | 17 | 111.111 | 34.510 | 62.193 | 1.00 | 12.76 | A |
| ATOM | 136 | CD1 | TYR | A | 17 | 110.382 | 35.588 | 62.699 | 1.00 | 13.33 | A |
| ATOM | 137 | CE1 | TYR | A | 17 | 110.659 | 36.123 | 63.957 | 1.00 | 13.76 | A |
| ATOM | 138 | CD2 | TYR | A | 17 | 112.128 | 33.976 | 62.980 | 1.00 | 13.44 | A |
| ATOM | 139 | CE2 | TYR | A | 17 | 112.416 | 34.504 | 64.238 | 1.00 | 13.28 | A |
| ATOM | 140 | CZ | TYR | A | 17 | 111.679 | 35.574 | 64.720 | 1.00 | 13.77 | A |
| ATOM | 141 | OH | TYR | A | 17 | 111.954 | 36.093 | 65.962 | 1.00 | 14.00 | A |
| ATOM | 142 | C | TYR | A | 17 | 112.818 | 35.075 | 59.791 | 1.00 | 12.72 | A |
| ATOM | 143 | O | TYR | A | 17 | 113.566 | 34.110 | 59.627 | 1.00 | 13.04 | A |
| ATOM | 144 | N | GLU | A | 18 | 113.257 | 36.303 | 60.052 | 1.00 | 11.89 | A |
| ATOM | 145 | CA | GLU | A | 18 | 114.681 | 36.598 | 60.155 | 1.00 | 12.82 | A |
| ATOM | 146 | CB | GLU | A | 18 | 115.164 | 37.328 | 58.897 | 1.00 | 14.15 | A |
| ATOM | 147 | CG | GLU | A | 18 | 114.896 | 36.583 | 57.588 | 1.00 | 18.18 | A |
| ATOM | 148 | CD | GLU | A | 18 | 115.523 | 37.274 | 56.386 | 1.00 | 20.81 | A |
| ATOM | 149 | OE1 | GLU | A | 18 | 116.754 | 37.139 | 56.193 | 1.00 | 24.65 | A |
| ATOM | 150 | OE2 | GLU | A | 18 | 114.794 | 37.955 | 55.635 | 1.00 | 21.41 | A |
| ATOM | 151 | C | GLU | A | 18 | 114.986 | 37.436 | 61.392 | 1.00 | 12.64 | A |
| ATOM | 152 | O | GLU | A | 18 | 115.973 | 38.169 | 61.426 | 1.00 | 12.69 | A |
| ATOM | 153 | N | SER | A | 19 | 114.130 | 37.319 | 62.401 | 1.00 | 11.40 | A |
| ATOM | 154 | CA | SER | A | 19 | 114.290 | 38.034 | 63.665 | 1.00 | 10.90 | A |
| ATOM | 155 | CB | SER | A | 19 | 115.651 | 37.712 | 64.284 | 1.00 | 13.30 | A |
| ATOM | 156 | OG | SER | A | 19 | 115.718 | 36.339 | 64.639 | 1.00 | 18.47 | A |
| ATOM | 157 | C | SER | A | 19 | 114.106 | 39.545 | 63.604 | 1.00 | 10.80 | A |
| ATOM | 158 | O | SER | A | 19 | 114.594 | 40.269 | 64.470 | 1.00 | 11.14 | A |
| ATOM | 159 | N | TYR | A | 20 | 113.418 | 40.022 | 62.573 | 1.00 | 9.82 | A |
| ATOM | 160 | CA | TYR | A | 20 | 113.127 | 41.450 | 62.452 | 1.00 | 8.80 | A |
| ATOM | 161 | CB | TYR | A | 20 | 113.182 | 41.913 | 60.989 | 1.00 | 9.02 | A |
| ATOM | 162 | CG | TYR | A | 20 | 114.574 | 42.093 | 60.438 | 1.00 | 7.73 | A |
| ATOM | 163 | CD1 | TYR | A | 20 | 115.208 | 41.067 | 59.742 | 1.00 | 8.09 | A |
| ATOM | 164 | CE1 | TYR | A | 20 | 116.511 | 41.217 | 59.268 | 1.00 | 9.34 | A |
| ATOM | 165 | CD2 | TYR | A | 20 | 115.272 | 43.282 | 60.647 | 1.00 | 7.92 | A |
| ATOM | 166 | CE2 | TYR | A | 20 | 116.573 | 43.444 | 60.182 | 1.00 | 8.90 | A |
| ATOM | 167 | CZ | TYR | A | 20 | 117.188 | 42.408 | 59.496 | 1.00 | 9.33 | A |
| ATOM | 168 | OH | TYR | A | 20 | 118.486 | 42.553 | 59.062 | 1.00 | 10.07 | A |
| ATOM | 169 | C | TYR | A | 20 | 111.691 | 41.588 | 62.914 | 1.00 | 9.15 | A |
| ATOM | 170 | O | TYR | A | 20 | 111.089 | 40.621 | 63.377 | 1.00 | 9.54 | A |
| ATOM | 171 | N | TYR | A | 21 | 111.153 | 42.797 | 62.815 | 1.00 | 8.83 | A |
| ATOM | 172 | CA | TYR | A | 21 | 109.749 | 42.989 | 63.111 | 1.00 | 8.48 | A |
| ATOM | 173 | CB | TYR | A | 21 | 109.466 | 44.235 | 63.966 | 1.00 | 8.03 | A |
| ATOM | 174 | CG | TYR | A | 21 | 108.060 | 44.207 | 64.543 | 1.00 | 8.63 | A |
| ATOM | 175 | CD1 | TYR | A | 21 | 107.775 | 43.485 | 65.707 | 1.00 | 9.47 | A |
| ATOM | 176 | CE1 | TYR | A | 21 | 106.460 | 43.350 | 66.176 | 1.00 | 9.39 | A |
| ATOM | 177 | CD2 | TYR | A | 21 | 106.991 | 44.801 | 63.864 | 1.00 | 8.64 | A |
| ATOM | 178 | CE2 | TYR | A | 21 | 105.676 | 44.669 | 64.323 | 1.00 | 8.46 | A |
| ATOM | 179 | CZ | TYR | A | 21 | 105.419 | 43.944 | 65.476 | 1.00 | 8.52 | A |
| ATOM | 180 | OH | TYR | A | 21 | 104.128 | 43.805 | 65.938 | 1.00 | 10.50 | A |
| ATOM | 181 | C | TYR | A | 21 | 109.205 | 43.195 | 61.703 | 1.00 | 8.38 | A |
| ATOM | 182 | O | TYR | A | 21 | 109.186 | 44.316 | 61.182 | 1.00 | 10.09 | A |
| ATOM | 183 | N | ASP | A | 22 | 108.835 | 42.087 | 61.067 | 1.00 | 8.92 | A |
| ATOM | 184 | CA | ASP | A | 22 | 108.276 | 42.096 | 59.713 | 1.00 | 8.89 | A |
| ATOM | 185 | CB | ASP | A | 22 | 108.817 | 40.910 | 58.911 | 1.00 | 9.49 | A |
| ATOM | 186 | CG | ASP | A | 22 | 110.296 | 41.055 | 58.583 | 1.00 | 8.99 | A |
| ATOM | 187 | OD1 | ASP | A | 22 | 110.745 | 42.196 | 58.320 | 1.00 | 8.62 | A |
| ATOM | 188 | OD2 | ASP | A | 22 | 110.999 | 40.024 | 58.565 | 1.00 | 9.51 | A |
| ATOM | 189 | C | ASP | A | 22 | 106.765 | 41.986 | 59.886 | 1.00 | 8.83 | A |
| ATOM | 190 | O | ASP | A | 22 | 106.278 | 41.071 | 60.549 | 1.00 | 9.47 | A |
| ATOM | 191 | N | HIS | A | 23 | 106.017 | 42.900 | 59.274 | 1.00 | 8.00 | A |
| ATOM | 192 | CA | HIS | A | 23 | 104.571 | 42.904 | 59.480 | 1.00 | 7.50 | A |
| ATOM | 193 | CB | HIS | A | 23 | 104.313 | 43.390 | 60.910 | 1.00 | 8.22 | A |
| ATOM | 194 | CG | HIS | A | 23 | 103.147 | 42.746 | 61.593 | 1.00 | 8.86 | A |
| ATOM | 195 | CD2 | HIS | A | 23 | 102.984 | 42.354 | 62.877 | 1.00 | 10.76 | A |
| ATOM | 196 | ND1 | HIS | A | 23 | 101.935 | 42.537 | 60.970 | 1.00 | 9.80 | A |
| ATOM | 197 | CE1 | HIS | A | 23 | 101.074 | 42.048 | 61.844 | 1.00 | 9.84 | A |
| ATOM | 198 | NE2 | HIS | A | 23 | 101.683 | 41.929 | 63.010 | 1.00 | 11.30 | A |
| ATOM | 199 | C | HIS | A | 23 | 103.826 | 43.834 | 58.527 | 1.00 | 8.65 | A |
| ATOM | 200 | O | HIS | A | 23 | 104.424 | 44.477 | 57.670 | 1.00 | 8.82 | A |
| ATOM | 201 | N | PHE | A | 24 | 102.508 | 43.882 | 58.707 | 1.00 | 9.05 | A |
| ATOM | 202 | CA | PHE | A | 24 | 101.607 | 44.766 | 57.971 | 1.00 | 8.96 | A |
| ATOM | 203 | CB | PHE | A | 24 | 101.785 | 46.197 | 58.492 | 1.00 | 9.75 | A |
| ATOM | 204 | CG | PHE | A | 24 | 102.021 | 46.279 | 59.976 | 1.00 | 11.69 | A |
| ATOM | 205 | CD1 | PHE | A | 24 | 103.132 | 46.954 | 60.469 | 1.00 | 11.28 | A |
| ATOM | 206 | CD2 | PHE | A | 24 | 101.133 | 45.700 | 60.877 | 1.00 | 10.69 | A |
| ATOM | 207 | CE1 | PHE | A | 24 | 103.363 | 47.051 | 61.834 | 1.00 | 11.72 | A |
| ATOM | 208 | CE2 | PHE | A | 24 | 101.352 | 45.791 | 62.252 | 1.00 | 12.58 | A |
| ATOM | 209 | CZ | PHE | A | 24 | 102.471 | 46.472 | 62.729 | 1.00 | 11.92 | A |
| ATOM | 210 | C | PHE | A | 24 | 101.723 | 44.783 | 56.450 | 1.00 | 9.83 | A |
| ATOM | 211 | O | PHE | A | 24 | 101.564 | 45.835 | 55.829 | 1.00 | 11.34 | A |
| ATOM | 212 | N | GLY | A | 25 | 101.957 | 43.622 | 55.846 | 1.00 | 8.09 | A |
| ATOM | 213 | CA | GLY | A | 25 | 102.103 | 43.579 | 54.403 | 1.00 | 8.55 | A |
| ATOM | 214 | C | GLY | A | 25 | 101.067 | 42.766 | 53.652 | 1.00 | 8.75 | A |
| ATOM | 215 | O | GLY | A | 25 | 100.032 | 42.367 | 54.197 | 1.00 | 8.65 | A |
| ATOM | 216 | N | GLN | A | 26 | 101.375 | 42.510 | 52.386 | 1.00 | 8.19 | A |
| ATOM | 217 | CA | GLN | A | 26 | 100.500 | 41.761 | 51.494 | 1.00 | 8.57 | A |
| ATOM | 218 | CB | GLN | A | 26 | 99.869 | 42.712 | 50.475 | 1.00 | 9.10 | A |
| ATOM | 219 | CG | GLN | A | 26 | 99.017 | 43.816 | 51.055 | 1.00 | 8.68 | A |
| ATOM | 220 | CD | GLN | A | 26 | 97.770 | 43.272 | 51.723 | 1.00 | 10.22 | A |
| ATOM | 221 | OE1 | GLN | A | 26 | 97.165 | 42.303 | 51.243 | 1.00 | 9.80 | A |
| ATOM | 222 | NE2 | GLN | A | 26 | 97.368 | 43.896 | 52.828 | 1.00 | 9.60 | A |
| ATOM | 223 | C | GLN | A | 26 | 101.244 | 40.696 | 50.698 | 1.00 | 8.79 | A |
| ATOM | 224 | O | GLN | A | 26 | 102.391 | 40.894 | 50.302 | 1.00 | 8.80 | A |
| ATOM | 225 | N | MET | A | 27 | 100.583 | 39.570 | 50.465 | 1.00 | 8.05 | A |
| ATOM | 226 | CA | MET | A | 27 | 101.131 | 38.521 | 49.621 | 1.00 | 8.72 | A |
| ATOM | 227 | CB | MET | A | 27 | 100.956 | 37.152 | 50.266 | 1.00 | 8.47 | A |
| ATOM | 228 | CG | MET | A | 27 | 101.468 | 35.987 | 49.375 | 1.00 | 8.31 | A |
| ATOM | 229 | SD | MET | A | 27 | 101.373 | 34.345 | 50.101 | 1.00 | 10.08 | A |
| ATOM | 230 | CE | MET | A | 27 | 99.578 | 34.059 | 50.098 | 1.00 | 11.88 | A |
| ATOM | 231 | C | MET | A | 27 | 100.316 | 38.533 | 48.324 | 1.00 | 8.74 | A |
| ATOM | 232 | O | MET | A | 27 | 99.107 | 38.746 | 48.356 | 1.00 | 8.76 | A |
| ATOM | 233 | N | ASP | A | 28 | 100.977 | 38.314 | 47.193 | 1.00 | 8.83 | A |
| ATOM | 234 | CA | ASP | A | 28 | 100.282 | 38.242 | 45.910 | 1.00 | 9.48 | A |
| ATOM | 235 | CB | ASP | A | 28 | 100.076 | 39.625 | 45.274 | 1.00 | 10.08 | A |
| ATOM | 236 | CG | ASP | A | 28 | 98.780 | 39.707 | 44.463 | 1.00 | 11.04 | A |
| ATOM | 237 | OD1 | ASP | A | 28 | 98.334 | 38.666 | 43.934 | 1.00 | 11.69 | A |
| ATOM | 238 | OD2 | ASP | A | 28 | 98.190 | 40.808 | 44.350 | 1.00 | 12.11 | A |
| ATOM | 239 | C | ASP | A | 28 | 101.133 | 37.370 | 44.999 | 1.00 | 10.56 | A |
| ATOM | 240 | O | ASP | A | 28 | 102.149 | 36.827 | 45.428 | 1.00 | 10.43 | A |
| ATOM | 241 | N | ILE | A | 29 | 100.726 | 37.243 | 43.743 | 1.00 | 10.48 | A |
| ATOM | 242 | CA | ILE | A | 29 | 101.443 | 36.395 | 42.807 | 1.00 | 11.32 | A |
| ATOM | 243 | CB | ILE | A | 29 | 100.528 | 35.213 | 42.392 | 1.00 | 10.88 | A |
| ATOM | 244 | CG2 | ILE | A | 29 | 99.394 | 35.729 | 41.532 | 1.00 | 11.58 | A |
| ATOM | 245 | CG1 | ILE | A | 29 | 101.329 | 34.128 | 41.671 | 1.00 | 10.72 | A |
| ATOM | 246 | CD1 | ILE | A | 29 | 100.560 | 32.807 | 41.523 | 1.00 | 12.03 | A |
| ATOM | 247 | C | ILE | A | 29 | 101.927 | 37.171 | 41.584 | 1.00 | 10.71 | A |
| ATOM | 248 | O | ILE | A | 29 | 101.275 | 38.114 | 41.134 | 1.00 | 11.53 | A |
| ATOM | 249 | N | LEU | A | 30 | 103.091 | 36.780 | 41.072 | 1.00 | 12.29 | A |
| ATOM | 250 | CA | LEU | A | 30 | 103.676 | 37.411 | 39.892 | 1.00 | 12.96 | A |
| ATOM | 251 | CB | LEU | A | 30 | 105.196 | 37.232 | 39.888 | 1.00 | 13.81 | A |
| ATOM | 252 | CG | LEU | A | 30 | 105.966 | 37.913 | 41.022 | 1.00 | 16.02 | A |
| ATOM | 253 | CD1 | LEU | A | 30 | 107.422 | 37.466 | 41.016 | 1.00 | 16.66 | A |
| ATOM | 254 | CD2 | LEU | A | 30 | 105.858 | 39.413 | 40.859 | 1.00 | 16.93 | A |
| ATOM | 255 | C | LEU | A | 30 | 103.102 | 36.756 | 38.646 | 1.00 | 13.94 | A |
| ATOM | 256 | O | LEU | A | 30 | 102.513 | 35.678 | 38.717 | 1.00 | 14.27 | A |
| ATOM | 257 | N | SER | A | 31 | 103.286 | 37.401 | 37.501 | 1.00 | 15.27 | A |
| ATOM | 258 | CA | SER | A | 31 | 102.787 | 36.862 | 36.248 | 1.00 | 15.55 | A |
| ATOM | 259 | CB | SER | A | 31 | 103.075 | 37.835 | 35.103 | 1.00 | 16.73 | A |
| ATOM | 260 | OG | SER | A | 31 | 102.329 | 39.025 | 35.259 | 1.00 | 19.23 | A |
| ATOM | 261 | C | SER | A | 31 | 103.406 | 35.506 | 35.939 | 1.00 | 16.05 | A |
| ATOM | 262 | O | SER | A | 31 | 102.784 | 34.678 | 35.273 | 1.00 | 17.05 | A |
| ATOM | 263 | N | ASP | A | 32 | 104.622 | 35.270 | 36.426 | 1.00 | 15.81 | A |
| ATOM | 264 | CA | ASP | A | 32 | 105.292 | 33.998 | 36.164 | 1.00 | 16.76 | A |
| ATOM | 265 | CB | ASP | A | 32 | 106.822 | 34.157 | 36.183 | 1.00 | 17.07 | A |
| ATOM | 266 | CG | ASP | A | 32 | 107.365 | 34.590 | 37.532 | 1.00 | 20.59 | A |
| ATOM | 267 | OD1 | ASP | A | 32 | 106.674 | 34.425 | 38.555 | 1.00 | 19.34 | A |
| ATOM | 268 | OD2 | ASP | A | 32 | 108.512 | 35.088 | 37.565 | 1.00 | 24.22 | A |
| ATOM | 269 | C | ASP | A | 32 | 104.876 | 32.906 | 37.140 | 1.00 | 15.56 | A |
| ATOM | 270 | O | ASP | A | 32 | 105.362 | 31.780 | 37.063 | 1.00 | 16.13 | A |
| ATOM | 271 | N | GLY | A | 33 | 103.973 | 33.241 | 38.056 | 1.00 | 14.55 | A |
| ATOM | 272 | CA | GLY | A | 33 | 103.506 | 32.255 | 39.015 | 1.00 | 14.38 | A |
| ATOM | 273 | C | GLY | A | 33 | 104.196 | 32.257 | 40.369 | 1.00 | 14.73 | A |
| ATOM | 274 | O | GLY | A | 33 | 103.771 | 31.545 | 41.275 | 1.00 | 14.14 | A |
| ATOM | 275 | N | SER | A | 34 | 105.257 | 33.042 | 40.522 | 1.00 | 14.25 | A |
| ATOM | 276 | CA | SER | A | 34 | 105.959 | 33.089 | 41.799 | 1.00 | 13.59 | A |
| ATOM | 277 | CB | SER | A | 34 | 107.384 | 33.618 | 41.608 | 1.00 | 14.59 | A |
| ATOM | 278 | OG | SER | A | 34 | 108.154 | 32.725 | 40.823 | 1.00 | 18.31 | A |
| ATOM | 279 | C | SER | A | 34 | 105.222 | 33.968 | 42.802 | 1.00 | 11.73 | A |
| ATOM | 280 | O | SER | A | 34 | 104.731 | 35.043 | 42.458 | 1.00 | 13.00 | A |
| ATOM | 281 | N | LEU | A | 35 | 105.134 | 33.494 | 44.040 | 1.00 | 10.82 | A |
| ATOM | 282 | CA | LEU | A | 35 | 104.476 | 34.246 | 45.098 | 1.00 | 9.31 | A |
| ATOM | 283 | CB | LEU | A | 35 | 103.994 | 33.304 | 46.201 | 1.00 | 9.99 | A |
| ATOM | 284 | CG | LEU | A | 35 | 102.775 | 32.435 | 45.896 | 1.00 | 9.48 | A |
| ATOM | 285 | CD1 | LEU | A | 35 | 102.632 | 31.363 | 46.962 | 1.00 | 8.89 | A |
| ATOM | 286 | CD2 | LEU | A | 35 | 101.524 | 33.309 | 45.844 | 1.00 | 9.67 | A |
| ATOM | 287 | C | LEU | A | 35 | 105.474 | 35.225 | 45.681 | 1.00 | 9.39 | A |
| ATOM | 288 | O | LEU | A | 35 | 106.675 | 34.960 | 45.688 | 1.00 | 9.58 | A |
| ATOM | 289 | N | TYR | A | 36 | 104.974 | 36.354 | 46.170 | 1.00 | 8.29 | A |
| ATOM | 290 | CA | TYR | A | 36 | 105.843 | 37.358 | 46.768 | 1.00 | 8.76 | A |
| ATOM | 291 | CB | TYR | A | 36 | 106.281 | 38.378 | 45.718 | 1.00 | 9.37 | A |
| ATOM | 292 | CG | TYR | A | 36 | 105.185 | 39.314 | 45.265 | 1.00 | 9.05 | A |
| ATOM | 293 | CD1 | TYR | A | 36 | 104.955 | 40.530 | 45.916 | 1.00 | 9.44 | A |
| ATOM | 294 | CE1 | TYR | A | 36 | 103.953 | 41.402 | 45.484 | 1.00 | 9.70 | A |
| ATOM | 295 | CD2 | TYR | A | 36 | 104.383 | 38.991 | 44.176 | 1.00 | 9.17 | A |
| ATOM | 296 | CE2 | TYR | A | 36 | 103.380 | 39.851 | 43.737 | 1.00 | 9.73 | A |
| ATOM | 297 | CZ | TYR | A | 36 | 103.168 | 41.052 | 44.389 | 1.00 | 10.05 | A |
| ATOM | 298 | OH | TYR | A | 36 | 102.182 | 41.898 | 43.933 | 1.00 | 10.60 | A |
| ATOM | 299 | C | TYR | A | 36 | 105.156 | 38.070 | 47.916 | 1.00 | 9.27 | A |
| ATOM | 300 | O | TYR | A | 36 | 103.943 | 37.967 | 48.096 | 1.00 | 9.16 | A |
| ATOM | 301 | N | LEU | A | 37 | 105.955 | 38.779 | 48.700 | 1.00 | 8.55 | A |
| ATOM | 302 | CA | LEU | A | 37 | 105.454 | 39.544 | 49.827 | 1.00 | 8.42 | A |
| ATOM | 303 | CB | LEU | A | 37 | 105.912 | 38.922 | 51.153 | 1.00 | 9.00 | A |
| ATOM | 304 | CG | LEU | A | 37 | 105.198 | 37.689 | 51.708 | 1.00 | 7.83 | A |
| ATOM | 305 | CD1 | LEU | A | 37 | 106.048 | 37.044 | 52.811 | 1.00 | 9.49 | A |
| ATOM | 306 | CD2 | LEU | A | 37 | 103.835 | 38.099 | 52.239 | 1.00 | 8.47 | A |
| ATOM | 307 | C | LEU | A | 37 | 106.010 | 40.956 | 49.754 | 1.00 | 9.58 | A |
| ATOM | 308 | O | LEU | A | 37 | 107.171 | 41.157 | 49.397 | 1.00 | 10.32 | A |
| ATOM | 309 | N | ILE | A | 38 | 105.162 | 41.930 | 50.049 | 1.00 | 9.45 | A |
| ATOM | 310 | CA | ILE | A | 38 | 105.598 | 43.314 | 50.119 | 1.00 | 8.98 | A |
| ATOM | 311 | CB | ILE | A | 38 | 104.917 | 44.239 | 49.073 | 1.00 | 9.69 | A |
| ATOM | 312 | CG2 | ILE | A | 38 | 105.640 | 44.115 | 47.745 | 1.00 | 10.02 | A |
| ATOM | 313 | CG1 | ILE | A | 38 | 103.425 | 43.923 | 48.940 | 1.00 | 10.44 | A |
| ATOM | 314 | CD1 | ILE | A | 38 | 102.687 | 44.913 | 48.027 | 1.00 | 13.40 | A |
| ATOM | 315 | C | ILE | A | 38 | 105.163 | 43.660 | 51.526 | 1.00 | 8.80 | A |
| ATOM | 316 | O | ILE | A | 38 | 103.997 | 43.495 | 51.889 | 1.00 | 9.26 | A |
| ATOM | 317 | N | TYR | A | 39 | 106.108 | 44.111 | 52.338 | 1.00 | 9.13 | A |
| ATOM | 318 | CA | TYR | A | 39 | 105.790 | 44.392 | 53.723 | 1.00 | 8.74 | A |
| ATOM | 319 | CB | TYR | A | 39 | 105.832 | 43.081 | 54.519 | 1.00 | 9.85 | A |
| ATOM | 320 | CG | TYR | A | 39 | 107.165 | 42.352 | 54.431 | 1.00 | 9.53 | A |
| ATOM | 321 | CD1 | TYR | A | 39 | 107.530 | 41.642 | 53.281 | 1.00 | 10.38 | A |
| ATOM | 322 | CE1 | TYR | A | 39 | 108.763 | 40.988 | 53.192 | 1.00 | 10.06 | A |
| ATOM | 323 | CD2 | TYR | A | 39 | 108.072 | 42.389 | 55.493 | 1.00 | 10.11 | A |
| ATOM | 324 | CE2 | TYR | A | 39 | 109.306 | 41.739 | 55.413 | 1.00 | 9.43 | A |
| ATOM | 325 | CZ | TYR | A | 39 | 109.645 | 41.040 | 54.258 | 1.00 | 10.24 | A |
| ATOM | 326 | OH | TYR | A | 39 | 110.863 | 40.393 | 54.160 | 1.00 | 12.23 | A |
| ATOM | 327 | C | TYR | A | 39 | 106.730 | 45.390 | 54.372 | 1.00 | 8.39 | A |
| ATOM | 328 | O | TYR | A | 39 | 107.689 | 45.852 | 53.758 | 1.00 | 8.64 | A |
| ATOM | 329 | N | ARG | A | 40 | 106.443 | 45.705 | 55.631 | 1.00 | 8.24 | A |
| ATOM | 330 | CA | ARG | A | 40 | 107.259 | 46.635 | 56.402 | 1.00 | 7.68 | A |
| ATOM | 331 | CB | ARG | A | 40 | 106.380 | 47.455 | 57.354 | 1.00 | 7.90 | A |
| ATOM | 332 | CG | ARG | A | 40 | 107.168 | 48.314 | 58.349 | 1.00 | 9.06 | A |
| ATOM | 333 | CD | ARG | A | 40 | 106.244 | 49.017 | 59.337 | 1.00 | 9.31 | A |
| ATOM | 334 | NE | ARG | A | 40 | 106.967 | 49.931 | 60.231 | 1.00 | 10.08 | A |
| ATOM | 335 | CZ | ARG | A | 40 | 107.487 | 49.590 | 61.408 | 1.00 | 11.46 | A |
| ATOM | 336 | NH1 | ARG | A | 40 | 107.367 | 48.347 | 61.857 | 1.00 | 10.19 | A |
| ATOM | 337 | NH2 | ARG | A | 40 | 108.138 | 50.493 | 62.133 | 1.00 | 10.39 | A |
| ATOM | 338 | C | ARG | A | 40 | 108.266 | 45.847 | 57.222 | 1.00 | 7.81 | A |
| ATOM | 339 | O | ARG | A | 40 | 107.885 | 44.981 | 58.006 | 1.00 | 7.67 | A |
| ATOM | 340 | N | ARG | A | 41 | 109.549 | 46.129 | 57.016 | 1.00 | 8.35 | A |
| ATOM | 341 | CA | ARG | A | 41 | 110.596 | 45.489 | 57.796 | 1.00 | 7.39 | A |
| ATOM | 342 | CB | ARG | A | 41 | 111.721 | 44.937 | 56.913 | 1.00 | 8.20 | A |
| ATOM | 343 | CG | ARG | A | 41 | 112.984 | 44.600 | 57.709 | 1.00 | 8.97 | A |
| ATOM | 344 | CD | ARG | A | 41 | 113.841 | 43.555 | 57.017 | 1.00 | 9.28 | A |
| ATOM | 345 | NE | ARG | A | 41 | 113.190 | 42.247 | 57.058 | 1.00 | 9.80 | A |
| ATOM | 346 | CZ | ARG | A | 41 | 113.713 | 41.133 | 56.561 | 1.00 | 11.03 | A |
| ATOM | 347 | NH1 | ARG | A | 41 | 114.906 | 41.156 | 55.974 | 1.00 | 12.78 | A |
| ATOM | 348 | NH2 | ARG | A | 41 | 113.043 | 39.994 | 56.659 | 1.00 | 11.14 | A |
| ATOM | 349 | C | ARG | A | 41 | 111.146 | 46.565 | 58.717 | 1.00 | 8.79 | A |
| ATOM | 350 | O | ARG | A | 41 | 111.349 | 47.709 | 58.304 | 1.00 | 8.89 | A |
| ATOM | 351 | N | ALA | A | 42 | 111.373 | 46.197 | 59.971 | 1.00 | 8.90 | A |
| ATOM | 352 | CA | ALA | A | 42 | 111.899 | 47.136 | 60.954 | 1.00 | 8.74 | A |
| ATOM | 353 | CB | ALA | A | 42 | 110.794 | 48.081 | 61.406 | 1.00 | 9.69 | A |
| ATOM | 354 | C | ALA | A | 42 | 112.428 | 46.350 | 62.139 | 1.00 | 9.33 | A |
| ATOM | 355 | O | ALA | A | 42 | 112.493 | 45.118 | 62.101 | 1.00 | 8.78 | A |
| ATOM | 356 | N | THR | A | 43 | 112.840 | 47.062 | 63.181 | 1.00 | 8.52 | A |
| ATOM | 357 | CA | THR | A | 43 | 113.322 | 46.409 | 64.387 | 1.00 | 8.95 | A |
| ATOM | 358 | CB | THR | A | 43 | 114.822 | 46.718 | 64.662 | 1.00 | 8.92 | A |
| ATOM | 359 | OG1 | THR | A | 43 | 115.026 | 48.135 | 64.721 | 1.00 | 10.06 | A |
| ATOM | 360 | CG2 | THR | A | 43 | 115.701 | 46.116 | 63.569 | 1.00 | 8.99 | A |
| ATOM | 361 | C | THR | A | 43 | 112.467 | 46.833 | 65.583 | 1.00 | 10.18 | A |
| ATOM | 362 | O | THR | A | 43 | 112.859 | 46.652 | 66.731 | 1.00 | 10.95 | A |
| ATOM | 363 | N | GLU | A | 44 | 111.294 | 47.393 | 65.290 | 1.00 | 9.72 | A |
| ATOM | 364 | CA | CLU | A | 44 | 110.319 | 47.819 | 66.302 | 1.00 | 11.40 | A |
| ATOM | 365 | CB | GLU | A | 44 | 110.743 | 49.131 | 66.977 | 1.00 | 12.90 | A |
| ATOM | 366 | CG | GLU | A | 44 | 110.982 | 49.053 | 68.493 | 1.00 | 13.46 | A |
| ATOM | 367 | CD | GLU | A | 44 | 109.715 | 48.817 | 69.319 | 1.00 | 14.58 | A |
| ATOM | 368 | OE1 | GLU | A | 44 | 108.595 | 48.938 | 68.778 | 1.00 | 15.00 | A |
| ATOM | 369 | OE2 | GLU | A | 44 | 109.837 | 48.518 | 70.530 | 1.00 | 15.03 | A |
| ATOM | 370 | C | GLU | A | 44 | 108.987 | 48.039 | 65.594 | 1.00 | 12.41 | A |
| ATOM | 371 | O | GLU | A | 44 | 108.925 | 48.066 | 64.364 | 1.00 | 12.02 | A |
| ATOM | 372 | N | HIS | A | 45 | 107.927 | 48.205 | 66.378 | 1.00 | 13.31 | A |
| ATOM | 373 | CA | HIS | A | 45 | 106.598 | 48.440 | 65.824 | 1.00 | 14.34 | A |
| ATOM | 374 | CB | HIS | A | 45 | 105.605 | 47.440 | 66.419 | 1.00 | 13.99 | A |
| ATOM | 375 | CG | HIS | A | 45 | 105.496 | 47.520 | 67.910 | 1.00 | 16.19 | A |
| ATOM | 376 | CD2 | HIS | A | 45 | 106.074 | 46.781 | 68.886 | 1.00 | 16.83 | A |
| ATOM | 377 | ND1 | HIS | A | 45 | 104.746 | 48.482 | 68.551 | 1.00 | 16.77 | A |
| ATOM | 378 | CE1 | HIS | A | 45 | 104.866 | 48.332 | 69.858 | 1.00 | 17.59 | A |
| ATOM | 379 | NE2 | HIS | A | 45 | 105.668 | 47.307 | 70.088 | 1.00 | 18.48 | A |
| ATOM | 380 | C | HIS | A | 45 | 106.115 | 49.866 | 66.093 | 1.00 | 14.78 | A |
| ATOM | 381 | O | HIS | A | 45 | 105.275 | 50.384 | 65.361 | 1.00 | 14.43 | A |
| ATOM | 382 | N | VAL | A | 46 | 106.656 | 50.501 | 67.131 | 1.00 | 14.63 | A |
| ATOM | 383 | CA | VAL | A | 46 | 106.243 | 51.856 | 67.490 | 1.00 | 14.76 | A |
| ATOM | 384 | CB | VAL | A | 46 | 107.032 | 52.389 | 68.720 | 1.00 | 15.26 | A |
| ATOM | 385 | CG1 | VAL | A | 46 | 106.751 | 51.521 | 69.932 | 1.00 | 16.77 | A |
| ATOM | 386 | CG2 | VAL | A | 46 | 108.526 | 52.430 | 68.414 | 1.00 | 15.98 | A |
| ATOM | 387 | C | VAL | A | 46 | 106.382 | 52.868 | 66.356 | 1.00 | 14.44 | A |
| ATOM | 388 | O | VAL | A | 46 | 107.275 | 52.760 | 65.515 | 1.00 | 13.40 | A |
| ATOM | 389 | N | GLY | A | 47 | 105.483 | 53.849 | 66.336 | 1.00 | 15.04 | A |
| ATOM | 390 | CA | GLY | A | 47 | 105.533 | 54.874 | 65.307 | 1.00 | 14.89 | A |
| ATOM | 391 | C | GLY | A | 47 | 106.816 | 55.672 | 65.433 | 1.00 | 14.00 | A |
| ATOM | 392 | O | GLY | A | 47 | 107.227 | 56.022 | 66.540 | 1.00 | 16.91 | A |
| ATOM | 393 | N | GLY | A | 48 | 107.451 | 55.960 | 64.304 | 1.00 | 14.91 | A |
| ATOM | 394 | CA | GLY | A | 48 | 108.685 | 56.716 | 64.335 | 1.00 | 13.98 | A |
| ATOM | 395 | C | GLY | A | 48 | 109.910 | 55.835 | 64.465 | 1.00 | 14.51 | A |
| ATOM | 396 | O | GLY | A | 48 | 110.990 | 56.320 | 64.814 | 1.00 | 13.73 | A |
| ATOM | 397 | N | SER | A | 49 | 109.754 | 54.540 | 64.201 | 1.00 | 13.72 | A |
| ATOM | 398 | CA | SER | A | 49 | 110.890 | 53.633 | 64.276 | 1.00 | 13.63 | A |
| ATOM | 399 | CB | SER | A | 49 | 110.463 | 52.251 | 64.803 | 1.00 | 15.82 | A |
| ATOM | 400 | OG | SER | A | 49 | 109.354 | 51.717 | 64.106 | 1.00 | 15.84 | A |
| ATOM | 401 | C | SER | A | 49 | 111.599 | 53.528 | 62.921 | 1.00 | 12.65 | A |
| ATOM | 402 | O | SER | A | 49 | 111.647 | 54.504 | 62.175 | 1.00 | 13.78 | A |
| ATOM | 403 | N | ASP | A | 50 | 112.134 | 52.357 | 62.593 | 1.00 | 11.46 | A |
| ATOM | 404 | CA | ASP | A | 50 | 112.890 | 52.196 | 61.355 | 1.00 | 10.80 | A |
| ATOM | 405 | CB | ASP | A | 50 | 114.255 | 51.585 | 61.689 | 1.00 | 10.81 | A |
| ATOM | 406 | CG | ASP | A | 50 | 114.139 | 50.266 | 62.440 | 1.00 | 11.23 | A |
| ATOM | 407 | OD1 | ASP | A | 50 | 113.031 | 49.935 | 62.932 | 1.00 | 10.47 | A |
| ATOM | 408 | OD2 | ASP | A | 50 | 115.171 | 49.571 | 62.548 | 1.00 | 11.54 | A |
| ATOM | 409 | C | ASP | A | 50 | 112.224 | 51.387 | 60.250 | 1.00 | 10.65 | A |
| ATOM | 410 | O | ASP | A | 50 | 112.863 | 50.548 | 59.617 | 1.00 | 11.06 | A |
| ATOM | 411 | N | GLY | A | 51 | 110.949 | 51.659 | 60.000 | 1.00 | 10.48 | A |
| ATOM | 412 | CA | GLY | A | 51 | 110.242 | 50.921 | 58.971 | 1.00 | 10.58 | A |
| ATOM | 413 | C | GLY | A | 51 | 110.634 | 51.242 | 57.540 | 1.00 | 11.09 | A |
| ATOM | 414 | O | GLY | A | 51 | 110.749 | 52.406 | 57.151 | 1.00 | 11.29 | A |
| ATOM | 415 | N | ARG | A | 52 | 110.878 | 50.196 | 56.759 | 1.00 | 10.33 | A |
| ATOM | 416 | CA | ARG | A | 52 | 111.195 | 50.362 | 55.353 | 1.00 | 10.27 | A |
| ATOM | 417 | CB | ARG | A | 52 | 112.690 | 50.144 | 55.056 | 1.00 | 11.09 | A |
| ATOM | 418 | CG | ARG | A | 52 | 113.306 | 48.850 | 55.563 | 1.00 | 9.57 | A |
| ATOM | 419 | CD | ARG | A | 52 | 113.831 | 49.012 | 56.984 | 1.00 | 10.29 | A |
| ATOM | 420 | NE | ARG | A | 52 | 114.772 | 47.943 | 57.325 | 1.00 | 10.82 | A |
| ATOM | 421 | CZ | ARG | A | 52 | 115.317 | 47.775 | 58.527 | 1.00 | 11.95 | A |
| ATOM | 422 | NH1 | ARG | A | 52 | 115.017 | 48.606 | 59.518 | 1.00 | 11.13 | A |
| ATOM | 423 | NH2 | ARG | A | 52 | 116.170 | 46.778 | 58.734 | 1.00 | 11.72 | A |
| ATOM | 424 | C | ARG | A | 52 | 110.343 | 49.363 | 54.585 | 1.00 | 10.86 | A |
| ATOM | 425 | O | ARG | A | 52 | 110.007 | 48.290 | 55.095 | 1.00 | 11.19 | A |
| ATOM | 426 | N | VAL | A | 53 | 109.969 | 49.736 | 53.368 | 1.00 | 10.24 | A |
| ATOM | 427 | CA | VAL | A | 53 | 109.147 | 48.878 | 52.523 | 1.00 | 10.73 | A |
| ATOM | 428 | CB | VAL | A | 53 | 108.380 | 49.721 | 51.479 | 1.00 | 10.37 | A |
| ATOM | 429 | CG1 | VAL | A | 53 | 107.413 | 48.838 | 50.698 | 1.00 | 11.57 | A |
| ATOM | 430 | CG2 | VAL | A | 53 | 107.649 | 50.860 | 52.166 | 1.00 | 12.22 | A |
| ATOM | 431 | C | VAL | A | 53 | 110.064 | 47.898 | 51.803 | 1.00 | 11.13 | A |
| ATOM | 432 | O | VAL | A | 53 | 111.022 | 48.312 | 51.155 | 1.00 | 11.16 | A |
| ATOM | 433 | N | VAL | A | 54 | 109.785 | 46.602 | 51.921 | 1.00 | 10.10 | A |
| ATOM | 434 | CA | VAL | A | 54 | 110.616 | 45.608 | 51.257 | 1.00 | 9.87 | A |
| ATOM | 435 | CB | VAL | A | 54 | 111.533 | 44.869 | 52.257 | 1.00 | 10.29 | A |
| ATOM | 436 | CG1 | VAL | A | 54 | 112.388 | 45.876 | 53.001 | 1.00 | 11.61 | A |
| ATOM | 437 | CG2 | VAL | A | 54 | 110.701 | 44.044 | 53.226 | 1.00 | 10.24 | A |
| ATOM | 438 | C | VAL | A | 54 | 109.807 | 44.583 | 50.474 | 1.00 | 9.86 | A |
| ATOM | 439 | O | VAL | A | 54 | 108.598 | 44.446 | 50.663 | 1.00 | 9.83 | A |
| ATOM | 440 | N | PHE | A | 55 | 110.505 | 43.870 | 49.596 | 1.00 | 10.04 | A |
| ATOM | 441 | CA | PHE | A | 55 | 109.919 | 42.860 | 48.726 | 1.00 | 10.45 | A |
| ATOM | 442 | CB | PHE | A | 55 | 109.974 | 43.368 | 47.278 | 1.00 | 10.48 | A |
| ATOM | 443 | CG | PHE | A | 55 | 109.703 | 42.311 | 46.239 | 1.00 | 9.96 | A |
| ATOM | 444 | CD1 | PHE | A | 55 | 108.412 | 42.103 | 45.753 | 1.00 | 10.64 | A |
| ATOM | 445 | CD2 | PHE | A | 55 | 110.743 | 41.534 | 45.737 | 1.00 | 9.84 | A |
| ATOM | 446 | CE1 | PHE | A | 55 | 108.162 | 41.135 | 44.779 | 1.00 | 10.89 | A |
| ATOM | 447 | CE2 | PHE | A | 55 | 110.510 | 40.563 | 44.765 | 1.00 | 12.27 | A |
| ATOM | 448 | CZ | PHE | A | 55 | 109.214 | 40.365 | 44.285 | 1.00 | 12.00 | A |
| ATOM | 449 | C | PHE | A | 55 | 110.696 | 41.550 | 48.838 | 1.00 | 11.23 | A |
| ATOM | 450 | O | PHE | A | 55 | 111.926 | 41.548 | 48.791 | 1.00 | 12.02 | A |
| ATOM | 451 | N | SER | A | 56 | 109.981 | 40.440 | 49.002 | 1.00 | 10.18 | A |
| ATOM | 452 | CA | SER | A | 56 | 110.617 | 39.127 | 49.075 | 1.00 | 11.30 | A |
| ATOM | 453 | CB | SER | A | 56 | 110.599 | 38.573 | 50.502 | 1.00 | 10.38 | A |
| ATOM | 454 | OG | SER | A | 56 | 111.240 | 37.296 | 50.550 | 1.00 | 13.25 | A |
| ATOM | 455 | C | SER | A | 56 | 109.886 | 38.167 | 48.142 | 1.00 | 11.62 | A |
| ATOM | 456 | O | SER | A | 56 | 108.658 | 38.191 | 48.051 | 1.00 | 11.77 | A |
| ATOM | 457 | N | LYS | A | 57 | 110.655 | 37.327 | 47.456 | 1.00 | 11.56 | A |
| ATOM | 458 | CA | LYS | A | 57 | 110.111 | 36.363 | 46.504 | 1.00 | 12.78 | A |
| ATOM | 459 | CB | LYS | A | 57 | 110.831 | 36.522 | 45.161 | 1.00 | 14.24 | A |
| ATOM | 460 | CG | LYS | A | 57 | 110.232 | 35.750 | 44.003 | 1.00 | 19.11 | A |
| ATOM | 461 | CD | LYS | A | 57 | 111.047 | 36.000 | 42.736 | 1.00 | 21.15 | A |
| ATOM | 462 | CE | LYS | A | 57 | 110.468 | 35.271 | 41.534 | 1.00 | 24.98 | A |
| ATOM | 463 | NZ | LYS | A | 57 | 111.370 | 35.350 | 40.347 | 1.00 | 28.71 | A |
| ATOM | 464 | C | LYS | A | 57 | 110.293 | 34.940 | 47.032 | 1.00 | 11.93 | A |
| ATOM | 465 | O | LYS | A | 57 | 111.349 | 34.606 | 47.559 | 1.00 | 11.86 | A |
| ATOM | 466 | N | LEU | A | 58 | 109.261 | 34.107 | 46.907 | 1.00 | 11.09 | A |
| ATOM | 467 | CA | LEU | A | 58 | 109.352 | 32.732 | 47.379 | 1.00 | 11.40 | A |
| ATOM | 468 | CB | LEU | A | 58 | 107.969 | 32.211 | 47.805 | 1.00 | 11.00 | A |
| ATOM | 469 | CG | LEU | A | 58 | 107.909 | 30.809 | 48.430 | 1.00 | 10.39 | A |
| ATOM | 470 | CD1 | LEU | A | 58 | 108.800 | 30.748 | 49.665 | 1.00 | 11.61 | A |
| ATOM | 471 | CD2 | LEU | A | 58 | 106.463 | 30.471 | 48.802 | 1.00 | 12.43 | A |
| ATOM | 472 | C | LEU | A | 58 | 109.932 | 31.862 | 46.263 | 1.00 | 12.97 | A |
| ATOM | 473 | O | LEU | A | 58 | 109.275 | 31.612 | 45.251 | 1.00 | 13.39 | A |
| ATOM | 474 | N | GLU | A | 59 | 111.173 | 31.415 | 46.452 | 1.00 | 13.54 | A |
| ATOW | 475 | CA | GLU | A | 59 | 111.861 | 30.590 | 45.460 | 1.00 | 15.80 | A |
| ATOM | 476 | CB | GLU | A | 59 | 113.056 | 31.352 | 44.879 | 1.00 | 19.31 | A |
| ATOM | 477 | CG | GLU | A | 59 | 112.755 | 32.796 | 44.513 | 1.00 | 22.32 | A |
| ATOM | 478 | CD | GLU | A | 59 | 113.952 | 33.504 | 43.906 | 1.00 | 27.43 | A |
| ATOM | 479 | OE1 | GLU | A | 59 | 114.256 | 33.255 | 42.721 | 1.00 | 30.87 | A |
| ATOM | 480 | OE2 | GLU | A | 59 | 114.590 | 34.305 | 44.617 | 1.00 | 27.96 | A |
| ATOM | 481 | C | GLU | A | 59 | 112.356 | 29.290 | 46.084 | 1.00 | 15.02 | A |
| ATOM | 482 | O | GLU | A | 59 | 113.051 | 29.307 | 47.098 | 1.00 | 15.40 | A |
| ATOM | 483 | N | GLY | A | 60 | 111.998 | 28.163 | 45.479 | 1.00 | 15.48 | A |
| ATOM | 484 | CA | GLY | A | 60 | 112.429 | 26.880 | 46.006 | 1.00 | 15.66 | A |
| ATOM | 485 | C | GLY | A | 60 | 112.005 | 26.670 | 47.446 | 1.00 | 15.47 | A |
| ATOM | 486 | O | GLY | A | 60 | 112.700 | 26.014 | 48.222 | 1.00 | 16.56 | A |
| ATOM | 487 | N | GLY | A | 61 | 110.864 | 27.244 | 47.811 | 1.00 | 14.14 | A |
| ATOM | 488 | CA | GLY | A | 61 | 110.366 | 27.085 | 49.164 | 1.00 | 13.46 | A |
| ATOM | 489 | C | GLY | A | 61 | 111.021 | 27.969 | 50.208 | 1.00 | 12.58 | A |
| ATOM | 490 | O | GLY | A | 61 | 110.765 | 27.814 | 51.399 | 1.00 | 12.91 | A |
| ATOM | 491 | N | ILE | A | 62 | 111.870 | 28.896 | 49.780 | 1.00 | 13.62 | A |
| ATOM | 492 | CA | ILE | A | 62 | 112.523 | 29.785 | 50.735 | 1.00 | 14.89 | A |
| ATOM | 493 | CB | ILE | A | 62 | 114.021 | 29.427 | 50.896 | 1.00 | 18.68 | A |
| ATOM | 494 | CG2 | ILE | A | 62 | 114.655 | 30.299 | 51.970 | 1.00 | 19.41 | A |
| ATOM | 495 | CG1 | ILE | A | 62 | 114.162 | 27.950 | 51.283 | 1.00 | 18.81 | A |
| ATOM | 496 | CD1 | ILE | A | 62 | 115.590 | 27.445 | 51.315 | 1.00 | 22.99 | A |
| ATOM | 497 | C | ILE | A | 62 | 112.378 | 31.239 | 50.296 | 1.00 | 13.47 | A |
| ATOM | 498 | O | ILE | A | 62 | 112.604 | 31.577 | 49.133 | 1.00 | 12.92 | A |
| ATOM | 499 | N | TRP | A | 63 | 111.982 | 32.098 | 51.228 | 1.00 | 13.16 | A |
| ATOM | 500 | CA | TRP | A | 63 | 111.820 | 33.510 | 50.917 | 1.00 | 13.21 | A |
| ATOM | 501 | CB | TRP | A | 63 | 111.064 | 34.216 | 52.043 | 1.00 | 12.16 | A |
| ATOM | 502 | CG | TRP | A | 63 | 109.649 | 33.734 | 52.168 | 1.00 | 11.76 | A |
| ATOM | 503 | CD2 | TRP | A | 63 | 108.527 | 34.186 | 51.402 | 1.00 | 12.07 | A |
| ATOM | 504 | CE2 | TRP | A | 63 | 107.409 | 33.420 | 51.804 | 1.00 | 11.91 | A |
| ATOM | 505 | CE3 | TRP | A | 63 | 108.358 | 35.160 | 50.410 | 1.00 | 11.96 | A |
| ATOM | 506 | CD1 | TRP | A | 63 | 109.182 | 32.744 | 52.986 | 1.00 | 11.02 | A |
| ATOM | 507 | NE1 | TRP | A | 63 | 107.832 | 32.551 | 52.773 | 1.00 | 11.20 | A |
| ATOM | 508 | CZ2 | TRP | A | 63 | 106.139 | 33.600 | 51.251 | 1.00 | 11.94 | A |
| ATOM | 509 | CZ3 | TRP | A | 63 | 107.087 | 35.338 | 49.862 | 1.00 | 12.01 | A |
| ATOM | 510 | CH2 | TRP | A | 63 | 105.998 | 34.558 | 50.285 | 1.00 | 11.47 | A |
| ATOM | 511 | C | TRP | A | 63 | 113.184 | 34.147 | 50.700 | 1.00 | 13.73 | A |
| ATOM | 512 | O | TRP | A | 63 | 114.087 | 34.014 | 51.529 | 1.00 | 14.78 | A |
| ATOM | 513 | N | SER | A | 64 | 113.322 | 34.834 | 49.573 | 1.00 | 14.66 | A |
| ATOM | 514 | CA | SER | A | 64 | 114.580 | 35.474 | 49.209 | 1.00 | 15.78 | A |
| ATOM | 515 | CB | SER | A | 64 | 114.535 | 35.927 | 47.746 | 1.00 | 17.42 | A |
| ATOM | 516 | OG | SER | A | 64 | 113.594 | 36.978 | 47.566 | 1.00 | 19.83 | A |
| ATOM | 517 | C | SER | A | 64 | 114.945 | 36.663 | 50.082 | 1.00 | 16.22 | A |
| ATOM | 518 | O | SER | A | 64 | 114.096 | 37.235 | 50.768 | 1.00 | 15.24 | A |
| ATOM | 519 | N | ALA | A | 65 | 116.227 | 37.020 | 50.053 | 1.00 | 16.56 | A |
| ATOM | 520 | CA | ALA | A | 65 | 116.725 | 38.164 | 50.806 | 1.00 | 16.23 | A |
| ATOM | 521 | CB | ALA | A | 65 | 118.212 | 38.359 | 50.546 | 1.00 | 17.16 | A |
| ATOM | 522 | C | ALA | A | 65 | 115.931 | 39.344 | 50.269 | 1.00 | 15.11 | A |
| ATOM | 523 | O | ALA | A | 65 | 115.962 | 39.633 | 49.072 | 1.00 | 15.86 | A |
| ATOM | 524 | N | PRO | A | 66 | 115.200 | 40.039 | 51.146 | 1.00 | 16.41 | A |
| ATOM | 525 | CD | PRO | A | 66 | 115.014 | 39.736 | 52.575 | 1.00 | 17.33 | A |
| ATOM | 526 | CA | PRO | A | 66 | 114.383 | 41.187 | 50.750 | 1.00 | 16.37 | A |
| ATOM | 527 | CB | PRO | A | 66 | 113.851 | 41.701 | 52.084 | 1.00 | 17.21 | A |
| ATOM | 528 | CG | PRO | A | 66 | 113.713 | 40.439 | 52.880 | 1.00 | 17.97 | A |
| ATOM | 529 | C | PRO | A | 66 | 115.062 | 42.282 | 49.938 | 1.00 | 16.65 | A |
| ATOM | 530 | O | PRO | A | 66 | 116.203 | 42.665 | 50.200 | 1.00 | 17.26 | A |
| ATOM | 531 | N | THR | A | 67 | 114.338 | 42.767 | 48.936 | 1.00 | 15.73 | A |
| ATOM | 532 | CA | THR | A | 67 | 114.801 | 43.850 | 48.084 | 1.00 | 15.77 | A |
| ATOM | 533 | CB | THR | A | 67 | 114.288 | 43.693 | 46.635 | 1.00 | 15.11 | A |
| ATOM | 534 | OG1 | THR | A | 67 | 114.872 | 42.529 | 46.039 | 1.00 | 19.26 | A |
| ATOM | 535 | CG2 | THR | A | 67 | 114.647 | 44.917 | 45.804 | 1.00 | 17.66 | A |
| ATOM | 536 | C | THR | A | 67 | 114.183 | 45.105 | 48.688 | 1.00 | 14.83 | A |
| ATOM | 537 | O | THR | A | 67 | 113.014 | 45.101 | 49.071 | 1.00 | 13.75 | A |
| ATOM | 538 | N | ILE | A | 68 | 114.963 | 46.175 | 48.785 | 1.00 | 14.95 | A |
| ATOM | 539 | CA | ILE | A | 68 | 114.458 | 47.418 | 49.351 | 1.00 | 15.65 | A |
| ATOM | 540 | CB | ILE | A | 68 | 115.611 | 48.350 | 49.781 | 1.00 | 17.47 | A |
| ATOM | 541 | CG2 | ILE | A | 68 | 115.041 | 49.625 | 50.383 | 1.00 | 18.85 | A |
| ATOM | 542 | CG1 | ILE | A | 68 | 116.527 | 47.632 | 50.772 | 1.00 | 19.39 | A |
| ATOM | 543 | CD1 | ILE | A | 68 | 115.842 | 47.188 | 52.044 | 1.00 | 21.28 | A |
| ATOM | 544 | C | ILE | A | 68 | 113.598 | 48.162 | 48.338 | 1.00 | 15.87 | A |
| ATOM | 545 | O | ILE | A | 68 | 114.071 | 48.516 | 47.259 | 1.00 | 18.38 | A |
| ATOM | 546 | N | VAL | A | 69 | 112.334 | 48.389 | 48.684 | 1.00 | 14.01 | A |
| ATOM | 547 | CA | VAL | A | 69 | 111.416 | 49.104 | 47.802 | 1.00 | 14.26 | A |
| ATOM | 548 | CB | VAL | A | 69 | 109.959 | 48.632 | 48.002 | 1.00 | 13.07 | A |
| ATOM | 549 | CG1 | VAL | A | 69 | 109.018 | 49.452 | 47.121 | 1.00 | 14.58 | A |
| ATOM | 550 | CG2 | VAL | A | 69 | 109.841 | 47.155 | 47.659 | 1.00 | 12.82 | A |
| ATOM | 551 | C | VAL | A | 69 | 111.482 | 50.601 | 48.092 | 1.00 | 15.51 | A |
| ATOM | 552 | O | VAL | A | 69 | 111.655 | 51.418 | 47.184 | 1.00 | 16.81 | A |
| ATOM | 553 | N | ALA | A | 70 | 111.346 | 50.948 | 49.368 | 1.00 | 14.85 | A |
| ATOM | 554 | CA | ALA | A | 70 | 111.382 | 52.337 | 49.798 | 1.00 | 15.92 | A |
| ATOM | 555 | CB | ALA | A | 70 | 109.971 | 52.903 | 49.859 | 1.00 | 16.79 | A |
| ATOM | 556 | C | ALA | A | 70 | 112.047 | 52.456 | 51.160 | 1.00 | 17.15 | A |
| ATOM | 557 | O | ALA | A | 70 | 111.698 | 51.747 | 52.104 | 1.00 | 15.18 | A |
| ATOM | 558 | N | GLN | A | 71 | 113.007 | 53.365 | 51.254 | 1.00 | 18.89 | A |
| ATOM | 559 | CA | GLN | A | 71 | 113.734 | 53.577 | 52.493 | 1.00 | 20.77 | A |
| ATOM | 560 | CB | GLN | A | 71 | 114.879 | 52.568 | 52.590 | 1.00 | 21.56 | A |
| ATOM | 561 | CG | GLN | A | 71 | 115.723 | 52.682 | 53.841 | 1.00 | 24.47 | A |
| ATOM | 562 | CD | GLN | A | 71 | 116.659 | 51.502 | 54.010 | 1.00 | 25.73 | A |
| ATOM | 563 | OE1 | GLN | A | 71 | 117.461 | 51.194 | 53.125 | 1.00 | 27.78 | A |
| ATOM | 564 | NE2 | GLN | A | 71 | 116.560 | 50.831 | 55.150 | 1.00 | 24.65 | A |
| ATOM | 565 | C | GLN | A | 71 | 114.275 | 54.998 | 52.525 | 1.00 | 21.43 | A |
| ATOM | 566 | O | GLN | A | 71 | 114.726 | 55.524 | 51.508 | 1.00 | 23.23 | A |
| ATOM | 567 | N | ALA | A | 72 | 114.215 | 55.615 | 53.698 | 1.00 | 21.70 | A |
| ATOM | 568 | CA | ALA | A | 72 | 114.700 | 56.976 | 53.878 | 1.00 | 22.85 | A |
| ATOM | 569 | CB | ALA | A | 72 | 113.633 | 57.977 | 53.440 | 1.00 | 22.72 | A |
| ATOM | 570 | C | ALA | A | 72 | 115.047 | 57.186 | 55.345 | 1.00 | 22.94 | A |
| ATOM | 571 | O | ALA | A | 72 | 114.237 | 56.908 | 56.227 | 1.00 | 24.07 | A |
| ATOM | 572 | N | GLY | A | 73 | 116.259 | 57.669 | 55.598 | 1.00 | 24.47 | A |
| ATOM | 573 | CA | GLY | A | 73 | 116.688 | 57.902 | 56.964 | 1.00 | 24.92 | A |
| ATOM | 574 | C | GLY | A | 73 | 115.828 | 58.932 | 57.663 | 1.00 | 25.46 | A |
| ATOM | 575 | O | GLY | A | 73 | 115.590 | 60.017 | 57.132 | 1.00 | 25.54 | A |
| ATOM | 576 | N | GLY | A | 74 | 115.358 | 58.590 | 58.857 | 1.00 | 25.06 | A |
| ATOM | 577 | CA | GLY | A | 74 | 114.523 | 59.506 | 59.609 | 1.00 | 26.31 | A |
| ATOM | 578 | C | GLY | A | 74 | 113.043 | 59.330 | 59.337 | 1.00 | 26.46 | A |
| ATOM | 579 | O | GLY | A | 74 | 112.212 | 59.927 | 60.022 | 1.00 | 28.60 | A |
| ATOM | 580 | N | GLN | A | 75 | 112.709 | 58.516 | 58.337 | 1.00 | 25.76 | A |
| ATOM | 581 | CA | GLN | A | 75 | 111.313 | 58.266 | 57.976 | 1.00 | 24.91 | A |
| ATOM | 582 | CB | GLN | A | 75 | 111.096 | 58.499 | 56.474 | 1.00 | 27.35 | A |
| ATOM | 583 | CG | GLN | A | 75 | 111.460 | 59.901 | 55.989 | 1.00 | 31.13 | A |
| ATOM | 584 | CD | GLN | A | 75 | 111.103 | 60.136 | 54.529 | 1.00 | 33.05 | A |
| ATOM | 585 | OE1 | GLN | A | 75 | 111.493 | 61.145 | 53.940 | 1.00 | 36.08 | A |
| ATOM | 586 | NE2 | GLN | A | 75 | 110.353 | 59.209 | 53.940 | 1.00 | 34.31 | A |
| ATOM | 587 | C | GLN | A | 75 | 110.912 | 56.832 | 58.330 | 1.00 | 22.61 | A |
| ATOM | 588 | O | GLN | A | 75 | 111.669 | 55.892 | 58.089 | 1.00 | 22.90 | A |
| ATOM | 589 | N | ASP | A | 76 | 109.718 | 56.674 | 58.895 | 1.00 | 17.92 | A |
| ATOM | 590 | CA | ASP | A | 76 | 109.215 | 55.361 | 59.291 | 1.00 | 14.89 | A |
| ATOM | 591 | CB | ASP | A | 76 | 108.859 | 55.376 | 60.782 | 1.00 | 12.54 | A |
| ATOM | 592 | CG | ASP | A | 76 | 108.196 | 54.087 | 61.249 | 1.00 | 12.77 | A |
| ATOM | 593 | OD1 | ASP | A | 76 | 108.612 | 52.995 | 60.807 | 1.00 | 12.86 | A |
| ATOM | 594 | OD2 | ASP | A | 76 | 107.267 | 54.168 | 62.079 | 1.00 | 12.91 | A |
| ATOM | 595 | C | ASP | A | 76 | 108.005 | 54.932 | 58.469 | 1.00 | 13.07 | A |
| ATOM | 596 | O | ASP | A | 76 | 106.889 | 55.386 | 58.709 | 1.00 | 12.92 | A |
| ATOM | 597 | N | PHE | A | 77 | 108.233 | 54.061 | 57.491 | 1.00 | 12.87 | A |
| ATOM | 598 | CA | PHE | A | 77 | 107.141 | 53.571 | 56.657 | 1.00 | 12.67 | A |
| ATOM | 599 | CB | PHE | A | 77 | 107.690 | 52.842 | 55.432 | 1.00 | 12.06 | A |
| ATOM | 600 | CG | PHE | A | 77 | 108.169 | 53.763 | 54.347 | 1.00 | 12.79 | A |
| ATOM | 601 | CD1 | PHE | A | 77 | 107.258 | 54.452 | 53.548 | 1.00 | 13.95 | A |
| ATOM | 602 | CD2 | PHE | A | 77 | 109.530 | 53.963 | 54.139 | 1.00 | 14.78 | A |
| ATOM | 603 | CE1 | PHE | A | 77 | 107.696 | 55.325 | 52.559 | 1.00 | 15.41 | A |
| ATOM | 604 | CE2 | PHE | A | 77 | 109.980 | 54.834 | 53.152 | 1.00 | 16.02 | A |
| ATOM | 605 | CZ | PHE | A | 77 | 109.062 | 55.518 | 52.360 | 1.00 | 15.98 | A |
| ATOM | 606 | C | PHE | A | 77 | 106.285 | 52.630 | 57.491 | 1.00 | 13.17 | A |
| ATOM | 607 | O | PHE | A | 77 | 106.802 | 51.802 | 58.242 | 1.00 | 12.06 | A |
| ATOM | 608 | N | ARG | A | 78 | 104.971 | 52.761 | 57.359 | 1.00 | 12.12 | A |
| ATOM | 609 | CA | ARG | A | 78 | 104.049 | 51.937 | 58.119 | 1.00 | 10.82 | A |
| ATOM | 610 | CB | ARG | A | 78 | 102.914 | 52.809 | 58.663 | 1.00 | 10.88 | A |
| ATOM | 611 | CG | ARG | A | 78 | 103.399 | 53.946 | 59.570 | 1.00 | 11.71 | A |
| ATOM | 612 | CD | ARG | A | 78 | 104.354 | 53.442 | 60.656 | 1.00 | 12.10 | A |
| ATOM | 613 | NE | ARG | A | 78 | 103.795 | 52.312 | 61.401 | 1.00 | 13.23 | A |
| ATOM | 614 | CZ | ARG | A | 78 | 104.369 | 51.740 | 62.456 | 1.00 | 13.43 | A |
| ATOM | 615 | NH1 | ARG | A | 78 | 105.535 | 52.183 | 62.916 | 1.00 | 13.62 | A |
| ATOM | 616 | NH2 | ARG | A | 78 | 103.773 | 50.719 | 63.055 | 1.00 | 14.95 | A |
| ATOM | 617 | C | ARG | A | 78 | 103.501 | 50.765 | 57.301 | 1.00 | 10.21 | A |
| ATOM | 618 | O | ARG | A | 78 | 104.267 | 50.062 | 56.645 | 1.00 | 10.94 | A |
| ATOM | 619 | N | ASP | A | 79 | 102.190 | 50.544 | 57.339 | 1.00 | 10.42 | A |
| ATOM | 620 | CA | ASP | A | 79 | 101.596 | 49.432 | 56.591 | 1.00 | 11.40 | A |
| ATOM | 621 | CB | ASP | A | 79 | 100.069 | 49.453 | 56.710 | 1.00 | 12.26 | A |
| ATOM | 622 | CG | ASP | A | 79 | 99.585 | 49.091 | 58.091 | 1.00 | 13.79 | A |
| ATOM | 623 | OD1 | ASP | A | 79 | 100.376 | 49.209 | 59.056 | 1.00 | 14.71 | A |
| ATOM | 624 | OD2 | ASP | A | 79 | 98.399 | 48.707 | 58.219 | 1.00 | 13.93 | A |
| ATOM | 625 | C | ASP | A | 79 | 101.965 | 49.484 | 55.114 | 1.00 | 11.14 | A |
| ATOM | 626 | O | ASP | A | 79 | 102.179 | 50.557 | 54.552 | 1.00 | 10.24 | A |
| ATOM | 627 | N | VAL | A | 80 | 102.039 | 48.316 | 54.485 | 1.00 | 9.58 | A |
| ATOM | 628 | CA | VAL | A | 80 | 102.345 | 48.251 | 53.068 | 1.00 | 10.10 | A |
| ATOM | 629 | CB | VAL | A | 80 | 103.593 | 47.385 | 52.782 | 1.00 | 10.20 | A |
| ATOM | 630 | CG1 | VAL | A | 80 | 103.842 | 47.323 | 51.282 | 1.00 | 10.94 | A |
| ATOM | 631 | CG2 | VAL | A | 80 | 104.810 | 47.984 | 53.480 | 1.00 | 10.43 | A |
| ATOM | 632 | C | VAL | A | 80 | 101.136 | 47.671 | 52.343 | 1.00 | 11.03 | A |
| ATOM | 633 | O | VAL | A | 80 | 100.739 | 46.524 | 52.573 | 1.00 | 11.08 | A |
| ATOM | 634 | N | ALA | A | 81 | 100.538 | 48.501 | 51.493 | 1.00 | 10.14 | A |
| ATOM | 635 | CA | ALA | A | 81 | 99.376 | 48.122 | 50.702 | 1.00 | 9.73 | A |
| ATOM | 636 | CB | ALA | A | 81 | 98.344 | 49.248 | 50.712 | 1.00 | 10.20 | A |
| ATOM | 637 | C | ALA | A | 81 | 99.844 | 47.864 | 49.280 | 1.00 | 10.26 | A |
| ATOM | 638 | O | ALA | A | 81 | 100.918 | 48.309 | 48.885 | 1.00 | 9.90 | A |
| ATOM | 639 | N | GLY | A | 82 | 99.049 | 47.137 | 48.504 | 1.00 | 10.26 | A |
| ATOM | 640 | CA | GLY | A | 82 | 99.449 | 46.885 | 47.136 | 1.00 | 9.56 | A |
| ATOM | 641 | C | GLY | A | 82 | 99.105 | 45.506 | 46.626 | 1.00 | 9.44 | A |
| ATOM | 642 | O | GLY | A | 82 | 98.298 | 44.786 | 47.219 | 1.00 | 9.41 | A |
| ATOM | 643 | N | GLY | A | 83 | 99.743 | 45.140 | 45.522 | 1.00 | 10.14 | A |
| ATOM | 644 | CA | GLY | A | 83 | 99.498 | 43.852 | 44.909 | 1.00 | 10.27 | A |
| ATOM | 645 | C | GLY | A | 83 | 99.861 | 43.896 | 43.438 | 1.00 | 11.28 | A |
| ATOM | 646 | O | GLY | A | 83 | 100.454 | 44.871 | 42.963 | 1.00 | 10.62 | A |
| ATOM | 647 | N | THR | A | 84 | 99.497 | 42.841 | 42.715 | 1.00 | 10.98 | A |
| ATOM | 648 | CA | THR | A | 84 | 99.793 | 42.728 | 41.290 | 1.00 | 11.47 | A |
| ATOM | 649 | CB | THR | A | 84 | 100.049 | 41.259 | 40.898 | 1.00 | 11.91 | A |
| ATOM | 650 | OG1 | THR | A | 84 | 100.909 | 40.646 | 41.868 | 1.00 | 12.41 | A |
| ATOM | 651 | CG2 | THR | A | 84 | 100.710 | 41.179 | 39.530 | 1.00 | 12.50 | A |
| ATOM | 652 | C | THR | A | 84 | 98.656 | 43.248 | 40.424 | 1.00 | 12.33 | A |
| ATOM | 653 | O | THR | A | 84 | 97.498 | 42.884 | 40.622 | 1.00 | 13.58 | A |
| ATOM | 654 | N | MET | A | 85 | 98.993 | 44.098 | 39.462 | 1.00 | 11.16 | A |
| ATOM | 655 | CA | MET | A | 85 | 97.996 | 44.652 | 38.556 | 1.00 | 12.05 | A |
| ATOM | 656 | CB | MET | A | 85 | 98.499 | 45.971 | 37.959 | 1.00 | 12.12 | A |
| ATOM | 657 | CG | MET | A | 85 | 98.783 | 47.061 | 38.978 | 1.00 | 13.06 | A |
| ATOM | 658 | SD | MET | A | 85 | 99.343 | 48.597 | 38.210 | 1.00 | 15.14 | A |
| ATOM | 659 | CE | MET | A | 85 | 97.811 | 49.247 | 37.615 | 1.00 | 16.29 | A |
| ATOM | 660 | C | MET | A | 85 | 97.741 | 43.658 | 37.428 | 1.00 | 13.12 | A |
| ATOM | 661 | O | MET | A | 85 | 98.491 | 42.697 | 37.254 | 1.00 | 13.71 | A |
| ATOM | 662 | N | PRO | A | 86 | 96.666 | 43.871 | 36.651 | 1.00 | 13.87 | A |
| ATOM | 663 | CD | PRO | A | 86 | 95.565 | 44.820 | 36.894 | 1.00 | 14.65 | A |
| ATOM | 664 | CA | PRO | A | 86 | 96.345 | 42.974 | 35.536 | 1.00 | 14.98 | A |
| ATOM | 665 | CB | PRO | A | 86 | 95.091 | 43.608 | 34.939 | 1.00 | 15.52 | A |
| ATOM | 666 | CG | PRO | A | 86 | 94.411 | 44.179 | 36.147 | 1.00 | 16.16 | A |
| ATOM | 667 | C | PRO | A | 86 | 97.491 | 42.911 | 34.527 | 1.00 | 14.48 | A |
| ATOM | 668 | O | PRO | A | 86 | 97.694 | 41.899 | 33.857 | 1.00 | 15.47 | A |
| ATOM | 669 | N | SER | A | 87 | 98.242 | 44.003 | 34.437 | 1.00 | 13.86 | A |
| ATOM | 670 | CA | SER | A | 87 | 99.367 | 44.110 | 33.509 | 1.00 | 14.16 | A |
| ATOM | 671 | CB | SER | A | 87 | 99.762 | 45.574 | 33.348 | 1.00 | 14.46 | A |
| ATOM | 672 | OG | SER | A | 87 | 100.351 | 46.044 | 34.557 | 1.00 | 14.23 | A |
| ATOM | 673 | C | SER | A | 87 | 100.606 | 43.339 | 33.947 | 1.00 | 14.45 | A |
| ATOM | 674 | O | SER | A | 87 | 101.513 | 43.110 | 33.148 | 1.00 | 15.74 | A |
| ATOM | 675 | N | GLY | A | 88 | 100.651 | 42.957 | 35.218 | 1.00 | 13.84 | A |
| ATOM | 676 | CA | GLY | A | 88 | 101.810 | 42.244 | 35.718 | 1.00 | 13.28 | A |
| ATOM | 677 | C | GLY | A | 88 | 102.671 | 43.175 | 36.551 | 1.00 | 13.69 | A |
| ATOM | 678 | O | GLY | A | 88 | 103.584 | 42.732 | 37.250 | 1.00 | 13.41 | A |
| ATOM | 679 | N | ARG | A | 89 | 102.388 | 44.472 | 36.468 | 1.00 | 12.61 | A |
| ATOM | 680 | CA | ARG | A | 89 | 103.123 | 45.467 | 37.244 | 1.00 | 12.83 | A |
| ATOM | 681 | CB | ARG | A | 89 | 102.752 | 46.884 | 36.784 | 1.00 | 13.66 | A |
| ATOM | 682 | CG | ARG | A | 89 | 103.142 | 47.984 | 37.763 | 1.00 | 13.16 | A |
| ATOM | 683 | CD | ARG | A | 89 | 102.820 | 49.374 | 37.221 | 1.00 | 15.07 | A |
| ATOM | 684 | NE | ARG | A | 89 | 103.832 | 49.838 | 36.277 | 1.00 | 16.02 | A |
| ATOM | 685 | CZ | ARG | A | 89 | 103.858 | 51.059 | 35.749 | 1.00 | 17.18 | A |
| ATOM | 686 | NH1 | ARG | A | 89 | 102.923 | 51.944 | 36.067 | 1.00 | 17.23 | A |
| ATOM | 687 | NH2 | ARG | A | 89 | 104.833 | 51.400 | 34.918 | 1.00 | 17.84 | A |
| ATOM | 688 | C | ARG | A | 89 | 102.759 | 45.291 | 38.713 | 1.00 | 12.39 | A |
| ATOM | 689 | O | ARG | A | 89 | 101.619 | 44.955 | 39.037 | 1.00 | 13.04 | A |
| ATOM | 690 | N | ILE | A | 90 | 103.727 | 45.504 | 39.598 | 1.00 | 12.05 | A |
| ATOM | 691 | CA | ILE | A | 90 | 103.485 | 45.385 | 41.034 | 1.00 | 12.73 | A |
| ATOM | 692 | CB | ILE | A | 90 | 104.630 | 44.655 | 41.761 | 1.00 | 12.77 | A |
| ATOM | 693 | CG2 | ILE | A | 90 | 104.330 | 44.589 | 43.250 | 1.00 | 13.71 | A |
| ATOM | 694 | CG1 | ILE | A | 90 | 104.802 | 43.244 | 41.206 | 1.00 | 15.81 | A |
| ATOM | 695 | CD1 | ILE | A | 90 | 105.995 | 42.520 | 41.807 | 1.00 | 18.25 | A |
| ATOM | 696 | C | ILE | A | 90 | 103.378 | 46.773 | 41.647 | 1.00 | 11.75 | A |
| ATOM | 697 | O | ILE | A | 90 | 104.152 | 47.668 | 41.311 | 1.00 | 13.19 | A |
| ATOM | 698 | N | VAL | A | 91 | 102.418 | 46.941 | 42.549 | 1.00 | 11.28 | A |
| ATOM | 699 | CA | VAL | A | 91 | 102.209 | 48.210 | 43.230 | 1.00 | 11.40 | A |
| ATOM | 700 | CB | VAL | A | 91 | 100.769 | 48.740 | 43.004 | 1.00 | 12.02 | A |
| ATOM | 701 | CG1 | VAL | A | 91 | 100.523 | 49.979 | 43.858 | 1.00 | 13.17 | A |
| ATOM | 702 | CG2 | VAL | A | 91 | 100.556 | 49.059 | 41.534 | 1.00 | 13.27 | A |
| ATOM | 703 | C | VAL | A | 91 | 102.431 | 48.050 | 44.735 | 1.00 | 11.62 | A |
| ATOM | 704 | O | VAL | A | 91 | 101.911 | 47.121 | 45.355 | 1.00 | 11.07 | A |
| ATOM | 705 | N | ALA | A | 92 | 103.215 | 48.955 | 45.314 | 1.00 | 12.12 | A |
| ATOM | 706 | CA | ALA | A | 92 | 103.491 | 48.949 | 46.749 | 1.00 | 11.90 | A |
| ATOM | 707 | CB | ALA | A | 92 | 104.913 | 48.444 | 47.023 | 1.00 | 12.88 | A |
| ATOM | 708 | C | ALA | A | 92 | 103.333 | 50.384 | 47.239 | 1.00 | 11.97 | A |
| ATOM | 709 | O | ALA | A | 92 | 103.961 | 51.298 | 46.705 | 1.00 | 13.47 | A |
| ATOM | 710 | N | ALA | A | 93 | 102.488 | 50.579 | 48.246 | 1.00 | 11.85 | A |
| ATOM | 711 | CA | ALA | A | 93 | 102.238 | 51.910 | 48.792 | 1.00 | 11.98 | A |
| ATOM | 712 | CB | ALA | A | 93 | 100.872 | 52.411 | 48.339 | 1.00 | 11.71 | A |
| ATOM | 713 | C | ALA | A | 93 | 102.306 | 51.879 | 50.313 | 1.00 | 12.55 | A |
| ATOM | 714 | O | ALA | A | 93 | 101.831 | 50.934 | 50.949 | 1.00 | 12.43 | A |
| ATOM | 715 | N | SER | A | 94 | 102.877 | 52.927 | 50.897 | 1.00 | 11.43 | A |
| ATOM | 716 | CA | SER | A | 94 | 103.021 | 52.989 | 52.345 | 1.00 | 12.01 | A |
| ATOM | 717 | CB | SER | A | 94 | 104.335 | 52.312 | 52.757 | 1.00 | 11.41 | A |
| ATOM | 718 | OG | SER | A | 94 | 104.428 | 52.169 | 54.156 | 1.00 | 11.00 | A |
| ATOM | 719 | C | SER | A | 94 | 102.995 | 54.425 | 52.849 | 1.00 | 13.13 | A |
| ATOM | 720 | O | SER | A | 94 | 103.448 | 55.343 | 52.168 | 1.00 | 14.06 | A |
| ATOM | 721 | N | THR | A | 95 | 102.455 | 54.611 | 54.048 | 1.00 | 12.46 | A |
| ATOM | 722 | CA | THR | A | 95 | 102.375 | 55.934 | 54.653 | 1.00 | 12.94 | A |
| ATOM | 723 | CB | THR | A | 95 | 101.102 | 56.069 | 55.516 | 1.00 | 13.47 | A |
| ATOM | 724 | OG1 | THR | A | 95 | 99.941 | 55.947 | 54.682 | 1.00 | 13.30 | A |
| ATOM | 725 | CG2 | THR | A | 95 | 101.067 | 57.424 | 56.216 | 1.00 | 13.18 | A |
| ATOM | 726 | C | THR | A | 95 | 103.594 | 56.192 | 55.529 | 1.00 | 13.42 | A |
| ATOM | 727 | O | THR | A | 95 | 104.040 | 55.313 | 56.268 | 1.00 | 13.58 | A |
| ATOM | 728 | N | VAL | A | 96 | 104.140 | 57.400 | 55.424 | 1.00 | 12.17 | A |
| ATOM | 729 | CA | VAL | A | 96 | 105.290 | 57.798 | 56.227 | 1.00 | 13.84 | A |
| ATOM | 730 | CB | VAL | A | 96 | 106.098 | 58.917 | 55.532 | 1.00 | 14.25 | A |
| ATOM | 731 | CG1 | VAL | A | 96 | 107.277 | 59.332 | 56.401 | 1.00 | 15.75 | A |
| ATOM | 732 | CG2 | VAL | A | 96 | 106.588 | 58.432 | 54.173 | 1.00 | 14.65 | A |
| ATOM | 733 | C | VAL | A | 96 | 104.715 | 58.320 | 57.537 | 1.00 | 13.87 | A |
| ATOM | 734 | O | VAL | A | 96 | 103.958 | 59.289 | 57.544 | 1.00 | 15.08 | A |
| ATOM | 735 | N | TYR | A | 97 | 105.063 | 57.668 | 58.641 | 1.00 | 14.50 | A |
| ATOM | 736 | CA | TYR | A | 97 | 104.548 | 58.047 | 59.952 | 1.00 | 14.20 | A |
| ATOM | 737 | CB | TYR | A | 97 | 105.222 | 57.203 | 61.036 | 1.00 | 15.89 | A |
| ATOM | 738 | CG | TYR | A | 97 | 104.764 | 57.528 | 62.437 | 1.00 | 17.77 | A |
| ATOM | 739 | CD1 | TYR | A | 97 | 103.528 | 57.088 | 62.909 | 1.00 | 19.66 | A |
| ATOM | 740 | CE1 | TYR | A | 97 | 103.103 | 57.393 | 64.202 | 1.00 | 22.46 | A |
| ATOM | 741 | CD2 | TYR | A | 97 | 105.565 | 58.286 | 63.291 | 1.00 | 19.98 | A |
| ATOM | 742 | CE2 | TYR | A | 97 | 105.149 | 58.597 | 64.582 | 1.00 | 21.04 | A |
| ATOM | 743 | CZ | TYR | A | 97 | 103.921 | 58.148 | 65.031 | 1.00 | 22.37 | A |
| ATOM | 744 | OH | TYR | A | 97 | 103.509 | 58.445 | 66.311 | 1.00 | 26.46 | A |
| ATOM | 745 | C | TYR | A | 97 | 104.727 | 59.525 | 60.288 | 1.00 | 15.35 | A |
| ATOM | 746 | O | TYR | A | 97 | 103.784 | 60.193 | 60.711 | 1.00 | 15.68 | A |
| ATOM | 747 | N | GLU | A | 98 | 105.943 | 60.021 | 60.093 | 1.00 | 16.68 | A |
| ATOM | 748 | CA | GLU | A | 98 | 106.283 | 61.405 | 60.398 | 1.00 | 17.49 | A |
| ATOM | 749 | CB | GLU | A | 98 | 107.785 | 61.634 | 60.188 | 1.00 | 17.80 | A |
| ATOM | 750 | CG | GLU | A | 98 | 108.700 | 60.932 | 61.191 | 1.00 | 17.52 | A |
| ATOM | 751 | CD | GLU | A | 98 | 108.934 | 59.462 | 60.876 | 1.00 | 17.50 | A |
| ATOM | 752 | OE1 | GLU | A | 98 | 108.476 | 58.992 | 59.812 | 1.00 | 15.81 | A |
| ATOM | 753 | OE2 | GLU | A | 98 | 109.587 | 58.782 | 61.695 | 1.00 | 17.16 | A |
| ATOM | 754 | C | GLU | A | 98 | 105.518 | 62.481 | 59.626 | 1.00 | 18.07 | A |
| ATOM | 755 | O | GLU | A | 98 | 105.303 | 63.579 | 60.144 | 1.00 | 19.23 | A |
| ATOM | 756 | N | THR | A | 99 | 105.104 | 62.174 | 58.401 | 1.00 | 17.76 | A |
| ATOM | 757 | CA | THR | A | 99 | 104.411 | 63.156 | 57.570 | 1.00 | 17.26 | A |
| ATOM | 758 | CB | THR | A | 99 | 105.134 | 63.339 | 56.233 | 1.00 | 18.41 | A |
| ATOM | 759 | OG1 | THR | A | 99 | 105.068 | 62.115 | 55.489 | 1.00 | 17.88 | A |
| ATOM | 760 | CG2 | THR | A | 99 | 106.592 | 63.706 | 56.462 | 1.00 | 19.12 | A |
| ATOM | 761 | C | THR | A | 99 | 102.957 | 62.860 | 57.245 | 1.00 | 18.06 | A |
| ATOM | 762 | O | THR | A | 99 | 102.211 | 63.756 | 56.843 | 1.00 | 16.88 | A |
| ATOM | 763 | N | GLY | A | 100 | 102.554 | 61.607 | 57.406 | 1.00 | 15.99 | A |
| ATOM | 764 | CA | GLY | A | 100 | 101.184 | 61.244 | 57.096 | 1.00 | 15.99 | A |
| ATOM | 765 | C | GLY | A | 100 | 100.953 | 61.216 | 55.595 | 1.00 | 15.26 | A |
| ATOM | 766 | O | GLY | A | 100 | 99.813 | 61.202 | 55.136 | 1.00 | 16.46 | A |
| ATOM | 767 | N | GLU | A | 101 | 102.033 | 61.215 | 54.821 | 1.00 | 15.67 | A |
| ATOM | 768 | CA | GLU | A | 101 | 101.907 | 61.178 | 53.372 | 1.00 | 15.74 | A |
| ATOM | 769 | CB | GLU | A | 101 | 103.001 | 62.012 | 52.707 | 1.00 | 18.28 | A |
| ATOM | 770 | CG | GLU | A | 101 | 102.958 | 63.487 | 53.060 | 1.00 | 20.54 | A |
| ATOM | 771 | CD | GLU | A | 101 | 103.894 | 64.306 | 52.204 | 1.00 | 23.37 | A |
| ATOM | 772 | OE1 | GLU | A | 101 | 105.062 | 63.888 | 52.037 | 1.00 | 22.75 | A |
| ATOM | 773 | OE2 | GLU | A | 101 | 103.470 | 65.374 | 51.702 | 1.00 | 25.57 | A |
| ATOM | 774 | C | GLU | A | 101 | 101.994 | 59.746 | 52.862 | 1.00 | 15.76 | A |
| ATOM | 775 | O | GLU | A | 101 | 102.691 | 58.910 | 53.440 | 1.00 | 15.42 | A |
| ATOM | 776 | N | VAL | A | 102 | 101.281 | 59.472 | 51.776 | 1.00 | 14.64 | A |
| ATOM | 777 | CA | VAL | A | 102 | 101.275 | 58.148 | 51.173 | 1.00 | 14.19 | A |
| ATOM | 778 | CB | VAL | A | 102 | 99.849 | 57.752 | 50.716 | 1.00 | 14.08 | A |
| ATOM | 779 | CG1 | VAL | A | 102 | 99.865 | 56.359 | 50.100 | 1.00 | 15.37 | A |
| ATOM | 780 | CG2 | VAL | A | 102 | 98.892 | 57.803 | 51.894 | 1.00 | 15.16 | A |
| ATOM | 781 | C | VAL | A | 102 | 102.198 | 58.144 | 49.965 | 1.00 | 14.18 | A |
| ATOM | 782 | O | VAL | A | 102 | 102.077 | 58.989 | 49.078 | 1.00 | 14.73 | A |
| ATOM | 783 | N | LYS | A | 103 | 103.135 | 57.204 | 49.937 | 1.00 | 13.82 | A |
| ATOM | 784 | CA | LYS | A | 103 | 104.052 | 57.105 | 48.814 | 1.00 | 12.99 | A |
| ATOM | 785 | CB | LYS | A | 103 | 105.499 | 57.175 | 49.303 | 1.00 | 13.98 | A |
| ATOM | 786 | CG | LYS | A | 103 | 105.825 | 58.532 | 49.923 | 1.00 | 15.54 | A |
| ATOM | 787 | CD | LYS | A | 103 | 107.308 | 58.735 | 50.169 | 1.00 | 16.25 | A |
| ATOM | 788 | CE | LYS | A | 103 | 107.561 | 60.139 | 50.709 | 1.00 | 18.61 | A |
| ATOM | 789 | NZ | LYS | A | 103 | 109.011 | 60.421 | 50.894 | 1.00 | 19.08 | A |
| ATOM | 790 | C | LYS | A | 103 | 103.785 | 55.814 | 48.052 | 1.00 | 14.30 | A |
| ATOM | 791 | O | LYS | A | 103 | 103.667 | 54.739 | 48.644 | 1.00 | 13.53 | A |
| ATOM | 792 | N | VAL | A | 104 | 103.676 | 55.943 | 46.733 | 1.00 | 13.28 | A |
| ATOM | 793 | CA | VAL | A | 104 | 103.384 | 54.823 | 45.846 | 1.00 | 13.24 | A |
| ATOM | 794 | CB | VAL | A | 104 | 102.189 | 55.167 | 44.932 | 1.00 | 13.19 | A |
| ATOM | 795 | CG1 | VAL | A | 104 | 101.772 | 53.944 | 44.127 | 1.00 | 13.70 | A |
| ATOM | 796 | CG2 | VAL | A | 104 | 101.040 | 55.688 | 45.771 | 1.00 | 12.66 | A |
| ATOM | 797 | C | VAL | A | 104 | 104.567 | 54.447 | 44.961 | 1.00 | 13.76 | A |
| ATOM | 798 | O | VAL | A | 104 | 105.136 | 55.295 | 44.271 | 1.00 | 14.89 | A |
| ATOM | 799 | N | TYR | A | 105 | 104.923 | 53.167 | 44.981 | 1.00 | 13.39 | A |
| ATOM | 800 | CA | TYR | A | 105 | 106.027 | 52.663 | 44.180 | 1.00 | 13.88 | A |
| ATOM | 801 | CB | TYR | A | 105 | 107.160 | 52.165 | 45.084 | 1.00 | 14.03 | A |
| ATOM | 802 | CG | TYR | A | 105 | 107.596 | 53.173 | 46.123 | 1.00 | 14.05 | A |
| ATOM | 803 | CD1 | TYR | A | 105 | 106.881 | 53.332 | 47.312 | 1.00 | 14.04 | A |
| ATOM | 804 | CE1 | TYR | A | 105 | 107.256 | 54.287 | 48.255 | 1.00 | 15.86 | A |
| ATOM | 805 | CD2 | TYR | A | 105 | 108.701 | 53.994 | 45.900 | 1.00 | 13.95 | A |
| ATOM | 806 | CE2 | TYR | A | 105 | 109.083 | 54.954 | 46.832 | 1.00 | 16.06 | A |
| ATOM | 807 | CZ | TYR | A | 105 | 108.358 | 55.096 | 48.003 | 1.00 | 15.47 | A |
| ATOM | 808 | OH | TYR | A | 105 | 108.723 | 56.056 | 48.913 | 1.00 | 17.33 | A |
| ATOM | 809 | C | TYR | A | 105 | 105.540 | 51.517 | 43.306 | 1.00 | 14.30 | A |
| ATOM | 810 | O | TYR | A | 105 | 104.640 | 50.775 | 43.695 | 1.00 | 15.27 | A |
| ATOM | 811 | N | VAL | A | 106 | 106.130 | 51.379 | 42.124 | 1.00 | 13.86 | A |
| ATOM | 812 | CA | VAL | A | 106 | 105.751 | 50.308 | 41.210 | 1.00 | 14.02 | A |
| ATOM | 813 | CB | VAL | A | 106 | 104.907 | 50.841 | 40.023 | 1.00 | 14.52 | A |
| ATOM | 814 | CG1 | VAL | A | 106 | 103.663 | 51.531 | 40.536 | 1.00 | 14.58 | A |
| ATOM | 815 | CG2 | VAL | A | 106 | 105.737 | 51.799 | 39.178 | 1.00 | 15.86 | A |
| ATOM | 816 | C | VAL | A | 106 | 106.983 | 49.613 | 40.649 | 1.00 | 14.69 | A |
| ATOM | 817 | O | VAL | A | 106 | 108.078 | 50.177 | 40.631 | 1.00 | 15.19 | A |
| ATOM | 818 | N | SER | A | 107 | 106.797 | 48.378 | 40.202 | 1.00 | 12.97 | A |
| ATOM | 819 | CA | SER | A | 107 | 107.879 | 47.602 | 39.609 | 1.00 | 13.73 | A |
| ATOM | 820 | CB | SER | A | 107 | 108.438 | 46.592 | 40.608 | 1.00 | 13.74 | A |
| ATOM | 821 | OG | SER | A | 107 | 109.342 | 45.713 | 39.957 | 1.00 | 13.27 | A |
| ATOM | 822 | C | SER | A | 107 | 107.351 | 46.859 | 38.390 | 1.00 | 14.26 | A |
| ATOM | 823 | O | SER | A | 107 | 106.313 | 46.199 | 38.457 | 1.00 | 13.99 | A |
| ATOM | 824 | N | ASP | A | 108 | 108.077 | 46.966 | 37.282 | 1.00 | 13.81 | A |
| ATOM | 825 | CA | ASP | A | 108 | 107.686 | 46.313 | 36.041 | 1.00 | 15.53 | A |
| ATOM | 826 | CB | ASP | A | 108 | 107.790 | 47.295 | 34.875 | 1.00 | 16.53 | A |
| ATOM | 827 | CG | ASP | A | 108 | 106.770 | 48.405 | 34.967 | 1.00 | 18.99 | A |
| ATOM | 828 | OD1 | ASP | A | 108 | 105.562 | 48.092 | 35.074 | 1.00 | 19.31 | A |
| ATOM | 829 | OD2 | ASP | A | 108 | 107.167 | 49.591 | 34.929 | 1.00 | 20.35 | A |
| ATOM | 830 | C | ASP | A | 108 | 108.509 | 45.073 | 35.729 | 1.00 | 16.33 | A |
| ATOM | 831 | O | ASP | A | 108 | 108.264 | 44.403 | 34.725 | 1.00 | 17.53 | A |
| ATOM | 832 | N | ASP | A | 109 | 109.485 | 44.768 | 36.579 | 1.00 | 15.91 | A |
| ATOM | 833 | CA | ASP | A | 109 | 110.329 | 43.600 | 36.361 | 1.00 | 16.74 | A |
| ATOM | 834 | CB | ASP | A | 109 | 111.787 | 44.020 | 36.146 | 1.00 | 17.40 | A |
| ATOM | 835 | CG | ASP | A | 109 | 112.315 | 44.904 | 37.251 | 1.00 | 17.59 | A |
| ATOM | 836 | OD1 | ASP | A | 109 | 111.663 | 45.002 | 38.309 | 1.00 | 16.99 | A |
| ATOM | 837 | OD2 | ASP | A | 109 | 113.399 | 45.502 | 37.060 | 1.00 | 19.87 | A |
| ATOM | 838 | C | ASP | A | 109 | 110.239 | 42.590 | 37.499 | 1.00 | 16.89 | A |
| ATOM | 839 | O | ASP | A | 109 | 111.248 | 42.054 | 37.954 | 1.00 | 16.91 | A |
| ATOM | 840 | N | SER | A | 110 | 109.015 | 42.343 | 37.953 | 1.00 | 17.36 | A |
| ATOM | 841 | CA | SER | A | 110 | 108.749 | 41.380 | 39.014 | 1.00 | 18.26 | A |
| ATOM | 842 | CB | SER | A | 110 | 109.080 | 39.971 | 38.513 | 1.00 | 19.68 | A |
| ATOM | 843 | OG | SER | A | 110 | 108.238 | 39.623 | 37.431 | 1.00 | 23.69 | A |
| ATOM | 844 | C | SER | A | 110 | 109.445 | 41.637 | 40.347 | 1.00 | 17.69 | A |
| ATOM | 845 | O | SER | A | 110 | 109.847 | 40.698 | 41.037 | 1.00 | 18.27 | A |
| ATOM | 846 | N | GLY | A | 111 | 109.588 | 42.910 | 40.706 | 1.00 | 17.10 | A |
| ATOM | 847 | CA | GLY | A | 111 | 110.196 | 43.254 | 41.981 | 1.00 | 16.28 | A |
| ATOM | 848 | C | GLY | A | 111 | 111.691 | 43.508 | 42.014 | 1.00 | 18.11 | A |
| ATOM | 849 | O | GLY | A | 111 | 112.251 | 43.742 | 43.088 | 1.00 | 16.80 | A |
| ATOM | 850 | N | VAL | A | 112 | 112.345 | 43.464 | 40.859 | 1.00 | 17.52 | A |
| ATOM | 851 | CA | VAL | A | 112 | 113.784 | 43.699 | 40.808 | 1.00 | 19.77 | A |
| ATOM | 852 | CB | VAL | A | 112 | 114.384 | 43.236 | 39.467 | 1.00 | 19.94 | A |
| ATOM | 853 | CG1 | VAL | A | 112 | 115.880 | 43.528 | 39.440 | 1.00 | 21.34 | A |
| ATOM | 854 | CG2 | VAL | A | 112 | 114.136 | 41.746 | 39.278 | 1.00 | 22.11 | A |
| ATOM | 855 | C | VAL | A | 112 | 114.109 | 45.175 | 41.011 | 1.00 | 19.00 | A |
| ATOM | 856 | O | VAL | A | 112 | 114.991 | 45.525 | 41.800 | 1.00 | 19.50 | A |
| ATOM | 857 | N | THR | A | 113 | 113.396 | 46.040 | 40.298 | 1.00 | 19.36 | A |
| ATOM | 858 | CA | THR | A | 113 | 113.615 | 47.474 | 40.410 | 1.00 | 19.88 | A |
| ATOM | 859 | CB | THR | A | 113 | 114.190 | 48.058 | 39.103 | 1.00 | 21.59 | A |
| ATOM | 860 | OG1 | THR | A | 113 | 113.230 | 47.923 | 38.049 | 1.00 | 24.29 | A |
| ATOM | 861 | CG2 | THR | A | 113 | 115.465 | 47.325 | 38.714 | 1.00 | 23.01 | A |
| ATOM | 862 | C | THR | A | 113 | 112.309 | 48.181 | 40.741 | 1.00 | 19.15 | A |
| ATOM | 863 | O | THR | A | 113 | 111.238 | 47.768 | 40.296 | 1.00 | 18.90 | A |
| ATOM | 864 | N | TRP | A | 114 | 112.403 | 49.244 | 41.530 | 1.00 | 17.82 | A |
| ATOM | 865 | CA | TRP | A | 114 | 111.227 | 50.000 | 41.925 | 1.00 | 17.76 | A |
| ATOM | 866 | CB | TRP | A | 114 | 110.988 | 49.835 | 43.425 | 1.00 | 16.50 | A |
| ATOM | 867 | CG | TRP | A | 114 | 110.690 | 48.422 | 43.791 | 1.00 | 15.27 | A |
| ATOM | 868 | CD2 | TRP | A | 114 | 109.394 | 47.838 | 43.930 | 1.00 | 15.36 | A |
| ATOM | 869 | CE2 | TRP | A | 114 | 109.582 | 46.464 | 44.200 | 1.00 | 14.55 | A |
| ATOM | 870 | CE3 | TRP | A | 114 | 108.088 | 48.341 | 43.846 | 1.00 | 15.10 | A |
| ATOM | 871 | CD1 | TRP | A | 114 | 111.590 | 47.413 | 43.982 | 1.00 | 14.32 | A |
| ATOM | 872 | NE1 | TRP | A | 114 | 110.933 | 46.233 | 44.228 | 1.00 | 14.49 | A |
| ATOM | 873 | CZ2 | TRP | A | 114 | 108.510 | 45.584 | 44.392 | 1.00 | 14.31 | A |
| ATOM | 874 | CZ3 | TRP | A | 114 | 107.025 | 47.468 | 44.036 | 1.00 | 14.50 | A |
| ATOM | 875 | CH2 | TRP | A | 114 | 107.244 | 46.103 | 44.305 | 1.00 | 12.54 | A |
| ATOM | 876 | C | TRP | A | 114 | 111.353 | 51.471 | 41.576 | 1.00 | 18.48 | A |
| ATOM | 877 | O | TRP | A | 114 | 112.437 | 52.051 | 41.657 | 1.00 | 19.19 | A |
| ATOM | 878 | N | VAL | A | 115 | 110.236 | 52.069 | 41.181 | 1.00 | 18.44 | A |
| ATOM | 879 | CA | VAL | A | 115 | 110.218 | 53.478 | 40.818 | 1.00 | 19.71 | A |
| ATOM | 880 | CB | VAL | A | 115 | 109.975 | 53.663 | 39.303 | 1.00 | 20.11 | A |
| ATOM | 881 | CG1 | VAL | A | 115 | 109.936 | 55.147 | 38.961 | 1.00 | 21.12 | A |
| ATOM | 882 | CG2 | VAL | A | 115 | 111.076 | 52.969 | 38.512 | 1.00 | 20.67 | A |
| ATOM | 883 | C | VAL | A | 115 | 109.131 | 54.221 | 41.580 | 1.00 | 19.62 | A |
| ATOM | 884 | O | VAL | A | 115 | 108.003 | 53.742 | 41.697 | 1.00 | 19.62 | A |
| ATOM | 885 | N | HIS | A | 116 | 109.484 | 55.389 | 42.107 | 1.00 | 20.01 | A |
| ATOM | 886 | CA | HIS | A | 116 | 108.544 | 56.222 | 42.844 | 1.00 | 20.97 | A |
| ATOM | 887 | CB | HIS | A | 116 | 109.302 | 57.286 | 43.640 | 1.00 | 22.54 | A |
| ATOM | 888 | CG | HIS | A | 116 | 108.413 | 58.275 | 44.325 | 1.00 | 24.51 | A |
| ATOM | 889 | CD2 | HIS | A | 116 | 108.375 | 59.628 | 44.274 | 1.00 | 25.76 | A |
| ATOM | 890 | ND1 | HIS | A | 116 | 107.419 | 57.897 | 45.201 | 1.00 | 24.78 | A |
| ATOM | 891 | CE1 | HIS | A | 116 | 106.807 | 58.973 | 45.661 | 1.00 | 24.56 | A |
| ATOM | 892 | NE2 | HIS | A | 116 | 107.368 | 60.037 | 45.114 | 1.00 | 28.11 | A |
| ATOM | 893 | C | HIS | A | 116 | 107.617 | 56.888 | 41.832 | 1.00 | 21.25 | A |
| ATOM | 894 | O | HIS | A | 116 | 108.079 | 57.551 | 40.903 | 1.00 | 21.92 | A |
| ATOM | 895 | N | LYS | A | 117 | 106.312 | 56.716 | 42.013 | 1.00 | 19.82 | A |
| ATOM | 896 | CA | LYS | A | 117 | 105.344 | 57.284 | 41.083 | 1.00 | 19.82 | A |
| ATOM | 897 | CB | LYS | A | 117 | 104.463 | 56.170 | 40.512 | 1.00 | 20.35 | A |
| ATOM | 898 | CG | LYS | A | 117 | 105.190 | 55.201 | 39.599 | 1.00 | 20.63 | A |
| ATOM | 899 | CD | LYS | A | 117 | 105.615 | 55.879 | 38.307 | 1.00 | 20.10 | A |
| ATOM | 900 | CE | LYS | A | 117 | 106.293 | 54.899 | 37.366 | 1.00 | 21.83 | A |
| ATOM | 901 | NZ | LYS | A | 117 | 106.664 | 55.545 | 36.077 | 1.00 | 22.39 | A |
| ATOM | 902 | C | LYS | A | 117 | 104.441 | 58.375 | 41.641 | 1.00 | 19.81 | A |
| ATOM | 903 | O | LYS | A | 117 | 103.940 | 59.208 | 40.885 | 1.00 | 20.98 | A |
| ATOM | 904 | N | PHE | A | 118 | 104.230 | 58.383 | 42.953 | 1.00 | 18.35 | A |
| ATOM | 905 | CA | PHE | A | 118 | 103.337 | 59.376 | 43.534 | 1.00 | 17.46 | A |
| ATOM | 906 | CB | PHE | A | 118 | 101.891 | 58.990 | 43.205 | 1.00 | 18.22 | A |
| ATOM | 907 | CG | PHE | A | 118 | 100.867 | 59.999 | 43.642 | 1.00 | 19.77 | A |
| ATOM | 908 | CD1 | PHE | A | 118 | 100.790 | 61.242 | 43.023 | 1.00 | 21.15 | A |
| ATOM | 909 | CD2 | PHE | A | 118 | 99.953 | 59.692 | 44.646 | 1.00 | 18.89 | A |
| ATOM | 910 | CE1 | PHE | A | 118 | 99.816 | 62.164 | 43.395 | 1.00 | 22.24 | A |
| ATOM | 911 | CE2 | PHE | A | 118 | 98.977 | 60.607 | 45.025 | 1.00 | 19.79 | A |
| ATOM | 912 | CZ | PHE | A | 118 | 98.906 | 61.845 | 44.397 | 1.00 | 21.59 | A |
| ATOM | 913 | C | PHE | A | 118 | 103.468 | 59.538 | 45.042 | 1.00 | 17.87 | A |
| ATOM | 914 | O | PHE | A | 118 | 103.848 | 58.609 | 45.754 | 1.00 | 16.54 | A |
| ATOM | 915 | N | THR | A | 119 | 103.148 | 60.738 | 45.514 | 1.00 | 17.39 | A |
| ATOM | 916 | CA | THR | A | 119 | 103.157 | 61.048 | 46.935 | 1.00 | 16.92 | A |
| ATOM | 917 | CB | THR | A | 119 | 104.371 | 61.913 | 47.340 | 1.00 | 16.66 | A |
| ATOM | 918 | OG1 | THR | A | 119 | 105.571 | 61.140 | 47.221 | 1.00 | 15.94 | A |
| ATOM | 919 | CG2 | THR | A | 119 | 104.228 | 62.393 | 48.779 | 1.00 | 16.97 | A |
| ATOM | 920 | C | THR | A | 119 | 101.879 | 61.833 | 47.197 | 1.00 | 17.49 | A |
| ATOM | 921 | O | THR | A | 119 | 101.654 | 62.887 | 46.594 | 1.00 | 17.41 | A |
| ATOM | 922 | N | LEU | A | 120 | 101.036 | 61.307 | 48.078 | 1.00 | 16.30 | A |
| ATOM | 923 | CA | LEU | A | 120 | 99.778 | 61.957 | 48.416 | 1.00 | 17.68 | A |
| ATOM | 924 | CB | LEU | A | 120 | 98.699 | 60.912 | 48.722 | 1.00 | 17.97 | A |
| ATOM | 925 | CG | LEU | A | 120 | 97.337 | 61.461 | 49.170 | 1.00 | 18.94 | A |
| ATOM | 926 | CD1 | LEU | A | 120 | 96.705 | 62.250 | 48.035 | 1.00 | 19.16 | A |
| ATOM | 927 | CD2 | LEU | A | 120 | 96.425 | 60.315 | 49.589 | 1.00 | 18.14 | A |
| ATOM | 928 | C | LEU | A | 120 | 99.937 | 62.872 | 49.620 | 1.00 | 18.22 | A |
| ATOM | 929 | O | LEU | A | 120 | 100.214 | 62.411 | 50.728 | 1.00 | 17.72 | A |
| ATOM | 930 | N | ALA | A | 121 | 99.768 | 64.171 | 49.399 | 1.00 | 19.46 | A |
| ATOM | 931 | CA | ALA | A | 121 | 99.873 | 65.140 | 50.482 | 1.00 | 21.04 | A |
| ATOM | 932 | CB | ALA | A | 121 | 99.919 | 66.555 | 49.915 | 1.00 | 21.23 | A |
| ATOM | 933 | C | ALA | A | 121 | 98.650 | 64.969 | 51.376 | 1.00 | 21.73 | A |
| ATOM | 934 | O | ALA | A | 121 | 97.551 | 64.718 | 50.885 | 1.00 | 22.55 | A |
| ATOM | 935 | N | ARG | A | 122 | 98.835 | 65.106 | 52.685 | 1.00 | 23.36 | A |
| ATOM | 936 | CA | ARG | A | 122 | 97.726 | 64.943 | 53.618 | 1.00 | 24.95 | A |
| ATOM | 937 | CB | ARG | A | 122 | 98.250 | 64.816 | 55.054 | 1.00 | 26.32 | A |
| ATOM | 938 | CG | ARG | A | 122 | 98.867 | 66.078 | 55.635 | 1.00 | 28.30 | A |
| ATOM | 939 | CD | ARG | A | 122 | 99.514 | 65.783 | 56.983 | 1.00 | 30.66 | A |
| ATOM | 940 | NE | ARG | A | 122 | 99.991 | 66.990 | 57.652 | 1.00 | 33.28 | A |
| ATOM | 941 | CZ | ARG | A | 122 | 99.201 | 67.876 | 58.252 | 1.00 | 34.92 | A |
| ATOM | 942 | NH1 | ARG | A | 122 | 97.888 | 67.693 | 58.272 | 1.00 | 35.57 | A |
| ATOM | 943 | NH2 | ARG | A | 122 | 99.724 | 68.947 | 58.832 | 1.00 | 36.62 | A |
| ATOM | 944 | C | ARG | A | 122 | 96.730 | 66.089 | 53.524 | 1.00 | 26.19 | A |
| ATOM | 945 | O | ARG | A | 122 | 95.540 | 65.908 | 53.774 | 1.00 | 25.14 | A |
| ATOM | 946 | N | GLY | A | 123 | 97.220 | 67.268 | 53.154 | 1.00 | 27.46 | A |
| ATOM | 947 | CA | GLY | A | 123 | 96.348 | 68.421 | 53.040 | 1.00 | 28.77 | A |
| ATOM | 948 | C | GLY | A | 123 | 95.754 | 68.815 | 54.378 | 1.00 | 28.48 | A |
| ATOM | 949 | O | GLY | A | 123 | 96.481 | 69.092 | 55.332 | 1.00 | 30.08 | A |
| ATOM | 950 | N | GLY | A | 124 | 94.427 | 68.835 | 54.449 | 1.00 | 28.27 | A |
| ATOM | 951 | CA | GLY | A | 124 | 93.757 | 69.202 | 55.683 | 1.00 | 27.63 | A |
| ATOM | 952 | C | GLY | A | 124 | 93.541 | 68.041 | 56.635 | 1.00 | 26.98 | A |
| ATOM | 953 | O | GLY | A | 124 | 93.210 | 68.244 | 57.802 | 1.00 | 27.21 | A |
| ATOM | 954 | N | ALA | A | 125 | 93.724 | 66.820 | 56.141 | 1.00 | 26.44 | A |
| ATOM | 955 | CA | ALA | A | 125 | 93.545 | 65.634 | 56.971 | 1.00 | 24.57 | A |
| ATOM | 956 | CB | ALA | A | 125 | 93.296 | 64.412 | 56.092 | 1.00 | 25.40 | A |
| ATOM | 957 | C | ALA | A | 125 | 94.779 | 65.411 | 57.836 | 1.00 | 23.60 | A |
| ATOM | 958 | O | ALA | A | 125 | 95.897 | 65.723 | 57.426 | 1.00 | 23.64 | A |
| ATOM | 959 | N | ASP | A | 126 | 94.577 | 64.877 | 59.035 | 1.00 | 22.65 | A |
| ATOM | 960 | CA | ASP | A | 126 | 95.696 | 64.622 | 59.930 | 1.00 | 21.78 | A |
| ATOM | 961 | CB | ASP | A | 126 | 95.212 | 64.037 | 61.261 | 1.00 | 22.89 | A |
| ATOM | 962 | CG | ASP | A | 126 | 94.246 | 64.955 | 61.986 | 1.00 | 24.95 | A |
| ATOM | 963 | OD1 | ASP | A | 126 | 94.350 | 66.189 | 61.808 | 1.00 | 26.65 | A |
| ATOM | 964 | OD2 | ASP | A | 126 | 93.396 | 64.443 | 62.746 | 1.00 | 27.55 | A |
| ATOM | 965 | C | ASP | A | 126 | 96.673 | 63.657 | 59.270 | 1.00 | 21.38 | A |
| ATOM | 966 | O | ASP | A | 126 | 97.880 | 63.714 | 59.514 | 1.00 | 20.71 | A |
| ATOM | 967 | N | TYR | A | 127 | 96.144 | 62.774 | 58.426 | 1.00 | 19.24 | A |
| ATOM | 968 | CA | TYR | A | 127 | 96.966 | 61.790 | 57.729 | 1.00 | 17.63 | A |
| ATOM | 969 | CB | TYR | A | 127 | 97.661 | 60.878 | 58.749 | 1.00 | 19.50 | A |
| ATOM | 970 | CG | TYR | A | 127 | 96.699 | 60.104 | 59.636 | 1.00 | 19.37 | A |
| ATOM | 971 | CD1 | TYR | A | 127 | 95.970 | 59.020 | 59.139 | 1.00 | 19.51 | A |
| ATOM | 972 | CE1 | TYR | A | 127 | 95.066 | 58.325 | 59.943 | 1.00 | 19.33 | A |
| ATOM | 973 | CD2 | TYR | A | 127 | 96.498 | 60.473 | 60.967 | 1.00 | 20.65 | A |
| ATOM | 974 | CE2 | TYR | A | 127 | 95.595 | 59.785 | 61.780 | 1.00 | 20.27 | A |
| ATOM | 975 | CZ | TYR | A | 127 | 94.883 | 58.713 | 61.261 | 1.00 | 21.57 | A |
| ATOM | 976 | OH | TYR | A | 127 | 93.991 | 58.036 | 62.061 | 1.00 | 22.90 | A |
| ATOM | 977 | C | TYR | A | 127 | 96.123 | 60.925 | 56.801 | 1.00 | 16.28 | A |
| ATOM | 978 | O | TYR | A | 127 | 94.899 | 60.895 | 56.907 | 1.00 | 16.70 | A |
| ATOM | 979 | N | ASN | A | 128 | 96.794 | 60.240 | 55.883 | 1.00 | 14.82 | A |
| ATOM | 980 | CA | ASN | A | 128 | 96.151 | 59.301 | 54.971 | 1.00 | 14.64 | A |
| ATOM | 981 | CB | ASN | A | 128 | 96.250 | 59.760 | 53.518 | 1.00 | 15.13 | A |
| ATOM | 982 | CG | ASN | A | 128 | 95.215 | 60.814 | 53.170 | 1.00 | 16.92 | A |
| ATOM | 983 | OD1 | ASN | A | 128 | 94.007 | 60.588 | 53.307 | 1.00 | 17.90 | A |
| ATOM | 984 | ND2 | ASN | A | 128 | 95.680 | 61.971 | 52.713 | 1.00 | 17.93 | A |
| ATOM | 985 | C | ASN | A | 128 | 96.957 | 58.030 | 55.180 | 1.00 | 13.76 | A |
| ATOM | 986 | O | ASN | A | 128 | 98.176 | 58.025 | 55.000 | 1.00 | 13.93 | A |
| ATOM | 987 | N | PHE | A | 129 | 96.277 | 56.959 | 55.571 | 1.00 | 13.25 | A |
| ATOM | 988 | CA | PHE | A | 129 | 96.946 | 55.698 | 55.857 | 1.00 | 12.85 | A |
| ATOM | 989 | CB | PHE | A | 129 | 96.551 | 55.240 | 57.261 | 1.00 | 13.65 | A |
| ATOM | 990 | CG | PHE | A | 129 | 97.566 | 54.355 | 57.920 | 1.00 | 17.42 | A |
| ATOM | 991 | CD1 | PHE | A | 129 | 97.607 | 52.993 | 57.649 | 1.00 | 18.64 | A |
| ATOM | 992 | CD2 | PHE | A | 129 | 98.489 | 54.889 | 58.810 | 1.00 | 18.37 | A |
| ATOM | 993 | CE1 | PHE | A | 129 | 98.556 | 52.170 | 58.265 | 1.00 | 19.40 | A |
| ATOM | 994 | CE2 | PHE | A | 129 | 99.440 | 54.079 | 59.431 | 1.00 | 19.59 | A |
| ATOM | 995 | CZ | PHE | A | 129 | 99.473 | 52.717 | 59.155 | 1.00 | 19.15 | A |
| ATOM | 996 | C | PHE | A | 129 | 96.636 | 54.604 | 54.844 | 1.00 | 11.29 | A |
| ATOM | 997 | O | PHE | A | 129 | 95.500 | 54.152 | 54.736 | 1.00 | 12.59 | A |
| ATOM | 998 | N | ALA | A | 130 | 97.653 | 54.189 | 54.097 | 1.00 | 11.32 | A |
| ATOM | 999 | CA | ALA | A | 130 | 97.483 | 53.133 | 53.106 | 1.00 | 10.44 | A |
| ATOM | 1000 | CB | ALA | A | 130 | 98.615 | 53.177 | 52.089 | 1.00 | 10.33 | A |
| ATOM | 1001 | C | ALA | A | 130 | 97.462 | 51.779 | 53.809 | 1.00 | 11.51 | A |
| ATOM | 1002 | O | ALA | A | 130 | 98.286 | 51.513 | 54.680 | 1.00 | 11.91 | A |
| ATOM | 1003 | N | HIS | A | 131 | 96.514 | 50.925 | 53.430 | 1.00 | 10.27 | A |
| ATOM | 1004 | CA | HIS | A | 131 | 96.402 | 49.598 | 54.025 | 1.00 | 8.80 | A |
| ATOM | 1005 | CB | HIS | A | 131 | 95.783 | 49.679 | 55.424 | 1.00 | 9.22 | A |
| ATOM | 1006 | CG | HIS | A | 131 | 95.492 | 48.339 | 56.030 | 1.00 | 10.16 | A |
| ATOM | 1007 | CD2 | HIS | A | 131 | 96.206 | 47.584 | 56.897 | 1.00 | 9.68 | A |
| ATOM | 1008 | ND1 | HIS | A | 131 | 94.372 | 47.599 | 55.712 | 1.00 | 11.64 | A |
| ATOM | 1009 | CE1 | HIS | A | 131 | 94.412 | 46.445 | 56.356 | 1.00 | 9.01 | A |
| ATOM | 1010 | NE2 | HIS | A | 131 | 95.515 | 46.411 | 57.081 | 1.00 | 12.62 | A |
| ATOM | 1011 | C | HIS | A | 131 | 95.548 | 48.667 | 53.185 | 1.00 | 9.59 | A |
| ATOM | 1012 | O | HIS | A | 131 | 94.496 | 49.064 | 52.693 | 1.00 | 10.07 | A |
| ATOM | 1013 | N | GLY | A | 132 | 96.001 | 47.426 | 53.026 | 1.00 | 9.53 | A |
| ATOM | 1014 | CA | GLY | A | 132 | 95.214 | 46.455 | 52.285 | 1.00 | 9.47 | A |
| ATOM | 1015 | C | GLY | A | 132 | 95.681 | 46.044 | 50.905 | 1.00 | 9.24 | A |
| ATOM | 1016 | O | GLY | A | 132 | 96.635 | 46.592 | 50.357 | 1.00 | 10.20 | A |
| ATOM | 1017 | N | LYS | A | 133 | 94.986 | 45.053 | 50.355 | 1.00 | 9.64 | A |
| ATOM | 1018 | CA | LYS | A | 133 | 95.266 | 44.532 | 49.026 | 1.00 | 8.76 | A |
| ATOM | 1019 | CB | LYS | A | 133 | 94.672 | 43.123 | 48.889 | 1.00 | 8.62 | A |
| ATOM | 1020 | CG | LYS | A | 133 | 94.725 | 42.522 | 47.482 | 1.00 | 8.25 | A |
| ATOM | 1021 | CD | LYS | A | 133 | 96.151 | 42.404 | 46.939 | 1.00 | 9.45 | A |
| ATOM | 1022 | CE | LYS | A | 133 | 97.043 | 41.516 | 47.808 | 1.00 | 9.67 | A |
| ATOM | 1023 | NZ | LYS | A | 133 | 96.620 | 40.078 | 47.843 | 1.00 | 9.58 | A |
| ATOM | 1024 | C | LYS | A | 133 | 94.646 | 45.463 | 47.989 | 1.00 | 8.89 | A |
| ATOM | 1025 | O | LYS | A | 133 | 93.464 | 45.810 | 48.080 | 1.00 | 9.54 | A |
| ATOM | 1026 | N | SER | A | 134 | 95.448 | 45.894 | 47.020 | 1.00 | 9.04 | A |
| ATOM | 1027 | CA | SER | A | 134 | 94.934 | 46.767 | 45.974 | 1.00 | 9.67 | A |
| ATOM | 1028 | CB | SER | A | 134 | 96.086 | 47.425 | 45.201 | 1.00 | 10.23 | A |
| ATOM | 1029 | OG | SER | A | 134 | 97.145 | 46.527 | 44.941 | 1.00 | 11.37 | A |
| ATOM | 1030 | C | SER | A | 134 | 94.050 | 45.924 | 45.059 | 1.00 | 11.08 | A |
| ATOM | 1031 | O | SER | A | 134 | 94.138 | 44.694 | 45.059 | 1.00 | 11.02 | A |
| ATOM | 1032 | N | PHE | A | 135 | 93.194 | 46.581 | 44.286 | 1.00 | 10.74 | A |
| ATOM | 1033 | CA | PHE | A | 135 | 92.270 | 45.862 | 43.419 | 1.00 | 10.61 | A |
| ATOM | 1034 | CB | PHE | A | 135 | 91.027 | 45.476 | 44.223 | 1.00 | 10.53 | A |
| ATOM | 1035 | CG | PHE | A | 135 | 90.411 | 46.628 | 44.971 | 1.00 | 10.41 | A |
| ATOM | 1036 | CD1 | PHE | A | 135 | 90.967 | 47.078 | 46.166 | 1.00 | 10.82 | A |
| ATOM | 1037 | CD2 | PHE | A | 135 | 89.292 | 47.280 | 44.466 | 1.00 | 11.06 | A |
| ATOM | 1038 | CE1 | PHE | A | 135 | 90.419 | 48.164 | 46.844 | 1.00 | 10.93 | A |
| ATOM | 1039 | CE2 | PHE | A | 135 | 88.733 | 48.370 | 45.136 | 1.00 | 11.39 | A |
| ATOM | 1040 | CZ | PHE | A | 135 | 89.297 | 48.812 | 46.327 | 1.00 | 11.22 | A |
| ATOM | 1041 | C | PHE | A | 135 | 91.851 | 46.660 | 42.191 | 1.00 | 11.31 | A |
| ATOM | 1042 | O | PHE | A | 135 | 92.036 | 47.874 | 42.130 | 1.00 | 11.81 | A |
| ATOM | 1043 | N | GLN | A | 136 | 91.269 | 45.960 | 41.220 | 1.00 | 11.14 | A |
| ATOM | 1044 | CA | GLN | A | 136 | 90.821 | 46.574 | 39.975 | 1.00 | 11.93 | A |
| ATOM | 1045 | CB | GLN | A | 136 | 91.189 | 45.668 | 38.796 | 1.00 | 12.95 | A |
| ATOM | 1046 | CG | GLN | A | 136 | 90.873 | 46.232 | 37.413 | 1.00 | 13.64 | A |
| ATOM | 1047 | CD | GLN | A | 136 | 91.882 | 47.272 | 36.963 | 1.00 | 14.32 | A |
| ATOM | 1048 | OE1 | GLN | A | 136 | 93.051 | 47.223 | 37.346 | 1.00 | 14.86 | A |
| ATOM | 1049 | NE2 | GLN | A | 136 | 91.440 | 48.207 | 36.128 | 1.00 | 16.43 | A |
| ATOM | 1050 | C | GLN | A | 136 | 89.314 | 46.808 | 39.976 | 1.00 | 12.34 | A |
| ATOM | 1051 | O | GLN | A | 136 | 88.542 | 45.945 | 40.391 | 1.00 | 12.45 | A |
| ATOM | 1052 | N | VAL | A | 137 | 88.905 | 47.982 | 39.507 | 1.00 | 12.59 | A |
| ATOM | 1053 | CA | VAL | A | 137 | 87.492 | 48.334 | 39.418 | 1.00 | 13.55 | A |
| ATOM | 1054 | CB | VAL | A | 137 | 87.075 | 49.331 | 40.522 | 1.00 | 13.88 | A |
| ATOM | 1055 | CG1 | VAL | A | 137 | 85.578 | 49.615 | 40.424 | 1.00 | 14.64 | A |
| ATOM | 1056 | CG2 | VAL | A | 137 | 87.414 | 48.762 | 41.891 | 1.00 | 15.69 | A |
| ATOM | 1057 | C | VAL | A | 137 | 87.300 | 48.985 | 38.053 | 1.00 | 14.00 | A |
| ATOM | 1058 | O | VAL | A | 137 | 87.508 | 50.188 | 37.890 | 1.00 | 14.57 | A |
| ATOM | 1059 | N | GLY | A | 138 | 86.916 | 48.178 | 37.071 | 1.00 | 15.30 | A |
| ATOM | 1060 | CA | GLY | A | 138 | 86.726 | 48.698 | 35.731 | 1.00 | 16.20 | A |
| ATOM | 1061 | C | GLY | A | 138 | 88.073 | 49.004 | 35.106 | 1.00 | 16.43 | A |
| ATOM | 1062 | O | GLY | A | 138 | 88.872 | 48.100 | 34.860 | 1.00 | 16.78 | A |
| ATOM | 1063 | N | ALA | A | 139 | 88.333 | 50.281 | 34.852 | 1.00 | 17.13 | A |
| ATOM | 1064 | CA | ALA | A | 139 | 89.600 | 50.694 | 34.259 | 1.00 | 17.89 | A |
| ATOM | 1065 | CB | ALA | A | 139 | 89.347 | 51.681 | 33.125 | 1.00 | 19.40 | A |
| ATOM | 1066 | C | ALA | A | 139 | 90.497 | 51.334 | 35.315 | 1.00 | 18.15 | A |
| ATOM | 1067 | O | ALA | A | 139 | 91.565 | 51.864 | 35.000 | 1.00 | 19.45 | A |
| ATOM | 1068 | N | ARG | A | 140 | 90.064 | 51.269 | 36.570 | 1.00 | 16.85 | A |
| ATOM | 1069 | CA | ARG | A | 140 | 90.820 | 51.864 | 37.665 | 1.00 | 15.60 | A |
| ATOM | 1070 | CB | ARG | A | 140 | 89.903 | 52.763 | 38.501 | 1.00 | 15.97 | A |
| ATOM | 1071 | CG | ARG | A | 140 | 89.063 | 53.734 | 37.688 | 1.00 | 15.78 | A |
| ATOM | 1072 | CD | ARG | A | 140 | 87.616 | 53.721 | 38.160 | 1.00 | 16.68 | A |
| ATOM | 1073 | NE | ARG | A | 140 | 87.480 | 54.201 | 39.530 | 1.00 | 17.58 | A |
| ATOM | 1074 | CZ | ARG | A | 140 | 86.381 | 54.068 | 40.266 | 1.00 | 17.18 | A |
| ATOM | 1075 | NH1 | ARG | A | 140 | 85.312 | 53.462 | 39.768 | 1.00 | 17.71 | A |
| ATOM | 1076 | NH2 | ARG | A | 140 | 86.348 | 54.543 | 41.506 | 1.00 | 18.33 | A |
| ATOM | 1077 | C | ARG | A | 140 | 91.450 | 50.829 | 38.588 | 1.00 | 15.02 | A |
| ATOM | 1078 | O | ARG | A | 140 | 90.847 | 49.798 | 38.882 | 1.00 | 15.57 | A |
| ATOM | 1079 | N | TYR | A | 141 | 92.672 | 51.105 | 39.028 | 1.00 | 14.56 | A |
| ATOM | 1080 | CA | TYR | A | 141 | 93.348 | 50.229 | 39.976 | 1.00 | 14.05 | A |
| ATOM | 1081 | CB | TYR | A | 141 | 94.777 | 49.907 | 39.536 | 1.00 | 13.32 | A |
| ATOM | 1082 | CG | TYR | A | 141 | 95.360 | 48.733 | 40.296 | 1.00 | 13.50 | A |
| ATOM | 1083 | CD1 | TYR | A | 141 | 96.413 | 48.908 | 41.193 | 1.00 | 13.36 | A |
| ATOM | 1084 | CE1 | TYR | A | 141 | 96.919 | 47.834 | 41.927 | 1.00 | 12.84 | A |
| ATOM | 1085 | CD2 | TYR | A | 141 | 94.824 | 47.450 | 40.147 | 1.00 | 13.12 | A |
| ATOM | 1086 | CE2 | TYR | A | 141 | 95.322 | 46.370 | 40.876 | 1.00 | 13.54 | A |
| ATOM | 1087 | CZ | TYR | A | 141 | 96.369 | 46.572 | 41.763 | 1.00 | 12.28 | A |
| ATOM | 1088 | OH | TYR | A | 141 | 96.866 | 45.506 | 42.475 | 1.00 | 12.32 | A |
| ATOM | 1089 | C | TYR | A | 141 | 93.336 | 51.056 | 41.255 | 1.00 | 14.27 | A |
| ATOM | 1090 | O | TYR | A | 141 | 93.686 | 52.240 | 41.243 | 1.00 | 14.40 | A |
| ATOM | 1091 | N | VAL | A | 142 | 92.940 | 50.427 | 42.355 | 1.00 | 13.53 | A |
| ATOM | 1092 | CA | VAL | A | 142 | 92.785 | 51.126 | 43.624 | 1.00 | 13.02 | A |
| ATOM | 1093 | CB | VAL | A | 142 | 91.310 | 51.055 | 44.068 | 1.00 | 13.70 | A |
| ATOM | 1094 | CG1 | VAL | A | 142 | 91.081 | 51.931 | 45.286 | 1.00 | 14.49 | A |
| ATOM | 1095 | CG2 | VAL | A | 142 | 90.411 | 51.464 | 42.913 | 1.00 | 15.14 | A |
| ATOM | 1096 | C | VAL | A | 142 | 93.624 | 50.686 | 44.814 | 1.00 | 13.06 | A |
| ATOM | 1097 | O | VAL | A | 142 | 93.840 | 49.495 | 45.035 | 1.00 | 11.84 | A |
| ATOM | 1098 | N | ILE | A | 143 | 94.085 | 51.675 | 45.575 | 1.00 | 11.57 | A |
| ATOM | 1099 | CA | ILE | A | 143 | 94.848 | 51.455 | 46.798 | 1.00 | 12.41 | A |
| ATOM | 1100 | CB | ILE | A | 143 | 96.179 | 52.249 | 46.810 | 1.00 | 11.44 | A |
| ATOM | 1101 | CG2 | ILE | A | 143 | 96.862 | 52.088 | 48.169 | 1.00 | 12.74 | A |
| ATOM | 1102 | CG1 | ILE | A | 143 | 97.088 | 51.761 | 45.680 | 1.00 | 12.30 | A |
| ATOM | 1103 | CD1 | ILE | A | 143 | 98.341 | 52.607 | 45.487 | 1.00 | 14.53 | A |
| ATOM | 1104 | C | ILE | A | 143 | 93.950 | 52.003 | 47.905 | 1.00 | 12.37 | A |
| ATOM | 1105 | O | ILE | A | 143 | 93.627 | 53.193 | 47.915 | 1.00 | 12.40 | A |
| ATOM | 1106 | N | PRO | A | 144 | 93.514 | 51.140 | 48.834 | 1.00 | 12.63 | A |
| ATOM | 1107 | CD | PRO | A | 144 | 93.676 | 49.678 | 48.791 | 1.00 | 13.98 | A |
| ATOM | 1108 | CA | PRO | A | 144 | 92.647 | 51.536 | 49.948 | 1.00 | 13.44 | A |
| ATOM | 1109 | CB | PRO | A | 144 | 92.267 | 50.203 | 50.591 | 1.00 | 15.82 | A |
| ATOM | 1110 | CG | PRO | A | 144 | 92.444 | 49.211 | 49.499 | 1.00 | 16.35 | A |
| ATOM | 1111 | C | PRO | A | 144 | 93.371 | 52.433 | 50.944 | 1.00 | 13.29 | A |
| ATOM | 1112 | O | PRO | A | 144 | 94.535 | 52.196 | 51.259 | 1.00 | 13.25 | A |
| ATOM | 1113 | N | LEU | A | 145 | 92.675 | 53.447 | 51.448 | 1.00 | 13.56 | A |
| ATOM | 1114 | CA | LEU | A | 145 | 93.260 | 54.369 | 52.416 | 1.00 | 14.21 | A |
| ATOM | 1115 | CB | LEU | A | 145 | 93.736 | 55.643 | 51.711 | 1.00 | 17.96 | A |
| ATOM | 1116 | CG | LEU | A | 145 | 94.592 | 55.533 | 50.448 | 1.00 | 19.42 | A |
| ATOM | 1117 | CD1 | LEU | A | 145 | 94.731 | 56.908 | 49.819 | 1.00 | 23.67 | A |
| ATOM | 1118 | CD2 | LEU | A | 145 | 95.948 | 54.959 | 50.794 | 1.00 | 21.57 | A |
| ATOM | 1119 | C | LEU | A | 145 | 92.233 | 54.777 | 53.462 | 1.00 | 13.24 | A |
| ATOM | 1120 | O | LEU | A | 145 | 91.037 | 54.830 | 53.181 | 1.00 | 13.31 | A |
| ATOM | 1121 | N | TYR | A | 146 | 92.691 | 55.042 | 54.679 | 1.00 | 12.19 | A |
| ATOM | 1122 | CA | TYR | A | 146 | 91.784 | 55.528 | 55.704 | 1.00 | 12.30 | A |
| ATOM | 1123 | CB | TYR | A | 146 | 91.474 | 54.461 | 56.774 | 1.00 | 12.91 | A |
| ATOM | 1124 | CG | TYR | A | 146 | 92.616 | 53.947 | 57.622 | 1.00 | 12.37 | A |
| ATOM | 1125 | CD1 | TYR | A | 146 | 93.065 | 54.663 | 58.732 | 1.00 | 12.92 | A |
| ATOM | 1126 | CE1 | TYR | A | 146 | 94.048 | 54.149 | 59.568 | 1.00 | 12.51 | A |
| ATOM | 1127 | CD2 | TYR | A | 146 | 93.190 | 52.701 | 57.364 | 1.00 | 11.37 | A |
| ATOM | 1128 | CE2 | TYR | A | 146 | 94.177 | 52.177 | 58.193 | 1.00 | 11.75 | A |
| ATOM | 1129 | CZ | TYR | A | 146 | 94.598 | 52.906 | 59.295 | 1.00 | 12.10 | A |
| ATOM | 1130 | OH | TYR | A | 146 | 95.563 | 52.394 | 60.129 | 1.00 | 14.09 | A |
| ATOM | 1131 | C | TYR | A | 146 | 92.446 | 56.767 | 56.278 | 1.00 | 13.68 | A |
| ATOM | 1132 | O | TYR | A | 146 | 93.674 | 56.852 | 56.349 | 1.00 | 14.11 | A |
| ATOM | 1133 | N | ALA | A | 147 | 91.631 | 57.743 | 56.650 | 1.00 | 14.71 | A |
| ATOM | 1134 | CA | ALA | A | 147 | 92.160 | 58.994 | 57.164 | 1.00 | 15.18 | A |
| ATOM | 1135 | CB | ALA | A | 147 | 92.179 | 60.032 | 56.045 | 1.00 | 15.91 | A |
| ATOM | 1136 | C | ALA | A | 147 | 91.350 | 59.515 | 58.330 | 1.00 | 16.58 | A |
| ATOM | 1137 | O | ALA | A | 147 | 90.271 | 59.010 | 58.633 | 1.00 | 16.43 | A |
| ATOM | 1138 | N | ALA | A | 148 | 91.884 | 60.537 | 58.984 | 1.00 | 17.45 | A |
| ATOM | 1139 | CA | ALA | A | 148 | 91.208 | 61.149 | 60.111 | 1.00 | 18.77 | A |
| ATOM | 1140 | CB | ALA | A | 148 | 91.634 | 60.478 | 61.413 | 1.00 | 20.02 | A |
| ATOM | 1141 | C | ALA | A | 148 | 91.546 | 62.628 | 60.150 | 1.00 | 19.97 | A |
| ATOM | 1142 | O | ALA | A | 148 | 92.622 | 63.042 | 59.722 | 1.00 | 19.16 | A |
| ATOM | 1143 | N | THR | A | 149 | 90.605 | 63.416 | 60.651 | 1.00 | 22.02 | A |
| ATOM | 1144 | CA | THR | A | 149 | 90.776 | 64.856 | 60.778 | 1.00 | 23.74 | A |
| ATOM | 1145 | CB | THR | A | 149 | 90.127 | 65.610 | 59.601 | 1.00 | 23.74 | A |
| ATOM | 1146 | OG1 | THR | A | 149 | 90.711 | 65.169 | 58.368 | 1.00 | 23.87 | A |
| ATOM | 1147 | CG2 | THR | A | 149 | 90.345 | 67.108 | 59.746 | 1.00 | 24.91 | A |
| ATOM | 1148 | C | THR | A | 149 | 90.072 | 65.231 | 62.072 | 1.00 | 24.92 | A |
| ATOM | 1149 | O | THR | A | 149 | 88.863 | 65.458 | 62.089 | 1.00 | 25.67 | A |
| ATOM | 1150 | N | GLY | A | 150 | 90.836 | 65.273 | 63.159 | 1.00 | 26.61 | A |
| ATOM | 1151 | CA | GLY | A | 150 | 90.262 | 65.597 | 64.451 | 1.00 | 28.76 | A |
| ATOM | 1152 | C | GLY | A | 150 | 89.414 | 64.437 | 64.930 | 1.00 | 30.02 | A |
| ATOM | 1153 | O | GLY | A | 150 | 89.938 | 63.390 | 65.313 | 1.00 | 31.94 | A |
| ATOM | 1154 | N | VAL | A | 151 | 88.098 | 64.619 | 64.903 | 1.00 | 30.18 | A |
| ATOM | 1155 | CA | VAL | A | 151 | 87.172 | 63.578 | 65.325 | 1.00 | 29.70 | A |
| ATOM | 1156 | CB | VAL | A | 151 | 86.138 | 64.121 | 66.337 | 1.00 | 31.14 | A |
| ATOM | 1157 | CG1 | VAL | A | 151 | 86.836 | 64.519 | 67.629 | 1.00 | 31.95 | A |
| ATOM | 1158 | CG2 | VAL | A | 151 | 85.408 | 65.312 | 65.742 | 1.00 | 31.58 | A |
| ATOM | 1159 | C | VAL | A | 151 | 86.439 | 63.013 | 64.114 | 1.00 | 28.03 | A |
| ATOM | 1160 | O | VAL | A | 151 | 85.536 | 62.185 | 64.249 | 1.00 | 27.91 | A |
| ATOM | 1161 | N | ASN | A | 152 | 86.833 | 63.471 | 62.929 | 1.00 | 26.29 | A |
| ATOM | 1162 | CA | ASN | A | 152 | 86.232 | 63.005 | 61.684 | 1.00 | 24.30 | A |
| ATOM | 1163 | CB | ASN | A | 152 | 86.084 | 64.163 | 60.692 | 1.00 | 25.89 | A |
| ATOM | 1164 | CG | ASN | A | 152 | 85.743 | 63.690 | 59.287 | 1.00 | 27.72 | A |
| ATOM | 1165 | OD1 | ASN | A | 152 | 86.626 | 63.309 | 58.516 | 1.00 | 28.09 | A |
| ATOM | 1166 | ND2 | ASN | A | 152 | 84.457 | 63.696 | 58.955 | 1.00 | 28.42 | A |
| ATOM | 1167 | C | ASN | A | 152 | 87.090 | 61.904 | 61.068 | 1.00 | 22.57 | A |
| ATOM | 1168 | O | ASN | A | 152 | 88.317 | 62.001 | 61.044 | 1.00 | 21.50 | A |
| ATOM | 1169 | N | TYR | A | 153 | 86.434 | 60.862 | 60.568 | 1.00 | 20.06 | A |
| ATOM | 1170 | CA | TYR | A | 153 | 87.124 | 59.727 | 59.963 | 1.00 | 18.03 | A |
| ATOM | 1171 | CB | TYR | A | 153 | 86.921 | 58.485 | 60.829 | 1.00 | 17.67 | A |
| ATOM | 1172 | CG | TYR | A | 153 | 87.405 | 58.658 | 62.246 | 1.00 | 17.67 | A |
| ATOM | 1173 | CD1 | TYR | A | 153 | 88.757 | 58.542 | 62.560 | 1.00 | 18.65 | A |
| ATOM | 1174 | CE1 | TYR | A | 153 | 89.211 | 58.729 | 63.861 | 1.00 | 18.91 | A |
| ATOM | 1175 | CD2 | TYR | A | 153 | 86.513 | 58.967 | 63.271 | 1.00 | 17.73 | A |
| ATOM | 1176 | CE2 | TYR | A | 153 | 86.956 | 59.158 | 64.576 | 1.00 | 18.88 | A |
| ATOM | 1177 | CZ | TYR | A | 153 | 88.304 | 59.038 | 64.863 | 1.00 | 19.08 | A |
| ATOM | 1178 | OH | TYR | A | 153 | 88.749 | 59.229 | 66.152 | 1.00 | 21.81 | A |
| ATOM | 1179 | C | TYR | A | 153 | 86.600 | 59.451 | 58.564 | 1.00 | 16.80 | A |
| ATOM | 1180 | O | TYR | A | 153 | 85.413 | 59.634 | 58.288 | 1.00 | 16.13 | A |
| ATOM | 1181 | N | GLU | A | 154 | 87.483 | 59.002 | 57.681 | 1.00 | 16.48 | A |
| ATOM | 1182 | CA | GLU | A | 154 | 87.070 | 58.703 | 56.320 | 1.00 | 17.39 | A |
| ATOM | 1183 | CB | GLU | A | 154 | 87.311 | 59.912 | 55.411 | 1.00 | 19.58 | A |
| ATOM | 1184 | CG | GLU | A | 154 | 86.509 | 61.150 | 55.786 | 1.00 | 23.41 | A |
| ATOM | 1185 | CD | GLU | A | 154 | 86.571 | 62.232 | 54.721 | 1.00 | 27.47 | A |
| ATOM | 1186 | OE1 | GLU | A | 154 | 87.690 | 62.637 | 54.346 | 1.00 | 29.41 | A |
| ATOM | 1187 | OE2 | GLU | A | 154 | 85.498 | 62.681 | 54.263 | 1.00 | 29.91 | A |
| ATOM | 1188 | C | GLU | A | 154 | 87.767 | 57.493 | 55.716 | 1.00 | 16.02 | A |
| ATOM | 1189 | O | GLU | A | 154 | 88.883 | 57.140 | 56.097 | 1.00 | 16.24 | A |
| ATOM | 1190 | N | LEU | A | 155 | 87.075 | 56.854 | 54.781 | 1.00 | 15.40 | A |
| ATOM | 1191 | CA | LEU | A | 155 | 87.611 | 55.715 | 54.055 | 1.00 | 14.53 | A |
| ATOM | 1192 | CB | LEU | A | 155 | 86.682 | 54.504 | 54.159 | 1.00 | 13.51 | A |
| ATOM | 1193 | CG | LEU | A | 155 | 86.474 | 53.921 | 55.556 | 1.00 | 13.30 | A |
| ATOM | 1194 | CD1 | LEU | A | 155 | 85.540 | 52.726 | 55.472 | 1.00 | 13.30 | A |
| ATOM | 1195 | CD2 | LEU | A | 155 | 87.812 | 53.512 | 56.162 | 1.00 | 13.99 | A |
| ATOM | 1196 | C | LEU | A | 155 | 87.662 | 56.219 | 52.621 | 1.00 | 15.30 | A |
| ATOM | 1197 | O | LEU | A | 155 | 86.644 | 56.655 | 52.078 | 1.00 | 16.68 | A |
| ATOM | 1198 | N | LYS | A | 156 | 88.846 | 56.169 | 52.017 | 1.00 | 15.07 | A |
| ATOM | 1199 | CA | LYS | A | 156 | 89.037 | 56.664 | 50.659 | 1.00 | 16.34 | A |
| ATOM | 1200 | CB | LYS | A | 156 | 89.893 | 57.935 | 50.687 | 1.00 | 18.53 | A |
| ATOM | 1201 | CG | LYS | A | 156 | 89.453 | 58.998 | 51.690 | 1.00 | 22.97 | A |
| ATOM | 1202 | CD | LYS | A | 156 | 90.522 | 60.081 | 51.812 | 1.00 | 24.90 | A |
| ATOM | 1203 | CE | LYS | A | 156 | 90.229 | 61.052 | 52.945 | 1.00 | 27.77 | A |
| ATOM | 1204 | NZ | LYS | A | 156 | 91.320 | 62.057 | 53.109 | 1.00 | 29.34 | A |
| ATOM | 1205 | C | LYS | A | 156 | 89.715 | 55.660 | 49.731 | 1.00 | 15.77 | A |
| ATOM | 1206 | O | LYS | A | 156 | 90.214 | 54.620 | 50.164 | 1.00 | 14.23 | A |
| ATOM | 1207 | N | TRP | A | 157 | 89.729 | 55.999 | 48.447 | 1.00 | 14.64 | A |
| ATOM | 1208 | CA | TRP | A | 157 | 90.357 | 55.182 | 47.416 | 1.00 | 14.86 | A |
| ATOM | 1209 | CB | TRP | A | 157 | 89.314 | 54.661 | 46.421 | 1.00 | 14.24 | A |
| ATOM | 1210 | CG | TRP | A | 157 | 88.561 | 53.440 | 46.858 | 1.00 | 15.14 | A |
| ATOM | 1211 | CD2 | TRP | A | 157 | 87.598 | 52.712 | 46.085 | 1.00 | 14.50 | A |
| ATOM | 1212 | CE2 | TRP | A | 157 | 87.147 | 51.636 | 46.882 | 1.00 | 14.19 | A |
| ATOM | 1213 | CE3 | TRP | A | 157 | 87.067 | 52.870 | 44.796 | 1.00 | 15.53 | A |
| ATOM | 1214 | CD1 | TRP | A | 157 | 88.658 | 52.792 | 48.057 | 1.00 | 13.97 | A |
| ATOM | 1215 | NE1 | TRP | A | 157 | 87.812 | 51.706 | 48.078 | 1.00 | 13.62 | A |
| ATOM | 1216 | CZ2 | TRP | A | 157 | 86.193 | 50.717 | 46.432 | 1.00 | 14.33 | A |
| ATOM | 1217 | CZ3 | TRP | A | 157 | 86.116 | 51.953 | 44.349 | 1.00 | 15.06 | A |
| ATOM | 1218 | CH2 | TRP | A | 157 | 85.690 | 50.893 | 45.169 | 1.00 | 15.34 | A |
| ATOM | 1219 | C | TRP | A | 157 | 91.370 | 56.024 | 46.648 | 1.00 | 15.17 | A |
| ATOM | 1220 | O | TRP | A | 157 | 91.049 | 57.114 | 46.170 | 1.00 | 15.95 | A |
| ATOM | 1221 | N | LEU | A | 158 | 92.595 | 55.520 | 46.545 | 1.00 | 14.38 | A |
| ATOM | 1222 | CA | LEU | A | 158 | 93.646 | 56.193 | 45.789 | 1.00 | 15.27 | A |
| ATOM | 1223 | CB | LEU | A | 158 | 94.986 | 56.111 | 46.524 | 1.00 | 14.95 | A |
| ATOM | 1224 | CG | LEU | A | 158 | 96.171 | 56.796 | 45.840 | 1.00 | 15.40 | A |
| ATOM | 1225 | CD1 | LEU | A | 158 | 95.834 | 58.253 | 45.553 | 1.00 | 16.72 | A |
| ATOM | 1226 | CD2 | LEU | A | 158 | 97.401 | 56.698 | 46.734 | 1.00 | 15.73 | A |
| ATOM | 1227 | C | LEU | A | 158 | 93.699 | 55.390 | 44.498 | 1.00 | 16.02 | A |
| ATOM | 1228 | O | LEU | A | 158 | 94.116 | 54.233 | 44.498 | 1.00 | 15.97 | A |
| ATOM | 1229 | N | GLU | A | 159 | 93.279 | 56.002 | 43.397 | 1.00 | 16.36 | A |
| ATOM | 1230 | CA | GLU | A | 159 | 93.219 | 55.282 | 42.131 | 1.00 | 16.87 | A |
| ATOM | 1231 | CB | GLU | A | 159 | 91.751 | 55.177 | 41.683 | 1.00 | 18.16 | A |
| ATOM | 1232 | CG | GLU | A | 159 | 90.790 | 56.032 | 42.501 | 1.00 | 20.32 | A |
| ATOM | 1233 | CD | GLU | A | 159 | 89.342 | 55.942 | 42.035 | 1.00 | 19.57 | A |
| ATOM | 1234 | OE1 | GLU | A | 159 | 89.083 | 56.199 | 40.842 | 1.00 | 20.34 | A |
| ATOM | 1235 | OE2 | GLU | A | 159 | 88.466 | 55.625 | 42.871 | 1.00 | 20.24 | A |
| ATOM | 1236 | C | GLU | A | 159 | 94.054 | 55.789 | 40.962 | 1.00 | 16.76 | A |
| ATOM | 1237 | O | GLU | A | 159 | 94.500 | 56.936 | 40.933 | 1.00 | 17.29 | A |
| ATOM | 1238 | N | SER | A | 160 | 94.260 | 54.894 | 40.001 | 1.00 | 16.05 | A |
| ATOM | 1239 | CA | SER | A | 160 | 94.989 | 55.192 | 38.779 | 1.00 | 16.07 | A |
| ATOM | 1240 | CB | SER | A | 160 | 96.406 | 54.631 | 38.819 | 1.00 | 15.74 | A |
| ATOM | 1241 | OG | SER | A | 160 | 97.030 | 54.803 | 37.551 | 1.00 | 16.34 | A |
| ATOM | 1242 | C | SER | A | 160 | 94.243 | 54.556 | 37.618 | 1.00 | 17.20 | A |
| ATOM | 1243 | O | SER | A | 160 | 93.852 | 53.384 | 37.678 | 1.00 | 14.83 | A |
| ATOM | 1244 | N | SER | A | 161 | 94.051 | 55.336 | 36.560 | 1.00 | 17.32 | A |
| ATOM | 1245 | CA | SER | A | 161 | 93.353 | 54.863 | 35.375 | 1.00 | 19.60 | A |
| ATOM | 1246 | CB | SER | A | 161 | 92.241 | 55.848 | 34.992 | 1.00 | 20.42 | A |
| ATOM | 1247 | OG | SER | A | 161 | 92.743 | 57.168 | 34.859 | 1.00 | 25.03 | A |
| ATOM | 1248 | C | SER | A | 161 | 94.295 | 54.662 | 34.192 | 1.00 | 19.68 | A |
| ATOM | 1249 | O | SER | A | 161 | 93.845 | 54.364 | 33.089 | 1.00 | 20.35 | A |
| ATOM | 1250 | N | ASP | A | 162 | 95.598 | 54.815 | 34.416 | 1.00 | 19.30 | A |
| ATOM | 1251 | CA | ASP | A | 162 | 96.564 | 54.632 | 33.339 | 1.00 | 19.16 | A |
| ATOM | 1252 | CB | ASP | A | 162 | 97.238 | 55.962 | 32.976 | 1.00 | 19.87 | A |
| ATOM | 1253 | CG | ASP | A | 162 | 97.917 | 56.624 | 34.157 | 1.00 | 21.01 | A |
| ATOM | 1254 | OD1 | ASP | A | 162 | 98.180 | 55.935 | 35.167 | 1.00 | 19.55 | A |
| ATOM | 1255 | OD2 | ASP | A | 162 | 98.201 | 57.842 | 34.067 | 1.00 | 20.38 | A |
| ATOM | 1256 | C | ASP | A | 162 | 97.625 | 53.584 | 33.653 | 1.00 | 19.43 | A |
| ATOM | 1257 | O | ASP | A | 162 | 98.778 | 53.713 | 33.241 | 1.00 | 19.94 | A |
| ATOM | 1258 | N | GLY | A | 163 | 97.229 | 52.546 | 34.385 | 1.00 | 18.78 | A |
| ATOM | 1259 | CA | GLY | A | 163 | 98.154 | 51.476 | 34.720 | 1.00 | 18.29 | A |
| ATOM | 1260 | C | GLY | A | 163 | 99.147 | 51.780 | 35.825 | 1.00 | 18.38 | A |
| ATOM | 1261 | O | GLY | A | 163 | 100.178 | 51.117 | 35.924 | 1.00 | 18.19 | A |
| ATOM | 1262 | N | GLY | A | 164 | 98.849 | 52.779 | 36.649 | 1.00 | 18.87 | A |
| ATOM | 1263 | CA | GLY | A | 164 | 99.742 | 53.119 | 37.743 | 1.00 | 19.12 | A |
| ATOM | 1264 | C | GLY | A | 164 | 100.742 | 54.230 | 37.475 | 1.00 | 20.78 | A |
| ATOM | 1265 | O | GLY | A | 164 | 101.654 | 54.442 | 38.274 | 1.00 | 20.17 | A |
| ATOM | 1266 | N | GLU | A | 165 | 100.585 | 54.941 | 36.363 | 1.00 | 21.17 | A |
| ATOM | 1267 | CA | GLU | A | 165 | 101.497 | 56.034 | 36.033 | 1.00 | 22.65 | A |
| ATOM | 1268 | CB | GLU | A | 165 | 101.443 | 56.346 | 34.536 | 1.00 | 24.06 | A |
| ATOM | 1269 | CG | GLU | A | 165 | 102.037 | 55.267 | 33.649 | 1.00 | 26.00 | A |
| ATOM | 1270 | CD | GLU | A | 165 | 103.517 | 55.045 | 33.904 | 1.00 | 29.27 | A |
| ATOM | 1271 | OE1 | GLU | A | 165 | 103.868 | 54.476 | 34.959 | 1.00 | 27.82 | A |
| ATOM | 1272 | OE2 | GLU | A | 165 | 104.334 | 55.450 | 33.049 | 1.00 | 31.50 | A |
| ATOM | 1273 | C | GLU | A | 165 | 101.148 | 57.285 | 36.832 | 1.00 | 22.89 | A |
| ATOM | 1274 | O | GLU | A | 165 | 102.029 | 57.951 | 37.379 | 1.00 | 23.85 | A |
| ATOM | 1275 | N | THR | A | 166 | 99.859 | 57.601 | 36.894 | 1.00 | 22.04 | A |
| ATOM | 1276 | CA | THR | A | 166 | 99.389 | 58.764 | 37.638 | 1.00 | 22.17 | A |
| ATOM | 1277 | CB | THR | A | 166 | 98.789 | 59.835 | 36.700 | 1.00 | 23.12 | A |
| ATOM | 1278 | OG1 | THR | A | 166 | 97.640 | 59.304 | 36.031 | 1.00 | 23.25 | A |
| ATOM | 1279 | CG2 | THR | A | 166 | 99.817 | 60.265 | 35.662 | 1.00 | 24.30 | A |
| ATOM | 1280 | C | THR | A | 166 | 98.330 | 58.327 | 38.640 | 1.00 | 21.55 | A |
| ATOM | 1281 | O | THR | A | 166 | 97.571 | 57.390 | 38.384 | 1.00 | 20.40 | A |
| ATOM | 1282 | N | TRP | A | 167 | 98.285 | 59.008 | 39.781 | 1.00 | 20.51 | A |
| ATOM | 1283 | CA | TRP | A | 167 | 97.331 | 58.680 | 40.834 | 1.00 | 20.47 | A |
| ATOM | 1284 | CB | TRP | A | 167 | 98.049 | 58.004 | 42.005 | 1.00 | 18.41 | A |
| ATOM | 1285 | CG | TRP | A | 167 | 98.756 | 56.745 | 41.623 | 1.00 | 17.60 | A |
| ATOM | 1286 | CD2 | TRP | A | 167 | 98.283 | 55.411 | 41.823 | 1.00 | 15.72 | A |
| ATOM | 1287 | CE2 | TRP | A | 167 | 99.244 | 54.539 | 41.265 | 1.00 | 16.22 | A |
| ATOM | 1288 | CE3 | TRP | A | 167 | 97.134 | 54.866 | 42.416 | 1.00 | 15.58 | A |
| ATOM | 1289 | CD1 | TRP | A | 167 | 99.954 | 56.635 | 40.974 | 1.00 | 16.45 | A |
| ATOM | 1290 | NE1 | TRP | A | 167 | 100.255 | 55.314 | 40.755 | 1.00 | 16.74 | A |
| ATOM | 1291 | CZ2 | TRP | A | 167 | 99.096 | 53.147 | 41.286 | 1.00 | 15.44 | A |
| ATOM | 1292 | CZ3 | TRP | A | 167 | 96.986 | 53.481 | 42.435 | 1.00 | 14.86 | A |
| ATOM | 1293 | CH2 | TRP | A | 167 | 97.963 | 52.639 | 41.871 | 1.00 | 14.83 | A |
| ATOM | 1294 | C | TRP | A | 167 | 96.583 | 59.900 | 41.352 | 1.00 | 21.35 | A |
| ATOM | 1295 | O | TRP | A | 167 | 96.973 | 61.037 | 41.097 | 1.00 | 23.24 | A |
| ATOM | 1296 | N | GLY | A | 168 | 95.504 | 59.645 | 42.086 | 1.00 | 22.09 | A |
| ATOM | 1297 | CA | GLY | A | 168 | 94.701 | 60.715 | 42.649 | 1.00 | 23.24 | A |
| ATOM | 1298 | C | GLY | A | 168 | 93.578 | 60.148 | 43.494 | 1.00 | 24.12 | A |
| ATOM | 1299 | O | GLY | A | 168 | 93.118 | 59.033 | 43.247 | 1.00 | 22.53 | A |
| ATOM | 1300 | N | GLU | A | 169 | 93.136 | 60.902 | 44.496 | 1.00 | 25.76 | A |
| ATOM | 1301 | CA | GLU | A | 169 | 92.054 | 60.444 | 45.358 | 1.00 | 27.57 | A |
| ATOM | 1302 | CB | GLU | A | 169 | 91.790 | 61.456 | 46.477 | 1.00 | 29.66 | A |
| ATOM | 1303 | CG | GLU | A | 169 | 92.958 | 61.644 | 47.431 | 1.00 | 32.98 | A |
| ATOM | 1304 | CD | GLU | A | 169 | 92.625 | 62.571 | 48.586 | 1.00 | 35.74 | A |
| ATOM | 1305 | OE1 | GLU | A | 169 | 91.720 | 62.233 | 49.380 | 1.00 | 36.78 | A |
| ATOM | 1306 | OE2 | GLU | A | 169 | 93.266 | 63.637 | 48.699 | 1.00 | 37.62 | A |
| ATOM | 1307 | C | GLU | A | 169 | 90.789 | 60.249 | 44.532 | 1.00 | 27.59 | A |
| ATOM | 1308 | O | GLU | A | 169 | 90.438 | 61.094 | 43.708 | 1.00 | 27.17 | A |
| ATOM | 1309 | N | GLY | A | 170 | 90.110 | 59.128 | 44.753 | 1.00 | 26.77 | A |
| ATOM | 1310 | CA | GLY | A | 170 | 88.898 | 58.841 | 44.011 | 1.00 | 26.68 | A |
| ATOM | 1311 | C | GLY | A | 170 | 87.645 | 58.801 | 44.863 | 1.00 | 26.73 | A |
| ATOM | 1312 | O | GLY | A | 170 | 87.296 | 59.780 | 45.521 | 1.00 | 27.56 | A |
| ATOM | 1313 | N | SER | A | 171 | 86.971 | 57.655 | 44.851 | 1.00 | 26.73 | A |
| ATOM | 1314 | CA | SER | A | 171 | 85.734 | 57.465 | 45.603 | 1.00 | 26.22 | A |
| ATOM | 1315 | CB | SER | A | 171 | 85.135 | 56.093 | 45.280 | 1.00 | 26.28 | A |
| ATOM | 1316 | OG | SER | A | 171 | 84.963 | 55.915 | 43.882 | 1.00 | 25.45 | A |
| ATOM | 1317 | C | SER | A | 171 | 85.910 | 57.580 | 47.115 | 1.00 | 26.31 | A |
| ATOM | 1318 | O | SER | A | 171 | 86.970 | 57.266 | 47.659 | 1.00 | 25.35 | A |
| ATOM | 1319 | N | THR | A | 172 | 84.858 | 58.037 | 47.785 | 1.00 | 25.96 | A |
| ATOM | 1320 | CA | THR | A | 172 | 84.861 | 58.163 | 49.237 | 1.00 | 25.38 | A |
| ATOM | 1321 | CB | THR | A | 172 | 84.458 | 59.580 | 49.694 | 1.00 | 26.90 | A |
| ATOM | 1322 | OG1 | THR | A | 172 | 85.404 | 60.532 | 49.191 | 1.00 | 27.62 | A |
| ATOM | 1323 | CG2 | THR | A | 172 | 84.437 | 59.660 | 51.216 | 1.00 | 26.99 | A |
| ATOM | 1324 | C | THR | A | 172 | 83.834 | 57.162 | 49.744 | 1.00 | 24.57 | A |
| ATOM | 1325 | O | THR | A | 172 | 82.641 | 57.288 | 49.473 | 1.00 | 24.30 | A |
| ATOM | 1326 | N | ILE | A | 173 | 84.308 | 56.158 | 50.470 | 1.00 | 22.96 | A |
| ATOM | 1327 | CA | ILE | A | 173 | 83.439 | 55.114 | 50.993 | 1.00 | 22.68 | A |
| ATOM | 1328 | CB | ILE | A | 173 | 84.246 | 53.831 | 51.306 | 1.00 | 21.60 | A |
| ATOM | 1329 | CG2 | ILE | A | 173 | 83.386 | 52.847 | 52.083 | 1.00 | 21.78 | A |
| ATOM | 1330 | CG1 | ILE | A | 173 | 84.737 | 53.189 | 50.006 | 1.00 | 21.73 | A |
| ATOM | 1331 | CD1 | ILE | A | 173 | 85.661 | 54.063 | 49.175 | 1.00 | 21.70 | A |
| ATOM | 1332 | C | ILE | A | 173 | 82.690 | 55.532 | 52.250 | 1.00 | 22.29 | A |
| ATOM | 1333 | O | ILE | A | 173 | 81.504 | 55.233 | 52.406 | 1.00 | 22.72 | A |
| ATOM | 1334 | N | TYR | A | 174 | 83.385 | 56.229 | 53.141 | 1.00 | 21.33 | A |
| ATOM | 1335 | CA | TYR | A | 174 | 82.788 | 56.661 | 54.397 | 1.00 | 20.30 | A |
| ATOM | 1336 | CB | TYR | A | 174 | 83.038 | 55.605 | 55.477 | 1.00 | 19.49 | A |
| ATOM | 1337 | CG | TYR | A | 174 | 82.555 | 56.011 | 56.850 | 1.00 | 19.24 | A |
| ATOM | 1338 | CD1 | TYR | A | 174 | 81.211 | 55.892 | 57.202 | 1.00 | 19.73 | A |
| ATOM | 1339 | CE1 | TYR | A | 174 | 80.757 | 56.297 | 58.454 | 1.00 | 19.65 | A |
| ATOM | 1340 | CD2 | TYR | A | 174 | 83.437 | 56.549 | 57.789 | 1.00 | 19.11 | A |
| ATOM | 1341 | CE2 | TYR | A | 174 | 82.992 | 56.959 | 59.042 | 1.00 | 18.10 | A |
| ATOM | 1342 | CZ | TYR | A | 174 | 81.652 | 56.831 | 59.367 | 1.00 | 19.54 | A |
| ATOM | 1343 | OH | TYR | A | 174 | 81.205 | 57.249 | 60.602 | 1.00 | 19.93 | A |
| ATOM | 1344 | C | TYR | A | 174 | 83.338 | 57.995 | 54.886 | 1.00 | 20.25 | A |
| ATOM | 1345 | O | TYR | A | 174 | 84.470 | 58.365 | 54.584 | 1.00 | 19.50 | A |
| ATOM | 1346 | N | SER | A | 175 | 82.516 | 58.707 | 55.649 | 1.00 | 20.74 | A |
| ATOM | 1347 | CA | SER | A | 175 | 82.895 | 59.987 | 56.230 | 1.00 | 20.11 | A |
| ATOM | 1348 | CB | SER | A | 175 | 82.786 | 61.106 | 55.195 | 1.00 | 21.01 | A |
| ATOM | 1349 | OG | SER | A | 175 | 83.292 | 62.325 | 55.718 | 1.00 | 21.51 | A |
| ATOM | 1350 | C | SER | A | 175 | 81.941 | 60.253 | 57.386 | 1.00 | 20.02 | A |
| ATOM | 1351 | O | SER | A | 175 | 80.777 | 60.594 | 57.176 | 1.00 | 20.24 | A |
| ATOM | 1352 | N | GLY | A | 176 | 82.439 | 60.078 | 58.606 | 1.00 | 19.40 | A |
| ATOM | 1353 | CA | GLY | A | 176 | 81.615 | 60.293 | 59.779 | 1.00 | 18.39 | A |
| ATOM | 1354 | C | GLY | A | 176 | 82.424 | 60.268 | 61.060 | 1.00 | 18.36 | A |
| ATOM | 1355 | O | GLY | A | 176 | 83.649 | 60.384 | 61.023 | 1.00 | 17.48 | A |
| ATOM | 1356 | N | ASN | A | 177 | 81.742 | 60.100 | 62.190 | 1.00 | 19.83 | A |
| ATOM | 1357 | CA | ASN | A | 177 | 82.401 | 60.082 | 63.494 | 1.00 | 21.50 | A |
| ATOM | 1358 | CB | ASN | A | 177 | 81.497 | 60.722 | 64.547 | 1.00 | 24.60 | A |
| ATOM | 1359 | CG | ASN | A | 177 | 81.097 | 62.136 | 64.187 | 1.00 | 28.93 | A |
| ATOM | 1360 | OD1 | ASN | A | 177 | 81.949 | 62.993 | 63.939 | 1.00 | 31.49 | A |
| ATOM | 1361 | ND2 | ASN | A | 177 | 79.794 | 62.391 | 64.162 | 1.00 | 31.03 | A |
| ATOM | 1362 | C | ASN | A | 177 | 82.818 | 58.702 | 63.983 | 1.00 | 20.07 | A |
| ATOM | 1363 | O | ASN | A | 177 | 83.310 | 58.567 | 65.103 | 1.00 | 21.32 | A |
| ATOM | 1364 | N | THR | A | 178 | 82.617 | 57.681 | 63.158 | 1.00 | 19.17 | A |
| ATOM | 1365 | CA | THR | A | 178 | 82.988 | 56.321 | 63.538 | 1.00 | 18.80 | A |
| ATOM | 1366 | CB | THR | A | 178 | 81.973 | 55.305 | 63.001 | 1.00 | 19.17 | A |
| ATOM | 1367 | OG1 | THR | A | 178 | 80.657 | 55.677 | 63.435 | 1.00 | 22.40 | A |
| ATOM | 1368 | CG2 | THR | A | 178 | 82.292 | 53.915 | 63.521 | 1.00 | 19.97 | A |
| ATOM | 1369 | C | THR | A | 178 | 84.375 | 56.008 | 62.979 | 1.00 | 17.50 | A |
| ATOM | 1370 | O | THR | A | 178 | 84.607 | 56.123 | 61.779 | 1.00 | 16.41 | A |
| ATOM | 1371 | N | PRO | A | 179 | 85.311 | 55.607 | 63.854 | 1.00 | 17.63 | A |
| ATOM | 1372 | CD | PRO | A | 179 | 85.105 | 55.567 | 65.311 | 1.00 | 19.22 | A |
| ATOM | 1373 | CA | PRO | A | 179 | 86.703 | 55.270 | 63.529 | 1.00 | 17.80 | A |
| ATOM | 1374 | CB | PRO | A | 179 | 87.350 | 55.099 | 64.907 | 1.00 | 19.37 | A |
| ATOM | 1375 | CG | PRO | A | 179 | 86.473 | 55.911 | 65.814 | 1.00 | 22.92 | A |
| ATOM | 1376 | C | PRO | A | 179 | 86.953 | 54.050 | 62.639 | 1.00 | 17.13 | A |
| ATOM | 1377 | O | PRO | A | 179 | 87.774 | 53.198 | 62.979 | 1.00 | 16.62 | A |
| ATOM | 1378 | N | TYR | A | 180 | 86.260 | 53.953 | 61.509 | 1.00 | 15.28 | A |
| ATOM | 1379 | CA | TYR | A | 180 | 86.495 | 52.828 | 60.607 | 1.00 | 14.20 | A |
| ATOM | 1380 | CB | TYR | A | 180 | 85.459 | 52.805 | 59.481 | 1.00 | 15.69 | A |
| ATOM | 1381 | CG | TYR | A | 180 | 84.060 | 52.498 | 59.951 | 1.00 | 15.12 | A |
| ATOM | 1382 | CD1 | TYR | A | 180 | 83.000 | 53.358 | 59.654 | 1.00 | 15.37 | A |
| ATOM | 1383 | CE1 | TYR | A | 180 | 81.712 | 53.096 | 60.114 | 1.00 | 15.34 | A |
| ATOM | 1384 | CD2 | TYR | A | 180 | 83.797 | 51.361 | 60.715 | 1.00 | 14.93 | A |
| ATOM | 1385 | CE2 | TYR | A | 180 | 82.514 | 51.089 | 61.178 | 1.00 | 15.21 | A |
| ATOM | 1386 | CZ | TYR | A | 180 | 81.478 | 51.960 | 60.877 | 1.00 | 15.80 | A |
| ATOM | 1387 | OH | TYR | A | 180 | 80.213 | 51.700 | 61.355 | 1.00 | 17.90 | A |
| ATOM | 1388 | C | TYR | A | 180 | 87.880 | 53.037 | 60.014 | 1.00 | 14.19 | A |
| ATOM | 1389 | O | TYR | A | 180 | 88.223 | 54.152 | 59.627 | 1.00 | 14.46 | A |
| ATOM | 1390 | N | ASN | A | 181 | 88.681 | 51.980 | 59.941 | 1.00 | 12.95 | A |
| ATOM | 1391 | CA | ASN | A | 181 | 90.018 | 52.124 | 59.384 | 1.00 | 12.11 | A |
| ATOM | 1392 | CB | ASN | A | 181 | 91.054 | 52.257 | 60.519 | 1.00 | 12.88 | A |
| ATOM | 1393 | CG | ASN | A | 181 | 90.824 | 51.277 | 61.658 | 1.00 | 13.61 | A |
| ATOM | 1394 | OD1 | ASN | A | 181 | 90.939 | 50.067 | 61.485 | 1.00 | 15.70 | A |
| ATOM | 1395 | ND2 | ASN | A | 181 | 90.506 | 51.802 | 62.836 | 1.00 | 15.04 | A |
| ATOM | 1396 | C | ASN | A | 181 | 90.416 | 51.025 | 58.398 | 1.00 | 11.89 | A |
| ATOM | 1397 | O | ASN | A | 181 | 90.131 | 51.135 | 57.208 | 1.00 | 12.08 | A |
| ATOM | 1398 | N | GLU | A | 182 | 91.070 | 49.975 | 58.884 | 1.00 | 11.87 | A |
| ATOM | 1399 | CA | GLU | A | 182 | 91.494 | 48.877 | 58.017 | 1.00 | 10.38 | A |
| ATOM | 1400 | CB | GLU | A | 182 | 92.233 | 47.822 | 58.844 | 1.00 | 10.95 | A |
| ATOM | 1401 | CG | GLU | A | 182 | 93.590 | 48.326 | 59.324 | 1.00 | 11.08 | A |
| ATOM | 1402 | CD | GLU | A | 182 | 94.172 | 47.534 | 60.477 | 1.00 | 11.68 | A |
| ATOM | 1403 | OE1 | GLU | A | 182 | 93.543 | 46.554 | 60.930 | 1.00 | 11.01 | A |
| ATOM | 1404 | OE2 | GLU | A | 182 | 95.276 | 47.902 | 60.937 | 1.00 | 11.36 | A |
| ATOM | 1405 | C | GLU | A | 182 | 90.277 | 48.279 | 57.325 | 1.00 | 10.97 | A |
| ATOM | 1406 | O | GLU | A | 182 | 89.413 | 47.692 | 57.974 | 1.00 | 11.78 | A |
| ATOM | 1407 | N | THR | A | 183 | 90.221 | 48.431 | 56.004 | 1.00 | 10.14 | A |
| ATOM | 1408 | CA | THR | A | 183 | 89.083 | 47.952 | 55.234 | 1.00 | 9.71 | A |
| ATOM | 1409 | CB | THR | A | 183 | 88.208 | 49.140 | 54.783 | 1.00 | 10.07 | A |
| ATOM | 1410 | OG1 | THR | A | 183 | 87.833 | 49.912 | 55.931 | 1.00 | 10.84 | A |
| ATOM | 1411 | CG2 | THR | A | 183 | 86.951 | 48.648 | 54.074 | 1.00 | 10.66 | A |
| ATOM | 1412 | C | THR | A | 183 | 89.483 | 47.146 | 54.004 | 1.00 | 9.75 | A |
| ATOM | 1413 | O | THR | A | 183 | 90.351 | 47.559 | 53.231 | 1.00 | 9.88 | A |
| ATOM | 1414 | N | SER | A | 184 | 88.848 | 45.988 | 53.840 | 1.00 | 9.83 | A |
| ATOM | 1415 | CA | SER | A | 184 | 89.099 | 45.119 | 52.696 | 1.00 | 9.48 | A |
| ATOM | 1416 | CB | SER | A | 184 | 89.070 | 43.646 | 53.124 | 1.00 | 10.39 | A |
| ATOM | 1417 | OG | SER | A | 184 | 89.156 | 42.780 | 51.992 | 1.00 | 11.05 | A |
| ATOM | 1418 | C | SER | A | 184 | 87.990 | 45.354 | 51.680 | 1.00 | 9.08 | A |
| ATOM | 1419 | O | SER | A | 184 | 86.817 | 45.395 | 52.043 | 1.00 | 9.90 | A |
| ATOM | 1420 | N | TYR | A | 185 | 88.361 | 45.515 | 50.416 | 1.00 | 9.44 | A |
| ATOM | 1421 | CA | TYR | A | 185 | 87.370 | 45.710 | 49.365 | 1.00 | 9.49 | A |
| ATOM | 1422 | CB | TYR | A | 185 | 87.607 | 47.018 | 48.606 | 1.00 | 8.45 | A |
| ATOM | 1423 | CG | TYR | A | 185 | 87.439 | 48.247 | 49.459 | 1.00 | 10.57 | A |
| ATOM | 1424 | CD1 | TYR | A | 185 | 88.513 | 48.774 | 50.175 | 1.00 | 10.71 | A |
| ATOM | 1425 | CE1 | TYR | A | 185 | 88.352 | 49.885 | 50.992 | 1.00 | 12.15 | A |
| ATOM | 1426 | CD2 | TYR | A | 185 | 86.195 | 48.866 | 49.579 | 1.00 | 10.29 | A |
| ATOM | 1427 | CE2 | TYR | A | 185 | 86.023 | 49.978 | 50.395 | 1.00 | 11.31 | A |
| ATOM | 1428 | CZ | TYR | A | 185 | 87.107 | 50.478 | 51.097 | 1.00 | 12.32 | A |
| ATOM | 1429 | OH | TYR | A | 185 | 86.938 | 51.566 | 51.918 | 1.00 | 13.32 | A |
| ATOM | 1430 | C | TYR | A | 185 | 87.481 | 44.551 | 48.404 | 1.00 | 9.56 | A |
| ATOM | 1431 | O | TYR | A | 185 | 88.582 | 44.186 | 47.996 | 1.00 | 11.36 | A |
| ATOM | 1432 | N | LEU | A | 186 | 86.345 | 43.968 | 48.043 | 1.00 | 7.78 | A |
| ATOM | 1433 | CA | LEU | A | 186 | 86.349 | 42.843 | 47.125 | 1.00 | 8.34 | A |
| ATOM | 1434 | CB | LEU | A | 186 | 85.928 | 41.558 | 47.852 | 1.00 | 7.94 | A |
| ATOM | 1435 | CG | LEU | A | 186 | 85.882 | 40.270 | 47.007 | 1.00 | 7.83 | A |
| ATOM | 1436 | CD1 | LEU | A | 186 | 87.296 | 39.862 | 46.610 | 1.00 | 9.12 | A |
| ATOM | 1437 | CD2 | LEU | A | 186 | 85.216 | 39.154 | 47.806 | 1.00 | 9.10 | A |
| ATOM | 1438 | C | LEU | A | 186 | 85.429 | 43.046 | 45.939 | 1.00 | 8.66 | A |
| ATOM | 1439 | O | LEU | A | 186 | 84.209 | 43.076 | 46.088 | 1.00 | 9.01 | A |
| ATOM | 1440 | N | PRO | A | 187 | 86.003 | 43.218 | 44.744 | 1.00 | 8.33 | A |
| ATOM | 1441 | CD | PRO | A | 187 | 87.393 | 43.547 | 44.387 | 1.00 | 9.25 | A |
| ATOM | 1442 | CA | PRO | A | 187 | 85.108 | 43.389 | 43.599 | 1.00 | 8.92 | A |
| ATOM | 1443 | CB | PRO | A | 187 | 86.067 | 43.721 | 42.458 | 1.00 | 9.23 | A |
| ATOM | 1444 | CG | PRO | A | 187 | 87.208 | 44.418 | 43.172 | 1.00 | 9.62 | A |
| ATOM | 1445 | C | PRO | A | 187 | 84.479 | 42.006 | 43.419 | 1.00 | 9.68 | A |
| ATOM | 1446 | O | PRO | A | 187 | 85.200 | 41.003 | 43.411 | 1.00 | 9.75 | A |
| ATOM | 1447 | N | VAL | A | 188 | 83.157 | 41.932 | 43.308 | 1.00 | 8.31 | A |
| ATOM | 1448 | CA | VAL | A | 188 | 82.517 | 40.633 | 43.135 | 1.00 | 9.15 | A |
| ATOM | 1449 | CB | VAL | A | 188 | 81.555 | 40.296 | 44.310 | 1.00 | 9.47 | A |
| ATOM | 1450 | CG1 | VAL | A | 188 | 82.364 | 40.026 | 45.566 | 1.00 | 10.26 | A |
| ATOM | 1451 | CG2 | VAL | A | 188 | 80.580 | 41.432 | 44.563 | 1.00 | 10.99 | A |
| ATOM | 1452 | C | VAL | A | 188 | 81.790 | 40.544 | 41.808 | 1.00 | 10.01 | A |
| ATOM | 1453 | O | VAL | A | 188 | 81.014 | 39.616 | 41.567 | 1.00 | 11.29 | A |
| ATOM | 1454 | N | GLY | A | 189 | 82.046 | 41.522 | 40.945 | 1.00 | 9.67 | A |
| ATOM | 1455 | CA | GLY | A | 189 | 81.435 | 41.504 | 39.633 | 1.00 | 9.96 | A |
| ATOM | 1456 | C | GLY | A | 189 | 80.510 | 42.628 | 39.208 | 1.00 | 9.21 | A |
| ATOM | 1457 | O | GLY | A | 189 | 79.605 | 43.048 | 39.931 | 1.00 | 9.56 | A |
| ATOM | 1458 | N | ASP | A | 190 | 80.777 | 43.098 | 37.998 | 1.00 | 10.08 | A |
| ATOM | 1459 | CA | ASP | A | 190 | 80.002 | 44.114 | 37.319 | 1.00 | 11.08 | A |
| ATOM | 1460 | CB | ASP | A | 190 | 78.991 | 43.397 | 36.426 | 1.00 | 11.56 | A |
| ATOM | 1461 | CG | ASP | A | 190 | 79.672 | 42.498 | 35.420 | 1.00 | 11.42 | A |
| ATOM | 1462 | OD1 | ASP | A | 190 | 80.328 | 43.047 | 34.512 | 1.00 | 12.61 | A |
| ATOM | 1463 | OD2 | ASP | A | 190 | 79.590 | 41.252 | 35.550 | 1.00 | 11.62 | A |
| ATOM | 1464 | C | ASP | A | 190 | 79.331 | 45.184 | 38.166 | 1.00 | 10.43 | A |
| ATOM | 1465 | O | ASP | A | 190 | 78.104 | 45.232 | 38.304 | 1.00 | 10.47 | A |
| ATOM | 1466 | N | GLY | A | 191 | 80.168 | 46.050 | 38.731 | 1.00 | 10.52 | A |
| ATOM | 1467 | CA | GLY | A | 191 | 79.680 | 47.152 | 39.541 | 1.00 | 10.91 | A |
| ATOM | 1468 | C | GLY | A | 191 | 79.573 | 46.928 | 41.035 | 1.00 | 10.70 | A |
| ATOM | 1469 | O | GLY | A | 191 | 79.554 | 47.891 | 41.801 | 1.00 | 11.07 | A |
| ATOM | 1470 | N | VAL | A | 192 | 79.508 | 45.674 | 41.466 | 1.00 | 10.43 | A |
| ATOM | 1471 | CA | VAL | A | 192 | 79.366 | 45.408 | 42.888 | 1.00 | 10.47 | A |
| ATOM | 1472 | CB | VAL | A | 192 | 78.461 | 44.180 | 43.139 | 1.00 | 10.54 | A |
| ATOM | 1473 | CG1 | VAL | A | 192 | 78.212 | 44.006 | 44.632 | 1.00 | 10.98 | A |
| ATOM | 1474 | CG2 | VAL | A | 192 | 77.148 | 44.352 | 42.397 | 1.00 | 12.46 | A |
| ATOM | 1475 | C | VAL | A | 192 | 80.693 | 45.213 | 43.610 | 1.00 | 10.31 | A |
| ATOM | 1476 | O | VAL | A | 192 | 81.552 | 44.436 | 43.185 | 1.00 | 10.15 | A |
| ATOM | 1477 | N | ILE | A | 193 | 80.850 | 45.945 | 44.708 | 1.00 | 10.55 | A |
| ATOM | 1478 | CA | ILE | A | 193 | 82.054 | 45.873 | 45.521 | 1.00 | 11.15 | A |
| ATOM | 1479 | CB | ILE | A | 193 | 82.922 | 47.136 | 45.339 | 1.00 | 13.17 | A |
| ATOM | 1480 | CG2 | ILE | A | 193 | 84.224 | 46.991 | 46.126 | 1.00 | 12.90 | A |
| ATOM | 1481 | CG1 | ILE | A | 193 | 83.231 | 47.329 | 43.843 | 1.00 | 15.76 | A |
| ATOM | 1482 | CD1 | ILE | A | 193 | 83.777 | 48.689 | 43.487 | 1.00 | 22.49 | A |
| ATOM | 1483 | C | ILE | A | 193 | 81.646 | 45.735 | 46.983 | 1.00 | 10.65 | A |
| ATOM | 1484 | O | ILE | A | 193 | 80.893 | 46.552 | 47.513 | 1.00 | 10.53 | A |
| ATOM | 1485 | N | LEU | A | 194 | 82.125 | 44.675 | 47.626 | 1.00 | 8.43 | A |
| ATOM | 1486 | CA | LEU | A | 194 | 81.815 | 44.428 | 49.026 | 1.00 | 9.65 | A |
| ATOM | 1487 | CB | LEU | A | 194 | 81.607 | 42.930 | 49.262 | 1.00 | 10.13 | A |
| ATOM | 1488 | CG | LEU | A | 194 | 81.085 | 42.539 | 50.643 | 1.00 | 12.96 | A |
| ATOM | 1489 | CD1 | LEU | A | 194 | 79.641 | 43.008 | 50.775 | 1.00 | 12.83 | A |
| ATOM | 1490 | CD2 | LEU | A | 194 | 81.163 | 41.024 | 50.816 | 1.00 | 13.98 | A |
| ATOM | 1491 | C | LEU | A | 194 | 82.981 | 44.917 | 49.871 | 1.00 | 9.74 | A |
| ATOM | 1492 | O | LEU | A | 194 | 84.134 | 44.851 | 49.443 | 1.00 | 10.48 | A |
| ATOM | 1493 | N | ALA | A | 195 | 82.685 | 45.416 | 51.064 | 1.00 | 9.12 | A |
| ATOM | 1494 | CA | ALA | A | 195 | 83.739 | 45.897 | 51.948 | 1.00 | 9.42 | A |
| ATOM | 1495 | CB | ALA | A | 195 | 83.850 | 47.421 | 51.851 | 1.00 | 10.76 | A |
| ATOM | 1496 | C | ALA | A | 195 | 83.477 | 45.487 | 53.389 | 1.00 | 8.94 | A |
| ATOM | 1497 | O | ALA | A | 195 | 82.333 | 45.438 | 53.832 | 1.00 | 9.84 | A |
| ATOM | 1498 | N | VAL | A | 196 | 84.545 | 45.166 | 54.107 | 1.00 | 8.67 | A |
| ATOM | 1499 | CA | VAL | A | 196 | 84.439 | 44.804 | 55.514 | 1.00 | 9.33 | A |
| ATOM | 1500 | CB | VAL | A | 196 | 84.671 | 43.300 | 55.757 | 1.00 | 9.27 | A |
| ATOM | 1501 | CG1 | VAL | A | 196 | 84.693 | 43.020 | 57.254 | 1.00 | 9.98 | A |
| ATOM | 1502 | CG2 | VAL | A | 196 | 83.546 | 42.493 | 55.105 | 1.00 | 10.53 | A |
| ATOM | 1503 | C | VAL | A | 196 | 85.520 | 45.609 | 56.212 | 1.00 | 9.90 | A |
| ATOM | 1504 | O | VAL | A | 196 | 86.689 | 45.567 | 55.819 | 1.00 | 9.68 | A |
| ATOM | 1505 | N | ALA | A | 197 | 85.129 | 46.342 | 57.247 | 1.00 | 9.83 | A |
| ATOM | 1506 | CA | ALA | A | 197 | 86.072 | 47.197 | 57.947 | 1.00 | 10.10 | A |
| ATOM | 1507 | CB | ALA | A | 197 | 85.700 | 48.654 | 57.691 | 1.00 | 12.57 | A |
| ATOM | 1508 | C | ALA | A | 197 | 86.230 | 46.976 | 59.443 | 1.00 | 10.07 | A |
| ATOM | 1509 | O | ALA | A | 197 | 85.299 | 46.573 | 60.139 | 1.00 | 10.79 | A |
| ATOM | 1510 | N | ARG | A | 198 | 87.439 | 47.262 | 59.919 | 1.00 | 10.33 | A |
| ATOM | 1511 | CA | ARG | A | 198 | 87.776 | 47.177 | 61.329 | 1.00 | 9.88 | A |
| ATOM | 1512 | CB | ARG | A | 198 | 89.296 | 47.181 | 61.499 | 1.00 | 9.59 | A |
| ATOM | 1513 | CG | ARG | A | 198 | 89.771 | 47.432 | 62.920 | 1.00 | 9.51 | A |
| ATOM | 1514 | CD | ARG | A | 198 | 91.290 | 47.354 | 62.999 | 1.00 | 11.62 | A |
| ATOM | 1515 | NE | ARG | A | 198 | 91.798 | 47.697 | 64.328 | 1.00 | 13.13 | A |
| ATOM | 1516 | CZ | ARG | A | 198 | 93.077 | 47.603 | 64.685 | 1.00 | 13.63 | A |
| ATOM | 1517 | NH1 | ARG | A | 198 | 93.989 | 47.176 | 63.817 | 1.00 | 12.84 | A |
| ATOM | 1518 | NH2 | ARG | A | 198 | 93.448 | 47.936 | 65.914 | 1.00 | 14.21 | A |
| ATOM | 1519 | C | ARG | A | 198 | 87.181 | 48.437 | 61.952 | 1.00 | 10.64 | A |
| ATOM | 1520 | O | ARG | A | 198 | 87.176 | 49.501 | 61.328 | 1.00 | 11.66 | A |
| ATOM | 1521 | N | VAL | A | 199 | 86.680 | 48.315 | 63.173 | 1.00 | 10.85 | A |
| ATOM | 1522 | CA | VAL | A | 199 | 86.076 | 49.448 | 63.859 | 1.00 | 12.97 | A |
| ATOM | 1523 | CB | VAL | A | 199 | 84.707 | 49.052 | 64.464 | 1.00 | 13.43 | A |
| ATOM | 1524 | CG1 | VAL | A | 199 | 84.023 | 50.276 | 65.057 | 1.00 | 16.41 | A |
| ATOM | 1525 | CG2 | VAL | A | 199 | 83.827 | 48.416 | 63.388 | 1.00 | 15.51 | A |
| ATOM | 1526 | C | VAL | A | 199 | 86.985 | 49.953 | 64.975 | 1.00 | 12.57 | A |
| ATOM | 1527 | O | VAL | A | 199 | 87.070 | 49.337 | 66.034 | 1.00 | 13.34 | A |
| ATOM | 1528 | N | GLY | A | 200 | 87.666 | 51.070 | 64.733 | 1.00 | 13.28 | A |
| ATOM | 1529 | CA | GLY | A | 200 | 88.555 | 51.628 | 65.739 | 1.00 | 13.13 | A |
| ATOM | 1530 | C | GLY | A | 200 | 89.633 | 50.657 | 66.190 | 1.00 | 12.90 | A |
| ATOM | 1531 | O | GLY | A | 200 | 90.345 | 50.080 | 65.369 | 1.00 | 12.98 | A |
| ATOM | 1532 | N | SER | A | 201 | 89.748 | 50.473 | 67.501 | 1.00 | 13.19 | A |
| ATOM | 1533 | CA | SER | A | 201 | 90.745 | 49.571 | 68.073 | 1.00 | 12.90 | A |
| ATOM | 1534 | CB | SER | A | 201 | 90.759 | 49.698 | 69.593 | 1.00 | 14.93 | A |
| ATOM | 1535 | OG | SER | A | 201 | 89.565 | 49.167 | 70.142 | 1.00 | 15.35 | A |
| ATOM | 1536 | C | SER | A | 201 | 90.437 | 48.125 | 67.714 | 1.00 | 12.99 | A |
| ATOM | 1537 | O | SER | A | 201 | 91.302 | 47.254 | 67.801 | 1.00 | 13.15 | A |
| ATOM | 1538 | N | GLY | A | 202 | 89.193 | 47.877 | 67.326 | 1.00 | 12.98 | A |
| ATOM | 1539 | CA | GLY | A | 202 | 88.784 | 46.532 | 66.979 | 1.00 | 12.44 | A |
| ATOM | 1540 | C | GLY | A | 202 | 87.984 | 45.884 | 68.095 | 1.00 | 13.15 | A |
| ATOM | 1541 | O | GLY | A | 202 | 87.328 | 44.866 | 67.886 | 1.00 | 12.44 | A |
| ATOM | 1542 | N | ALA | A | 203 | 88.035 | 46.465 | 69.289 | 1.00 | 14.25 | A |
| ATOM | 1543 | CA | ALA | A | 203 | 87.299 | 45.920 | 70.422 | 1.00 | 14.17 | A |
| ATOM | 1544 | CB | ALA | A | 203 | 87.620 | 46.720 | 71.688 | 1.00 | 14.31 | A |
| ATOM | 1545 | C | ALA | A | 203 | 85.802 | 45.976 | 70.134 | 1.00 | 13.17 | A |
| ATOM | 1546 | O | ALA | A | 203 | 85.342 | 46.852 | 69.402 | 1.00 | 14.66 | A |
| ATOM | 1547 | N | GLY | A | 204 | 85.047 | 45.036 | 70.696 | 1.00 | 12.56 | A |
| ATOM | 1548 | CA | GLY | A | 204 | 83.608 | 45.028 | 70.488 | 1.00 | 13.13 | A |
| ATOM | 1549 | C | GLY | A | 204 | 83.067 | 43.819 | 69.746 | 1.00 | 13.27 | A |
| ATOM | 1550 | O | GLY | A | 204 | 81.853 | 43.633 | 69.669 | 1.00 | 13.85 | A |
| ATOM | 1551 | N | GLY | A | 205 | 83.964 | 43.013 | 69.184 | 1.00 | 13.74 | A |
| ATOM | 1552 | CA | GLY | A | 205 | 83.561 | 41.814 | 68.464 | 1.00 | 13.62 | A |
| ATOM | 1553 | C | GLY | A | 205 | 82.857 | 42.024 | 67.134 | 1.00 | 13.62 | A |
| ATOM | 1554 | O | GLY | A | 205 | 82.200 | 41.111 | 66.628 | 1.00 | 12.76 | A |
| ATOM | 1555 | N | ALA | A | 206 | 83.004 | 43.205 | 66.547 | 1.00 | 12.29 | A |
| ATOM | 1556 | CA | ALA | A | 206 | 82.337 | 43.486 | 65.284 | 1.00 | 13.04 | A |
| ATOM | 1557 | CB | ALA | A | 206 | 81.211 | 44.486 | 65.520 | 1.00 | 14.60 | A |
| ATOM | 1558 | C | ALA | A | 206 | 83.220 | 43.985 | 64.149 | 1.00 | 12.69 | A |
| ATOM | 1559 | O | ALA | A | 206 | 84.251 | 44.617 | 64.367 | 1.00 | 12.79 | A |
| ATOM | 1560 | N | LEU | A | 207 | 82.789 | 43.664 | 62.932 | 1.00 | 11.31 | A |
| ATOM | 1561 | CA | LEU | A | 207 | 83.431 | 44.093 | 61.696 | 1.00 | 10.60 | A |
| ATOM | 1562 | CB | LEU | A | 207 | 84.035 | 42.905 | 60.946 | 1.00 | 9.73 | A |
| ATOM | 1563 | CG | LEU | A | 207 | 85.315 | 42.341 | 61.575 | 1.00 | 10.60 | A |
| ATOM | 1564 | CD1 | LEU | A | 207 | 85.720 | 41.058 | 60.858 | 1.00 | 11.65 | A |
| ATOM | 1565 | CD2 | LEU | A | 207 | 86.439 | 43.383 | 61.488 | 1.00 | 11.81 | A |
| ATOM | 1566 | C | LEU | A | 207 | 82.257 | 44.682 | 60.923 | 1.00 | 10.09 | A |
| ATOM | 1567 | O | LEU | A | 207 | 81.168 | 44.099 | 60.910 | 1.00 | 10.92 | A |
| ATOM | 1568 | N | ARG | A | 208 | 82.461 | 45.837 | 60.300 | 1.00 | 9.88 | A |
| ATOM | 1569 | CA | ARG | A | 208 | 81.377 | 46.496 | 59.582 | 1.00 | 9.90 | A |
| ATOM | 1570 | CB | ARG | A | 208 | 81.489 | 48.013 | 59.754 | 1.00 | 9.93 | A |
| ATOM | 1571 | CG | ARG | A | 208 | 80.183 | 48.755 | 59.530 | 1.00 | 10.48 | A |
| ATOM | 1572 | CD | ARG | A | 208 | 79.217 | 48.524 | 60.683 | 1.00 | 11.45 | A |
| ATOM | 1573 | NE | ARG | A | 208 | 77.984 | 49.285 | 60.510 | 1.00 | 12.39 | A |
| ATOM | 1574 | CZ | ARG | A | 208 | 77.050 | 49.423 | 61.446 | 1.00 | 14.29 | A |
| ATOM | 1575 | NH1 | ARG | A | 208 | 77.202 | 48.851 | 62.633 | 1.00 | 14.68 | A |
| ATOM | 1576 | NH2 | ARG | A | 208 | 75.962 | 50.140 | 61.194 | 1.00 | 15.04 | A |
| ATOM | 1577 | C | ARG | A | 208 | 81.358 | 46.146 | 58.103 | 1.00 | 9.38 | A |
| ATOM | 1578 | O | ARG | A | 208 | 82.393 | 46.145 | 57.440 | 1.00 | 11.07 | A |
| ATOM | 1579 | N | GLN | A | 209 | 80.167 | 45.859 | 57.590 | 1.00 | 9.80 | A |
| ATOM | 1580 | CA | GLN | A | 209 | 80.000 | 45.495 | 56.191 | 1.00 | 9.48 | A |
| ATOM | 1581 | CB | GLN | A | 209 | 79.135 | 44.232 | 56.097 | 1.00 | 9.22 | A |
| ATOM | 1582 | CG | GLN | A | 209 | 79.116 | 43.577 | 54.722 | 1.00 | 8.99 | A |
| ATOM | 1583 | CD | GLN | A | 209 | 78.255 | 42.327 | 54.706 | 1.00 | 9.94 | A |
| ATOM | 1584 | OE1 | GLN | A | 209 | 78.388 | 41.456 | 55.569 | 1.00 | 9.51 | A |
| ATOM | 1585 | NE2 | GLN | A | 209 | 77.376 | 42.226 | 53.718 | 1.00 | 8.98 | A |
| ATOM | 1586 | C | GLN | A | 209 | 79.355 | 46.624 | 55.381 | 1.00 | 9.95 | A |
| ATOM | 1587 | O | GLN | A | 209 | 78.343 | 47.189 | 55.790 | 1.00 | 10.17 | A |
| ATOM | 1588 | N | PHE | A | 210 | 79.960 | 46.948 | 54.239 | 1.00 | 10.87 | A |
| ATOM | 1589 | CA | PHE | A | 210 | 79.452 | 47.982 | 53.334 | 1.00 | 12.20 | A |
| ATOM | 1590 | CB | PHE | A | 210 | 80.381 | 49.201 | 53.276 | 1.00 | 14.11 | A |
| ATOM | 1591 | CG | PHE | A | 210 | 80.583 | 49.893 | 54.590 | 1.00 | 16.85 | A |
| ATOM | 1592 | CD1 | PHE | A | 210 | 81.510 | 49.415 | 55.507 | 1.00 | 19.89 | A |
| ATOM | 1593 | CD2 | PHE | A | 210 | 79.868 | 51.048 | 54.895 | 1.00 | 18.45 | A |
| ATOM | 1594 | CE1 | PHE | A | 210 | 81.729 | 50.080 | 56.715 | 1.00 | 20.94 | A |
| ATOM | 1595 | CE2 | PHE | A | 210 | 80.077 | 51.721 | 56.099 | 1.00 | 19.52 | A |
| ATOM | 1596 | CZ | PHE | A | 210 | 81.011 | 51.235 | 57.009 | 1.00 | 20.24 | A |
| ATOM | 1597 | C | PHE | A | 210 | 79.391 | 47.390 | 51.931 | 1.00 | 11.23 | A |
| ATOM | 1598 | O | PHE | A | 210 | 80.094 | 46.425 | 51.630 | 1.00 | 11.23 | A |
| ATOM | 1599 | N | ILE | A | 211 | 78.567 | 47.969 | 51.065 | 1.00 | 11.74 | A |
| ATOM | 1600 | CA | ILE | A | 211 | 78.471 | 47.467 | 49.703 | 1.00 | 11.93 | A |
| ATOM | 1601 | CB | ILE | A | 211 | 77.423 | 46.332 | 49.592 | 1.00 | 11.48 | A |
| ATOM | 1602 | CG2 | ILE | A | 211 | 76.009 | 46.907 | 49.697 | 1.00 | 12.47 | A |
| ATOM | 1603 | CG1 | ILE | A | 211 | 77.584 | 45.609 | 48.255 | 1.00 | 12.57 | A |
| ATOM | 1604 | CD1 | ILE | A | 211 | 76.691 | 44.381 | 48.106 | 1.00 | 13.45 | A |
| ATOM | 1605 | C | ILE | A | 211 | 78.110 | 48.576 | 48.724 | 1.00 | 12.33 | A |
| ATOM | 1606 | O | ILE | A | 211 | 77.356 | 49.494 | 49.056 | 1.00 | 12.79 | A |
| ATOM | 1607 | N | SER | A | 212 | 78.685 | 48.493 | 47.528 | 1.00 | 11.74 | A |
| ATOM | 1608 | CA | SER | A | 212 | 78.422 | 49.450 | 46.459 | 1.00 | 11.64 | A |
| ATOM | 1609 | CB | SER | A | 212 | 79.680 | 50.246 | 46.100 | 1.00 | 11.65 | A |
| ATOM | 1610 | OG | SER | A | 212 | 79.454 | 51.037 | 44.937 | 1.00 | 11.74 | A |
| ATOM | 1611 | C | SER | A | 212 | 77.982 | 48.644 | 45.248 | 1.00 | 11.58 | A |
| ATOM | 1612 | O | SER | A | 212 | 78.483 | 47.542 | 45.015 | 1.00 | 11.31 | A |
| ATOM | 1613 | N | LEU | A | 213 | 77.036 | 49.185 | 44.489 | 1.00 | 11.69 | A |
| ATOM | 1614 | CA | LEU | A | 213 | 76.536 | 48.505 | 43.304 | 1.00 | 12.25 | A |
| ATOM | 1615 | CB | LEU | A | 213 | 75.014 | 48.364 | 43.375 | 1.00 | 12.06 | A |
| ATOM | 1616 | CG | LEU | A | 213 | 74.398 | 47.740 | 44.626 | 1.00 | 12.61 | A |
| ATOM | 1617 | CD1 | LEU | A | 213 | 72.873 | 47.762 | 44.503 | 1.00 | 14.01 | A |
| ATOM | 1618 | CD2 | LEU | A | 213 | 74.898 | 46.316 | 44.795 | 1.00 | 11.63 | A |
| ATOM | 1619 | C | LEU | A | 213 | 76.910 | 49.273 | 42.043 | 1.00 | 12.38 | A |
| ATOM | 1620 | O | LEU | A | 213 | 76.579 | 48.853 | 40.933 | 1.00 | 12.95 | A |
| ATOM | 1621 | N | ASP | A | 214 | 77.607 | 50.393 | 42.217 | 1.00 | 13.03 | A |
| ATOM | 1622 | CA | ASP | A | 214 | 78.008 | 51.229 | 41.089 | 1.00 | 12.83 | A |
| ATOM | 1623 | CB | ASP | A | 214 | 77.214 | 52.539 | 41.098 | 1.00 | 14.48 | A |
| ATOM | 1624 | CG | ASP | A | 214 | 77.180 | 53.194 | 42.465 | 1.00 | 15.89 | A |
| ATOM | 1625 | OD1 | ASP | A | 214 | 78.044 | 52.867 | 43.311 | 1.00 | 14.64 | A |
| ATOM | 1626 | OD2 | ASP | A | 214 | 76.291 | 54.050 | 42.692 | 1.00 | 17.34 | A |
| ATOM | 1627 | C | ASP | A | 214 | 79.501 | 51.546 | 41.036 | 1.00 | 13.59 | A |
| ATOM | 1628 | O | ASP | A | 214 | 79.895 | 52.701 | 40.859 | 1.00 | 13.59 | A |
| ATOM | 1629 | N | ASP | A | 215 | 80.331 | 50.519 | 41.186 | 1.00 | 12.87 | A |
| ATOM | 1630 | CA | ASP | A | 215 | 81.780 | 50.694 | 41.137 | 1.00 | 13.23 | A |
| ATOM | 1631 | CB | ASP | A | 215 | 82.214 | 51.074 | 39.715 | 1.00 | 14.72 | A |
| ATOM | 1632 | CG | ASP | A | 215 | 82.609 | 49.869 | 38.880 | 1.00 | 16.88 | A |
| ATOM | 1633 | OD1 | ASP | A | 215 | 82.259 | 48.736 | 39.264 | 1.00 | 16.48 | A |
| ATOM | 1634 | OD2 | ASP | A | 215 | 83.265 | 50.059 | 37.834 | 1.00 | 19.03 | A |
| ATOM | 1635 | C | ASP | A | 215 | 82.322 | 51.728 | 42.127 | 1.00 | 13.46 | A |
| ATOM | 1636 | O | ASP | A | 215 | 83.293 | 52.426 | 41.835 | 1.00 | 14.27 | A |
| ATOM | 1637 | N | GLY | A | 216 | 81.692 | 51.825 | 43.294 | 1.00 | 13.61 | A |
| ATOM | 1638 | CA | GLY | A | 216 | 82.154 | 52.758 | 44.309 | 1.00 | 14.29 | A |
| ATOM | 1639 | C | GLY | A | 216 | 81.546 | 54.149 | 44.280 | 1.00 | 14.56 | A |
| ATOM | 1640 | O | GLY | A | 216 | 81.921 | 55.004 | 45.081 | 1.00 | 15.86 | A |
| ATOM | 1641 | N | GLY | A | 217 | 80.621 | 54.384 | 43.356 | 1.00 | 14.92 | A |
| ATOM | 1642 | CA | GLY | A | 217 | 79.981 | 55.687 | 43.269 | 1.00 | 16.07 | A |
| ATOM | 1643 | C | GLY | A | 217 | 79.227 | 56.021 | 44.544 | 1.00 | 16.66 | A |
| ATOM | 1644 | O | GLY | A | 217 | 79.349 | 57.122 | 45.087 | 1.00 | 18.15 | A |
| ATOM | 1645 | N | THR | A | 218 | 78.434 | 55.064 | 45.018 | 1.00 | 14.58 | A |
| ATOM | 1646 | CA | THR | A | 218 | 77.662 | 55.227 | 46.242 | 1.00 | 14.20 | A |
| ATOM | 1647 | CB | THR | A | 218 | 76.166 | 55.439 | 45.957 | 1.00 | 14.96 | A |
| ATOM | 1648 | OG1 | THR | A | 218 | 75.654 | 54.331 | 45.209 | 1.00 | 14.76 | A |
| ATOM | 1649 | CG2 | THR | A | 218 | 75.957 | 56.721 | 45.169 | 1.00 | 15.78 | A |
| ATOM | 1650 | C | THR | A | 218 | 77.814 | 53.984 | 47.100 | 1.00 | 14.41 | A |
| ATOM | 1651 | O | THR | A | 218 | 78.094 | 52.895 | 46.590 | 1.00 | 13.58 | A |
| ATOM | 1652 | N | TRP | A | 219 | 77.616 | 54.152 | 48.403 | 1.00 | 13.91 | A |
| ATOM | 1653 | CA | TRP | A | 219 | 77.751 | 53.051 | 49.345 | 1.00 | 13.48 | A |
| ATOM | 1654 | CB | TRP | A | 219 | 79.075 | 53.177 | 50.102 | 1.00 | 12.71 | A |
| ATOM | 1655 | CG | TRP | A | 219 | 80.268 | 53.103 | 49.219 | 1.00 | 12.11 | A |
| ATOM | 1656 | CD2 | TRP | A | 219 | 81.055 | 51.939 | 48.957 | 1.00 | 11.99 | A |
| ATOM | 1657 | CE2 | TRP | A | 219 | 82.052 | 52.310 | 48.028 | 1.00 | 12.87 | A |
| ATOM | 1658 | CE3 | TRP | A | 219 | 81.017 | 50.617 | 49.422 | 1.00 | 13.63 | A |
| ATOM | 1659 | CD1 | TRP | A | 219 | 80.798 | 54.107 | 48.459 | 1.00 | 12.67 | A |
| ATOM | 1660 | NE1 | TRP | A | 219 | 81.871 | 53.637 | 47.738 | 1.00 | 13.30 | A |
| ATOM | 1661 | CZ2 | TRP | A | 219 | 83.002 | 51.402 | 47.548 | 1.00 | 12.88 | A |
| ATOM | 1662 | CZ3 | TRP | A | 219 | 81.959 | 49.714 | 48.945 | 1.00 | 11.68 | A |
| ATOM | 1663 | CH2 | TRP | A | 219 | 82.942 | 50.113 | 48.017 | 1.00 | 13.47 | A |
| ATOM | 1664 | C | TRP | A | 219 | 76.614 | 52.950 | 50.350 | 1.00 | 14.21 | A |
| ATOM | 1665 | O | TRP | A | 219 | 75.989 | 53.946 | 50.712 | 1.00 | 15.05 | A |
| ATOM | 1666 | N | THR | A | 220 | 76.358 | 51.726 | 50.794 | 1.00 | 13.30 | A |
| ATOM | 1667 | CA | THR | A | 220 | 75.321 | 51.453 | 51.775 | 1.00 | 13.57 | A |
| ATOM | 1668 | CB | THR | A | 220 | 74.205 | 50.564 | 51.184 | 1.00 | 14.09 | A |
| ATOM | 1669 | OG1 | THR | A | 220 | 73.613 | 51.224 | 50.059 | 1.00 | 15.36 | A |
| ATOM | 1670 | CG2 | THR | A | 220 | 73.129 | 50.293 | 52.227 | 1.00 | 15.53 | A |
| ATOM | 1671 | C | THR | A | 220 | 75.948 | 50.725 | 52.958 | 1.00 | 13.49 | A |
| ATOM | 1672 | O | THR | A | 220 | 76.710 | 49.775 | 52.779 | 1.00 | 13.42 | A |
| ATOM | 1673 | N | ASP | A | 221 | 75.646 | 51.195 | 54.161 | 1.00 | 12.59 | A |
| ATOM | 1674 | CA | ASP | A | 221 | 76.147 | 50.586 | 55.385 | 1.00 | 12.89 | A |
| ATOM | 1675 | CB | ASP | A | 221 | 76.150 | 51.634 | 56.508 | 1.00 | 13.49 | A |
| ATOM | 1676 | CG | ASP | A | 221 | 76.536 | 51.060 | 57.859 | 1.00 | 14.78 | A |
| ATOM | 1677 | OD1 | ASP | A | 221 | 77.039 | 49.917 | 57.915 | 1.00 | 13.69 | A |
| ATOM | 1678 | OD2 | ASP | A | 221 | 76.342 | 51.765 | 58.876 | 1.00 | 15.80 | A |
| ATOM | 1679 | C | ASP | A | 221 | 75.179 | 49.449 | 55.699 | 1.00 | 12.44 | A |
| ATOM | 1680 | O | ASP | A | 221 | 74.017 | 49.685 | 56.032 | 1.00 | 13.68 | A |
| ATOM | 1681 | N | GLN | A | 222 | 75.652 | 48.212 | 55.571 | 1.00 | 11.63 | A |
| ATOM | 1682 | CA | GLN | A | 222 | 74.812 | 47.046 | 55.819 | 1.00 | 12.01 | A |
| ATOM | 1683 | CB | GLN | A | 222 | 75.257 | 45.881 | 54.933 | 1.00 | 10.59 | A |
| ATOM | 1684 | CG | GLN | A | 222 | 74.884 | 46.060 | 53.473 | 1.00 | 12.35 | A |
| ATOM | 1685 | CD | GLN | A | 222 | 75.511 | 44.994 | 52.598 | 1.00 | 11.42 | A |
| ATOM | 1686 | OE1 | GLN | A | 222 | 76.737 | 44.865 | 52.540 | 1.00 | 10.45 | A |
| ATOM | 1687 | NE2 | GLN | A | 222 | 74.677 | 44.234 | 51.901 | 1.00 | 12.57 | A |
| ATOM | 1688 | C | GLN | A | 222 | 74.775 | 46.582 | 57.265 | 1.00 | 11.20 | A |
| ATOM | 1689 | O | GLN | A | 222 | 74.030 | 45.663 | 57.606 | 1.00 | 13.73 | A |
| ATOM | 1690 | N | GLY | A | 223 | 75.578 | 47.211 | 58.115 | 1.00 | 11.29 | A |
| ATOM | 1691 | CA | GLY | A | 223 | 75.607 | 46.822 | 59.512 | 1.00 | 10.80 | A |
| ATOM | 1692 | C | GLY | A | 223 | 76.782 | 45.918 | 59.819 | 1.00 | 10.46 | A |
| ATOM | 1693 | O | GLY | A | 223 | 77.671 | 45.745 | 58.987 | 1.00 | 11.91 | A |
| ATOM | 1694 | N | ASN | A | 224 | 76.785 | 45.332 | 61.012 | 1.00 | 10.46 | A |
| ATOM | 1695 | CA | ASN | A | 224 | 77.872 | 44.455 | 61.413 | 1.00 | 10.64 | A |
| ATOM | 1696 | CB | ASN | A | 224 | 77.909 | 44.303 | 62.935 | 1.00 | 11.65 | A |
| ATOM | 1697 | CG | ASN | A | 224 | 78.107 | 45.623 | 63.647 | 1.00 | 11.78 | A |
| ATOM | 1698 | OD1 | ASN | A | 224 | 78.921 | 46.446 | 63.237 | 1.00 | 13.55 | A |
| ATOM | 1699 | ND2 | ASN | A | 224 | 77.365 | 45.826 | 64.728 | 1.00 | 16.84 | A |
| ATOM | 1700 | C | ASN | A | 224 | 77.768 | 43.071 | 60.794 | 1.00 | 9.82 | A |
| ATOM | 1701 | O | ASN | A | 224 | 76.671 | 42.537 | 60.620 | 1.00 | 11.09 | A |
| ATOM | 1702 | N | VAL | A | 225 | 78.922 | 42.504 | 60.458 | 1.00 | 9.08 | A |
| ATOM | 1703 | CA | VAL | A | 225 | 78.993 | 41.155 | 59.906 | 1.00 | 9.61 | A |
| ATOM | 1704 | CB | VAL | A | 225 | 80.467 | 40.724 | 59.722 | 1.00 | 8.44 | A |
| ATOM | 1705 | CG1 | VAL | A | 225 | 80.562 | 39.219 | 59.509 | 1.00 | 9.22 | A |
| ATOM | 1706 | CG2 | VAL | A | 225 | 81.068 | 41.458 | 58.533 | 1.00 | 9.06 | A |
| ATOM | 1707 | C | VAL | A | 225 | 78.310 | 40.288 | 60.957 | 1.00 | 10.11 | A |
| ATOM | 1708 | O | VAL | A | 225 | 78.693 | 40.304 | 62.129 | 1.00 | 10.77 | A |
| ATOM | 1709 | N | THR | A | 226 | 77.298 | 39.533 | 60.538 | 1.00 | 9.65 | A |
| ATOM | 1710 | CA | THR | A | 226 | 76.535 | 38.709 | 61.471 | 1.00 | 10.49 | A |
| ATOM | 1711 | CB | THR | A | 226 | 75.117 | 39.295 | 61.639 | 1.00 | 12.33 | A |
| ATOM | 1712 | OG1 | THR | A | 226 | 75.220 | 40.659 | 62.072 | 1.00 | 14.38 | A |
| ATOM | 1713 | CG2 | THR | A | 226 | 74.326 | 38.506 | 62.671 | 1.00 | 12.77 | A |
| ATOM | 1714 | C | THR | A | 226 | 76.414 | 37.227 | 61.112 | 1.00 | 10.60 | A |
| ATOM | 1715 | O | THR | A | 226 | 76.156 | 36.871 | 59.962 | 1.00 | 9.94 | A |
| ATOM | 1716 | N | ALA | A | 227 | 76.609 | 36.379 | 62.118 | 1.00 | 10.95 | A |
| ATOM | 1717 | CA | ALA | A | 227 | 76.506 | 34.930 | 61.975 | 1.00 | 10.45 | A |
| ATOM | 1718 | CB | ALA | A | 227 | 77.896 | 34.299 | 61.941 | 1.00 | 11.60 | A |
| ATOM | 1719 | C | ALA | A | 227 | 75.732 | 34.435 | 63.191 | 1.00 | 11.41 | A |
| ATOM | 1720 | O | ALA | A | 227 | 75.469 | 35.210 | 64.111 | 1.00 | 12.67 | A |
| ATOM | 1721 | N | GLN | A | 228 | 75.355 | 33.161 | 63.208 | 1.00 | 10.15 | A |
| ATOM | 1722 | CA | GLN | A | 228 | 74.621 | 32.641 | 64.352 | 1.00 | 8.85 | A |
| ATOM | 1723 | CB | GLN | A | 228 | 74.199 | 31.191 | 64.113 | 1.00 | 7.13 | A |
| ATOM | 1724 | CG | GLN | A | 228 | 73.496 | 30.580 | 65.313 | 1.00 | 9.21 | A |
| ATOM | 1725 | CD | GLN | A | 228 | 72.689 | 29.353 | 64.957 | 1.00 | 9.58 | A |
| ATOM | 1726 | OE1 | GLN | A | 228 | 73.203 | 28.410 | 64.354 | 1.00 | 11.17 | A |
| ATOM | 1727 | NE2 | GLN | A | 228 | 71.419 | 29.351 | 65.344 | 1.00 | 10.00 | A |
| ATOM | 1728 | C | GLN | A | 228 | 75.472 | 32.743 | 65.608 | 1.00 | 10.01 | A |
| ATOM | 1729 | O | GLN | A | 228 | 74.987 | 33.143 | 66.669 | 1.00 | 10.99 | A |
| ATOM | 1730 | N | ASN | A | 229 | 76.742 | 32.376 | 65.483 | 1.00 | 10.12 | A |
| ATOM | 1731 | CA | ASN | A | 229 | 77.672 | 32.442 | 66.605 | 1.00 | 10.42 | A |
| ATOM | 1732 | CB | ASN | A | 229 | 78.178 | 31.039 | 66.936 | 1.00 | 11.06 | A |
| ATOM | 1733 | CG | ASN | A | 229 | 77.059 | 30.126 | 67.400 | 1.00 | 12.29 | A |
| ATOM | 1734 | OD1 | ASN | A | 229 | 76.400 | 30.403 | 68.407 | 1.00 | 12.97 | A |
| ATOM | 1735 | ND2 | ASN | A | 229 | 76.828 | 29.040 | 66.667 | 1.00 | 12.41 | A |
| ATOM | 1736 | C | ASN | A | 229 | 78.812 | 33.381 | 66.232 | 1.00 | 12.10 | A |
| ATOM | 1737 | O | ASN | A | 229 | 79.625 | 33.076 | 65.361 | 1.00 | 11.97 | A |
| ATOM | 1738 | N | GLY | A | 230 | 78.853 | 34.529 | 66.904 | 1.00 | 11.54 | A |
| ATOM | 1739 | CA | GLY | A | 230 | 79.854 | 35.541 | 66.621 | 1.00 | 12.41 | A |
| ATOM | 1740 | C | GLY | A | 230 | 81.172 | 35.419 | 67.354 | 1.00 | 11.84 | A |
| ATOM | 1741 | O | GLY | A | 230 | 81.673 | 34.318 | 67.579 | 1.00 | 12.57 | A |
| ATOM | 1742 | N | ASP | A | 231 | 81.731 | 36.568 | 67.723 | 1.00 | 12.50 | A |
| ATOM | 1743 | CA | ASP | A | 231 | 83.007 | 36.621 | 68.429 | 1.00 | 12.31 | A |
| ATOM | 1744 | CB | ASP | A | 231 | 84.085 | 37.230 | 67.528 | 1.00 | 12.37 | A |
| ATOM | 1745 | CG | ASP | A | 231 | 84.279 | 36.450 | 66.239 | 1.00 | 12.21 | A |
| ATOM | 1746 | OD1 | ASP | A | 231 | 83.402 | 36.535 | 65.353 | 1.00 | 12.06 | A |
| ATOM | 1747 | OD2 | ASP | A | 231 | 85.302 | 35.746 | 66.125 | 1.00 | 11.87 | A |
| ATOM | 1748 | C | ASP | A | 231 | 82.913 | 37.433 | 69.716 | 1.00 | 12.93 | A |
| ATOM | 1749 | O | ASP | A | 231 | 83.839 | 38.172 | 70.063 | 1.00 | 13.17 | A |
| ATOM | 1750 | N | SER | A | 232 | 81.796 | 37.295 | 70.423 | 1.00 | 13.04 | A |
| ATOM | 1751 | CA | SER | A | 232 | 81.597 | 38.017 | 71.677 | 1.00 | 13.96 | A |
| ATOM | 1752 | CB | SER | A | 232 | 82.477 | 37.384 | 72.764 | 1.00 | 14.72 | A |
| ATOM | 1753 | OG | SER | A | 232 | 82.229 | 37.950 | 74.041 | 1.00 | 16.46 | A |
| ATOM | 1754 | C | SER | A | 232 | 81.954 | 39.495 | 71.477 | 1.00 | 13.89 | A |
| ATOM | 1755 | O | SER | A | 232 | 81.499 | 40.125 | 70.521 | 1.00 | 14.76 | A |
| ATOM | 1756 | N | THR | A | 233 | 82.761 | 40.045 | 72.380 | 1.00 | 13.68 | A |
| ATOM | 1757 | CA | THR | A | 233 | 83.185 | 41.441 | 72.283 | 1.00 | 13.64 | A |
| ATOM | 1758 | CB | THR | A | 233 | 82.929 | 42.206 | 73.600 | 1.00 | 14.41 | A |
| ATOM | 1759 | OG1 | THR | A | 233 | 83.538 | 41.495 | 74.685 | 1.00 | 16.05 | A |
| ATOM | 1760 | CG2 | THR | A | 233 | 81.440 | 42.348 | 73.859 | 1.00 | 15.43 | A |
| ATOM | 1761 | C | THR | A | 233 | 84.684 | 41.495 | 71.998 | 1.00 | 13.99 | A |
| ATOM | 1762 | O | THR | A | 233 | 85.351 | 42.485 | 72.314 | 1.00 | 14.43 | A |
| ATOM | 1763 | N | ASP | A | 234 | 85.203 | 40.430 | 71.391 | 1.00 | 12.53 | A |
| ATOM | 1764 | CA | ASP | A | 234 | 86.632 | 40.314 | 71.087 | 1.00 | 12.44 | A |
| ATOM | 1765 | CB | ASP | A | 234 | 86.950 | 38.940 | 70.480 | 1.00 | 12.32 | A |
| ATOM | 1766 | CG | ASP | A | 234 | 86.481 | 37.790 | 71.341 | 1.00 | 13.29 | A |
| ATOM | 1767 | OD1 | ASP | A | 234 | 86.171 | 38.007 | 72.534 | 1.00 | 13.43 | A |
| ATOM | 1768 | OD2 | ASP | A | 234 | 86.435 | 36.651 | 70.823 | 1.00 | 13.24 | A |
| ATOM | 1769 | C | ASP | A | 234 | 87.188 | 41.375 | 70.148 | 1.00 | 11.55 | A |
| ATOM | 1770 | O | ASP | A | 234 | 86.462 | 41.992 | 69.374 | 1.00 | 11.84 | A |
| ATOM | 1771 | N | ILE | A | 235 | 88.500 | 41.563 | 70.224 | 1.00 | 11.29 | A |
| ATOM | 1772 | CA | ILE | A | 235 | 89.199 | 42.520 | 69.381 | 1.00 | 11.75 | A |
| ATOM | 1773 | CB | ILE | A | 235 | 90.570 | 42.874 | 69.981 | 1.00 | 12.34 | A |
| ATOM | 1774 | CG2 | ILE | A | 235 | 91.331 | 43.801 | 69.048 | 1.00 | 13.23 | A |
| ATOM | 1775 | CG1 | ILE | A | 235 | 90.373 | 43.524 | 71.354 | 1.00 | 12.59 | A |
| ATOM | 1776 | CD1 | ILE | A | 235 | 91.625 | 43.542 | 72.203 | 1.00 | 14.23 | A |
| ATOM | 1777 | C | ILE | A | 235 | 89.407 | 41.884 | 68.008 | 1.00 | 10.94 | A |
| ATOM | 1778 | O | ILE | A | 235 | 90.115 | 40.879 | 67.879 | 1.00 | 11.49 | A |
| ATOM | 1779 | N | LEU | A | 236 | 88.783 | 42.466 | 66.987 | 1.00 | 10.02 | A |
| ATOM | 1780 | CA | LEU | A | 236 | 88.898 | 41.956 | 65.620 | 1.00 | 9.85 | A |
| ATOM | 1781 | CB | LEU | A | 236 | 87.508 | 41.666 | 65.048 | 1.00 | 9.86 | A |
| ATOM | 1782 | CG | LEU | A | 236 | 86.592 | 40.793 | 65.908 | 1.00 | 10.27 | A |
| ATOM | 1783 | CD1 | LEU | A | 236 | 85.294 | 40.524 | 65.154 | 1.00 | 10.52 | A |
| ATOM | 1784 | CD2 | LEU | A | 236 | 87.287 | 39.478 | 66.260 | 1.00 | 10.20 | A |
| ATOM | 1785 | C | LEU | A | 236 | 89.613 | 42.973 | 64.744 | 1.00 | 10.62 | A |
| ATOM | 1786 | O | LEU | A | 236 | 89.214 | 44.134 | 64.680 | 1.00 | 11.36 | A |
| ATOM | 1787 | N | VAL | A | 237 | 90.662 | 42.531 | 64.059 | 1.00 | 11.08 | A |
| ATOM | 1788 | CA | VAL | A | 237 | 91.429 | 43.438 | 63.213 | 1.00 | 10.75 | A |
| ATOM | 1789 | CB | VAL | A | 237 | 92.757 | 43.844 | 63.897 | 1.00 | 11.48 | A |
| ATOM | 1790 | CG1 | VAL | A | 237 | 92.483 | 44.386 | 65.291 | 1.00 | 13.15 | A |
| ATOM | 1791 | CG2 | VAL | A | 237 | 93.697 | 42.648 | 63.961 | 1.00 | 11.99 | A |
| ATOM | 1792 | C | VAL | A | 237 | 91.790 | 42.910 | 61.829 | 1.00 | 9.98 | A |
| ATOM | 1793 | O | VAL | A | 237 | 91.645 | 41.723 | 61.530 | 1.00 | 10.07 | A |
| ATOM | 1794 | N | ALA | A | 238 | 92.256 | 43.833 | 60.995 | 1.00 | 10.65 | A |
| ATOM | 1795 | CA | ALA | A | 238 | 92.721 | 43.557 | 59.644 | 1.00 | 8.65 | A |
| ATOM | 1796 | CB | ALA | A | 238 | 94.181 | 43.138 | 59.699 | 1.00 | 9.73 | A |
| ATOM | 1797 | C | ALA | A | 238 | 91.943 | 42.546 | 58.812 | 1.00 | 9.60 | A |
| ATOM | 1798 | O | ALA | A | 238 | 92.471 | 41.498 | 58.454 | 1.00 | 9.44 | A |
| ATOM | 1799 | N | PRO | A | 239 | 90.685 | 42.850 | 58.481 | 1.00 | 8.32 | A |
| ATOM | 1800 | CD | PRO | A | 239 | 89.894 | 44.046 | 58.820 | 1.00 | 8.62 | A |
| ATOM | 1801 | CA | PRO | A | 239 | 89.901 | 41.911 | 57.673 | 1.00 | 8.20 | A |
| ATOM | 1802 | CB | PRO | A | 239 | 88.496 | 42.507 | 57.723 | 1.00 | 8.83 | A |
| ATOM | 1803 | CG | PRO | A | 239 | 88.779 | 43.993 | 57.794 | 1.00 | 8.25 | A |
| ATOM | 1804 | C | PRO | A | 239 | 90.431 | 41.838 | 56.238 | 1.00 | 7.87 | A |
| ATOM | 1805 | O | PRO | A | 239 | 91.033 | 42.790 | 55.737 | 1.00 | 8.65 | A |
| ATOM | 1806 | N | SER | A | 240 | 90.234 | 40.693 | 55.591 | 1.00 | 8.04 | A |
| ATOM | 1807 | CA | SER | A | 240 | 90.634 | 40.522 | 54.197 | 1.00 | 8.45 | A |
| ATOM | 1808 | CB | SER | A | 240 | 92.068 | 39.974 | 54.069 | 1.00 | 8.39 | A |
| ATOM | 1809 | OG | SER | A | 240 | 92.254 | 38.724 | 54.705 | 1.00 | 9.38 | A |
| ATOM | 1810 | C | SER | A | 240 | 89.607 | 39.603 | 53.540 | 1.00 | 8.92 | A |
| ATOM | 1811 | O | SER | A | 240 | 89.067 | 38.703 | 54.182 | 1.00 | 10.59 | A |
| ATOM | 1812 | N | LEU | A | 241 | 89.330 | 39.849 | 52.265 | 1.00 | 8.83 | A |
| ATOM | 1813 | CA | LEU | A | 241 | 88.315 | 39.100 | 51.535 | 1.00 | 8.08 | A |
| ATOM | 1814 | CB | LEU | A | 241 | 87.188 | 40.055 | 51.138 | 1.00 | 8.18 | A |
| ATOM | 1815 | CG | LEU | A | 241 | 86.417 | 40.789 | 52.236 | 1.00 | 8.94 | A |
| ATOM | 1816 | CD1 | LEU | A | 241 | 85.662 | 41.974 | 51.632 | 1.00 | 9.29 | A |
| ATOM | 1817 | CD2 | LEU | A | 241 | 85.459 | 39.818 | 52.899 | 1.00 | 8.86 | A |
| ATOM | 1818 | C | LEU | A | 241 | 88.779 | 38.385 | 50.274 | 1.00 | 7.85 | A |
| ATOM | 1819 | O | LEU | A | 241 | 89.695 | 38.834 | 49.587 | 1.00 | 9.09 | A |
| ATOM | 1820 | N | SER | A | 242 | 88.117 | 37.272 | 49.968 | 1.00 | 7.81 | A |
| ATOM | 1821 | CA | SER | A | 242 | 88.409 | 36.509 | 48.754 | 1.00 | 7.70 | A |
| ATOM | 1822 | CB | SER | A | 242 | 89.263 | 35.275 | 49.060 | 1.00 | 7.63 | A |
| ATOM | 1823 | OG | SER | A | 242 | 90.600 | 35.626 | 49.334 | 1.00 | 8.42 | A |
| ATOM | 1824 | C | SER | A | 242 | 87.107 | 36.064 | 48.101 | 1.00 | 7.36 | A |
| ATOM | 1825 | O | SER | A | 242 | 86.103 | 35.853 | 48.777 | 1.00 | 7.79 | A |
| ATOM | 1826 | N | TYR | A | 243 | 87.154 | 35.911 | 46.783 | 1.00 | 7.28 | A |
| ATOM | 1827 | CA | TYR | A | 243 | 86.008 | 35.495 | 45.981 | 1.00 | 7.31 | A |
| ATOM | 1828 | CB | TYR | A | 243 | 85.824 | 36.472 | 44.818 | 1.00 | 7.61 | A |
| ATOM | 1829 | CG | TYR | A | 243 | 84.713 | 36.132 | 43.844 | 1.00 | 6.97 | A |
| ATOM | 1830 | CD1 | TYR | A | 243 | 83.406 | 36.558 | 44.076 | 1.00 | 7.30 | A |
| ATOM | 1831 | CE1 | TYR | A | 243 | 82.384 | 36.289 | 43.164 | 1.00 | 8.72 | A |
| ATOM | 1832 | CD2 | TYR | A | 243 | 84.977 | 35.417 | 42.672 | 1.00 | 7.71 | A |
| ATOM | 1833 | CE2 | TYR | A | 243 | 83.961 | 35.137 | 41.752 | 1.00 | 9.70 | A |
| ATOM | 1834 | CZ | TYR | A | 243 | 82.668 | 35.580 | 42.008 | 1.00 | 7.43 | A |
| ATOM | 1835 | OH | TYR | A | 243 | 81.657 | 35.316 | 41.108 | 1.00 | 8.82 | A |
| ATOM | 1836 | C | TYR | A | 243 | 86.250 | 34.103 | 45.410 | 1.00 | 7.45 | A |
| ATOM | 1837 | O | TYR | A | 243 | 87.315 | 33.832 | 44.854 | 1.00 | 8.40 | A |
| ATOM | 1838 | N | ILE | A | 244 | 85.268 | 33.219 | 45.546 | 1.00 | 6.74 | A |
| ATOM | 1839 | CA | ILE | A | 244 | 85.388 | 31.874 | 44.991 | 1.00 | 7.26 | A |
| ATOM | 1840 | CB | ILE | A | 244 | 85.886 | 30.841 | 46.046 | 1.00 | 8.17 | A |
| ATOM | 1841 | CG2 | ILE | A | 244 | 87.270 | 31.216 | 46.535 | 1.00 | 9.63 | A |
| ATOM | 1842 | CG1 | ILE | A | 244 | 84.902 | 30.744 | 47.217 | 1.00 | 8.16 | A |
| ATOM | 1843 | CD1 | ILE | A | 244 | 83.853 | 29.658 | 47.050 | 1.00 | 9.90 | A |
| ATOM | 1844 | C | ILE | A | 244 | 84.029 | 31.415 | 44.487 | 1.00 | 6.73 | A |
| ATOM | 1845 | O | ILE | A | 244 | 83.003 | 32.014 | 44.808 | 1.00 | 7.47 | A |
| ATOM | 1846 | N | TYR | A | 245 | 84.019 | 30.368 | 43.672 | 1.00 | 6.64 | A |
| ATOM | 1847 | CA | TYR | A | 245 | 82.757 | 29.820 | 43.209 | 1.00 | 6.26 | A |
| ATOM | 1848 | CB | TYR | A | 245 | 82.331 | 30.407 | 41.854 | 1.00 | 7.59 | A |
| ATOM | 1849 | CG | TYR | A | 245 | 83.335 | 30.356 | 40.733 | 1.00 | 6.34 | A |
| ATOM | 1850 | CD1 | TYR | A | 245 | 83.294 | 29.336 | 39.782 | 1.00 | 7.30 | A |
| ATOM | 1851 | CE1 | TYR | A | 245 | 84.176 | 29.322 | 38.711 | 1.00 | 7.99 | A |
| ATOM | 1852 | CD2 | TYR | A | 245 | 84.294 | 31.360 | 40.588 | 1.00 | 8.06 | A |
| ATOM | 1853 | CE2 | TYR | A | 245 | 85.185 | 31.353 | 39.522 | 1.00 | 9.24 | A |
| ATOM | 1854 | CZ | TYR | A | 245 | 85.120 | 30.332 | 38.588 | 1.00 | 9.78 | A |
| ATOM | 1855 | OH | TYR | A | 245 | 86.002 | 30.313 | 37.531 | 1.00 | 11.10 | A |
| ATOM | 1856 | C | TYR | A | 245 | 82.801 | 28.302 | 43.197 | 1.00 | 7.72 | A |
| ATOM | 1857 | O | TYR | A | 245 | 83.841 | 27.690 | 42.940 | 1.00 | 7.73 | A |
| ATOM | 1858 | N | SER | A | 246 | 81.652 | 27.707 | 43.498 | 1.00 | 6.27 | A |
| ATOM | 1859 | CA | SER | A | 246 | 81.507 | 26.260 | 43.624 | 1.00 | 6.88 | A |
| ATOM | 1860 | CB | SER | A | 246 | 80.140 | 25.940 | 44.225 | 1.00 | 8.06 | A |
| ATOM | 1861 | OG | SER | A | 246 | 79.136 | 26.036 | 43.234 | 1.00 | 9.44 | A |
| ATOM | 1862 | C | SER | A | 246 | 81.695 | 25.436 | 42.356 | 1.00 | 7.17 | A |
| ATOM | 1863 | O | SER | A | 246 | 81.909 | 25.964 | 41.266 | 1.00 | 6.43 | A |
| ATOM | 1864 | N | GLU | A | 247 | 81.595 | 24.123 | 42.536 | 1.00 | 7.05 | A |
| ATOM | 1865 | CA | GLU | A | 247 | 81.758 | 23.150 | 41.462 | 1.00 | 6.65 | A |
| ATOM | 1866 | CB | GLU | A | 247 | 81.570 | 21.729 | 42.015 | 1.00 | 7.54 | A |
| ATOM | 1867 | CG | GLU | A | 247 | 80.129 | 21.409 | 42.424 | 1.00 | 9.44 | A |
| ATOM | 1868 | CD | GLU | A | 247 | 79.991 | 20.045 | 43.082 | 1.00 | 11.04 | A |
| ATOM | 1869 | OE1 | GLU | A | 247 | 80.639 | 19.083 | 42.611 | 1.00 | 11.49 | A |
| ATOM | 1870 | OE2 | GLU | A | 247 | 79.219 | 19.931 | 44.057 | 1.00 | 12.08 | A |
| ATOM | 1871 | C | GLU | A | 247 | 80.789 | 23.382 | 40.304 | 1.00 | 7.09 | A |
| ATOM | 1872 | O | GLU | A | 247 | 81.022 | 22.910 | 39.194 | 1.00 | 7.05 | A |
| ATOM | 1873 | N | GLY | A | 248 | 79.699 | 24.097 | 40.573 | 1.00 | 6.61 | A |
| ATOM | 1874 | CA | GLY | A | 248 | 78.717 | 24.369 | 39.540 | 1.00 | 6.96 | A |
| ATOM | 1875 | C | GLY | A | 248 | 78.480 | 25.848 | 39.303 | 1.00 | 7.17 | A |
| ATOM | 1876 | O | GLY | A | 248 | 77.490 | 26.225 | 38.678 | 1.00 | 7.41 | A |
| ATOM | 1877 | N | GLY | A | 249 | 79.358 | 26.697 | 39.833 | 1.00 | 6.06 | A |
| ATOM | 1878 | CA | GLY | A | 249 | 79.230 | 28.127 | 39.600 | 1.00 | 6.52 | A |
| ATOM | 1879 | C | GLY | A | 249 | 78.669 | 29.049 | 40.666 | 1.00 | 6.52 | A |
| ATOM | 1880 | O | GLY | A | 249 | 78.618 | 30.256 | 40.444 | 1.00 | 7.70 | A |
| ATOM | 1881 | N | THR | A | 250 | 78.251 | 28.522 | 41.812 | 1.00 | 5.97 | A |
| ATOM | 1882 | CA | THR | A | 250 | 77.695 | 29.379 | 42.860 | 1.00 | 6.78 | A |
| ATOM | 1883 | CB | THR | A | 250 | 77.006 | 28.551 | 43.940 | 1.00 | 6.71 | A |
| ATOM | 1884 | OG1 | THR | A | 250 | 75.951 | 27.781 | 43.350 | 1.00 | 6.68 | A |
| ATOM | 1885 | CG2 | THR | A | 250 | 76.445 | 29.466 | 45.030 | 1.00 | 7.65 | A |
| ATOM | 1886 | C | THR | A | 250 | 78.781 | 30.223 | 43.535 | 1.00 | 6.67 | A |
| ATOM | 1887 | O | THR | A | 250 | 79.742 | 29.680 | 44.069 | 1.00 | 6.61 | A |
| ATOM | 1888 | N | PRO | A | 251 | 78.639 | 31.562 | 43.526 | 1.00 | 7.17 | A |
| ATOM | 1889 | CD | PRO | A | 251 | 77.699 | 32.347 | 42.704 | 1.00 | 9.02 | A |
| ATOM | 1890 | CA | PRO | A | 251 | 79.638 | 32.442 | 44.151 | 1.00 | 8.24 | A |
| ATOM | 1891 | CB | PRO | A | 251 | 79.449 | 33.759 | 43.404 | 1.00 | 9.36 | A |
| ATOM | 1892 | CG | PRO | A | 251 | 77.977 | 33.774 | 43.155 | 1.00 | 13.19 | A |
| ATOM | 1893 | C | PRO | A | 251 | 79.498 | 32.626 | 45.658 | 1.00 | 7.10 | A |
| ATOM | 1894 | O | PRO | A | 251 | 78.394 | 32.741 | 46.188 | 1.00 | 7.63 | A |
| ATOM | 1895 | N | HIS | A | 252 | 80.643 | 32.639 | 46.335 | 1.00 | 7.30 | A |
| ATOM | 1896 | CA | HIS | A | 252 | 80.707 | 32.840 | 47.778 | 1.00 | 6.73 | A |
| ATOM | 1897 | CB | HIS | A | 252 | 81.041 | 31.543 | 48.518 | 1.00 | 7.43 | A |
| ATOM | 1898 | CG | HIS | A | 252 | 79.927 | 30.548 | 48.555 | 1.00 | 6.86 | A |
| ATOM | 1899 | CD2 | HIS | A | 252 | 79.009 | 30.285 | 49.514 | 1.00 | 5.13 | A |
| ATOM | 1900 | ND1 | HIS | A | 252 | 79.678 | 29.658 | 47.531 | 1.00 | 10.06 | A |
| ATOM | 1901 | CE1 | HIS | A | 252 | 78.655 | 28.889 | 47.861 | 1.00 | 5.05 | A |
| ATOM | 1902 | NE2 | HIS | A | 252 | 78.232 | 29.249 | 49.060 | 1.00 | 10.25 | A |
| ATOM | 1903 | C | HIS | A | 252 | 81.828 | 33.823 | 48.091 | 1.00 | 7.22 | A |
| ATOM | 1904 | O | HIS | A | 252 | 82.786 | 33.959 | 47.326 | 1.00 | 8.04 | A |
| ATOM | 1905 | N | VAL | A | 253 | 81.704 | 34.490 | 49.232 | 1.00 | 6.33 | A |
| ATOM | 1906 | CA | VAL | A | 253 | 82.721 | 35.420 | 49.699 | 1.00 | 8.08 | A |
| ATOM | 1907 | CB | VAL | A | 253 | 82.112 | 36.797 | 50.068 | 1.00 | 7.94 | A |
| ATOM | 1908 | CG1 | VAL | A | 253 | 83.166 | 37.663 | 50.750 | 1.00 | 8.12 | A |
| ATOM | 1909 | CG2 | VAL | A | 253 | 81.590 | 37.497 | 48.814 | 1.00 | 7.81 | A |
| ATOM | 1910 | C | VAL | A | 253 | 83.356 | 34.802 | 50.938 | 1.00 | 7.70 | A |
| ATOM | 1911 | O | VAL | A | 253 | 82.658 | 34.248 | 51.786 | 1.00 | 7.12 | A |
| ATOM | 1912 | N | VAL | A | 254 | 84.680 | 34.878 | 51.020 | 1.00 | 7.01 | A |
| ATOM | 1913 | CA | VAL | A | 254 | 85.419 | 34.333 | 52.151 | 1.00 | 7.29 | A |
| ATOM | 1914 | CB | VAL | A | 254 | 86.520 | 33.366 | 51.678 | 1.00 | 7.45 | A |
| ATOM | 1915 | CG1 | VAL | A | 254 | 87.260 | 32.794 | 52.870 | 1.00 | 7.69 | A |
| ATOM | 1916 | CG2 | VAL | A | 254 | 85.895 | 32.240 | 50.854 | 1.00 | 10.06 | A |
| ATOM | 1917 | C | VAL | A | 254 | 86.056 | 35.492 | 52.909 | 1.00 | 7.01 | A |
| ATOM | 1918 | O | VAL | A | 254 | 86.762 | 36.316 | 52.327 | 1.00 | 7.95 | A |
| ATOM | 1919 | N | LEU | A | 255 | 85.783 | 35.565 | 54.206 | 1.00 | 7.16 | A |
| ATOM | 1920 | CA | LEU | A | 255 | 86.325 | 36.624 | 55.049 | 1.00 | 7.93 | A |
| ATOM | 1921 | CB | LEU | A | 255 | 85.193 | 37.323 | 55.823 | 1.00 | 8.00 | A |
| ATOM | 1922 | CG | LEU | A | 255 | 85.612 | 38.220 | 56.996 | 1.00 | 8.46 | A |
| ATOM | 1923 | CD1 | LEU | A | 255 | 86.322 | 39.461 | 56.471 | 1.00 | 9.40 | A |
| ATOM | 1924 | CD2 | LEU | A | 255 | 84.390 | 38.614 | 57.822 | 1.00 | 9.10 | A |
| ATOM | 1925 | C | LEU | A | 255 | 87.349 | 36.087 | 56.045 | 1.00 | 8.15 | A |
| ATOM | 1926 | O | LEU | A | 255 | 87.067 | 35.167 | 56.814 | 1.00 | 8.01 | A |
| ATOM | 1927 | N | LEU | A | 256 | 88.547 | 36.656 | 56.017 | 1.00 | 8.54 | A |
| ATOM | 1928 | CA | LEU | A | 256 | 89.589 | 36.271 | 56.959 | 1.00 | 8.91 | A |
| ATOM | 1929 | CB | LEU | A | 256 | 90.926 | 36.025 | 56.246 | 1.00 | 10.72 | A |
| ATOM | 1930 | CG | LEU | A | 256 | 91.249 | 34.646 | 55.655 | 1.00 | 13.71 | A |
| ATOM | 1931 | CD1 | LEU | A | 256 | 91.455 | 33.629 | 56.769 | 1.00 | 16.67 | A |
| ATOM | 1932 | CD2 | LEU | A | 256 | 90.144 | 34.220 | 54.716 | 1.00 | 17.74 | A |
| ATOM | 1933 | C | LEU | A | 256 | 89.742 | 37.441 | 57.911 | 1.00 | 8.94 | A |
| ATOM | 1934 | O | LEU | A | 256 | 89.628 | 38.599 | 57.512 | 1.00 | 8.93 | A |
| ATOM | 1935 | N | TYR | A | 257 | 89.974 | 37.150 | 59.181 | 1.00 | 8.03 | A |
| ATOM | 1936 | CA | TYR | A | 257 | 90.164 | 38.224 | 60.145 | 1.00 | 8.66 | A |
| ATOM | 1937 | CB | TYR | A | 257 | 88.821 | 38.814 | 60.592 | 1.00 | 8.88 | A |
| ATOM | 1938 | CG | TYR | A | 257 | 87.923 | 37.861 | 61.347 | 1.00 | 9.63 | A |
| ATOM | 1939 | CD1 | TYR | A | 257 | 87.138 | 36.926 | 60.674 | 1.00 | 9.87 | A |
| ATOM | 1940 | CE1 | TYR | A | 257 | 86.300 | 36.055 | 61.369 | 1.00 | 9.21 | A |
| ATOM | 1941 | CD2 | TYR | A | 257 | 87.853 | 37.902 | 62.741 | 1.00 | 8.94 | A |
| ATOM | 1942 | CE2 | TYR | A | 257 | 87.020 | 37.035 | 63.445 | 1.00 | 8.67 | A |
| ATOM | 1943 | CZ | TYR | A | 257 | 86.244 | 36.113 | 62.749 | 1.00 | 8.69 | A |
| ATOM | 1944 | OH | TYR | A | 257 | 85.413 | 35.253 | 63.427 | 1.00 | 10.55 | A |
| ATOM | 1945 | C | TYR | A | 257 | 90.916 | 37.685 | 61.339 | 1.00 | 9.66 | A |
| ATOM | 1946 | O | TYR | A | 257 | 90.964 | 36.477 | 61.559 | 1.00 | 9.81 | A |
| ATOM | 1947 | N | THR | A | 258 | 91.512 | 38.583 | 62.109 | 1.00 | 9.30 | A |
| ATOM | 1948 | CA | THR | A | 258 | 92.263 | 38.161 | 63.277 | 1.00 | 9.28 | A |
| ATOM | 1949 | CB | THR | A | 258 | 93.704 | 38.702 | 63.229 | 1.00 | 9.94 | A |
| ATOM | 1950 | OG1 | THR | A | 258 | 94.335 | 38.249 | 62.022 | 1.00 | 12.00 | A |
| ATOM | 1951 | CG2 | THR | A | 258 | 94.498 | 38.190 | 64.421 | 1.00 | 10.62 | A |
| ATOM | 1952 | C | THR | A | 258 | 91.585 | 38.586 | 64.572 | 1.00 | 9.56 | A |
| ATOM | 1953 | O | THR | A | 258 | 91.216 | 39.748 | 64.758 | 1.00 | 10.85 | A |
| ATOM | 1954 | N | ASN | A | 259 | 91.401 | 37.613 | 65.452 | 1.00 | 8.16 | A |
| ATOM | 1955 | CA | ASN | A | 259 | 90.770 | 37.826 | 66.748 | 1.00 | 8.73 | A |
| ATOM | 1956 | CB | ASN | A | 259 | 89.814 | 36.664 | 67.025 | 1.00 | 9.31 | A |
| ATOM | 1957 | CG | ASN | A | 259 | 89.034 | 36.839 | 68.303 | 1.00 | 9.60 | A |
| ATOM | 1958 | OD1 | ASN | A | 259 | 89.578 | 37.262 | 69.324 | 1.00 | 9.89 | A |
| ATOM | 1959 | ND2 | ASN | A | 259 | 87.754 | 36.489 | 68.265 | 1.00 | 10.00 | A |
| ATOM | 1960 | C | ASN | A | 259 | 91.901 | 37.854 | 67.773 | 1.00 | 9.11 | A |
| ATOM | 1961 | O | ASN | A | 259 | 92.499 | 36.821 | 68.078 | 1.00 | 9.51 | A |
| ATOM | 1962 | N | ARG | A | 260 | 92.200 | 39.036 | 68.299 | 1.00 | 10.27 | A |
| ATOM | 1963 | CA | ARG | A | 260 | 93.285 | 39.176 | 69.258 | 1.00 | 10.61 | A |
| ATOM | 1964 | CB | ARG | A | 260 | 93.899 | 40.566 | 69.140 | 1.00 | 11.32 | A |
| ATOM | 1965 | CG | ARG | A | 260 | 94.751 | 40.706 | 67.879 | 1.00 | 10.86 | A |
| ATOM | 1966 | CD | ARG | A | 260 | 95.375 | 42.083 | 67.758 | 1.00 | 11.14 | A |
| ATOM | 1967 | NE | ARG | A | 260 | 96.353 | 42.137 | 66.671 | 1.00 | 10.67 | A |
| ATOM | 1968 | CZ | ARG | A | 260 | 96.963 | 43.251 | 66.278 | 1.00 | 12.43 | A |
| ATOM | 1969 | NH1 | ARG | A | 260 | 96.692 | 44.401 | 66.881 | 1.00 | 14.78 | A |
| ATOM | 1970 | NH2 | ARG | A | 260 | 97.848 | 43.217 | 65.292 | 1.00 | 12.81 | A |
| ATOM | 1971 | C | ARG | A | 260 | 92.908 | 38.871 | 70.702 | 1.00 | 11.70 | A |
| ATOM | 1972 | O | ARG | A | 260 | 93.728 | 39.028 | 71.614 | 1.00 | 12.29 | A |
| ATOM | 1973 | N | THR | A | 261 | 91.669 | 38.435 | 70.909 | 1.00 | 10.62 | A |
| ATOM | 1974 | CA | THR | A | 261 | 91.216 | 38.057 | 72.241 | 1.00 | 11.01 | A |
| ATOM | 1975 | CB | THR | A | 261 | 89.734 | 38.429 | 72.462 | 1.00 | 10.49 | A |
| ATOM | 1976 | OG1 | THR | A | 261 | 89.588 | 39.855 | 72.415 | 1.00 | 11.15 | A |
| ATOM | 1977 | CG2 | THR | A | 261 | 89.253 | 37.918 | 73.819 | 1.00 | 11.81 | A |
| ATOM | 1978 | C | THR | A | 261 | 91.409 | 36.546 | 72.383 | 1.00 | 11.11 | A |
| ATOM | 1979 | O | THR | A | 261 | 91.966 | 36.076 | 73.375 | 1.00 | 12.81 | A |
| ATOM | 1980 | N | THR | A | 262 | 90.965 | 35.783 | 71.385 | 1.00 | 11.30 | A |
| ATOM | 1981 | CA | THR | A | 262 | 91.139 | 34.331 | 71.426 | 1.00 | 9.87 | A |
| ATOM | 1982 | CB | THR | A | 262 | 90.045 | 33.584 | 70.626 | 1.00 | 10.30 | A |
| ATOM | 1983 | OG1 | THR | A | 262 | 90.149 | 33.921 | 69.238 | 1.00 | 10.31 | A |
| ATOM | 1984 | CG2 | THR | A | 262 | 88.656 | 33.951 | 71.142 | 1.00 | 11.62 | A |
| ATOM | 1985 | C | THR | A | 262 | 92.504 | 33.940 | 70.855 | 1.00 | 9.84 | A |
| ATOM | 1986 | O | THR | A | 262 | 92.956 | 32.810 | 71.033 | 1.00 | 10.81 | A |
| ATOM | 1987 | N | HIS | A | 263 | 93.138 | 34.884 | 70.159 | 1.00 | 10.51 | A |
| ATOM | 1988 | CA | HIS | A | 263 | 94.462 | 34.698 | 69.554 | 1.00 | 10.36 | A |
| ATOM | 1989 | CB | HIS | A | 263 | 95.469 | 34.235 | 70.609 | 1.00 | 12.28 | A |
| ATOM | 1990 | CG | HIS | A | 263 | 95.432 | 35.046 | 71.865 | 1.00 | 13.94 | A |
| ATOM | 1991 | CD2 | HIS | A | 263 | 95.240 | 34.682 | 73.155 | 1.00 | 15.51 | A |
| ATOM | 1992 | ND1 | HIS | A | 263 | 95.586 | 36.415 | 71.871 | 1.00 | 14.59 | A |
| ATOM | 1993 | CE1 | HIS | A | 263 | 95.491 | 36.861 | 73.112 | 1.00 | 16.00 | A |
| ATOM | 1994 | NE2 | HIS | A | 263 | 95.281 | 35.830 | 73.910 | 1.00 | 15.05 | A |
| ATOM | 1995 | C | HIS | A | 263 | 94.466 | 33.722 | 68.385 | 1.00 | 10.46 | A |
| ATOM | 1996 | O | HIS | A | 263 | 95.296 | 32.811 | 68.313 | 1.00 | 10.67 | A |
| ATOM | 1997 | N | PHE | A | 264 | 93.527 | 33.923 | 67.468 | 1.00 | 10.04 | A |
| ATOM | 1998 | CA | PHE | A | 264 | 93.410 | 33.085 | 66.285 | 1.00 | 9.49 | A |
| ATOM | 1999 | CB | PHE | A | 264 | 92.342 | 32.002 | 66.483 | 1.00 | 10.04 | A |
| ATOM | 2000 | CG | PHE | A | 264 | 92.824 | 30.799 | 67.232 | 1.00 | 9.94 | A |
| ATOM | 2001 | CD1 | PHE | A | 264 | 93.662 | 29.872 | 66.622 | 1.00 | 11.92 | A |
| ATOM | 2002 | CD2 | PHE | A | 264 | 92.449 | 30.598 | 68.556 | 1.00 | 12.08 | A |
| ATOM | 2003 | CE1 | PHE | A | 264 | 94.123 | 28.758 | 67.322 | 1.00 | 13.38 | A |
| ATOM | 2004 | CE2 | PHE | A | 264 | 92.905 | 29.489 | 69.265 | 1.00 | 12.24 | A |
| ATOM | 2005 | CZ | PHE | A | 264 | 93.744 | 28.568 | 68.646 | 1.00 | 13.74 | A |
| ATOM | 2006 | C | PHE | A | 264 | 93.011 | 33.901 | 65.076 | 1.00 | 9.38 | A |
| ATOM | 2007 | O | PHE | A | 264 | 92.349 | 34.928 | 65.198 | 1.00 | 9.65 | A |
| ATOM | 2008 | N | CYS | A | 265 | 93.444 | 33.449 | 63.908 | 1.00 | 8.71 | A |
| ATOM | 2009 | CA | CYS | A | 265 | 93.053 | 34.074 | 62.655 | 1.00 | 9.57 | A |
| ATOM | 2010 | CB | CYS | A | 265 | 94.212 | 34.069 | 61.657 | 1.00 | 11.00 | A |
| ATOM | 2011 | SG | CYS | A | 265 | 93.733 | 34.308 | 59.932 | 1.00 | 16.52 | A |
| ATOM | 2012 | C | CYS | A | 265 | 91.970 | 33.100 | 62.211 | 1.00 | 9.93 | A |
| ATOM | 2013 | O | CYS | A | 265 | 92.193 | 31.884 | 62.198 | 1.00 | 9.83 | A |
| ATOM | 2014 | N | TYR | A | 266 | 90.798 | 33.623 | 61.873 | 1.00 | 7.86 | A |
| ATOM | 2015 | CA | TYR | A | 266 | 89.673 | 32.791 | 61.448 | 1.00 | 8.81 | A |
| ATOM | 2016 | CB | TYR | A | 266 | 88.460 | 33.003 | 62.356 | 1.00 | 8.91 | A |
| ATOM | 2017 | CG | TYR | A | 266 | 88.606 | 32.690 | 63.818 | 1.00 | 8.36 | A |
| ATOM | 2018 | CD1 | TYR | A | 266 | 88.560 | 31.375 | 64.276 | 1.00 | 8.64 | A |
| ATOM | 2019 | CE1 | TYR | A | 266 | 88.603 | 31.087 | 65.635 | 1.00 | 8.77 | A |
| ATOM | 2020 | CD2 | TYR | A | 266 | 88.707 | 33.716 | 64.755 | 1.00 | 8.83 | A |
| ATOM | 2021 | CE2 | TYR | A | 266 | 88.753 | 33.442 | 66.114 | 1.00 | 9.06 | A |
| ATOM | 2022 | CZ | TYR | A | 266 | 88.702 | 32.130 | 66.546 | 1.00 | 9.23 | A |
| ATOM | 2023 | OH | TYR | A | 266 | 88.774 | 31.855 | 67.889 | 1.00 | 10.21 | A |
| ATOM | 2024 | C | TYR | A | 266 | 89.182 | 33.202 | 60.076 | 1.00 | 8.79 | A |
| ATOM | 2025 | O | TYR | A | 266 | 89.536 | 34.267 | 59.569 | 1.00 | 8.86 | A |
| ATOM | 2026 | N | TYR | A | 267 | 88.349 | 32.346 | 59.486 | 1.00 | 9.24 | A |
| ATOM | 2027 | CA | TYR | A | 267 | 87.687 | 32.693 | 58.240 | 1.00 | 7.57 | A |
| ATOM | 2028 | CB | TYR | A | 267 | 88.323 | 32.037 | 56.998 | 1.00 | 8.24 | A |
| ATOM | 2029 | CG | TYR | A | 267 | 87.903 | 30.611 | 56.693 | 1.00 | 7.65 | A |
| ATOM | 2030 | CD1 | TYR | A | 267 | 86.714 | 30.332 | 56.003 | 1.00 | 7.53 | A |
| ATOM | 2031 | CE1 | TYR | A | 267 | 86.355 | 29.019 | 55.688 | 1.00 | 7.75 | A |
| ATOM | 2032 | CD2 | TYR | A | 267 | 88.716 | 29.544 | 57.065 | 1.00 | 10.14 | A |
| ATOM | 2033 | CE2 | TYR | A | 267 | 88.369 | 28.227 | 56.759 | 1.00 | 9.55 | A |
| ATOM | 2034 | CZ | TYR | A | 267 | 87.184 | 27.977 | 56.069 | 1.00 | 9.37 | A |
| ATOM | 2035 | OH | TYR | A | 267 | 86.818 | 26.688 | 55.762 | 1.00 | 10.00 | A |
| ATOM | 2036 | C | TYR | A | 267 | 86.239 | 32.263 | 58.387 | 1.00 | 7.54 | A |
| ATOM | 2037 | O | TYR | A | 267 | 85.908 | 31.380 | 59.182 | 1.00 | 7.86 | A |
| ATOM | 2038 | N | ARG | A | 268 | 85.377 | 32.932 | 57.641 | 1.00 | 7.93 | A |
| ATOM | 2039 | CA | ARG | A | 268 | 83.954 | 32.631 | 57.622 | 1.00 | 8.04 | A |
| ATOM | 2040 | CB | ARG | A | 268 | 83.161 | 33.650 | 58.451 | 1.00 | 8.32 | A |
| ATOM | 2041 | CG | ARG | A | 268 | 83.304 | 33.529 | 59.972 | 1.00 | 7.46 | A |
| ATOM | 2042 | CD | ARG | A | 268 | 82.517 | 34.642 | 60.674 | 1.00 | 8.18 | A |
| ATOM | 2043 | NE | ARG | A | 268 | 82.678 | 34.641 | 62.133 | 1.00 | 8.32 | A |
| ATOM | 2044 | CZ | ARG | A | 268 | 81.914 | 33.953 | 62.977 | 1.00 | 8.76 | A |
| ATOM | 2045 | NH1 | ARG | A | 268 | 80.922 | 33.201 | 62.515 | 1.00 | 9.75 | A |
| ATOM | 2046 | NH2 | ARG | A | 268 | 82.135 | 34.022 | 64.283 | 1.00 | 10.07 | A |
| ATOM | 2047 | C | ARG | A | 268 | 83.607 | 32.798 | 56.156 | 1.00 | 8.14 | A |
| ATOM | 2048 | O | ARG | A | 268 | 84.377 | 33.392 | 55.405 | 1.00 | 9.00 | A |
| ATOM | 2049 | N | THR | A | 269 | 82.468 | 32.266 | 55.735 | 1.00 | 8.38 | A |
| ATOM | 2050 | CA | THR | A | 269 | 82.057 | 32.413 | 54.345 | 1.00 | 7.38 | A |
| ATOM | 2051 | CB | THR | A | 269 | 82.123 | 31.089 | 53.562 | 1.00 | 8.63 | A |
| ATOM | 2052 | OG1 | THR | A | 269 | 81.066 | 30.224 | 53.995 | 1.00 | 9.04 | A |
| ATOM | 2053 | CG2 | THR | A | 269 | 83.457 | 30.396 | 53.784 | 1.00 | 8.97 | A |
| ATOM | 2054 | C | THR | A | 269 | 80.618 | 32.888 | 54.313 | 1.00 | 8.11 | A |
| ATOM | 2055 | O | THR | A | 269 | 79.930 | 32.898 | 55.334 | 1.00 | 8.20 | A |
| ATOM | 2056 | N | ILE | A | 270 | 80.166 | 33.284 | 53.133 | 1.00 | 7.58 | A |
| ATOM | 2057 | CA | ILE | A | 270 | 78.794 | 33.724 | 52.960 | 1.00 | 7.21 | A |
| ATOM | 2058 | CB | ILE | A | 270 | 78.594 | 35.176 | 53.465 | 1.00 | 8.09 | A |
| ATOM | 2059 | CG2 | ILE | A | 270 | 79.412 | 36.147 | 52.620 | 1.00 | 9.37 | A |
| ATOM | 2060 | CG1 | ILE | A | 270 | 77.109 | 35.557 | 53.386 | 1.00 | 9.53 | A |
| ATOM | 2061 | CD1 | ILE | A | 270 | 76.781 | 36.890 | 54.063 | 1.00 | 9.52 | A |
| ATOM | 2062 | C | ILE | A | 270 | 78.452 | 33.635 | 51.483 | 1.00 | 7.48 | A |
| ATOM | 2063 | O | ILE | A | 270 | 79.303 | 33.864 | 50.621 | 1.00 | 8.47 | A |
| ATOM | 2064 | N | LEU | A | 271 | 77.215 | 33.255 | 51.194 | 1.00 | 8.11 | A |
| ATOM | 2065 | CA | LEU | A | 271 | 76.753 | 33.177 | 49.819 | 1.00 | 8.53 | A |
| ATOM | 2066 | CB | LEU | A | 271 | 75.316 | 32.665 | 49.783 | 1.00 | 11.68 | A |
| ATOM | 2067 | CG | LEU | A | 271 | 75.073 | 31.166 | 49.804 | 1.00 | 15.42 | A |
| ATOM | 2068 | CD1 | LEU | A | 271 | 73.625 | 30.893 | 50.198 | 1.00 | 16.15 | A |
| ATOM | 2069 | CD2 | LEU | A | 271 | 75.363 | 30.602 | 48.413 | 1.00 | 14.86 | A |
| ATOM | 2070 | C | LEU | A | 271 | 76.781 | 34.609 | 49.309 | 1.00 | 8.81 | A |
| ATOM | 2071 | O | LEU | A | 271 | 76.317 | 35.514 | 50.004 | 1.00 | 9.64 | A |
| ATOM | 2072 | N | LEU | A | 272 | 77.311 | 34.839 | 48.114 | 1.00 | 8.59 | A |
| ATOM | 2073 | CA | LEU | A | 272 | 77.344 | 36.207 | 47.616 | 1.00 | 9.12 | A |
| ATOM | 2074 | CB | LEU | A | 272 | 77.950 | 36.275 | 46.213 | 1.00 | 9.57 | A |
| ATOM | 2075 | CG | LEU | A | 272 | 78.008 | 37.677 | 45.596 | 1.00 | 8.28 | A |
| ATOM | 2076 | CD1 | LEU | A | 272 | 78.777 | 38.619 | 46.520 | 1.00 | 9.56 | A |
| ATOM | 2077 | CD2 | LEU | A | 272 | 78.677 | 37.613 | 44.236 | 1.00 | 9.30 | A |
| ATOM | 2078 | C | LEU | A | 272 | 75.938 | 36.806 | 47.598 | 1.00 | 10.27 | A |
| ATOM | 2079 | O | LEU | A | 272 | 75.754 | 37.968 | 47.954 | 1.00 | 10.94 | A |
| ATOM | 2080 | N | ALA | A | 273 | 74.951 | 36.010 | 47.195 | 1.00 | 10.29 | A |
| ATOM | 2081 | CA | ALA | A | 273 | 73.573 | 36.486 | 47.141 | 1.00 | 10.49 | A |
| ATOM | 2082 | CB | ALA | A | 273 | 72.651 | 35.369 | 46.689 | 1.00 | 10.19 | A |
| ATOM | 2083 | C | ALA | A | 273 | 73.134 | 37.014 | 48.503 | 1.00 | 11.16 | A |
| ATOM | 2084 | O | ALA | A | 273 | 72.438 | 38.029 | 48.592 | 1.00 | 11.60 | A |
| ATOM | 2085 | N | ARG | A | 274 | 73.542 | 36.332 | 49.568 | 1.00 | 9.79 | A |
| ATOM | 2086 | CA | ARG | A | 274 | 73.184 | 36.768 | 50.916 | 1.00 | 10.84 | A |
| ATOM | 2087 | CB | ARG | A | 274 | 73.485 | 35.666 | 51.941 | 1.00 | 13.02 | A |
| ATOM | 2088 | CG | ARG | A | 274 | 72.452 | 34.539 | 51.975 | 1.00 | 17.07 | A |
| ATOM | 2089 | CD | ARG | A | 274 | 71.081 | 35.065 | 52.391 | 1.00 | 21.57 | A |
| ATOM | 2090 | NE | ARG | A | 274 | 71.102 | 35.717 | 53.702 | 1.00 | 23.91 | A |
| ATOM | 2091 | CZ | ARG | A | 274 | 71.009 | 35.077 | 54.867 | 1.00 | 25.83 | A |
| ATOM | 2092 | NH1 | ARG | A | 274 | 70.885 | 33.759 | 54.899 | 1.00 | 24.91 | A |
| ATOM | 2093 | NH2 | ARG | A | 274 | 71.037 | 35.757 | 56.006 | 1.00 | 27.76 | A |
| ATOM | 2094 | C | ARG | A | 274 | 73.932 | 38.047 | 51.286 | 1.00 | 10.60 | A |
| ATOM | 2095 | O | ARG | A | 274 | 73.354 | 38.960 | 51.878 | 1.00 | 11.15 | A |
| ATOM | 2096 | N | ALA | A | 275 | 75.209 | 38.125 | 50.925 | 1.00 | 10.33 | A |
| ATOM | 2097 | CA | ALA | A | 275 | 76.000 | 39.312 | 51.236 | 1.00 | 9.80 | A |
| ATOM | 2098 | CB | ALA | A | 275 | 77.457 | 39.099 | 50.832 | 1.00 | 9.87 | A |
| ATOM | 2099 | C | ALA | A | 275 | 75.447 | 40.558 | 50.553 | 1.00 | 10.92 | A |
| ATOM | 2100 | O | ALA | A | 275 | 75.520 | 41.658 | 51.101 | 1.00 | 11.08 | A |
| ATOM | 2101 | N | VAL | A | 276 | 74.899 | 40.392 | 49.354 | 1.00 | 10.21 | A |
| ATOM | 2102 | CA | VAL | A | 276 | 74.336 | 41.529 | 48.633 | 1.00 | 11.47 | A |
| ATOM | 2103 | CB | VAL | A | 276 | 74.034 | 41.158 | 47.154 | 1.00 | 12.44 | A |
| ATOM | 2104 | CG1 | VAL | A | 276 | 73.278 | 42.286 | 46.466 | 1.00 | 14.62 | A |
| ATOM | 2105 | CG2 | VAL | A | 276 | 75.343 | 40.888 | 46.418 | 1.00 | 13.01 | A |
| ATOM | 2106 | C | VAL | A | 276 | 73.052 | 41.985 | 49.332 | 1.00 | 12.22 | A |
| ATOM | 2107 | O | VAL | A | 276 | 72.744 | 43.175 | 49.360 | 1.00 | 13.08 | A |
| ATOM | 2108 | N | ALA | A | 277 | 72.322 | 41.034 | 49.908 | 1.00 | 11.85 | A |
| ATOM | 2109 | CA | ALA | A | 277 | 71.069 | 41.324 | 50.601 | 1.00 | 12.38 | A |
| ATOM | 2110 | CB | ALA | A | 277 | 70.237 | 40.054 | 50.710 | 1.00 | 10.87 | A |
| ATOM | 2111 | C | ALA | A | 277 | 71.247 | 41.943 | 51.991 | 1.00 | 13.05 | A |
| ATOM | 2112 | O | ALA | A | 277 | 70.363 | 42.652 | 52.478 | 1.00 | 14.11 | A |
| ATOM | 2113 | N | GLY | A | 278 | 72.381 | 41.674 | 52.632 | 1.00 | 11.82 | A |
| ATOM | 2114 | CA | GLY | A | 278 | 72.603 | 42.212 | 53.962 | 1.00 | 12.03 | A |
| ATOM | 2115 | C | GLY | A | 278 | 73.843 | 41.672 | 54.644 | 1.00 | 11.42 | A |
| ATOM | 2116 | O | GLY | A | 278 | 74.602 | 40.904 | 54.051 | 1.00 | 10.41 | A |
| ATOM | 2117 | N | SER | A | 279 | 74.042 | 42.058 | 55.901 | 1.00 | 10.43 | A |
| ATOM | 2118 | CA | SER | A | 279 | 75.219 | 41.631 | 56.654 | 1.00 | 11.07 | A |
| ATOM | 2119 | CB | SER | A | 279 | 75.643 | 42.740 | 57.624 | 1.00 | 10.95 | A |
| ATOM | 2120 | OG | SER | A | 279 | 74.665 | 42.969 | 58.618 | 1.00 | 13.55 | A |
| ATOM | 2121 | C | SER | A | 279 | 75.055 | 40.315 | 57.421 | 1.00 | 10.69 | A |
| ATOM | 2122 | O | SER | A | 279 | 75.998 | 39.859 | 58.058 | 1.00 | 10.71 | A |
| ATOM | 2123 | N | SER | A | 280 | 73.871 | 39.711 | 57.369 | 1.00 | 11.18 | A |
| ATOM | 2124 | CA | SER | A | 280 | 73.651 | 38.447 | 58.066 | 1.00 | 10.71 | A |
| ATOM | 2125 | CB | SER | A | 280 | 72.245 | 38.398 | 58.668 | 1.00 | 13.45 | A |
| ATOM | 2126 | OG | SER | A | 280 | 71.261 | 38.439 | 57.655 | 1.00 | 14.46 | A |
| ATOM | 2127 | C | SER | A | 280 | 73.827 | 37.289 | 57.087 | 1.00 | 10.74 | A |
| ATOM | 2128 | O | SER | A | 280 | 73.808 | 37.488 | 55.871 | 1.00 | 11.10 | A |
| ATOM | 2129 | N | GLY | A | 281 | 74.008 | 36.080 | 57.616 | 1.00 | 9.55 | A |
| ATOM | 2130 | CA | GLY | A | 281 | 74.163 | 34.919 | 56.755 | 1.00 | 9.26 | A |
| ATOM | 2131 | C | GLY | A | 281 | 75.550 | 34.309 | 56.723 | 1.00 | 9.36 | A |
| ATOM | 2132 | O | GLY | A | 281 | 75.770 | 33.303 | 56.039 | 1.00 | 10.38 | A |
| ATOM | 2133 | N | TRP | A | 282 | 76.493 | 34.898 | 57.452 | 1.00 | 9.09 | A |
| ATOM | 2134 | CA | TRP | A | 282 | 77.853 | 34.367 | 57.475 | 1.00 | 8.95 | A |
| ATOM | 2135 | CB | TRP | A | 282 | 78.806 | 35.359 | 58.153 | 1.00 | 8.13 | A |
| ATOM | 2136 | CG | TRP | A | 282 | 78.989 | 36.634 | 57.383 | 1.00 | 7.49 | A |
| ATOM | 2137 | CD2 | TRP | A | 282 | 80.030 | 36.922 | 56.445 | 1.00 | 8.47 | A |
| ATOM | 2138 | CE2 | TRP | A | 282 | 79.799 | 38.228 | 55.952 | 1.00 | 8.82 | A |
| ATOM | 2139 | CE3 | TRP | A | 282 | 81.133 | 36.200 | 55.963 | 1.00 | 8.03 | A |
| ATOM | 2140 | CD1 | TRP | A | 282 | 78.192 | 37.742 | 57.425 | 1.00 | 8.68 | A |
| ATOM | 2141 | NE1 | TRP | A | 282 | 78.671 | 38.704 | 56.570 | 1.00 | 8.07 | A |
| ATOM | 2142 | CZ2 | TRP | A | 282 | 80.640 | 38.836 | 55.010 | 1.00 | 8.84 | A |
| ATOM | 2143 | CZ3 | TRP | A | 282 | 81.966 | 36.802 | 55.027 | 1.00 | 7.87 | A |
| ATOM | 2144 | CH2 | TRP | A | 282 | 81.713 | 38.108 | 54.559 | 1.00 | 8.98 | A |
| ATOM | 2145 | C | TRP | A | 282 | 77.892 | 33.028 | 58.207 | 1.00 | 8.56 | A |
| ATOM | 2146 | O | TRP | A | 282 | 77.108 | 32.797 | 59.122 | 1.00 | 9.45 | A |
| ATOM | 2147 | N | THR | A | 283 | 78.808 | 32.154 | 57.796 | 1.00 | 7.92 | A |
| ATOM | 2148 | CA | THR | A | 283 | 78.941 | 30.836 | 58.407 | 1.00 | 9.55 | A |
| ATOM | 2149 | CB | THR | A | 283 | 79.542 | 29.823 | 57.409 | 1.00 | 10.70 | A |
| ATOM | 2150 | OG1 | THR | A | 283 | 80.826 | 30.288 | 56.979 | 1.00 | 13.36 | A |
| ATOM | 2151 | CG2 | THR | A | 283 | 78.632 | 29.671 | 56.201 | 1.00 | 12.85 | A |
| ATOM | 2152 | C | THR | A | 283 | 79.804 | 30.872 | 59.664 | 1.00 | 9.75 | A |
| ATOM | 2153 | O | THR | A | 283 | 80.376 | 31.912 | 60.008 | 1.00 | 9.83 | A |
| ATOM | 2154 | N | GLU | A | 284 | 79.893 | 29.735 | 60.351 | 1.00 | 10.31 | A |
| ATOM | 2155 | CA | GLU | A | 284 | 80.685 | 29.659 | 61.576 | 1.00 | 11.22 | A |
| ATOM | 2156 | CB | GLU | A | 284 | 80.509 | 28.296 | 62.247 | 1.00 | 16.10 | A |
| ATOM | 2157 | CG | GLU | A | 284 | 81.181 | 27.143 | 61.540 | 1.00 | 24.75 | A |
| ATOM | 2158 | CD | GLU | A | 284 | 81.153 | 25.879 | 62.375 | 1.00 | 29.53 | A |
| ATOM | 2159 | OE1 | GLU | A | 284 | 80.045 | 25.361 | 62.629 | 1.00 | 33.76 | A |
| ATOM | 2160 | OE2 | GLU | A | 284 | 82.237 | 25.410 | 62.789 | 1.00 | 34.00 | A |
| ATOM | 2161 | C | GLU | A | 284 | 82.163 | 29.905 | 61.301 | 1.00 | 10.56 | A |
| ATOM | 2162 | O | GLU | A | 284 | 82.654 | 29.650 | 60.199 | 1.00 | 10.83 | A |
| ATOM | 2163 | N | ARG | A | 285 | 82.873 | 30.401 | 62.307 | 1.00 | 8.98 | A |
| ATOM | 2164 | CA | ARG | A | 285 | 84.290 | 30.676 | 62.143 | 1.00 | 9.23 | A |
| ATOM | 2165 | CB | ARG | A | 285 | 84.782 | 31.651 | 63.223 | 1.00 | 9.07 | A |
| ATOM | 2166 | CG | ARG | A | 285 | 84.847 | 31.060 | 64.622 | 1.00 | 9.31 | A |
| ATOM | 2167 | CD | ARG | A | 285 | 85.162 | 32.131 | 65.660 | 1.00 | 9.70 | A |
| ATOM | 2168 | NE | ARG | A | 285 | 85.286 | 31.545 | 66.993 | 1.00 | 11.67 | A |
| ATOM | 2169 | CZ | ARG | A | 285 | 85.409 | 32.247 | 68.116 | 1.00 | 11.78 | A |
| ATOM | 2170 | NH1 | ARG | A | 285 | 85.426 | 33.574 | 68.080 | 1.00 | 11.61 | A |
| ATOM | 2171 | NH2 | ARG | A | 285 | 85.518 | 31.619 | 69.277 | 1.00 | 11.86 | A |
| ATOM | 2172 | C | ARG | A | 285 | 85.118 | 29.400 | 62.187 | 1.00 | 8.79 | A |
| ATOM | 2173 | O | ARG | A | 285 | 84.789 | 28.449 | 62.901 | 1.00 | 10.49 | A |
| ATOM | 2174 | N | VAL | A | 286 | 86.192 | 29.396 | 61.403 | 1.00 | 7.06 | A |
| ATOM | 2175 | CA | VAL | A | 286 | 87.116 | 28.272 | 61.326 | 1.00 | 8.06 | A |
| ATOM | 2176 | CB | VAL | A | 286 | 87.067 | 27.595 | 59.940 | 1.00 | 7.88 | A |
| ATOM | 2177 | CG1 | VAL | A | 286 | 88.102 | 26.476 | 59.869 | 1.00 | 8.63 | A |
| ATOM | 2178 | CG2 | VAL | A | 286 | 85.671 | 27.041 | 59.678 | 1.00 | 9.70 | A |
| ATOM | 2179 | C | VAL | A | 286 | 88.516 | 28.827 | 61.541 | 1.00 | 8.39 | A |
| ATOM | 2180 | O | VAL | A | 286 | 88.934 | 29.753 | 60.850 | 1.00 | 8.45 | A |
| ATOM | 2181 | N | PRO | A | 287 | 89.257 | 28.277 | 62.515 | 1.00 | 9.34 | A |
| ATOM | 2182 | CD | PRO | A | 287 | 88.890 | 27.218 | 63.471 | 1.00 | 10.67 | A |
| ATOM | 2183 | CA | PRO | A | 287 | 90.612 | 28.771 | 62.764 | 1.00 | 10.12 | A |
| ATOM | 2184 | CB | PRO | A | 287 | 90.976 | 28.120 | 64.096 | 1.00 | 10.30 | A |
| ATOM | 2185 | CG | PRO | A | 287 | 90.230 | 26.819 | 64.044 | 1.00 | 12.41 | A |
| ATOM | 2186 | C | PRO | A | 287 | 91.568 | 28.391 | 61.639 | 1.00 | 10.32 | A |
| ATOM | 2187 | O | PRO | A | 287 | 91.676 | 27.219 | 61.264 | 1.00 | 11.88 | A |
| ATOM | 2188 | N | ALA | A | 288 | 92.249 | 29.390 | 61.089 | 1.00 | 9.62 | A |
| ATOM | 2189 | CA | ALA | A | 288 | 93.197 | 29.160 | 60.005 | 1.00 | 9.53 | A |
| ATOM | 2190 | CB | ALA | A | 288 | 93.034 | 30.240 | 58.927 | 1.00 | 11.04 | A |
| ATOM | 2191 | C | ALA | A | 288 | 94.638 | 29.145 | 60.506 | 1.00 | 10.41 | A |
| ATOM | 2192 | O | ALA | A | 288 | 95.504 | 28.513 | 59.903 | 1.00 | 11.22 | A |
| ATOM | 2193 | N | TYR | A | 289 | 94.896 | 29.844 | 61.607 | 1.00 | 8.93 | A |
| ATOM | 2194 | CA | TYR | A | 289 | 96.246 | 29.907 | 62.153 | 1.00 | 8.94 | A |
| ATOM | 2195 | CB | TYR | A | 289 | 97.148 | 30.738 | 61.230 | 1.00 | 8.92 | A |
| ATOM | 2196 | CG | TYR | A | 289 | 98.559 | 30.205 | 61.062 | 1.00 | 8.49 | A |
| ATOM | 2197 | CD1 | TYR | A | 289 | 98.997 | 29.733 | 59.827 | 1.00 | 8.90 | A |
| ATOM | 2198 | CE1 | TYR | A | 289 | 100.294 | 29.247 | 59.652 | 1.00 | 9.47 | A |
| ATOM | 2199 | CD2 | TYR | A | 289 | 99.455 | 30.179 | 62.129 | 1.00 | 9.81 | A |
| ATOM | 2200 | CE2 | TYR | A | 289 | 100.757 | 29.693 | 61.967 | 1.00 | 9.35 | A |
| ATOM | 2201 | CZ | TYR | A | 289 | 101.167 | 29.230 | 60.726 | 1.00 | 9.05 | A |
| ATOM | 2202 | OH | TYR | A | 289 | 102.444 | 28.747 | 60.565 | 1.00 | 9.86 | A |
| ATOM | 2203 | C | TYR | A | 289 | 96.225 | 30.567 | 63.516 | 1.00 | 10.07 | A |
| ATOM | 2204 | O | TYR | A | 289 | 95.375 | 31.418 | 63.790 | 1.00 | 10.77 | A |
| ATOM | 2205 | N | SER | A | 290 | 97.161 | 30.177 | 64.372 | 1.00 | 9.72 | A |
| ATOM | 2206 | CA | SER | A | 290 | 97.269 | 30.795 | 65.683 | 1.00 | 10.31 | A |
| ATOM | 2207 | CB | SER | A | 290 | 98.336 | 30.088 | 66.522 | 1.00 | 10.67 | A |
| ATOM | 2208 | OG | SER | A | 290 | 99.597 | 30.169 | 65.879 | 1.00 | 12.16 | A |
| ATOM | 2209 | C | SER | A | 290 | 97.706 | 32.225 | 65.397 | 1.00 | 9.22 | A |
| ATOM | 2210 | O | SER | A | 290 | 98.363 | 32.491 | 64.391 | 1.00 | 9.81 | A |
| ATOM | 2211 | N | ALA | A | 291 | 97.341 | 33.145 | 66.279 | 1.00 | 9.58 | A |
| ATOM | 2212 | CA | ALA | A | 291 | 97.703 | 34.552 | 66.107 | 1.00 | 9.71 | A |
| ATOM | 2213 | CB | ALA | A | 291 | 96.558 | 35.303 | 65.440 | 1.00 | 10.01 | A |
| ATOM | 2214 | C | ALA | A | 291 | 97.998 | 35.144 | 67.479 | 1.00 | 9.76 | A |
| ATOM | 2215 | O | ALA | A | 291 | 97.171 | 35.853 | 68.049 | 1.00 | 9.33 | A |
| ATOM | 2216 | N | PRO | A | 292 | 99.192 | 34.867 | 68.022 | 1.00 | 9.67 | A |
| ATOM | 2217 | CD | PRO | A | 292 | 100.280 | 34.048 | 67.457 | 1.00 | 10.28 | A |
| ATOM | 2218 | CA | PRO | A | 292 | 99.559 | 35.385 | 69.345 | 1.00 | 10.05 | A |
| ATOM | 2219 | CB | PRO | A | 292 | 100.814 | 34.588 | 69.683 | 1.00 | 10.51 | A |
| ATOM | 2220 | CG | PRO | A | 292 | 101.462 | 34.422 | 68.349 | 1.00 | 10.85 | A |
| ATOM | 2221 | C | PRO | A | 292 | 99.798 | 36.888 | 69.397 | 1.00 | 10.03 | A |
| ATOM | 2222 | O | PRO | A | 292 | 100.280 | 37.491 | 68.440 | 1.00 | 10.33 | A |
| ATOM | 2223 | N | ALA | A | 293 | 99.447 | 37.486 | 70.530 | 1.00 | 10.40 | A |
| ATOM | 2224 | CA | ALA | A | 293 | 99.632 | 38.914 | 70.747 | 1.00 | 11.04 | A |
| ATOM | 2225 | CB | ALA | A | 293 | 101.080 | 39.188 | 71.139 | 1.00 | 12.27 | A |
| ATOM | 2226 | C | ALA | A | 293 | 99.225 | 39.781 | 69.555 | 1.00 | 11.36 | A |
| ATOM | 2227 | O | ALA | A | 293 | 98.112 | 39.656 | 69.047 | 1.00 | 11.51 | A |
| ATOM | 2228 | N | ALA | A | 294 | 100.123 | 40.654 | 69.103 | 1.00 | 10.59 | A |
| ATOM | 2229 | CA | ALA | A | 294 | 99.816 | 41.556 | 67.993 | 1.00 | 10.10 | A |
| ATOM | 2230 | CB | ALA | A | 294 | 100.614 | 42.854 | 68.147 | 1.00 | 11.08 | A |
| ATOM | 2231 | C | ALA | A | 294 | 100.024 | 40.975 | 66.593 | 1.00 | 11.34 | A |
| ATOM | 2232 | O | ALA | A | 294 | 100.669 | 41.585 | 65.738 | 1.00 | 12.50 | A |
| ATOM | 2233 | N | SER | A | 295 | 99.457 | 39.796 | 66.367 | 1.00 | 12.05 | A |
| ATOM | 2234 | CA | SER | A | 295 | 99.548 | 39.123 | 65.073 | 1.00 | 11.44 | A |
| ATOM | 2235 | CB | SER | A | 295 | 99.576 | 37.605 | 65.268 | 1.00 | 11.29 | A |
| ATOM | 2236 | OG | SER | A | 295 | 100.738 | 37.191 | 65.964 | 1.00 | 10.63 | A |
| ATOM | 2237 | C | SER | A | 295 | 98.342 | 39.487 | 64.211 | 1.00 | 12.41 | A |
| ATOM | 2238 | O | SER | A | 295 | 97.441 | 40.200 | 64.657 | 1.00 | 12.32 | A |
| ATOM | 2239 | N | GLY | A | 296 | 98.334 | 38.988 | 62.976 | 1.00 | 11.59 | A |
| ATOM | 2240 | CA | GLY | A | 296 | 97.229 | 39.247 | 62.067 | 1.00 | 10.98 | A |
| ATOM | 2241 | C | GLY | A | 296 | 97.680 | 39.784 | 60.721 | 1.00 | 10.12 | A |
| ATOM | 2242 | O | GLY | A | 296 | 98.804 | 39.532 | 60.291 | 1.00 | 11.03 | A |
| ATOM | 2243 | N | TYR | A | 297 | 96.792 | 40.525 | 60.064 | 1.00 | 9.66 | A |
| ATOM | 2244 | CA | TYR | A | 297 | 97.066 | 41.131 | 58.766 | 1.00 | 9.50 | A |
| ATOM | 2245 | CB | TYR | A | 297 | 98.283 | 42.058 | 58.860 | 1.00 | 10.37 | A |
| ATOM | 2246 | CG | TYR | A | 297 | 97.998 | 43.262 | 59.737 | 1.00 | 10.72 | A |
| ATOM | 2247 | CD1 | TYR | A | 297 | 97.829 | 43.118 | 61.114 | 1.00 | 11.86 | A |
| ATOM | 2248 | CE1 | TYR | A | 297 | 97.490 | 44.203 | 61.920 | 1.00 | 11.74 | A |
| ATOM | 2249 | CD2 | TYR | A | 297 | 97.825 | 44.531 | 59.182 | 1.00 | 10.92 | A |
| ATOM | 2250 | CE2 | TYR | A | 297 | 97.483 | 45.631 | 59.980 | 1.00 | 10.28 | A |
| ATOM | 2251 | CZ | TYR | A | 297 | 97.317 | 45.454 | 61.348 | 1.00 | 11.36 | A |
| ATOM | 2252 | OH | TYR | A | 297 | 96.967 | 46.515 | 62.155 | 1.00 | 13.31 | A |
| ATOM | 2253 | C | TYR | A | 297 | 97.235 | 40.092 | 57.669 | 1.00 | 9.55 | A |
| ATOM | 2254 | O | TYR | A | 297 | 98.330 | 39.823 | 57.178 | 1.00 | 9.52 | A |
| ATOM | 2255 | N | THR | A | 298 | 96.093 | 39.538 | 57.282 | 1.00 | 9.60 | A |
| ATOM | 2256 | CA | THR | A | 298 | 96.002 | 38.501 | 56.272 | 1.00 | 8.58 | A |
| ATOM | 2257 | CB | THR | A | 298 | 94.814 | 37.589 | 56.588 | 1.00 | 8.85 | A |
| ATOM | 2258 | OG1 | THR | A | 298 | 93.674 | 38.393 | 56.926 | 1.00 | 9.73 | A |
| ATOM | 2259 | CG2 | THR | A | 298 | 95.141 | 36.681 | 57.749 | 1.00 | 11.39 | A |
| ATOM | 2260 | C | THR | A | 298 | 95.847 | 38.985 | 54.836 | 1.00 | 8.15 | A |
| ATOM | 2261 | O | THR | A | 298 | 95.351 | 40.082 | 54.577 | 1.00 | 9.54 | A |
| ATOM | 2262 | N | SER | A | 299 | 96.298 | 38.148 | 53.907 | 1.00 | 7.54 | A |
| ATOM | 2263 | CA | SER | A | 299 | 96.183 | 38.415 | 52.478 | 1.00 | 7.71 | A |
| ATOM | 2264 | CB | SER | A | 299 | 97.400 | 39.171 | 51.939 | 1.00 | 8.51 | A |
| ATOM | 2265 | OG | SER | A | 299 | 98.615 | 38.532 | 52.253 | 1.00 | 8.56 | A |
| ATOM | 2266 | C | SER | A | 299 | 96.062 | 37.049 | 51.817 | 1.00 | 8.50 | A |
| ATOM | 2267 | O | SER | A | 299 | 96.472 | 36.036 | 52.395 | 1.00 | 8.55 | A |
| ATOM | 2268 | N | GLN | A | 300 | 95.504 | 37.027 | 50.612 | 1.00 | 7.71 | A |
| ATOM | 2269 | CA | GLN | A | 300 | 95.293 | 35.781 | 49.896 | 1.00 | 7.99 | A |
| ATOM | 2270 | CB | GLN | A | 300 | 93.853 | 35.289 | 50.109 | 1.00 | 9.03 | A |
| ATOM | 2271 | CG | GLN | A | 300 | 93.356 | 35.232 | 51.547 | 1.00 | 8.98 | A |
| ATOM | 2272 | CD | GLN | A | 300 | 92.851 | 36.574 | 52.068 | 1.00 | 11.05 | A |
| ATOM | 2273 | OE1 | GLN | A | 300 | 93.405 | 37.127 | 53.016 | 1.00 | 10.27 | A |
| ATOM | 2274 | NE2 | GLN | A | 300 | 91.782 | 37.093 | 51.459 | 1.00 | 12.35 | A |
| ATOM | 2275 | C | GLN | A | 300 | 95.507 | 35.881 | 48.396 | 1.00 | 7.77 | A |
| ATOM | 2276 | O | GLN | A | 300 | 95.538 | 36.967 | 47.818 | 1.00 | 8.51 | A |
| ATOM | 2277 | N | VAL | A | 301 | 95.645 | 34.710 | 47.784 | 1.00 | 7.84 | A |
| ATOM | 2278 | CA | VAL | A | 301 | 95.772 | 34.558 | 46.341 | 1.00 | 7.73 | A |
| ATOM | 2279 | CB | VAL | A | 301 | 97.214 | 34.202 | 45.909 | 1.00 | 8.38 | A |
| ATOM | 2280 | CG1 | VAL | A | 301 | 97.223 | 33.771 | 44.445 | 1.00 | 10.50 | A |
| ATOM | 2281 | CG2 | VAL | A | 301 | 98.130 | 35.412 | 46.102 | 1.00 | 8.34 | A |
| ATOM | 2282 | C | VAL | A | 301 | 94.845 | 33.374 | 46.066 | 1.00 | 9.23 | A |
| ATOM | 2283 | O | VAL | A | 301 | 94.914 | 32.361 | 46.761 | 1.00 | 9.28 | A |
| ATOM | 2284 | N | VAL | A | 302 | 93.959 | 33.507 | 45.086 | 1.00 | 9.11 | A |
| ATOM | 2285 | CA | VAL | A | 302 | 93.030 | 32.429 | 44.767 | 1.00 | 7.46 | A |
| ATOM | 2286 | CB | VAL | A | 302 | 91.567 | 32.934 | 44.720 | 1.00 | 7.20 | A |
| ATOM | 2287 | CG1 | VAL | A | 302 | 90.634 | 31.784 | 44.364 | 1.00 | 10.03 | A |
| ATOM | 2288 | CG2 | VAL | A | 302 | 91.173 | 33.535 | 46.065 | 1.00 | 8.24 | A |
| ATOM | 2289 | C | VAL | A | 302 | 93.354 | 31.788 | 43.428 | 1.00 | 6.83 | A |
| ATOM | 2290 | O | VAL | A | 302 | 93.525 | 32.477 | 42.426 | 1.00 | 8.77 | A |
| ATOM | 2291 | N | LEU | A | 303 | 93.439 | 30.462 | 43.417 | 1.00 | 7.13 | A |
| ATOM | 2292 | CA | LEU | A | 303 | 93.716 | 29.727 | 42.186 | 1.00 | 7.61 | A |
| ATOM | 2293 | CB | LEU | A | 303 | 94.879 | 28.754 | 42.390 | 1.00 | 8.13 | A |
| ATOM | 2294 | CG | LEU | A | 303 | 96.223 | 29.324 | 42.847 | 1.00 | 8.45 | A |
| ATOM | 2295 | CD1 | LEU | A | 303 | 97.210 | 28.179 | 43.024 | 1.00 | 9.92 | A |
| ATOM | 2296 | CD2 | LEU | A | 303 | 96.745 | 30.344 | 41.843 | 1.00 | 10.52 | A |
| ATOM | 2297 | C | LEU | A | 303 | 92.488 | 28.927 | 41.749 | 1.00 | 8.29 | A |
| ATOM | 2298 | O | LEU | A | 303 | 91.767 | 28.381 | 42.580 | 1.00 | 7.95 | A |
| ATOM | 2299 | N | GLY | A | 304 | 92.259 | 28.862 | 40.442 | 1.00 | 8.87 | A |
| ATOM | 2300 | CA | GLY | A | 304 | 91.142 | 28.097 | 39.919 | 1.00 | 8.60 | A |
| ATOM | 2301 | C | GLY | A | 304 | 89.791 | 28.472 | 40.483 | 1.00 | 9.20 | A |
| ATOM | 2302 | O | GLY | A | 304 | 88.870 | 27.658 | 40.459 | 1.00 | 8.48 | A |
| ATOM | 2303 | N | GLY | A | 305 | 89.669 | 29.701 | 40.979 | 1.00 | 7.63 | A |
| ATOM | 2304 | CA | GLY | A | 305 | 88.418 | 30.165 | 41.555 | 1.00 | 8.18 | A |
| ATOM | 2305 | C | GLY | A | 305 | 87.967 | 29.362 | 42.762 | 1.00 | 7.63 | A |
| ATOM | 2306 | O | GLY | A | 305 | 86.803 | 29.450 | 43.159 | 1.00 | 7.61 | A |
| ATOM | 2307 | N | ARG | A | 306 | 88.875 | 28.603 | 43.374 | 1.00 | 7.30 | A |
| ATOM | 2308 | CA | ARG | A | 306 | 88.483 | 27.767 | 44.505 | 1.00 | 6.71 | A |
| ATOM | 2309 | CB | ARG | A | 306 | 87.895 | 26.465 | 43.974 | 1.00 | 6.47 | A |
| ATOM | 2310 | CG | ARG | A | 306 | 88.935 | 25.596 | 43.239 | 1.00 | 7.18 | A |
| ATOM | 2311 | CD | ARG | A | 306 | 88.255 | 24.466 | 42.477 | 1.00 | 6.01 | A |
| ATOM | 2312 | NE | ARG | A | 306 | 89.156 | 23.436 | 41.937 | 1.00 | 7.00 | A |
| ATOM | 2313 | CZ | ARG | A | 306 | 89.731 | 23.482 | 40.737 | 1.00 | 7.92 | A |
| ATOM | 2314 | NH1 | ARG | A | 306 | 89.519 | 24.516 | 39.933 | 1.00 | 7.27 | A |
| ATOM | 2315 | NH2 | ARG | A | 306 | 90.491 | 22.473 | 40.322 | 1.00 | 7.86 | A |
| ATOM | 2316 | C | ARG | A | 306 | 89.571 | 27.390 | 45.500 | 1.00 | 5.45 | A |
| ATOM | 2317 | O | ARG | A | 306 | 89.272 | 26.769 | 46.520 | 1.00 | 7.13 | A |
| ATOM | 2318 | N | ARG | A | 307 | 90.817 | 27.738 | 45.201 | 1.00 | 6.46 | A |
| ATOM | 2319 | CA | ARG | A | 307 | 91.936 | 27.376 | 46.061 | 1.00 | 5.64 | A |
| ATOM | 2320 | CB | ARG | A | 307 | 92.928 | 26.529 | 45.255 | 1.00 | 5.76 | A |
| ATOM | 2321 | CG | ARG | A | 307 | 94.244 | 26.213 | 45.953 | 1.00 | 6.62 | A |
| ATOM | 2322 | CD | ARG | A | 307 | 94.876 | 24.965 | 45.345 | 1.00 | 6.88 | A |
| ATOM | 2323 | NE | ARG | A | 307 | 96.294 | 24.860 | 45.671 | 1.00 | 7.07 | A |
| ATOM | 2324 | CZ | ARG | A | 307 | 96.763 | 24.561 | 46.875 | 1.00 | 5.97 | A |
| ATOM | 2325 | NH1 | ARG | A | 307 | 95.917 | 24.329 | 47.873 | 1.00 | 7.56 | A |
| ATOM | 2326 | NH2 | ARG | A | 307 | 98.074 | 24.500 | 47.085 | 1.00 | 6.20 | A |
| ATOM | 2327 | C | ARG | A | 307 | 92.618 | 28.597 | 46.653 | 1.00 | 6.76 | A |
| ATOM | 2328 | O | ARG | A | 307 | 93.379 | 29.291 | 45.979 | 1.00 | 7.09 | A |
| ATOM | 2329 | N | ILE | A | 308 | 92.333 | 28.846 | 47.927 | 1.00 | 7.02 | A |
| ATOM | 2330 | CA | ILE | A | 308 | 92.887 | 29.992 | 48.636 | 1.00 | 6.86 | A |
| ATOM | 2331 | CB | ILE | A | 308 | 91.921 | 30.458 | 49.750 | 1.00 | 7.69 | A |
| ATOM | 2332 | CG2 | ILE | A | 308 | 92.600 | 31.510 | 50.627 | 1.00 | 9.71 | A |
| ATOM | 2333 | CG1 | ILE | A | 308 | 90.643 | 31.014 | 49.112 | 1.00 | 8.89 | A |
| ATOM | 2334 | CD1 | ILE | A | 308 | 89.524 | 31.284 | 50.095 | 1.00 | 10.87 | A |
| ATOM | 2335 | C | ILE | A | 308 | 94.256 | 29.721 | 49.257 | 1.00 | 7.03 | A |
| ATOM | 2336 | O | ILE | A | 308 | 94.441 | 28.747 | 49.987 | 1.00 | 7.70 | A |
| ATOM | 2337 | N | LEU | A | 309 | 95.206 | 30.591 | 48.935 | 1.00 | 7.11 | A |
| ATOM | 2338 | CA | LEU | A | 309 | 96.556 | 30.533 | 49.475 | 1.00 | 7.00 | A |
| ATOM | 2339 | CB | LEU | A | 309 | 97.591 | 30.686 | 48.360 | 1.00 | 7.77 | A |
| ATOM | 2340 | CG | LEU | A | 309 | 97.523 | 29.638 | 47.241 | 1.00 | 9.08 | A |
| ATOM | 2341 | CD1 | LEU | A | 309 | 98.569 | 29.952 | 46.182 | 1.00 | 10.12 | A |
| ATOM | 2342 | CD2 | LEU | A | 309 | 97.764 | 28.256 | 47.823 | 1.00 | 10.17 | A |
| ATOM | 2343 | C | LEU | A | 309 | 96.583 | 31.755 | 50.379 | 1.00 | 8.01 | A |
| ATOM | 2344 | O | LEU | A | 309 | 96.178 | 32.842 | 49.957 | 1.00 | 8.66 | A |
| ATOM | 2345 | N | GLY | A | 310 | 97.040 | 31.596 | 51.615 | 1.00 | 7.27 | A |
| ATOM | 2346 | CA | GLY | A | 310 | 97.042 | 32.735 | 52.512 | 1.00 | 8.71 | A |
| ATOM | 2347 | C | GLY | A | 310 | 98.329 | 33.048 | 53.237 | 1.00 | 7.88 | A |
| ATOM | 2348 | O | GLY | A | 310 | 99.272 | 32.259 | 53.260 | 1.00 | 7.77 | A |
| ATOM | 2349 | N | ASN | A | 311 | 98.348 | 34.238 | 53.824 | 1.00 | 8.15 | A |
| ATOM | 2350 | CA | ASN | A | 311 | 99.482 | 34.721 | 54.598 | 1.00 | 8.07 | A |
| ATOM | 2351 | CB | ASN | A | 311 | 100.433 | 35.555 | 53.737 | 1.00 | 9.12 | A |
| ATOM | 2352 | CG | ASN | A | 311 | 101.541 | 36.205 | 54.555 | 1.00 | 9.62 | A |
| ATOM | 2353 | OD1 | ASN | A | 311 | 101.470 | 37.391 | 54.927 | 1.00 | 12.21 | A |
| ATOM | 2354 | ND2 | ASN | A | 311 | 102.559 | 35.426 | 54.862 | 1.00 | 7.14 | A |
| ATOM | 2355 | C | ASN | A | 311 | 99.004 | 35.609 | 55.722 | 1.00 | 8.26 | A |
| ATOM | 2356 | O | ASN | A | 311 | 97.993 | 36.292 | 55.597 | 1.00 | 7.95 | A |
| ATOM | 2357 | N | LEU | A | 312 | 99.733 | 35.590 | 56.828 | 1.00 | 8.48 | A |
| ATOM | 2358 | CA | LEU | A | 312 | 99.437 | 36.470 | 57.950 | 1.00 | 8.96 | A |
| ATOM | 2359 | CB | LEU | A | 312 | 98.459 | 35.843 | 58.952 | 1.00 | 9.59 | A |
| ATOM | 2360 | CG | LEU | A | 312 | 98.964 | 34.877 | 60.028 | 1.00 | 9.51 | A |
| ATOM | 2361 | CD1 | LEU | A | 312 | 98.076 | 34.942 | 61.281 | 1.00 | 11.10 | A |
| ATOM | 2362 | CD2 | LEU | A | 312 | 98.983 | 33.473 | 59.458 | 1.00 | 10.36 | A |
| ATOM | 2363 | C | LEU | A | 312 | 100.774 | 36.690 | 58.609 | 1.00 | 8.93 | A |
| ATOM | 2364 | O | LEU | A | 312 | 101.748 | 36.008 | 58.290 | 1.00 | 8.95 | A |
| ATOM | 2365 | N | PHE | A | 313 | 100.840 | 37.661 | 59.506 | 1.00 | 7.99 | A |
| ATOM | 2366 | CA | PHE | A | 313 | 102.085 | 37.914 | 60.220 | 1.00 | 8.14 | A |
| ATOM | 2367 | CB | PHE | A | 313 | 102.496 | 39.384 | 60.090 | 1.00 | 8.99 | A |
| ATOM | 2368 | CG | PHE | A | 313 | 102.843 | 39.781 | 58.681 | 1.00 | 7.35 | A |
| ATOM | 2369 | CD1 | PHE | A | 313 | 101.842 | 40.109 | 57.767 | 1.00 | 9.01 | A |
| ATOM | 2370 | CD2 | PHE | A | 313 | 104.170 | 39.789 | 58.251 | 1.00 | 7.71 | A |
| ATOM | 2371 | CE1 | PHE | A | 313 | 102.157 | 40.434 | 56.444 | 1.00 | 9.34 | A |
| ATOM | 2372 | CE2 | PHE | A | 313 | 104.496 | 40.114 | 56.930 | 1.00 | 7.29 | A |
| ATOM | 2373 | CZ | PHE | A | 313 | 103.487 | 40.440 | 56.023 | 1.00 | 9.24 | A |
| ATOM | 2374 | C | PHE | A | 313 | 101.863 | 37.541 | 61.679 | 1.00 | 8.42 | A |
| ATOM | 2375 | O | PHE | A | 313 | 100.779 | 37.747 | 62.222 | 1.00 | 10.18 | A |
| ATOM | 2376 | N | ARG | A | 314 | 102.880 | 36.963 | 62.304 | 1.00 | 8.74 | A |
| ATOM | 2377 | CA | ARG | A | 314 | 102.775 | 36.553 | 63.697 | 1.00 | 8.79 | A |
| ATOM | 2378 | CB | ARG | A | 314 | 102.637 | 35.030 | 63.789 | 1.00 | 9.27 | A |
| ATOM | 2379 | CG | ARG | A | 314 | 101.352 | 34.490 | 63.179 | 1.00 | 9.66 | A |
| ATOM | 2380 | CD | ARG | A | 314 | 101.356 | 32.959 | 63.053 | 1.00 | 9.84 | A |
| ATOM | 2381 | NE | ARG | A | 314 | 101.375 | 32.261 | 64.334 | 1.00 | 10.06 | A |
| ATOM | 2382 | CZ | ARG | A | 314 | 102.469 | 31.768 | 64.907 | 1.00 | 11.62 | A |
| ATOM | 2383 | NH1 | ARG | A | 314 | 103.646 | 31.897 | 64.308 | 1.00 | 12.88 | A |
| ATOM | 2384 | NH2 | ARG | A | 314 | 102.387 | 31.136 | 66.078 | 1.00 | 12.05 | A |
| ATOM | 2385 | C | ARG | A | 314 | 103.983 | 36.992 | 64.503 | 1.00 | 9.37 | A |
| ATOM | 2386 | O | ARG | A | 314 | 105.120 | 36.872 | 64.042 | 1.00 | 10.21 | A |
| ATOM | 2387 | N | GLU | A | 315 | 103.742 | 37.503 | 65.707 | 1.00 | 9.99 | A |
| ATOM | 2388 | CA | GLU | A | 315 | 104.842 | 37.932 | 66.568 | 1.00 | 10.30 | A |
| ATOM | 2389 | CB | GLU | A | 315 | 104.365 | 38.924 | 67.632 | 1.00 | 10.59 | A |
| ATOM | 2390 | CG | GLU | A | 315 | 103.870 | 40.266 | 67.141 | 1.00 | 10.88 | A |
| ATOM | 2391 | CD | GLU | A | 315 | 103.645 | 41.233 | 68.291 | 1.00 | 11.11 | A |
| ATOM | 2392 | OE1 | GLU | A | 315 | 103.174 | 40.792 | 69.365 | 1.00 | 12.49 | A |
| ATOM | 2393 | OE2 | GLU | A | 315 | 103.920 | 42.443 | 68.129 | 1.00 | 12.40 | A |
| ATOM | 2394 | C | GLU | A | 315 | 105.448 | 36.744 | 67.304 | 1.00 | 12.37 | A |
| ATOM | 2395 | O | GLU | A | 315 | 104.747 | 35.792 | 67.657 | 1.00 | 12.29 | A |
| ATOM | 2396 | N | THR | A | 316 | 106.756 | 36.806 | 67.524 | 1.00 | 12.37 | A |
| ATOM | 2397 | CA | THR | A | 316 | 107.465 | 35.778 | 68.280 | 1.00 | 13.48 | A |
| ATOM | 2398 | CB | THR | A | 316 | 108.762 | 35.320 | 67.568 | 1.00 | 13.95 | A |
| ATOM | 2399 | OG1 | THR | A | 316 | 109.513 | 36.466 | 67.146 | 1.00 | 17.23 | A |
| ATOM | 2400 | CG2 | THR | A | 316 | 108.433 | 34.454 | 66.363 | 1.00 | 12.28 | A |
| ATOM | 2401 | C | THR | A | 316 | 107.809 | 36.444 | 69.611 | 1.00 | 13.94 | A |
| ATOM | 2402 | O | THR | A | 316 | 108.117 | 35.781 | 70.600 | 1.00 | 16.29 | A |
| ATOM | 2403 | N | SER | A | 317 | 107.749 | 37.773 | 69.606 | 1.00 | 13.50 | A |
| ATOM | 2404 | CA | SER | A | 317 | 107.995 | 38.598 | 70.791 | 1.00 | 13.72 | A |
| ATOM | 2405 | CB | SER | A | 317 | 109.495 | 38.703 | 71.101 | 1.00 | 12.93 | A |
| ATOM | 2406 | OG | SER | A | 317 | 110.113 | 39.731 | 70.343 | 1.00 | 12.47 | A |
| ATOM | 2407 | C | SER | A | 317 | 107.425 | 39.983 | 70.484 | 1.00 | 13.92 | A |
| ATOM | 2408 | O | SER | A | 317 | 106.991 | 40.251 | 69.359 | 1.00 | 13.24 | A |
| ATOM | 2409 | N | SER | A | 318 | 107.426 | 40.867 | 71.475 | 1.00 | 13.43 | A |
| ATOM | 2410 | CA | SER | A | 318 | 106.893 | 42.211 | 71.279 | 1.00 | 13.72 | A |
| ATOM | 2411 | CB | SER | A | 318 | 106.865 | 42.968 | 72.613 | 1.00 | 15.89 | A |
| ATOM | 2412 | OG | SER | A | 318 | 108.168 | 43.093 | 73.148 | 1.00 | 18.17 | A |
| ATOM | 2413 | C | SER | A | 318 | 107.688 | 43.017 | 70.255 | 1.00 | 12.72 | A |
| ATOM | 2414 | O | SER | A | 318 | 107.213 | 44.038 | 69.761 | 1.00 | 12.85 | A |
| ATOM | 2415 | N | THR | A | 319 | 108.890 | 42.549 | 69.928 | 1.00 | 11.62 | A |
| ATOM | 2416 | CA | THR | A | 319 | 109.747 | 43.252 | 68.975 | 1.00 | 11.43 | A |
| ATOM | 2417 | CB | THR | A | 319 | 110.988 | 43.839 | 69.673 | 1.00 | 12.76 | A |
| ATOM | 2418 | OG1 | THR | A | 319 | 111.695 | 42.793 | 70.346 | 1.00 | 14.66 | A |
| ATOM | 2419 | CG2 | THR | A | 319 | 110.576 | 44.906 | 70.680 | 1.00 | 13.70 | A |
| ATOM | 2420 | C | THR | A | 319 | 110.229 | 42.420 | 67.786 | 1.00 | 10.76 | A |
| ATOM | 2421 | O | THR | A | 319 | 111.071 | 42.876 | 67.015 | 1.00 | 10.23 | A |
| ATOM | 2422 | N | THR | A | 320 | 109.714 | 41.202 | 67.643 | 1.00 | 10.01 | A |
| ATOM | 2423 | CA | THR | A | 320 | 110.107 | 40.347 | 66.525 | 1.00 | 8.89 | A |
| ATOM | 2424 | CB | THR | A | 320 | 111.152 | 39.285 | 66.952 | 1.00 | 9.22 | A |
| ATOM | 2425 | OG1 | THR | A | 320 | 110.652 | 38.521 | 68.055 | 1.00 | 10.59 | A |
| ATOM | 2426 | CG2 | THR | A | 320 | 112.460 | 39.956 | 67.357 | 1.00 | 9.76 | A |
| ATOM | 2427 | C | THR | A | 320 | 108.874 | 39.653 | 65.961 | 1.00 | 9.56 | A |
| ATOM | 2428 | O | THR | A | 320 | 107.984 | 39.241 | 66.708 | 1.00 | 9.97 | A |
| ATOM | 2429 | N | SER | A | 321 | 108.815 | 39.538 | 64.639 | 1.00 | 8.60 | A |
| ATOM | 2430 | CA | SER | A | 321 | 107.673 | 38.913 | 63.981 | 1.00 | 9.29 | A |
| ATOM | 2431 | CB | SER | A | 321 | 106.515 | 39.909 | 63.905 | 1.00 | 12.30 | A |
| ATOM | 2432 | OG | SER | A | 321 | 106.879 | 41.023 | 63.105 | 1.00 | 11.62 | A |
| ATOM | 2433 | C | SER | A | 321 | 108.041 | 38.466 | 62.573 | 1.00 | 9.74 | A |
| ATOM | 2434 | O | SER | A | 321 | 109.033 | 38.924 | 62.004 | 1.00 | 10.24 | A |
| ATOM | 2435 | N | GLY | A | 322 | 107.231 | 37.573 | 62.015 | 1.00 | 9.46 | A |
| ATOM | 2436 | CA | GLY | A | 322 | 107.480 | 37.082 | 60.673 | 1.00 | 9.11 | A |
| ATOM | 2437 | C | GLY | A | 322 | 106.186 | 36.839 | 59.926 | 1.00 | 9.37 | A |
| ATOM | 2438 | O | GLY | A | 322 | 105.096 | 37.044 | 60.465 | 1.00 | 9.30 | A |
| ATOM | 2439 | N | ALA | A | 323 | 106.311 | 36.404 | 58.677 | 1.00 | 8.77 | A |
| ATOM | 2440 | CA | ALA | A | 323 | 105.152 | 36.109 | 57.842 | 1.00 | 8.28 | A |
| ATOM | 2441 | CB | ALA | A | 323 | 105.383 | 36.637 | 56.427 | 1.00 | 8.99 | A |
| ATOM | 2442 | C | ALA | A | 323 | 104.987 | 34.596 | 57.816 | 1.00 | 8.58 | A |
| ATOM | 2443 | O | ALA | A | 323 | 105.956 | 33.869 | 57.611 | 1.00 | 9.76 | A |
| ATOM | 2444 | N | TYR | A | 324 | 103.768 | 34.126 | 58.043 | 1.00 | 7.98 | A |
| ATOM | 2445 | CA | TYR | A | 324 | 103.491 | 32.695 | 58.042 | 1.00 | 8.09 | A |
| ATOM | 2446 | CB | TYR | A | 324 | 103.064 | 32.243 | 59.443 | 1.00 | 8.58 | A |
| ATOM | 2447 | CG | TYR | A | 324 | 104.167 | 32.434 | 60.460 | 1.00 | 9.70 | A |
| ATOM | 2448 | CD1 | TYR | A | 324 | 104.486 | 33.701 | 60.941 | 1.00 | 10.85 | A |
| ATOM | 2449 | CE1 | TYR | A | 324 | 105.576 | 33.895 | 61.790 | 1.00 | 10.62 | A |
| ATOM | 2450 | CD2 | TYR | A | 324 | 104.959 | 31.359 | 60.860 | 1.00 | 10.25 | A |
| ATOM | 2451 | CE2 | TYR | A | 324 | 106.048 | 31.541 | 61.707 | 1.00 | 11.14 | A |
| ATOM | 2452 | CZ | TYR | A | 324 | 106.350 | 32.813 | 62.163 | 1.00 | 11.43 | A |
| ATOM | 2453 | OH | TYR | A | 324 | 107.445 | 32.998 | 62.980 | 1.00 | 13.41 | A |
| ATOM | 2454 | C | TYR | A | 324 | 102.403 | 32.411 | 57.028 | 1.00 | 8.21 | A |
| ATOM | 2455 | O | TYR | A | 324 | 101.358 | 33.067 | 57.029 | 1.00 | 8.49 | A |
| ATOM | 2456 | N | GLN | A | 325 | 102.648 | 31.438 | 56.155 | 1.00 | 7.45 | A |
| ATOM | 2457 | CA | GLN | A | 325 | 101.673 | 31.113 | 55.118 | 1.00 | 7.93 | A |
| ATOM | 2458 | CB | GLN | A | 325 | 102.382 | 30.980 | 53.771 | 1.00 | 7.44 | A |
| ATOM | 2459 | CG | GLN | A | 325 | 103.047 | 32.287 | 53.319 | 1.00 | 7.92 | A |
| ATOM | 2460 | CD | GLN | A | 325 | 104.389 | 32.534 | 53.988 | 1.00 | 7.87 | A |
| ATOM | 2461 | OE1 | GLN | A | 325 | 104.647 | 33.614 | 54.530 | 1.00 | 12.10 | A |
| ATOM | 2462 | NE2 | GLN | A | 325 | 105.254 | 31.540 | 53.940 | 1.00 | 4.46 | A |
| ATOM | 2463 | C | GLN | A | 325 | 100.833 | 29.873 | 55.404 | 1.00 | 8.73 | A |
| ATOM | 2464 | O | GLN | A | 325 | 101.179 | 29.050 | 56.248 | 1.00 | 8.93 | A |
| ATOM | 2465 | N | PHE | A | 326 | 99.716 | 29.761 | 54.696 | 1.00 | 7.70 | A |
| ATOM | 2466 | CA | PHE | A | 326 | 98.799 | 28.644 | 54.875 | 1.00 | 8.00 | A |
| ATOM | 2467 | CB | PHE | A | 326 | 97.926 | 28.870 | 56.118 | 1.00 | 8.09 | A |
| ATOM | 2468 | CG | PHE | A | 326 | 97.157 | 30.172 | 56.097 | 1.00 | 7.59 | A |
| ATOM | 2469 | CD1 | PHE | A | 326 | 97.770 | 31.373 | 56.460 | 1.00 | 8.73 | A |
| ATOM | 2470 | CD2 | PHE | A | 326 | 95.809 | 30.194 | 55.737 | 1.00 | 8.03 | A |
| ATOM | 2471 | CE1 | PHE | A | 326 | 97.055 | 32.573 | 56.454 | 1.00 | 9.08 | A |
| ATOM | 2472 | CE2 | PHE | A | 326 | 95.090 | 31.389 | 55.728 | 1.00 | 8.41 | A |
| ATOM | 2473 | CZ | PHE | A | 326 | 95.712 | 32.580 | 56.095 | 1.00 | 10.11 | A |
| ATOM | 2474 | C | PHE | A | 326 | 97.896 | 28.477 | 53.661 | 1.00 | 7.58 | A |
| ATOM | 2475 | O | PHE | A | 326 | 97.985 | 29.238 | 52.701 | 1.00 | 7.59 | A |
| ATOM | 2476 | N | GLU | A | 327 | 97.030 | 27.470 | 53.723 | 1.00 | 7.76 | A |
| ATOM | 2477 | CA | GLU | A | 327 | 96.080 | 27.196 | 52.654 | 1.00 | 7.62 | A |
| ATOM | 2478 | CB | GLU | A | 327 | 96.511 | 25.955 | 51.870 | 1.00 | 7.35 | A |
| ATOM | 2479 | CG | GLU | A | 327 | 97.927 | 26.086 | 51.316 | 1.00 | 7.89 | A |
| ATOM | 2480 | CD | GLU | A | 327 | 98.349 | 24.918 | 50.437 | 1.00 | 9.39 | A |
| ATOM | 2481 | OE1 | GLU | A | 327 | 97.561 | 23.956 | 50.285 | 1.00 | 8.45 | A |
| ATOM | 2482 | OE2 | GLU | A | 327 | 99.471 | 24.963 | 49.896 | 1.00 | 10.21 | A |
| ATOM | 2483 | C | GLU | A | 327 | 94.740 | 26.967 | 53.330 | 1.00 | 6.99 | A |
| ATOM | 2484 | O | GLU | A | 327 | 94.691 | 26.464 | 54.451 | 1.00 | 8.05 | A |
| ATOM | 2485 | N | VAL | A | 328 | 93.662 | 27.370 | 52.667 | 1.00 | 6.86 | A |
| ATOM | 2486 | CA | VAL | A | 328 | 92.322 | 27.196 | 53.206 | 1.00 | 7.39 | A |
| ATOM | 2487 | CB | VAL | A | 328 | 91.517 | 28.508 | 53.148 | 1.00 | 8.25 | A |
| ATOM | 2488 | CG1 | VAL | A | 328 | 90.085 | 28.253 | 53.583 | 1.00 | 8.43 | A |
| ATOM | 2489 | CG2 | VAL | A | 328 | 92.167 | 29.557 | 54.046 | 1.00 | 9.51 | A |
| ATOM | 2490 | C | VAL | A | 328 | 91.622 | 26.157 | 52.350 | 1.00 | 6.54 | A |
| ATOM | 2491 | O | VAL | A | 328 | 91.581 | 26.285 | 51.129 | 1.00 | 7.51 | A |
| ATOM | 2492 | N | TYR | A | 329 | 91.093 | 25.123 | 52.991 | 1.00 | 7.62 | A |
| ATOM | 2493 | CA | TYR | A | 329 | 90.397 | 24.060 | 52.285 | 1.00 | 7.43 | A |
| ATOM | 2494 | CB | TYR | A | 329 | 90.928 | 22.704 | 52.759 | 1.00 | 8.00 | A |
| ATOM | 2495 | CG | TYR | A | 329 | 92.439 | 22.628 | 52.654 | 1.00 | 10.74 | A |
| ATOM | 2496 | CD1 | TYR | A | 329 | 93.236 | 22.430 | 53.787 | 1.00 | 13.12 | A |
| ATOM | 2497 | CE1 | TYR | A | 329 | 94.633 | 22.414 | 53.694 | 1.00 | 13.60 | A |
| ATOM | 2498 | CD2 | TYR | A | 329 | 93.075 | 22.806 | 51.426 | 1.00 | 8.39 | A |
| ATOM | 2499 | CE2 | TYR | A | 329 | 94.465 | 22.788 | 51.319 | 1.00 | 10.29 | A |
| ATOM | 2500 | CZ | TYR | A | 329 | 95.238 | 22.593 | 52.453 | 1.00 | 14.38 | A |
| ATOM | 2501 | OH | TYR | A | 329 | 96.614 | 22.575 | 52.350 | 1.00 | 15.91 | A |
| ATOM | 2502 | C | TYR | A | 329 | 88.905 | 24.219 | 52.568 | 1.00 | 6.14 | A |
| ATOM | 2503 | O | TYR | A | 329 | 88.407 | 23.818 | 53.622 | 1.00 | 6.61 | A |
| ATOM | 2504 | N | LEU | A | 330 | 88.207 | 24.834 | 51.617 | 1.00 | 6.85 | A |
| ATOM | 2505 | CA | LEU | A | 330 | 86.780 | 25.118 | 51.737 | 1.00 | 7.29 | A |
| ATOM | 2506 | CB | LEU | A | 330 | 86.389 | 26.211 | 50.744 | 1.00 | 7.57 | A |
| ATOM | 2507 | CG | LEU | A | 330 | 87.111 | 27.553 | 50.849 | 1.00 | 7.01 | A |
| ATOM | 2508 | CD1 | LEU | A | 330 | 86.740 | 28.408 | 49.644 | 1.00 | 8.86 | A |
| ATOM | 2509 | CD2 | LEU | A | 330 | 86.722 | 28.252 | 52.148 | 1.00 | 10.55 | A |
| ATOM | 2510 | C | LEU | A | 330 | 85.851 | 23.940 | 51.523 | 1.00 | 8.02 | A |
| ATOM | 2511 | O | LEU | A | 330 | 84.712 | 23.956 | 51.993 | 1.00 | 7.98 | A |
| ATOM | 2512 | N | GLY | A | 331 | 86.327 | 22.921 | 50.821 | 1.00 | 7.21 | A |
| ATOM | 2513 | CA | GLY | A | 331 | 85.474 | 21.784 | 50.535 | 1.00 | 6.80 | A |
| ATOM | 2514 | C | GLY | A | 331 | 84.339 | 22.243 | 49.634 | 1.00 | 7.82 | A |
| ATOM | 2515 | O | GLY | A | 331 | 84.479 | 23.219 | 48.893 | 1.00 | 7.06 | A |
| ATOM | 2516 | N | GLY | A | 332 | 83.211 | 21.544 | 49.691 | 1.00 | 7.28 | A |
| ATOM | 2517 | CA | GLY | A | 332 | 82.067 | 21.932 | 48.880 | 1.00 | 7.68 | A |
| ATOM | 2518 | C | GLY | A | 332 | 81.146 | 22.801 | 49.715 | 1.00 | 7.54 | A |
| ATOM | 2519 | O | GLY | A | 332 | 80.415 | 22.298 | 50.564 | 1.00 | 9.03 | A |
| ATOM | 2520 | N | VAL | A | 333 | 81.171 | 24.109 | 49.481 | 1.00 | 6.90 | A |
| ATOM | 2521 | CA | VAL | A | 333 | 80.336 | 25.018 | 50.261 | 1.00 | 6.88 | A |
| ATOM | 2522 | CB | VAL | A | 333 | 80.731 | 26.488 | 50.012 | 1.00 | 6.76 | A |
| ATOM | 2523 | CG1 | VAL | A | 333 | 80.016 | 27.396 | 51.012 | 1.00 | 8.90 | A |
| ATOM | 2524 | CG2 | VAL | A | 333 | 82.242 | 26.644 | 50.138 | 1.00 | 8.93 | A |
| ATOM | 2525 | C | VAL | A | 333 | 78.868 | 24.828 | 49.904 | 1.00 | 6.88 | A |
| ATOM | 2526 | O | VAL | A | 333 | 78.507 | 24.822 | 48.726 | 1.00 | 7.70 | A |
| ATOM | 2527 | N | PRO | A | 334 | 78.001 | 24.671 | 50.918 | 1.00 | 6.66 | A |
| ATOM | 2528 | CD | PRO | A | 334 | 78.291 | 24.600 | 52.361 | 1.00 | 7.10 | A |
| ATOM | 2529 | CA | PRO | A | 334 | 76.571 | 24.477 | 50.665 | 1.00 | 7.22 | A |
| ATOM | 2530 | CB | PRO | A | 334 | 75.983 | 24.338 | 52.067 | 1.00 | 7.40 | A |
| ATOM | 2531 | CG | PRO | A | 334 | 77.130 | 23.789 | 52.870 | 1.00 | 7.18 | A |
| ATOM | 2532 | C | PRO | A | 334 | 75.938 | 25.634 | 49.912 | 1.00 | 5.55 | A |
| ATOM | 2533 | O | PRO | A | 334 | 76.307 | 26.796 | 50.101 | 1.00 | 6.74 | A |
| ATOM | 2534 | N | ASP | A | 335 | 74.983 | 25.311 | 49.053 | 1.00 | 6.79 | A |
| ATOM | 2535 | CA | ASP | A | 335 | 74.279 | 26.344 | 48.310 | 1.00 | 7.28 | A |
| ATOM | 2536 | CB | ASP | A | 335 | 73.661 | 25.756 | 47.053 | 1.00 | 8.25 | A |
| ATOM | 2537 | CG | ASP | A | 335 | 74.706 | 25.414 | 46.022 | 1.00 | 9.16 | A |
| ATOM | 2538 | OD1 | ASP | A | 335 | 74.900 | 24.217 | 45.741 | 1.00 | 9.18 | A |
| ATOM | 2539 | OD2 | ASP | A | 335 | 75.338 | 26.359 | 45.516 | 1.00 | 11.04 | A |
| ATOM | 2540 | C | ASP | A | 335 | 73.206 | 26.927 | 49.201 | 1.00 | 8.56 | A |
| ATOM | 2541 | O | ASP | A | 335 | 72.724 | 28.038 | 48.977 | 1.00 | 8.31 | A |
| ATOM | 2542 | N | PHE | A | 336 | 72.830 | 26.154 | 50.210 | 1.00 | 8.54 | A |
| ATOM | 2543 | CA | PHE | A | 336 | 71.840 | 26.583 | 51.171 | 1.00 | 8.65 | A |
| ATOM | 2544 | CB | PHE | A | 336 | 70.423 | 26.199 | 50.738 | 1.00 | 10.96 | A |
| ATOM | 2545 | CG | PHE | A | 336 | 69.422 | 26.270 | 51.858 | 1.00 | 11.31 | A |
| ATOM | 2546 | CD1 | PHE | A | 336 | 68.923 | 27.497 | 52.281 | 1.00 | 11.85 | A |
| ATOM | 2547 | CD2 | PHE | A | 336 | 69.024 | 25.116 | 52.531 | 1.00 | 11.71 | A |
| ATOM | 2548 | CE1 | PHE | A | 336 | 68.044 | 27.581 | 53.362 | 1.00 | 11.07 | A |
| ATOM | 2549 | CE2 | PHE | A | 336 | 68.147 | 25.186 | 53.612 | 1.00 | 12.36 | A |
| ATOM | 2550 | CZ | PHE | A | 336 | 67.655 | 26.422 | 54.029 | 1.00 | 12.04 | A |
| ATOM | 2551 | C | PHE | A | 336 | 72.116 | 25.897 | 52.486 | 1.00 | 8.82 | A |
| ATOM | 2552 | O | PHE | A | 336 | 72.428 | 24.707 | 52.527 | 1.00 | 8.81 | A |
| ATOM | 2553 | N | GLU | A | 337 | 72.031 | 26.660 | 53.562 | 1.00 | 9.67 | A |
| ATOM | 2554 | CA | GLU | A | 337 | 72.179 | 26.077 | 54.876 | 1.00 | 9.07 | A |
| ATOM | 2555 | CB | GLU | A | 337 | 73.647 | 25.837 | 55.262 | 1.00 | 12.48 | A |
| ATOM | 2556 | CG | GLU | A | 337 | 74.605 | 27.001 | 55.232 | 1.00 | 11.71 | A |
| ATOM | 2557 | CD | GLU | A | 337 | 76.025 | 26.552 | 55.618 | 1.00 | 13.20 | A |
| ATOM | 2558 | OE1 | GLU | A | 337 | 76.191 | 26.002 | 56.724 | 1.00 | 11.09 | A |
| ATOM | 2559 | OE2 | GLU | A | 337 | 76.962 | 26.742 | 54.817 | 1.00 | 13.43 | A |
| ATOM | 2560 | C | GLU | A | 337 | 71.460 | 26.914 | 55.903 | 1.00 | 9.40 | A |
| ATOM | 2561 | O | GLU | A | 337 | 71.436 | 28.144 | 55.845 | 1.00 | 11.10 | A |
| ATOM | 2562 | N | SER | A | 338 | 70.824 | 26.213 | 56.825 | 1.00 | 9.47 | A |
| ATOM | 2563 | CA | SER | A | 338 | 70.071 | 26.852 | 57.880 | 1.00 | 10.43 | A |
| ATOM | 2564 | CB | SER | A | 338 | 68.906 | 25.957 | 58.308 | 1.00 | 9.23 | A |
| ATOM | 2565 | OG | SER | A | 338 | 69.379 | 24.798 | 58.971 | 1.00 | 7.90 | A |
| ATOM | 2566 | C | SER | A | 338 | 71.024 | 27.009 | 59.040 | 1.00 | 13.11 | A |
| ATOM | 2567 | O | SER | A | 338 | 72.104 | 26.413 | 59.066 | 1.00 | 16.33 | A |
| ATOM | 2568 | N | ASP | A | 339 | 70.650 | 27.832 | 59.999 | 1.00 | 17.05 | A |
| ATOM | 2569 | CA | ASP | A | 339 | 71.502 | 27.933 | 61.161 | 1.00 | 15.59 | A |
| ATOM | 2570 | CB | ASP | A | 339 | 71.293 | 29.264 | 61.888 | 1.00 | 20.47 | A |
| ATOM | 2571 | CG | ASP | A | 339 | 71.661 | 30.467 | 61.030 | 1.00 | 23.83 | A |
| ATOM | 2572 | OD1 | ASP | A | 339 | 72.804 | 30.527 | 60.524 | 1.00 | 21.50 | A |
| ATOM | 2573 | OD2 | ASP | A | 339 | 70.801 | 31.361 | 60.872 | 1.00 | 27.91 | A |
| ATOM | 2574 | C | ASP | A | 339 | 70.932 | 26.776 | 61.971 | 1.00 | 13.51 | A |
| ATOM | 2575 | O | ASP | A | 339 | 70.132 | 25.982 | 61.452 | 1.00 | 11.53 | A |
| ATOM | 2576 | N | TRP | A | 340 | 71.343 | 26.646 | 63.221 | 1.00 | 9.87 | A |
| ATOM | 2577 | CA | TRP | A | 340 | 70.778 | 25.599 | 64.060 | 1.00 | 8.92 | A |
| ATOM | 2578 | CB | TRP | A | 340 | 71.653 | 25.346 | 65.291 | 1.00 | 8.80 | A |
| ATOM | 2579 | CG | TRP | A | 340 | 72.929 | 24.629 | 64.969 | 1.00 | 8.23 | A |
| ATOM | 2580 | CD2 | TRP | A | 340 | 73.076 | 23.228 | 64.714 | 1.00 | 6.91 | A |
| ATOM | 2581 | CE2 | TRP | A | 340 | 74.435 | 22.998 | 64.403 | 1.00 | 7.83 | A |
| ATOM | 2582 | CE3 | TRP | A | 340 | 72.187 | 22.145 | 64.709 | 1.00 | 8.08 | A |
| ATOM | 2583 | CD1 | TRP | A | 340 | 74.169 | 25.179 | 64.814 | 1.00 | 8.75 | A |
| ATOM | 2584 | NE1 | TRP | A | 340 | 75.082 | 24.205 | 64.475 | 1.00 | 9.13 | A |
| ATOM | 2585 | CZ2 | TRP | A | 340 | 74.930 | 21.725 | 64.101 | 1.00 | 8.61 | A |
| ATOM | 2586 | CZ3 | TRP | A | 340 | 72.678 | 20.882 | 64.408 | 1.00 | 7.87 | A |
| ATOM | 2587 | CH2 | TRP | A | 340 | 74.038 | 20.684 | 64.103 | 1.00 | 8.86 | A |
| ATOM | 2588 | C | TRP | A | 340 | 69.422 | 26.122 | 64.506 | 1.00 | 9.55 | A |
| ATOM | 2589 | O | TRP | A | 340 | 69.319 | 27.260 | 64.958 | 1.00 | 11.36 | A |
| ATOM | 2590 | N | PHE | A | 341 | 68.380 | 25.312 | 64.360 | 1.00 | 8.33 | A |
| ATOM | 2591 | CA | PHE | A | 341 | 67.048 | 25.736 | 64.775 | 1.00 | 8.39 | A |
| ATOM | 2592 | CB | PHE | A | 341 | 66.167 | 26.045 | 63.553 | 1.00 | 8.81 | A |
| ATOM | 2593 | CG | PHE | A | 341 | 65.966 | 24.878 | 62.619 | 1.00 | 7.79 | A |
| ATOM | 2594 | CD1 | PHE | A | 341 | 64.895 | 24.003 | 62.792 | 1.00 | 9.18 | A |
| ATOM | 2595 | CD2 | PHE | A | 341 | 66.821 | 24.684 | 61.535 | 1.00 | 8.46 | A |
| ATOM | 2596 | CE1 | PHE | A | 341 | 64.673 | 22.950 | 61.892 | 1.00 | 9.05 | A |
| ATOM | 2597 | CE2 | PHE | A | 341 | 66.611 | 23.638 | 60.629 | 1.00 | 7.49 | A |
| ATOM | 2598 | CZ | PHE | A | 341 | 65.534 | 22.771 | 60.809 | 1.00 | 8.69 | A |
| ATOM | 2599 | C | PHE | A | 341 | 66.403 | 24.678 | 65.655 | 1.00 | 8.46 | A |
| ATOM | 2600 | O | PHE | A | 341 | 66.638 | 23.481 | 65.482 | 1.00 | 9.08 | A |
| ATOM | 2601 | N | SER | A | 342 | 65.590 | 25.120 | 66.608 | 1.00 | 8.45 | A |
| ATOM | 2602 | CA | SER | A | 342 | 64.946 | 24.191 | 67.526 | 1.00 | 9.16 | A |
| ATOM | 2603 | CB | SER | A | 342 | 64.295 | 24.954 | 68.684 | 1.00 | 11.88 | A |
| ATOM | 2604 | OG | SER | A | 342 | 63.282 | 25.819 | 68.214 | 1.00 | 17.59 | A |
| ATOM | 2605 | C | SER | A | 342 | 63.914 | 23.290 | 66.861 | 1.00 | 8.55 | A |
| ATOM | 2606 | O | SER | A | 342 | 63.139 | 23.725 | 66.008 | 1.00 | 9.19 | A |
| ATOM | 2607 | N | VAL | A | 343 | 63.928 | 22.020 | 67.250 | 1.00 | 9.62 | A |
| ATOM | 2608 | CA | VAL | A | 343 | 62.980 | 21.049 | 66.725 | 1.00 | 8.79 | A |
| ATOM | 2609 | CB | VAL | A | 343 | 63.645 | 20.080 | 65.717 | 1.00 | 8.23 | A |
| ATOM | 2610 | CG1 | VAL | A | 343 | 64.063 | 20.847 | 64.467 | 1.00 | 9.32 | A |
| ATOM | 2611 | CG2 | VAL | A | 343 | 64.862 | 19.402 | 66.345 | 1.00 | 8.55 | A |
| ATOM | 2612 | C | VAL | A | 343 | 62.362 | 20.243 | 67.857 | 1.00 | 9.81 | A |
| ATOM | 2613 | O | VAL | A | 343 | 62.942 | 20.107 | 68.940 | 1.00 | 10.31 | A |
| ATOM | 2614 | N | SER | A | 344 | 61.173 | 19.718 | 67.588 | 1.00 | 9.93 | A |
| ATOM | 2615 | CA | SER | A | 344 | 60.433 | 18.918 | 68.548 | 1.00 | 10.15 | A |
| ATOM | 2616 | CB | SER | A | 344 | 59.266 | 19.726 | 69.121 | 1.00 | 12.37 | A |
| ATOM | 2617 | OG | SER | A | 344 | 59.730 | 20.843 | 69.868 | 1.00 | 16.07 | A |
| ATOM | 2618 | C | SER | A | 344 | 59.905 | 17.665 | 67.857 | 1.00 | 9.81 | A |
| ATOM | 2619 | O | SER | A | 344 | 59.909 | 17.570 | 66.628 | 1.00 | 10.32 | A |
| ATOM | 2620 | N | SER | A | 345 | 59.443 | 16.715 | 68.660 | 1.00 | 9.38 | A |
| ATOM | 2621 | CA | SER | A | 345 | 58.931 | 15.445 | 68.165 | 1.00 | 9.16 | A |
| ATOM | 2622 | CB | SER | A | 345 | 58.716 | 14.489 | 69.342 | 1.00 | 11.80 | A |
| ATOM | 2623 | OG | SER | A | 345 | 57.755 | 15.021 | 70.237 | 1.00 | 14.09 | A |
| ATOM | 2624 | C | SER | A | 345 | 57.645 | 15.519 | 67.354 | 1.00 | 9.70 | A |
| ATOM | 2625 | O | SER | A | 345 | 56.813 | 16.413 | 67.541 | 1.00 | 9.78 | A |
| ATOM | 2626 | N | ASN | A | 346 | 57.500 | 14.550 | 66.455 | 1.00 | 8.41 | A |
| ATOM | 2627 | CA | ASN | A | 346 | 56.323 | 14.414 | 65.604 | 1.00 | 8.72 | A |
| ATOM | 2628 | CB | ASN | A | 346 | 55.231 | 13.677 | 66.380 | 1.00 | 9.59 | A |
| ATOM | 2629 | CG | ASN | A | 346 | 55.700 | 12.313 | 66.870 | 1.00 | 9.97 | A |
| ATOM | 2630 | OD1 | ASN | A | 346 | 55.965 | 12.120 | 68.059 | 1.00 | 12.04 | A |
| ATOM | 2631 | ND2 | ASN | A | 346 | 55.822 | 11.366 | 65.947 | 1.00 | 10.54 | A |
| ATOM | 2632 | C | ASN | A | 346 | 55.824 | 15.747 | 65.069 | 1.00 | 9.15 | A |
| ATOM | 2633 | O | ASN | A | 346 | 54.643 | 16.092 | 65.190 | 1.00 | 10.20 | A |
| ATOM | 2634 | N | SER | A | 347 | 56.750 | 16.479 | 64.456 | 1.00 | 7.99 | A |
| ATOM | 2635 | CA | SER | A | 347 | 56.467 | 17.790 | 63.894 | 1.00 | 7.76 | A |
| ATOM | 2636 | CB | SER | A | 347 | 57.148 | 18.871 | 64.738 | 1.00 | 7.82 | A |
| ATOM | 2637 | OG | SER | A | 347 | 56.715 | 18.816 | 66.086 | 1.00 | 9.43 | A |
| ATOM | 2638 | C | SER | A | 347 | 56.941 | 17.885 | 62.448 | 1.00 | 8.84 | A |
| ATOM | 2639 | O | SER | A | 347 | 57.635 | 16.999 | 61.951 | 1.00 | 9.55 | A |
| ATOM | 2640 | N | LEU | A | 348 | 56.552 | 18.967 | 61.782 | 1.00 | 8.43 | A |
| ATOM | 2641 | CA | LEU | A | 348 | 56.913 | 19.193 | 60.387 | 1.00 | 9.28 | A |
| ATOM | 2642 | CB | LEU | A | 348 | 55.655 | 19.156 | 59.519 | 1.00 | 8.29 | A |
| ATOM | 2643 | CG | LEU | A | 348 | 55.805 | 19.531 | 58.048 | 1.00 | 8.86 | A |
| ATOM | 2644 | CD1 | LEU | A | 348 | 56.652 | 18.490 | 57.330 | 1.00 | 10.18 | A |
| ATOM | 2645 | CD2 | LEU | A | 348 | 54.428 | 19.617 | 57.411 | 1.00 | 10.65 | A |
| ATOM | 2646 | C | LEU | A | 348 | 57.604 | 20.538 | 60.220 | 1.00 | 9.18 | A |
| ATOM | 2647 | O | LEU | A | 348 | 57.088 | 21.569 | 60.655 | 1.00 | 9.28 | A |
| ATOM | 2648 | N | TYR | A | 349 | 58.774 | 20.523 | 59.589 | 1.00 | 8.39 | A |
| ATOM | 2649 | CA | TYR | A | 349 | 59.532 | 21.748 | 59.358 | 1.00 | 8.32 | A |
| ATOM | 2650 | CB | TYR | A | 349 | 60.915 | 21.631 | 60.006 | 1.00 | 7.92 | A |
| ATOM | 2651 | CG | TYR | A | 349 | 60.786 | 21.453 | 61.496 | 1.00 | 8.63 | A |
| ATOM | 2652 | CD1 | TYR | A | 349 | 60.580 | 22.550 | 62.330 | 1.00 | 8.37 | A |
| ATOM | 2653 | CE1 | TYR | A | 349 | 60.292 | 22.383 | 63.681 | 1.00 | 8.57 | A |
| ATOM | 2654 | CD2 | TYR | A | 349 | 60.714 | 20.178 | 62.056 | 1.00 | 8.53 | A |
| ATOM | 2655 | CE2 | TYR | A | 349 | 60.425 | 19.996 | 63.407 | 1.00 | 9.53 | A |
| ATOM | 2656 | CZ | TYR | A | 349 | 60.210 | 21.108 | 64.210 | 1.00 | 7.92 | A |
| ATOM | 2657 | OH | TYR | A | 349 | 59.878 | 20.944 | 65.531 | 1.00 | 9.77 | A |
| ATOM | 2658 | C | TYR | A | 349 | 59.637 | 22.001 | 57.868 | 1.00 | 9.13 | A |
| ATOM | 2659 | O | TYR | A | 349 | 59.945 | 21.101 | 57.089 | 1.00 | 9.86 | A |
| ATOM | 2660 | N | THR | A | 350 | 59.360 | 23.240 | 57.483 | 1.00 | 8.04 | A |
| ATOM | 2661 | CA | THR | A | 350 | 59.375 | 23.635 | 56.088 | 1.00 | 9.18 | A |
| ATOM | 2662 | CB | THR | A | 350 | 57.979 | 24.143 | 55.669 | 1.00 | 9.25 | A |
| ATOM | 2663 | OG1 | THR | A | 350 | 56.997 | 23.148 | 55.989 | 1.00 | 9.76 | A |
| ATOM | 2664 | CG2 | THR | A | 350 | 57.932 | 24.389 | 54.185 | 1.00 | 10.21 | A |
| ATOM | 2665 | C | THR | A | 350 | 60.418 | 24.729 | 55.881 | 1.00 | 8.80 | A |
| ATOM | 2666 | O | THR | A | 350 | 60.311 | 25.824 | 56.444 | 1.00 | 11.00 | A |
| ATOM | 2667 | N | LEU | A | 351 | 61.437 | 24.420 | 55.083 | 1.00 | 8.10 | A |
| ATOM | 2668 | CA | LEU | A | 351 | 62.506 | 25.371 | 54.808 | 1.00 | 8.20 | A |
| ATOM | 2669 | CB | LEU | A | 351 | 63.860 | 24.798 | 55.240 | 1.00 | 8.52 | A |
| ATOM | 2670 | CG | LEU | A | 351 | 64.045 | 24.472 | 56.722 | 1.00 | 10.68 | A |
| ATOM | 2671 | CD1 | LEU | A | 351 | 63.417 | 23.124 | 57.043 | 1.00 | 13.23 | A |
| ATOM | 2672 | CD2 | LEU | A | 351 | 65.534 | 24.440 | 57.026 | 1.00 | 13.26 | A |
| ATOM | 2673 | C | LEU | A | 351 | 62.602 | 25.760 | 53.345 | 1.00 | 8.77 | A |
| ATOM | 2674 | O | LEU | A | 351 | 62.809 | 24.912 | 52.479 | 1.00 | 9.51 | A |
| ATOM | 2675 | N | SER | A | 352 | 62.450 | 27.047 | 53.065 | 1.00 | 9.42 | A |
| ATOM | 2676 | CA | SER | A | 352 | 62.574 | 27.531 | 51.695 | 1.00 | 9.78 | A |
| ATOM | 2677 | CB | SER | A | 352 | 62.028 | 28.958 | 51.583 | 1.00 | 11.99 | A |
| ATOM | 2678 | OG | SER | A | 352 | 62.151 | 29.449 | 50.263 | 1.00 | 18.34 | A |
| ATOM | 2679 | C | SER | A | 352 | 64.073 | 27.526 | 51.411 | 1.00 | 10.78 | A |
| ATOM | 2680 | O | SER | A | 352 | 64.827 | 28.218 | 52.094 | 1.00 | 11.98 | A |
| ATOM | 2681 | N | HIS | A | 353 | 64.517 | 26.754 | 50.420 | 1.00 | 9.89 | A |
| ATOM | 2682 | CA | HIS | A | 353 | 65.951 | 26.695 | 50.124 | 1.00 | 9.22 | A |
| ATOM | 2683 | CB | HIS | A | 353 | 66.330 | 25.306 | 49.563 | 1.00 | 8.55 | A |
| ATOM | 2684 | CG | HIS | A | 353 | 65.520 | 24.878 | 48.378 | 1.00 | 8.70 | A |
| ATOM | 2685 | CD2 | HIS | A | 353 | 64.734 | 23.793 | 48.182 | 1.00 | 8.21 | A |
| ATOM | 2686 | ND1 | HIS | A | 353 | 65.492 | 25.590 | 47.198 | 1.00 | 7.93 | A |
| ATOM | 2687 | CE1 | HIS | A | 353 | 64.726 | 24.961 | 46.325 | 1.00 | 8.00 | A |
| ATOM | 2688 | NE2 | HIS | A | 353 | 64.254 | 23.867 | 46.896 | 1.00 | 8.05 | A |
| ATOM | 2689 | C | HIS | A | 353 | 66.464 | 27.809 | 49.213 | 1.00 | 9.19 | A |
| ATOM | 2690 | O | HIS | A | 353 | 67.670 | 28.047 | 49.154 | 1.00 | 9.57 | A |
| ATOM | 2691 | N | GLY | A | 354 | 65.550 | 28.489 | 48.524 | 1.00 | 8.70 | A |
| ATOM | 2692 | CA | GLY | A | 354 | 65.922 | 29.587 | 47.646 | 1.00 | 10.23 | A |
| ATOM | 2693 | C | GLY | A | 354 | 66.840 | 29.266 | 46.479 | 1.00 | 9.22 | A |
| ATOM | 2694 | O | GLY | A | 354 | 67.466 | 30.165 | 45.915 | 1.00 | 10.47 | A |
| ATOM | 2695 | N | LEU | A | 355 | 66.910 | 27.996 | 46.097 | 1.00 | 8.66 | A |
| ATOM | 2696 | CA | LEU | A | 355 | 67.786 | 27.574 | 45.007 | 1.00 | 8.35 | A |
| ATOM | 2697 | CB | LEU | A | 355 | 68.291 | 26.157 | 45.276 | 1.00 | 6.95 | A |
| ATOM | 2698 | CG | LEU | A | 355 | 68.924 | 25.978 | 46.659 | 1.00 | 7.51 | A |
| ATOM | 2699 | CD1 | LEU | A | 355 | 69.302 | 24.518 | 46.858 | 1.00 | 8.82 | A |
| ATOM | 2700 | CD2 | LEU | A | 355 | 70.144 | 26.879 | 46.796 | 1.00 | 9.17 | A |
| ATOM | 2701 | C | LEU | A | 355 | 67.143 | 27.628 | 43.627 | 1.00 | 8.52 | A |
| ATOM | 2702 | O | LEU | A | 355 | 65.924 | 27.536 | 43.486 | 1.00 | 9.48 | A |
| ATOM | 2703 | N | GLN | A | 356 | 67.988 | 27.768 | 42.610 | 1.00 | 7.66 | A |
| ATOM | 2704 | CA | GLN | A | 356 | 67.540 | 27.842 | 41.225 | 1.00 | 8.81 | A |
| ATOM | 2705 | CB | GLN | A | 356 | 68.722 | 28.216 | 40.331 | 1.00 | 7.38 | A |
| ATOM | 2706 | CG | GLN | A | 356 | 68.369 | 28.446 | 38.872 | 1.00 | 8.63 | A |
| ATOM | 2707 | CD | GLN | A | 356 | 69.534 | 29.029 | 38.099 | 1.00 | 9.22 | A |
| ATOM | 2708 | OE1 | GLN | A | 356 | 69.546 | 30.221 | 37.755 | 1.00 | 13.17 | A |
| ATOM | 2709 | NE2 | GLN | A | 356 | 70.533 | 28.199 | 37.837 | 1.00 | 6.64 | A |
| ATOM | 2710 | C | GLN | A | 356 | 66.895 | 26.536 | 40.765 | 1.00 | 8.76 | A |
| ATOM | 2711 | O | GLN | A | 356 | 66.097 | 26.518 | 39.828 | 1.00 | 9.04 | A |
| ATOM | 2712 | N | ARG | A | 357 | 67.262 | 25.439 | 41.419 | 1.00 | 8.59 | A |
| ATOM | 2713 | CA | ARG | A | 357 | 66.680 | 24.131 | 41.123 | 1.00 | 8.52 | A |
| ATOM | 2714 | CB | ARG | A | 357 | 67.503 | 23.366 | 40.077 | 1.00 | 9.61 | A |
| ATOM | 2715 | CG | ARG | A | 357 | 68.972 | 23.131 | 40.426 | 1.00 | 8.94 | A |
| ATOM | 2716 | CD | ARG | A | 357 | 69.454 | 21.854 | 39.751 | 1.00 | 13.45 | A |
| ATOM | 2717 | NE | ARG | A | 357 | 68.803 | 20.718 | 40.394 | 1.00 | 14.81 | A |
| ATOM | 2718 | CZ | ARG | A | 357 | 68.456 | 19.587 | 39.791 | 1.00 | 15.05 | A |
| ATOM | 2719 | NH1 | ARG | A | 357 | 68.694 | 19.402 | 38.495 | 1.00 | 14.67 | A |
| ATOM | 2720 | NH2 | ARG | A | 357 | 67.839 | 18.648 | 40.500 | 1.00 | 14.22 | A |
| ATOM | 2721 | C | ARG | A | 357 | 66.642 | 23.351 | 42.426 | 1.00 | 8.47 | A |
| ATOM | 2722 | O | ARG | A | 357 | 67.262 | 23.758 | 43.413 | 1.00 | 9.07 | A |
| ATOM | 2723 | N | SER | A | 358 | 65.917 | 22.240 | 42.445 | 1.00 | 8.65 | A |
| ATOM | 2724 | CA | SER | A | 358 | 65.847 | 21.437 | 43.656 | 1.00 | 7.62 | A |
| ATOM | 2725 | CB | SER | A | 358 | 64.901 | 20.251 | 43.462 | 1.00 | 9.00 | A |
| ATOM | 2726 | OG | SER | A | 358 | 65.352 | 19.409 | 42.408 | 1.00 | 11.95 | A |
| ATOM | 2727 | C | SER | A | 358 | 67.249 | 20.930 | 43.973 | 1.00 | 8.93 | A |
| ATOM | 2728 | O | SER | A | 358 | 68.002 | 20.557 | 43.071 | 1.00 | 9.41 | A |
| ATOM | 2729 | N | PRO | A | 359 | 67.634 | 20.937 | 45.257 | 1.00 | 7.79 | A |
| ATOM | 2730 | CD | PRO | A | 359 | 66.928 | 21.448 | 46.445 | 1.00 | 7.95 | A |
| ATOM | 2731 | CA | PRO | A | 359 | 68.972 | 20.452 | 45.603 | 1.00 | 7.43 | A |
| ATOM | 2732 | CB | PRO | A | 359 | 69.129 | 20.881 | 47.060 | 1.00 | 7.98 | A |
| ATOM | 2733 | CG | PRO | A | 359 | 67.723 | 20.833 | 47.582 | 1.00 | 9.25 | A |
| ATOM | 2734 | C | PRO | A | 359 | 69.055 | 18.940 | 45.425 | 1.00 | 7.43 | A |
| ATOM | 2735 | O | PRO | A | 359 | 68.158 | 18.208 | 45.844 | 1.00 | 8.59 | A |
| ATOM | 2736 | N | ARG | A | 360 | 70.128 | 18.468 | 44.800 | 1.00 | 7.54 | A |
| ATOM | 2737 | CA | ARG | A | 360 | 70.279 | 17.033 | 44.587 | 1.00 | 6.64 | A |
| ATOM | 2738 | CB | ARG | A | 360 | 71.259 | 16.770 | 43.441 | 1.00 | 7.03 | A |
| ATOM | 2739 | CG | ARG | A | 360 | 70.887 | 17.381 | 42.084 | 1.00 | 6.71 | A |
| ATOM | 2740 | CD | ARG | A | 360 | 71.798 | 16.789 | 41.013 | 1.00 | 8.32 | A |
| ATOM | 2741 | NE | ARG | A | 360 | 71.773 | 17.474 | 39.715 | 1.00 | 7.87 | A |
| ATOM | 2742 | CZ | ARG | A | 360 | 72.353 | 18.645 | 39.462 | 1.00 | 9.26 | A |
| ATOM | 2743 | NH1 | ARG | A | 360 | 73.004 | 19.300 | 40.417 | 1.00 | 9.69 | A |
| ATOM | 2744 | NH2 | ARG | A | 360 | 72.325 | 19.135 | 38.231 | 1.00 | 12.27 | A |
| ATOM | 2745 | C | ARG | A | 360 | 70.772 | 16.319 | 45.847 | 1.00 | 7.48 | A |
| ATOM | 2746 | O | ARG | A | 360 | 70.604 | 15.107 | 45.979 | 1.00 | 7.86 | A |
| ATOM | 2747 | N | ARG | A | 361 | 71.373 | 17.073 | 46.767 | 1.00 | 7.46 | A |
| ATOM | 2748 | CA | ARG | A | 361 | 71.903 | 16.502 | 48.005 | 1.00 | 8.35 | A |
| ATOM | 2749 | CB | ARG | A | 361 | 73.434 | 16.467 | 47.945 | 1.00 | 8.78 | A |
| ATOM | 2750 | CG | ARG | A | 361 | 74.003 | 15.576 | 46.847 | 1.00 | 11.64 | A |
| ATOM | 2751 | CD | ARG | A | 361 | 75.137 | 16.273 | 46.093 | 1.00 | 16.84 | A |
| ATOM | 2752 | NE | ARG | A | 361 | 76.213 | 16.725 | 46.972 | 1.00 | 20.36 | A |
| ATOM | 2753 | CZ | ARG | A | 361 | 77.166 | 17.582 | 46.610 | 1.00 | 20.66 | A |
| ATOM | 2754 | NH1 | ARG | A | 361 | 77.180 | 18.087 | 45.384 | 1.00 | 20.34 | A |
| ATOM | 2755 | NH2 | ARG | A | 361 | 78.105 | 17.935 | 47.476 | 1.00 | 21.62 | A |
| ATOM | 2756 | C | ARG | A | 361 | 71.469 | 17.311 | 49.225 | 1.00 | 7.38 | A |
| ATOM | 2757 | O | ARG | A | 361 | 71.487 | 18.541 | 49.201 | 1.00 | 7.28 | A |
| ATOM | 2758 | N | VAL | A | 362 | 71.088 | 16.610 | 50.286 | 1.00 | 7.19 | A |
| ATOM | 2759 | CA | VAL | A | 362 | 70.654 | 17.246 | 51.523 | 1.00 | 7.92 | A |
| ATOM | 2760 | CB | VAL | A | 362 | 69.103 | 17.256 | 51.632 | 1.00 | 9.09 | A |
| ATOM | 2761 | CG1 | VAL | A | 362 | 68.666 | 17.856 | 52.970 | 1.00 | 9.25 | A |
| ATOM | 2762 | CG2 | VAL | A | 362 | 68.505 | 18.046 | 50.480 | 1.00 | 10.34 | A |
| ATOM | 2763 | C | VAL | A | 362 | 71.224 | 16.483 | 52.716 | 1.00 | 8.38 | A |
| ATOM | 2764 | O | VAL | A | 362 | 71.287 | 15.254 | 52.704 | 1.00 | 10.02 | A |
| ATOM | 2765 | N | VAL | A | 363 | 71.663 | 17.222 | 53.730 | 1.00 | 8.34 | A |
| ATOM | 2766 | CA | VAL | A | 363 | 72.190 | 16.625 | 54.955 | 1.00 | 9.01 | A |
| ATOM | 2767 | CB | VAL | A | 363 | 73.709 | 16.913 | 55.144 | 1.00 | 10.17 | A |
| ATOM | 2768 | CG1 | VAL | A | 363 | 74.137 | 16.587 | 56.577 | 1.00 | 13.71 | A |
| ATOM | 2769 | CG2 | VAL | A | 363 | 74.526 | 16.087 | 54.165 | 1.00 | 11.86 | A |
| ATOM | 2770 | C | VAL | A | 363 | 71.439 | 17.263 | 56.116 | 1.00 | 8.28 | A |
| ATOM | 2771 | O | VAL | A | 363 | 71.222 | 18.475 | 56.124 | 1.00 | 8.12 | A |
| ATOM | 2772 | N | VAL | A | 364 | 71.021 | 16.447 | 57.076 | 1.00 | 6.88 | A |
| ATOM | 2773 | CA | VAL | A | 364 | 70.329 | 16.954 | 58.252 | 1.00 | 7.03 | A |
| ATOM | 2774 | CB | VAL | A | 364 | 68.901 | 16.392 | 58.382 | 1.00 | 6.85 | A |
| ATOM | 2775 | CG1 | VAL | A | 364 | 68.238 | 16.973 | 59.624 | 1.00 | 8.41 | A |
| ATOM | 2776 | CG2 | VAL | A | 364 | 68.092 | 16.733 | 57.140 | 1.00 | 9.44 | A |
| ATOM | 2777 | C | VAL | A | 364 | 71.137 | 16.490 | 59.452 | 1.00 | 6.26 | A |
| ATOM | 2778 | O | VAL | A | 364 | 71.445 | 15.306 | 59.576 | 1.00 | 7.03 | A |
| ATOM | 2779 | N | GLU | A | 365 | 71.491 | 17.423 | 60.327 | 1.00 | 6.62 | A |
| ATOM | 2780 | CA | GLU | A | 365 | 72.260 | 17.080 | 61.508 | 1.00 | 6.96 | A |
| ATOM | 2781 | CB | GLU | A | 365 | 73.681 | 17.638 | 61.370 | 1.00 | 6.57 | A |
| ATOM | 2782 | CG | GLU | A | 365 | 74.474 | 16.834 | 60.326 | 1.00 | 8.30 | A |
| ATOM | 2783 | CD | GLU | A | 365 | 75.784 | 17.474 | 59.915 | 1.00 | 10.71 | A |
| ATOM | 2784 | OE1 | GLU | A | 365 | 75.799 | 18.697 | 59.660 | 1.00 | 9.49 | A |
| ATOM | 2785 | OE2 | GLU | A | 365 | 76.792 | 16.741 | 59.820 | 1.00 | 10.16 | A |
| ATOM | 2786 | C | GLU | A | 365 | 71.565 | 17.549 | 62.774 | 1.00 | 8.05 | A |
| ATOM | 2787 | O | GLU | A | 365 | 70.737 | 18.459 | 62.735 | 1.00 | 8.08 | A |
| ATOM | 2788 | N | PHE | A | 366 | 71.896 | 16.908 | 63.891 | 1.00 | 6.14 | A |
| ATOM | 2789 | CA | PHE | A | 366 | 71.275 | 17.203 | 65.180 | 1.00 | 8.06 | A |
| ATOM | 2790 | CB | PHE | A | 366 | 70.474 | 15.979 | 65.629 | 1.00 | 6.65 | A |
| ATOM | 2791 | CG | PHE | A | 366 | 70.034 | 16.038 | 67.055 | 1.00 | 8.41 | A |
| ATOM | 2792 | CD1 | PHE | A | 366 | 68.909 | 16.766 | 67.414 | 1.00 | 9.43 | A |
| ATOM | 2793 | CD2 | PHE | A | 366 | 70.771 | 15.399 | 68.048 | 1.00 | 9.20 | A |
| ATOM | 2794 | CE1 | PHE | A | 366 | 68.522 | 16.865 | 68.745 | 1.00 | 10.65 | A |
| ATOM | 2795 | CE2 | PHE | A | 366 | 70.394 | 15.491 | 69.382 | 1.00 | 10.36 | A |
| ATOM | 2796 | CZ | PHE | A | 366 | 69.267 | 16.226 | 69.732 | 1.00 | 10.43 | A |
| ATOM | 2797 | C | PHE | A | 366 | 72.283 | 17.566 | 66.268 | 1.00 | 8.10 | A |
| ATOM | 2798 | O | PHE | A | 366 | 73.353 | 16.964 | 66.364 | 1.00 | 8.23 | A |
| ATOM | 2799 | N | ALA | A | 367 | 71.928 | 18.548 | 67.090 | 1.00 | 7.54 | A |
| ATOM | 2800 | CA | ALA | A | 367 | 72.782 | 18.980 | 68.187 | 1.00 | 8.75 | A |
| ATOM | 2801 | CB | ALA | A | 367 | 73.648 | 20.163 | 67.759 | 1.00 | 8.16 | A |
| ATOM | 2802 | C | ALA | A | 367 | 71.904 | 19.368 | 69.371 | 1.00 | 9.44 | A |
| ATOM | 2803 | O | ALA | A | 367 | 70.710 | 19.624 | 69.211 | 1.00 | 8.92 | A |
| ATOM | 2804 | N | ARG | A | 368 | 72.498 | 19.411 | 70.559 | 1.00 | 10.68 | A |
| ATOM | 2805 | CA | ARG | A | 368 | 71.754 | 19.749 | 71.763 | 1.00 | 12.90 | A |
| ATOM | 2806 | CB | ARG | A | 368 | 72.306 | 18.950 | 72.945 | 1.00 | 15.51 | A |
| ATOM | 2807 | CG | ARG | A | 368 | 72.282 | 17.443 | 72.706 | 1.00 | 21.38 | A |
| ATOM | 2808 | CD | ARG | A | 368 | 72.221 | 16.667 | 74.009 | 1.00 | 26.94 | A |
| ATOM | 2809 | NE | ARG | A | 368 | 70.972 | 16.939 | 74.715 | 1.00 | 31.17 | A |
| ATOM | 2810 | CZ | ARG | A | 368 | 70.613 | 16.358 | 75.854 | 1.00 | 32.60 | A |
| ATOM | 2811 | NH1 | ARG | A | 368 | 71.411 | 15.466 | 76.425 | 1.00 | 32.62 | A |
| ATOM | 2812 | NH2 | ARG | A | 368 | 69.453 | 16.664 | 76.417 | 1.00 | 33.64 | A |
| ATOM | 2813 | C | ARG | A | 368 | 71.760 | 21.242 | 72.080 | 1.00 | 13.06 | A |
| ATOM | 2814 | O | ARG | A | 368 | 71.158 | 21.679 | 73.063 | 1.00 | 14.15 | A |
| ATOM | 2815 | N | SER | A | 369 | 72.442 | 22.018 | 71.245 | 1.00 | 10.96 | A |
| ATOM | 2816 | CA | SER | A | 369 | 72.507 | 23.466 | 71.414 | 1.00 | 11.34 | A |
| ATOM | 2817 | CB | SER | A | 369 | 73.641 | 23.859 | 72.364 | 1.00 | 12.47 | A |
| ATOM | 2818 | OG | SER | A | 369 | 74.899 | 23.830 | 71.708 | 1.00 | 12.71 | A |
| ATOM | 2819 | C | SER | A | 369 | 72.759 | 24.093 | 70.049 | 1.00 | 11.28 | A |
| ATOM | 2820 | O | SER | A | 369 | 73.067 | 23.387 | 69.090 | 1.00 | 11.19 | A |
| ATOM | 2821 | N | SER | A | 370 | 72.627 | 25.415 | 69.965 | 1.00 | 10.92 | A |
| ATOM | 2822 | CA | SER | A | 370 | 72.853 | 26.116 | 68.710 | 1.00 | 11.44 | A |
| ATOM | 2823 | CB | SER | A | 370 | 71.927 | 27.329 | 68.591 | 1.00 | 11.62 | A |
| ATOM | 2824 | OG | SER | A | 370 | 72.131 | 28.237 | 69.654 | 1.00 | 12.01 | A |
| ATOM | 2825 | C | SER | A | 370 | 74.313 | 26.543 | 68.550 | 1.00 | 10.92 | A |
| ATOM | 2826 | O | SER | A | 370 | 74.644 | 27.305 | 67.645 | 1.00 | 10.76 | A |
| ATOM | 2827 | N | SER | A | 371 | 75.180 | 26.060 | 69.438 | 1.00 | 11.37 | A |
| ATOM | 2828 | CA | SER | A | 371 | 76.618 | 26.337 | 69.364 | 1.00 | 11.70 | A |
| ATOM | 2829 | CB | SER | A | 371 | 76.980 | 27.526 | 70.255 | 1.00 | 14.13 | A |
| ATOM | 2830 | OG | SER | A | 371 | 78.305 | 27.955 | 69.991 | 1.00 | 21.19 | A |
| ATOM | 2831 | C | SER | A | 371 | 77.345 | 25.078 | 69.851 | 1.00 | 11.27 | A |
| ATOM | 2832 | O | SER | A | 371 | 78.160 | 25.131 | 70.771 | 1.00 | 11.31 | A |
| ATOM | 2833 | N | PRO | A | 372 | 77.066 | 23.930 | 69.217 | 1.00 | 11.10 | A |
| ATOM | 2834 | CD | PRO | A | 372 | 76.199 | 23.813 | 68.028 | 1.00 | 12.11 | A |
| ATOM | 2835 | CA | PRO | A | 372 | 77.647 | 22.631 | 69.555 | 1.00 | 11.22 | A |
| ATOM | 2836 | CB | PRO | A | 372 | 76.703 | 21.668 | 68.860 | 1.00 | 10.93 | A |
| ATOM | 2837 | CG | PRO | A | 372 | 76.464 | 22.380 | 67.566 | 1.00 | 11.71 | A |
| ATOM | 2838 | C | PRO | A | 372 | 79.089 | 22.382 | 69.147 | 1.00 | 11.56 | A |
| ATOM | 2839 | O | PRO | A | 372 | 79.595 | 22.974 | 68.195 | 1.00 | 12.85 | A |
| ATOM | 2840 | N | SER | A | 373 | 79.744 | 21.495 | 69.888 | 1.00 | 11.26 | A |
| ATOM | 2841 | CA | SER | A | 373 | 81.114 | 21.112 | 69.587 | 1.00 | 13.44 | A |
| ATOM | 2842 | CB | SER | A | 373 | 81.913 | 20.889 | 70.878 | 1.00 | 14.84 | A |
| ATOM | 2843 | OG | SER | A | 373 | 81.316 | 19.906 | 71.701 | 1.00 | 20.51 | A |
| ATOM | 2844 | C | SER | A | 373 | 81.052 | 19.832 | 68.755 | 1.00 | 12.56 | A |
| ATOM | 2845 | O | SER | A | 373 | 81.979 | 19.520 | 68.008 | 1.00 | 14.08 | A |
| ATOM | 2846 | N | THR | A | 374 | 79.951 | 19.096 | 68.894 | 1.00 | 12.24 | A |
| ATOM | 2847 | CA | THR | A | 374 | 79.729 | 17.868 | 68.125 | 1.00 | 11.10 | A |
| ATOM | 2848 | CB | THR | A | 374 | 80.135 | 16.583 | 68.900 | 1.00 | 11.89 | A |
| ATOM | 2849 | OG1 | THR | A | 374 | 79.277 | 16.402 | 70.033 | 1.00 | 12.97 | A |
| ATOM | 2850 | CG2 | THR | A | 374 | 81.582 | 16.668 | 69.361 | 1.00 | 13.54 | A |
| ATOM | 2851 | C | THR | A | 374 | 78.246 | 17.770 | 67.774 | 1.00 | 9.93 | A |
| ATOM | 2852 | O | THR | A | 374 | 77.403 | 18.381 | 68.430 | 1.00 | 10.01 | A |
| ATOM | 2853 | N | TRP | A | 375 | 77.934 | 17.008 | 66.732 | 1.00 | 9.16 | A |
| ATOM | 2854 | CA | TRP | A | 375 | 76.556 | 16.835 | 66.289 | 1.00 | 8.32 | A |
| ATOM | 2855 | CB | TRP | A | 375 | 76.165 | 17.970 | 65.334 | 1.00 | 9.18 | A |
| ATOM | 2856 | CG | TRP | A | 375 | 77.212 | 18.244 | 64.308 | 1.00 | 8.84 | A |
| ATOM | 2857 | CD2 | TRP | A | 375 | 78.357 | 19.088 | 64.465 | 1.00 | 10.79 | A |
| ATOM | 2858 | CE2 | TRP | A | 375 | 79.126 | 18.970 | 63.286 | 1.00 | 10.55 | A |
| ATOM | 2859 | CE3 | TRP | A | 375 | 78.810 | 19.932 | 65.491 | 1.00 | 11.13 | A |
| ATOM | 2860 | CD1 | TRP | A | 375 | 77.326 | 17.669 | 63.073 | 1.00 | 8.86 | A |
| ATOM | 2861 | NE1 | TRP | A | 375 | 78.472 | 18.098 | 62.455 | 1.00 | 9.59 | A |
| ATOM | 2862 | CZ2 | TRP | A | 375 | 80.329 | 19.661 | 63.103 | 1.00 | 13.06 | A |
| ATOM | 2863 | CZ3 | TRP | A | 375 | 80.007 | 20.620 | 65.308 | 1.00 | 13.00 | A |
| ATOM | 2864 | CH2 | TRP | A | 375 | 80.751 | 20.479 | 64.121 | 1.00 | 12.63 | A |
| ATOM | 2865 | C | TRP | A | 375 | 76.427 | 15.481 | 65.603 | 1.00 | 8.58 | A |
| ATOM | 2866 | O | TRP | A | 375 | 77.433 | 14.861 | 65.252 | 1.00 | 8.31 | A |
| ATOM | 2867 | N | ASN | A | 376 | 75.190 | 15.030 | 65.413 | 1.00 | 7.30 | A |
| ATOM | 2868 | CA | ASN | A | 376 | 74.933 | 13.729 | 64.796 | 1.00 | 7.59 | A |
| ATOM | 2869 | CB | ASN | A | 376 | 74.058 | 12.871 | 65.713 | 1.00 | 7.03 | A |
| ATOM | 2870 | CG | ASN | A | 376 | 74.624 | 12.755 | 67.106 | 1.00 | 9.32 | A |
| ATOM | 2871 | OD1 | ASN | A | 376 | 74.076 | 13.307 | 68.060 | 1.00 | 9.70 | A |
| ATOM | 2872 | ND2 | ASN | A | 376 | 75.739 | 12.047 | 67.231 | 1.00 | 8.22 | A |
| ATOM | 2873 | C | ASN | A | 376 | 74.237 | 13.842 | 63.462 | 1.00 | 7.37 | A |
| ATOM | 2874 | O | ASN | A | 376 | 73.532 | 14.813 | 63.201 | 1.00 | 7.98 | A |
| ATOM | 2875 | N | ILE | A | 377 | 74.438 | 12.843 | 62.613 | 1.00 | 6.38 | A |
| ATOM | 2876 | CA | ILE | A | 377 | 73.773 | 12.832 | 61.324 | 1.00 | 6.92 | A |
| ATOM | 2877 | CB | ILE | A | 377 | 74.552 | 11.984 | 60.295 | 1.00 | 7.75 | A |
| ATOM | 2878 | CG2 | ILE | A | 377 | 73.800 | 11.953 | 58.966 | 1.00 | 9.47 | A |
| ATOM | 2879 | CG1 | ILE | A | 377 | 75.954 | 12.573 | 60.108 | 1.00 | 9.20 | A |
| ATOM | 2880 | CD1 | ILE | A | 377 | 76.802 | 11.867 | 59.056 | 1.00 | 10.99 | A |
| ATOM | 2881 | C | ILE | A | 377 | 72.384 | 12.245 | 61.564 | 1.00 | 6.94 | A |
| ATOM | 2882 | O | ILE | A | 377 | 72.232 | 11.221 | 62.236 | 1.00 | 7.84 | A |
| ATOM | 2883 | N | VAL | A | 378 | 71.367 | 12.911 | 61.027 | 1.00 | 6.50 | A |
| ATOM | 2884 | CA | VAL | A | 378 | 69.985 | 12.482 | 61.203 | 1.00 | 6.69 | A |
| ATOM | 2885 | CB | VAL | A | 378 | 69.051 | 13.701 | 61.327 | 1.00 | 7.03 | A |
| ATOM | 2886 | CG1 | VAL | A | 378 | 67.612 | 13.240 | 61.489 | 1.00 | 7.99 | A |
| ATOM | 2887 | CG2 | VAL | A | 378 | 69.480 | 14.555 | 62.514 | 1.00 | 6.80 | A |
| ATOM | 2888 | C | VAL | A | 378 | 69.491 | 11.609 | 60.057 | 1.00 | 6.62 | A |
| ATOM | 2889 | O | VAL | A | 378 | 69.489 | 12.029 | 58.905 | 1.00 | 8.07 | A |
| ATOM | 2890 | N | MET | A | 379 | 69.067 | 10.396 | 60.397 | 1.00 | 9.39 | A |
| ATOM | 2891 | CA | MET | A | 379 | 68.557 | 9.429 | 59.431 | 1.00 | 11.50 | A |
| ATOM | 2892 | CB | MET | A | 379 | 69.660 | 8.416 | 59.084 | 1.00 | 12.25 | A |
| ATOM | 2893 | CG | MET | A | 379 | 70.814 | 9.035 | 58.283 | 1.00 | 16.24 | A |
| ATOM | 2894 | SD | MET | A | 379 | 72.289 | 7.984 | 58.090 | 1.00 | 24.50 | A |
| ATOM | 2895 | CE | MET | A | 379 | 73.250 | 8.452 | 59.551 | 1.00 | 21.41 | A |
| ATOM | 2896 | C | MET | A | 379 | 67.354 | 8.742 | 60.075 | 1.00 | 13.99 | A |
| ATOM | 2897 | O | MET | A | 379 | 67.361 | 8.484 | 61.278 | 1.00 | 18.71 | A |
| ATOM | 2898 | N | PRO | A | 380 | 66.305 | 8.450 | 59.289 | 1.00 | 12.52 | A |
| ATOM | 2899 | CD | PRO | A | 380 | 66.274 | 8.580 | 57.822 | 1.00 | 13.36 | A |
| ATOM | 2900 | CA | PRO | A | 380 | 65.085 | 7.798 | 59.777 | 1.00 | 14.60 | A |
| ATOM | 2901 | CB | PRO | A | 380 | 64.205 | 7.731 | 58.529 | 1.00 | 14.51 | A |
| ATOM | 2902 | CG | PRO | A | 380 | 65.204 | 7.580 | 57.432 | 1.00 | 12.98 | A |
| ATOM | 2903 | C | PRO | A | 380 | 65.333 | 6.438 | 60.406 | 1.00 | 16.92 | A |
| ATOM | 2904 | O | PRO | A | 380 | 65.612 | 5.461 | 59.714 | 1.00 | 20.94 | A |
| ATOM | 2905 | N | SER | A | 381 | 65.224 | 6.399 | 61.729 | 1.00 | 16.24 | A |
| ATOM | 2906 | CA | SER | A | 381 | 65.452 | 5.188 | 62.493 | 1.00 | 18.01 | A |
| ATOM | 2907 | CB | SER | A | 381 | 66.629 | 5.407 | 63.446 | 1.00 | 19.45 | A |
| ATOM | 2908 | OG | SER | A | 381 | 67.112 | 4.181 | 63.954 | 1.00 | 26.04 | A |
| ATOM | 2909 | C | SER | A | 381 | 64.191 | 4.816 | 63.271 | 1.00 | 17.09 | A |
| ATOM | 2910 | O | SER | A | 381 | 63.120 | 5.387 | 63.047 | 1.00 | 14.97 | A |
| ATOM | 2911 | N | TYR | A | 382 | 64.325 | 3.874 | 64.198 | 1.00 | 16.18 | A |
| ATOM | 2912 | CA | TYR | A | 382 | 63.188 | 3.405 | 64.977 | 1.00 | 14.94 | A |
| ATOM | 2913 | CB | TYR | A | 382 | 62.473 | 2.299 | 64.189 | 1.00 | 15.83 | A |
| ATOM | 2914 | CG | TYR | A | 382 | 61.519 | 1.446 | 65.003 | 1.00 | 15.62 | A |
| ATOM | 2915 | CD1 | TYR | A | 382 | 60.157 | 1.733 | 65.046 | 1.00 | 15.11 | A |
| ATOM | 2916 | CE1 | TYR | A | 382 | 59.283 | 0.960 | 65.809 | 1.00 | 17.13 | A |
| ATOM | 2917 | CD2 | TYR | A | 382 | 61.987 | 0.360 | 65.747 | 1.00 | 15.77 | A |
| ATOM | 2918 | CE2 | TYR | A | 382 | 61.122 | −0.416 | 66.514 | 1.00 | 16.33 | A |
| ATOM | 2919 | CZ | TYR | A | 382 | 59.773 | −0.110 | 66.540 | 1.00 | 16.83 | A |
| ATOM | 2920 | OH | TYR | A | 382 | 58.917 | −0.873 | 67.300 | 1.00 | 18.38 | A |
| ATOM | 2921 | C | TYR | A | 382 | 63.608 | 2.857 | 66.338 | 1.00 | 14.28 | A |
| ATOM | 2922 | O | TYR | A | 382 | 64.744 | 2.423 | 66.523 | 1.00 | 14.56 | A |
| ATOM | 2923 | N | PHE | A | 383 | 62.687 | 2.901 | 67.293 | 1.00 | 13.18 | A |
| ATOM | 2924 | CA | PHE | A | 383 | 62.928 | 2.339 | 68.614 | 1.00 | 12.38 | A |
| ATOM | 2925 | CB | PHE | A | 383 | 63.722 | 3.299 | 69.520 | 1.00 | 14.40 | A |
| ATOM | 2926 | CG | PHE | A | 383 | 62.946 | 4.497 | 69.981 | 1.00 | 14.84 | A |
| ATOM | 2927 | CD1 | PHE | A | 383 | 62.247 | 4.474 | 71.183 | 1.00 | 14.47 | A |
| ATOM | 2928 | CD2 | PHE | A | 383 | 62.926 | 5.655 | 69.216 | 1.00 | 16.86 | A |
| ATOM | 2929 | CE1 | PHE | A | 383 | 61.531 | 5.589 | 71.609 | 1.00 | 16.25 | A |
| ATOM | 2930 | CE2 | PHE | A | 383 | 62.213 | 6.775 | 69.630 | 1.00 | 16.80 | A |
| ATOM | 2931 | CZ | PHE | A | 383 | 61.519 | 6.743 | 70.831 | 1.00 | 16.40 | A |
| ATOM | 2932 | C | PHE | A | 383 | 61.575 | 2.002 | 69.211 | 1.00 | 12.55 | A |
| ATOM | 2933 | O | PHE | A | 383 | 60.543 | 2.492 | 68.753 | 1.00 | 11.35 | A |
| ATOM | 2934 | N | ASN | A | 384 | 61.586 | 1.138 | 70.216 | 1.00 | 11.42 | A |
| ATOM | 2935 | CA | ASN | A | 384 | 60.363 | 0.711 | 70.867 | 1.00 | 13.15 | A |
| ATOM | 2936 | CB | ASN | A | 384 | 60.104 | −0.762 | 70.544 | 1.00 | 14.20 | A |
| ATOM | 2937 | CG | ASN | A | 384 | 58.793 | −1.263 | 71.105 | 1.00 | 15.07 | A |
| ATOM | 2938 | OD1 | ASN | A | 384 | 58.604 | −1.308 | 72.318 | 1.00 | 16.43 | A |
| ATOM | 2939 | ND2 | ASN | A | 384 | 57.877 | −1.645 | 70.220 | 1.00 | 15.88 | A |
| ATOM | 2940 | C | ASN | A | 384 | 60.489 | 0.912 | 72.371 | 1.00 | 13.88 | A |
| ATOM | 2941 | O | ASN | A | 384 | 61.419 | 0.406 | 72.998 | 1.00 | 15.18 | A |
| ATOM | 2942 | N | ASP | A | 385 | 59.553 | 1.663 | 72.940 | 1.00 | 13.65 | A |
| ATOM | 2943 | CA | ASP | A | 385 | 59.544 | 1.941 | 74.372 | 1.00 | 16.24 | A |
| ATOM | 2944 | CB | ASP | A | 385 | 59.674 | 3.447 | 74.612 | 1.00 | 16.61 | A |
| ATOM | 2945 | CG | ASP | A | 385 | 58.479 | 4.229 | 74.089 | 1.00 | 16.68 | A |
| ATOM | 2946 | OD1 | ASP | A | 385 | 57.585 | 3.624 | 73.463 | 1.00 | 17.34 | A |
| ATOM | 2947 | OD2 | ASP | A | 385 | 58.431 | 5.460 | 74.305 | 1.00 | 19.27 | A |
| ATOM | 2948 | C | ASP | A | 385 | 58.223 | 1.441 | 74.946 | 1.00 | 17.71 | A |
| ATOM | 2949 | O | ASP | A | 385 | 57.729 | 1.958 | 75.949 | 1.00 | 19.06 | A |
| ATOM | 2950 | N | GLY | A | 386 | 57.669 | 0.423 | 74.299 | 1.00 | 18.40 | A |
| ATOM | 2951 | CA | GLY | A | 386 | 56.392 | −0.130 | 74.707 | 1.00 | 18.48 | A |
| ATOM | 2952 | C | GLY | A | 386 | 55.459 | 0.094 | 73.535 | 1.00 | 19.00 | A |
| ATOM | 2953 | O | GLY | A | 386 | 54.467 | −0.611 | 73.355 | 1.00 | 21.68 | A |
| ATOM | 2954 | N | GLY | A | 387 | 55.802 | 1.100 | 72.734 | 1.00 | 18.08 | A |
| ATOM | 2955 | CA | GLY | A | 387 | 55.035 | 1.440 | 71.550 | 1.00 | 16.98 | A |
| ATOM | 2956 | C | GLY | A | 387 | 55.999 | 1.638 | 70.393 | 1.00 | 16.72 | A |
| ATOM | 2957 | O | GLY | A | 387 | 57.204 | 1.765 | 70.609 | 1.00 | 15.28 | A |
| ATOM | 2958 | N | HIS | A | 388 | 55.476 | 1.666 | 69.172 | 1.00 | 16.68 | A |
| ATOM | 2959 | CA | HIS | A | 388 | 56.301 | 1.836 | 67.978 | 1.00 | 16.60 | A |
| ATOM | 2960 | CB | HIS | A | 388 | 55.592 | 1.214 | 66.774 | 1.00 | 17.24 | A |
| ATOM | 2961 | CG | HIS | A | 388 | 55.292 | −0.243 | 66.941 | 1.00 | 18.58 | A |
| ATOM | 2962 | CD2 | HIS | A | 388 | 54.118 | −0.919 | 66.933 | 1.00 | 20.08 | A |
| ATOM | 2963 | ND1 | HIS | A | 388 | 56.274 | −1.185 | 67.162 | 1.00 | 19.33 | A |
| ATOM | 2964 | CE1 | HIS | A | 388 | 55.718 | −2.377 | 67.284 | 1.00 | 20.39 | A |
| ATOM | 2965 | NE2 | HIS | A | 388 | 54.411 | −2.244 | 67.148 | 1.00 | 19.69 | A |
| ATOM | 2966 | C | HIS | A | 388 | 56.592 | 3.308 | 67.711 | 1.00 | 16.64 | A |
| ATOM | 2967 | O | HIS | A | 388 | 55.676 | 4.101 | 67.494 | 1.00 | 17.51 | A |
| ATOM | 2968 | N | LYS | A | 389 | 57.870 | 3.674 | 67.713 | 1.00 | 15.35 | A |
| ATOM | 2969 | CA | LYS | A | 389 | 58.236 | 5.065 | 67.496 | 1.00 | 14.37 | A |
| ATOM | 2970 | CB | LYS | A | 389 | 58.611 | 5.728 | 68.824 | 1.00 | 18.01 | A |
| ATOM | 2971 | CG | LYS | A | 389 | 57.428 | 6.027 | 69.745 | 1.00 | 21.05 | A |
| ATOM | 2972 | CD | LYS | A | 389 | 57.027 | 4.838 | 70.598 | 1.00 | 28.27 | A |
| ATOM | 2973 | CE | LYS | A | 389 | 55.951 | 5.245 | 71.600 | 1.00 | 29.39 | A |
| ATOM | 2974 | NZ | LYS | A | 389 | 56.369 | 6.429 | 72.413 | 1.00 | 30.94 | A |
| ATOM | 2975 | C | LYS | A | 389 | 59.362 | 5.294 | 66.505 | 1.00 | 13.39 | A |
| ATOM | 2976 | O | LYS | A | 389 | 60.287 | 4.493 | 66.387 | 1.00 | 15.06 | A |
| ATOM | 2977 | N | GLY | A | 390 | 59.268 | 6.406 | 65.789 | 1.00 | 11.07 | A |
| ATOM | 2978 | CA | GLY | A | 390 | 60.295 | 6.752 | 64.828 | 1.00 | 9.93 | A |
| ATOM | 2979 | C | GLY | A | 390 | 61.328 | 7.645 | 65.494 | 1.00 | 10.15 | A |
| ATOM | 2980 | O | GLY | A | 390 | 61.015 | 8.364 | 66.438 | 1.00 | 9.87 | A |
| ATOM | 2981 | N | SER | A | 391 | 62.567 | 7.584 | 65.021 | 1.00 | 8.97 | A |
| ATOM | 2982 | CA | SER | A | 391 | 63.632 | 8.412 | 65.571 | 1.00 | 10.24 | A |
| ATOM | 2983 | CB | SER | A | 391 | 64.655 | 7.558 | 66.321 | 1.00 | 11.10 | A |
| ATOM | 2984 | OG | SER | A | 391 | 65.757 | 8.353 | 66.744 | 1.00 | 11.89 | A |
| ATOM | 2985 | C | SER | A | 391 | 64.318 | 9.116 | 64.415 | 1.00 | 10.49 | A |
| ATOM | 2986 | O | SER | A | 391 | 64.615 | 8.493 | 63.400 | 1.00 | 12.83 | A |
| ATOM | 2987 | N | GLY | A | 392 | 64.565 | 10.413 | 64.568 | 1.00 | 9.56 | A |
| ATOM | 2988 | CA | GLY | A | 392 | 65.212 | 11.165 | 63.510 | 1.00 | 8.47 | A |
| ATOM | 2989 | C | GLY | A | 392 | 64.192 | 11.864 | 62.636 | 1.00 | 8.75 | A |
| ATOM | 2990 | O | GLY | A | 392 | 63.161 | 12.315 | 63.133 | 1.00 | 9.40 | A |
| ATOM | 2991 | N | ALA | A | 393 | 64.457 | 11.933 | 61.334 | 1.00 | 7.96 | A |
| ATOM | 2992 | CA | ALA | A | 393 | 63.541 | 12.601 | 60.420 | 1.00 | 8.07 | A |
| ATOM | 2993 | CB | ALA | A | 393 | 63.891 | 14.079 | 60.350 | 1.00 | 9.80 | A |
| ATOM | 2994 | C | ALA | A | 393 | 63.522 | 12.035 | 59.007 | 1.00 | 8.57 | A |
| ATOM | 2995 | O | ALA | A | 393 | 64.517 | 11.488 | 58.535 | 1.00 | 8.76 | A |
| ATOM | 2996 | N | GLN | A | 394 | 62.366 | 12.163 | 58.357 | 1.00 | 7.32 | A |
| ATOM | 2997 | CA | GLN | A | 394 | 62.194 | 11.768 | 56.965 | 1.00 | 7.06 | A |
| ATOM | 2998 | CB | GLN | A | 394 | 60.814 | 11.141 | 56.739 | 1.00 | 7.43 | A |
| ATOM | 2999 | CG | GLN | A | 394 | 60.664 | 9.736 | 57.325 | 1.00 | 6.83 | A |
| ATOM | 3000 | CD | GLN | A | 394 | 61.157 | 8.640 | 56.382 | 1.00 | 8.50 | A |
| ATOM | 3001 | OE1 | GLN | A | 394 | 62.240 | 8.751 | 55.784 | 1.00 | 9.08 | A |
| ATOM | 3002 | NE2 | GLN | A | 394 | 60.369 | 7.567 | 56.254 | 1.00 | 8.25 | A |
| ATOM | 3003 | C | GLN | A | 394 | 62.306 | 13.100 | 56.224 | 1.00 | 7.64 | A |
| ATOM | 3004 | O | GLN | A | 394 | 61.983 | 14.158 | 56.780 | 1.00 | 7.42 | A |
| ATOM | 3005 | N | VAL | A | 395 | 62.753 | 13.051 | 54.974 | 1.00 | 6.98 | A |
| ATOM | 3006 | CA | VAL | A | 395 | 62.952 | 14.262 | 54.188 | 1.00 | 6.45 | A |
| ATOM | 3007 | CB | VAL | A | 395 | 64.459 | 14.467 | 53.885 | 1.00 | 7.41 | A |
| ATOM | 3008 | CG1 | VAL | A | 395 | 64.668 | 15.771 | 53.134 | 1.00 | 8.08 | A |
| ATOM | 3009 | CG2 | VAL | A | 395 | 65.264 | 14.462 | 55.179 | 1.00 | 9.77 | A |
| ATOM | 3010 | C | VAL | A | 395 | 62.212 | 14.304 | 52.860 | 1.00 | 7.43 | A |
| ATOM | 3011 | O | VAL | A | 395 | 62.147 | 13.309 | 52.139 | 1.00 | 8.60 | A |
| ATOM | 3012 | N | GLU | A | 396 | 61.651 | 15.470 | 52.555 | 1.00 | 7.12 | A |
| ATOM | 3013 | CA | GLU | A | 396 | 60.965 | 15.705 | 51.291 | 1.00 | 7.54 | A |
| ATOM | 3014 | CB | GLU | A | 396 | 59.506 | 16.107 | 51.506 | 1.00 | 8.08 | A |
| ATOM | 3015 | CG | GLU | A | 396 | 58.824 | 16.551 | 50.218 | 1.00 | 8.65 | A |
| ATOM | 3016 | CD | GLU | A | 396 | 57.489 | 17.247 | 50.458 | 1.00 | 8.77 | A |
| ATOM | 3017 | OE1 | GLU | A | 396 | 56.507 | 16.562 | 50.800 | 1.00 | 11.22 | A |
| ATOM | 3018 | OE2 | GLU | A | 396 | 57.439 | 18.488 | 50.318 | 1.00 | 9.67 | A |
| ATOM | 3019 | C | GLU | A | 396 | 61.692 | 16.865 | 50.616 | 1.00 | 7.79 | A |
| ATOM | 3020 | O | GLU | A | 396 | 61.993 | 17.872 | 51.260 | 1.00 | 8.29 | A |
| ATOM | 3021 | N | VAL | A | 397 | 61.967 | 16.721 | 49.326 | 1.00 | 6.54 | A |
| ATOM | 3022 | CA | VAL | A | 397 | 62.640 | 17.766 | 48.564 | 1.00 | 8.14 | A |
| ATOM | 3023 | CB | VAL | A | 397 | 64.010 | 17.287 | 48.027 | 1.00 | 7.92 | A |
| ATOM | 3024 | CG1 | VAL | A | 397 | 64.690 | 18.409 | 47.253 | 1.00 | 10.06 | A |
| ATOM | 3025 | CG2 | VAL | A | 397 | 64.887 | 16.829 | 49.178 | 1.00 | 8.87 | A |
| ATOM | 3026 | C | VAL | A | 397 | 61.777 | 18.183 | 47.375 | 1.00 | 8.52 | A |
| ATOM | 3027 | O | VAL | A | 397 | 61.107 | 17.356 | 46.757 | 1.00 | 8.62 | A |
| ATOM | 3028 | N | GLY | A | 398 | 61.789 | 19.476 | 47.078 | 1.00 | 7.67 | A |
| ATOM | 3029 | CA | GLY | A | 398 | 61.031 | 19.997 | 45.957 | 1.00 | 8.58 | A |
| ATOM | 3030 | C | GLY | A | 398 | 61.762 | 21.193 | 45.376 | 1.00 | 9.63 | A |
| ATOM | 3031 | O | GLY | A | 398 | 62.871 | 21.528 | 45.809 | 1.00 | 9.53 | A |
| ATOM | 3032 | N | SER | A | 399 | 61.144 | 21.863 | 44.410 | 1.00 | 10.22 | A |
| ATOM | 3033 | CA | SER | A | 399 | 61.784 | 23.015 | 43.780 | 1.00 | 9.98 | A |
| ATOM | 3034 | CB | SER | A | 399 | 61.170 | 23.278 | 42.399 | 1.00 | 12.91 | A |
| ATOM | 3035 | OG | SER | A | 399 | 59.797 | 23.624 | 42.494 | 1.00 | 18.19 | A |
| ATOM | 3036 | C | SER | A | 399 | 61.699 | 24.286 | 44.620 | 1.00 | 9.21 | A |
| ATOM | 3037 | O | SER | A | 399 | 62.296 | 25.302 | 44.272 | 1.00 | 9.35 | A |
| ATOM | 3038 | N | LEU | A | 400 | 60.964 | 24.231 | 45.724 | 1.00 | 9.96 | A |
| ATOM | 3039 | CA | LEU | A | 400 | 60.825 | 25.396 | 46.587 | 1.00 | 10.61 | A |
| ATOM | 3040 | CB | LEU | A | 400 | 59.359 | 25.838 | 46.659 | 1.00 | 14.05 | A |
| ATOM | 3041 | CG | LEU | A | 400 | 58.764 | 26.649 | 45.501 | 1.00 | 16.92 | A |
| ATOM | 3042 | CD1 | LEU | A | 400 | 58.850 | 25.876 | 44.199 | 1.00 | 21.32 | A |
| ATOM | 3043 | CD2 | LEU | A | 400 | 57.312 | 26.977 | 45.825 | 1.00 | 19.57 | A |
| ATOM | 3044 | C | LEU | A | 400 | 61.330 | 25.157 | 48.005 | 1.00 | 9.25 | A |
| ATOM | 3045 | O | LEU | A | 400 | 62.105 | 25.951 | 48.545 | 1.00 | 9.14 | A |
| ATOM | 3046 | N | ASN | A | 401 | 60.890 | 24.057 | 48.606 | 1.00 | 8.52 | A |
| ATOM | 3047 | CA | ASN | A | 401 | 61.267 | 23.758 | 49.980 | 1.00 | 7.77 | A |
| ATOM | 3048 | CB | ASN | A | 401 | 60.043 | 23.835 | 50.897 | 1.00 | 10.84 | A |
| ATOM | 3049 | CG | ASN | A | 401 | 59.229 | 25.081 | 50.693 | 1.00 | 9.12 | A |
| ATOM | 3050 | OD1 | ASN | A | 401 | 58.034 | 25.006 | 50.386 | 1.00 | 14.79 | A |
| ATOM | 3051 | ND2 | ASN | A | 401 | 59.849 | 26.227 | 50.861 | 1.00 | 9.75 | A |
| ATOM | 3052 | C | ASN | A | 401 | 61.878 | 22.396 | 50.236 | 1.00 | 8.00 | A |
| ATOM | 3053 | O | ASN | A | 401 | 61.842 | 21.487 | 49.404 | 1.00 | 8.54 | A |
| ATOM | 3054 | N | ILE | A | 402 | 62.443 | 22.287 | 51.431 | 1.00 | 8.67 | A |
| ATOM | 3055 | CA | ILE | A | 402 | 62.964 | 21.036 | 51.942 | 1.00 | 7.98 | A |
| ATOM | 3056 | CB | ILE | A | 402 | 64.416 | 21.142 | 52.433 | 1.00 | 8.17 | A |
| ATOM | 3057 | CG2 | ILE | A | 402 | 64.769 | 19.907 | 53.266 | 1.00 | 9.05 | A |
| ATOM | 3058 | CG1 | ILE | A | 402 | 65.365 | 21.250 | 51.238 | 1.00 | 8.43 | A |
| ATOM | 3059 | CD1 | ILE | A | 402 | 66.807 | 21.602 | 51.618 | 1.00 | 7.77 | A |
| ATOM | 3060 | C | ILE | A | 402 | 62.053 | 20.906 | 53.157 | 1.00 | 8.31 | A |
| ATOM | 3061 | O | ILE | A | 402 | 61.934 | 21.844 | 53.943 | 1.00 | 8.51 | A |
| ATOM | 3062 | N | ARG | A | 403 | 61.376 | 19.777 | 53.293 | 1.00 | 7.72 | A |
| ATOM | 3063 | CA | ARG | A | 403 | 60.505 | 19.588 | 54.439 | 1.00 | 7.17 | A |
| ATOM | 3064 | CB | ARG | A | 403 | 59.050 | 19.392 | 53.991 | 1.00 | 8.87 | A |
| ATOM | 3065 | CG | ARG | A | 403 | 58.465 | 20.607 | 53.267 | 1.00 | 9.01 | A |
| ATOM | 3066 | CD | ARG | A | 403 | 56.942 | 20.656 | 53.393 | 1.00 | 9.39 | A |
| ATOM | 3067 | NE | ARG | A | 403 | 56.298 | 19.541 | 52.704 | 1.00 | 8.67 | A |
| ATOM | 3068 | CZ | ARG | A | 403 | 55.019 | 19.209 | 52.851 | 1.00 | 9.77 | A |
| ATOM | 3069 | NH1 | ARG | A | 403 | 54.239 | 19.911 | 53.670 | 1.00 | 10.32 | A |
| ATOM | 3070 | NH2 | ARG | A | 403 | 54.520 | 18.174 | 52.184 | 1.00 | 10.43 | A |
| ATOM | 3071 | C | ARG | A | 403 | 60.997 | 18.401 | 55.253 | 1.00 | 7.71 | A |
| ATOM | 3072 | O | ARG | A | 403 | 61.415 | 17.380 | 54.700 | 1.00 | 9.23 | A |
| ATOM | 3073 | N | LEU | A | 404 | 60.965 | 18.550 | 56.572 | 1.00 | 7.31 | A |
| ATOM | 3074 | CA | LEU | A | 404 | 61.427 | 17.499 | 57.472 | 1.00 | 7.81 | A |
| ATOM | 3075 | CB | LEU | A | 404 | 62.590 | 18.006 | 58.327 | 1.00 | 9.25 | A |
| ATOM | 3076 | CG | LEU | A | 404 | 63.738 | 18.744 | 57.641 | 1.00 | 9.01 | A |
| ATOM | 3077 | CD1 | LEU | A | 404 | 64.687 | 19.280 | 58.706 | 1.00 | 10.98 | A |
| ATOM | 3078 | CD2 | LEU | A | 404 | 64.468 | 17.802 | 56.693 | 1.00 | 10.79 | A |
| ATOM | 3079 | C | LEU | A | 404 | 60.324 | 17.050 | 58.410 | 1.00 | 7.42 | A |
| ATOM | 3080 | O | LEU | A | 404 | 59.625 | 17.869 | 58.999 | 1.00 | 9.26 | A |
| ATOM | 3081 | N | GLY | A | 405 | 60.179 | 15.742 | 58.545 | 1.00 | 6.70 | A |
| ATOM | 3082 | CA | GLY | A | 405 | 59.186 | 15.207 | 59.451 | 1.00 | 7.41 | A |
| ATOM | 3083 | C | GLY | A | 405 | 59.910 | 14.480 | 60.567 | 1.00 | 8.30 | A |
| ATOM | 3084 | O | GLY | A | 405 | 60.611 | 13.498 | 60.317 | 1.00 | 7.86 | A |
| ATOM | 3085 | N | THR | A | 406 | 59.758 | 14.954 | 61.800 | 1.00 | 8.02 | A |
| ATOM | 3086 | CA | THR | A | 406 | 60.432 | 14.324 | 62.932 | 1.00 | 8.05 | A |
| ATOM | 3087 | CB | THR | A | 406 | 60.771 | 15.351 | 64.034 | 1.00 | 8.47 | A |
| ATOM | 3088 | OG1 | THR | A | 406 | 59.575 | 16.034 | 64.438 | 1.00 | 7.99 | A |
| ATOM | 3089 | CG2 | THR | A | 406 | 61.796 | 16.347 | 63.529 | 1.00 | 7.16 | A |
| ATOM | 3090 | C | THR | A | 406 | 59.648 | 13.193 | 63.585 | 1.00 | 7.79 | A |
| ATOM | 3091 | O | THR | A | 406 | 58.418 | 13.164 | 63.544 | 1.00 | 7.98 | A |
| ATOM | 3092 | N | GLY | A | 407 | 60.379 | 12.259 | 64.187 | 1.00 | 8.13 | A |
| ATOM | 3093 | CA | GLY | A | 407 | 59.749 | 11.133 | 64.861 | 1.00 | 7.74 | A |
| ATOM | 3094 | C | GLY | A | 407 | 59.466 | 11.446 | 66.322 | 1.00 | 8.63 | A |
| ATOM | 3095 | O | GLY | A | 407 | 59.545 | 12.603 | 66.738 | 1.00 | 9.00 | A |
| ATOM | 3096 | N | ALA | A | 408 | 59.130 | 10.427 | 67.108 | 1.00 | 8.60 | A |
| ATOM | 3097 | CA | ALA | A | 408 | 58.857 | 10.627 | 68.534 | 1.00 | 9.05 | A |
| ATOM | 3098 | CB | ALA | A | 408 | 58.429 | 9.317 | 69.173 | 1.00 | 10.81 | A |
| ATOM | 3099 | C | ALA | A | 408 | 60.113 | 11.164 | 69.216 | 1.00 | 9.33 | A |
| ATOM | 3100 | O | ALA | A | 408 | 60.040 | 11.809 | 70.262 | 1.00 | 10.04 | A |
| ATOM | 3101 | N | ALA | A | 409 | 61.265 | 10.864 | 68.624 | 1.00 | 10.32 | A |
| ATOM | 3102 | CA | ALA | A | 409 | 62.544 | 11.357 | 69.111 | 1.00 | 9.43 | A |
| ATOM | 3103 | CB | ALA | A | 409 | 63.459 | 10.204 | 69.507 | 1.00 | 9.87 | A |
| ATOM | 3104 | C | ALA | A | 409 | 63.118 | 12.089 | 67.907 | 1.00 | 9.13 | A |
| ATOM | 3105 | O | ALA | A | 409 | 63.028 | 11.586 | 66.779 | 1.00 | 9.16 | A |
| ATOM | 3106 | N | VAL | A | 410 | 63.666 | 13.282 | 68.116 | 1.00 | 7.88 | A |
| ATOM | 3107 | CA | VAL | A | 410 | 64.260 | 14.017 | 67.001 | 1.00 | 7.82 | A |
| ATOM | 3108 | CB | VAL | A | 410 | 64.463 | 15.515 | 67.332 | 1.00 | 8.46 | A |
| ATOM | 3109 | CG1 | VAL | A | 410 | 63.096 | 16.218 | 67.387 | 1.00 | 10.90 | A |
| ATOM | 3110 | CG2 | VAL | A | 410 | 65.190 | 15.679 | 68.654 | 1.00 | 9.28 | A |
| ATOM | 3111 | C | VAL | A | 410 | 65.592 | 13.350 | 66.650 | 1.00 | 8.07 | A |
| ATOM | 3112 | O | VAL | A | 410 | 66.117 | 13.521 | 65.550 | 1.00 | 8.17 | A |
| ATOM | 3113 | N | TRP | A | 411 | 66.126 | 12.589 | 67.603 | 1.00 | 8.05 | A |
| ATOM | 3114 | CA | TRP | A | 411 | 67.355 | 11.816 | 67.407 | 1.00 | 7.81 | A |
| ATOM | 3115 | CB | TRP | A | 411 | 68.600 | 12.701 | 67.317 | 1.00 | 7.77 | A |
| ATOM | 3116 | CG | TRP | A | 411 | 69.780 | 11.955 | 66.701 | 1.00 | 7.86 | A |
| ATOM | 3117 | CD2 | TRP | A | 411 | 70.818 | 11.228 | 67.394 | 1.00 | 8.24 | A |
| ATOM | 3118 | CE2 | TRP | A | 411 | 71.657 | 10.657 | 66.413 | 1.00 | 7.88 | A |
| ATOM | 3119 | CE3 | TRP | A | 411 | 71.120 | 11.011 | 68.746 | 1.00 | 9.42 | A |
| ATOM | 3120 | CD1 | TRP | A | 411 | 70.030 | 11.790 | 65.371 | 1.00 | 8.58 | A |
| ATOM | 3121 | NE1 | TRP | A | 411 | 71.153 | 11.014 | 65.188 | 1.00 | 7.92 | A |
| ATOM | 3122 | CZ2 | TRP | A | 411 | 72.776 | 9.877 | 66.734 | 1.00 | 8.39 | A |
| ATOM | 3123 | CZ3 | TRP | A | 411 | 72.236 | 10.233 | 69.070 | 1.00 | 8.98 | A |
| ATOM | 3124 | CH2 | TRP | A | 411 | 73.051 | 9.679 | 68.062 | 1.00 | 8.18 | A |
| ATOM | 3125 | C | TRP | A | 411 | 67.529 | 10.868 | 68.586 | 1.00 | 8.79 | A |
| ATOM | 3126 | O | TRP | A | 411 | 67.090 | 11.164 | 69.697 | 1.00 | 9.25 | A |
| ATOM | 3127 | N | GLY | A | 412 | 68.165 | 9.729 | 68.338 | 1.00 | 8.48 | A |
| ATOM | 3128 | CA | GLY | A | 412 | 68.400 | 8.768 | 69.405 | 1.00 | 8.74 | A |
| ATOM | 3129 | C | GLY | A | 412 | 67.480 | 7.561 | 69.405 | 1.00 | 10.34 | A |
| ATOM | 3130 | O | GLY | A | 412 | 66.286 | 7.682 | 69.137 | 1.00 | 11.44 | A |
| ATOM | 3131 | N | THR | A | 413 | 68.047 | 6.396 | 69.718 | 1.00 | 9.68 | A |
| ATOM | 3132 | CA | THR | A | 413 | 67.302 | 5.139 | 69.754 | 1.00 | 9.98 | A |
| ATOM | 3133 | CB | THR | A | 413 | 67.557 | 4.301 | 68.495 | 1.00 | 9.30 | A |
| ATOM | 3134 | OG1 | THR | A | 413 | 68.928 | 3.878 | 68.482 | 1.00 | 9.87 | A |
| ATOM | 3135 | CG2 | THR | A | 413 | 67.263 | 5.117 | 67.236 | 1.00 | 9.15 | A |
| ATOM | 3136 | C | THR | A | 413 | 67.712 | 4.273 | 70.941 | 1.00 | 10.18 | A |
| ATOM | 3137 | O | THR | A | 413 | 66.932 | 3.445 | 71.417 | 1.00 | 10.62 | A |
| ATOM | 3138 | N | GLY | A | 414 | 68.948 | 4.460 | 71.396 | 1.00 | 10.13 | A |
| ATOM | 3139 | CA | GLY | A | 414 | 69.467 | 3.683 | 72.508 | 1.00 | 10.43 | A |
| ATOM | 3140 | C | GLY | A | 414 | 70.078 | 2.356 | 72.078 | 1.00 | 10.62 | A |
| ATOM | 3141 | O | GLY | A | 414 | 70.536 | 1.590 | 72.925 | 1.00 | 10.85 | A |
| ATOM | 3142 | N | TYR | A | 415 | 70.118 | 2.095 | 70.770 | 1.00 | 9.03 | A |
| ATOM | 3143 | CA | TYR | A | 415 | 70.637 | 0.829 | 70.251 | 1.00 | 8.92 | A |
| ATOM | 3144 | CB | TYR | A | 415 | 69.675 | 0.262 | 69.197 | 1.00 | 8.96 | A |
| ATOM | 3145 | CG | TYR | A | 415 | 68.229 | 0.143 | 69.630 | 1.00 | 9.94 | A |
| ATOM | 3146 | CD1 | TYR | A | 415 | 67.892 | −0.261 | 70.923 | 1.00 | 11.30 | A |
| ATOM | 3147 | CE1 | TYR | A | 415 | 66.554 | −0.427 | 71.298 | 1.00 | 11.48 | A |
| ATOM | 3148 | CD2 | TYR | A | 415 | 67.192 | 0.383 | 68.722 | 1.00 | 10.18 | A |
| ATOM | 3149 | CE2 | TYR | A | 415 | 65.857 | 0.218 | 69.090 | 1.00 | 12.20 | A |
| ATOM | 3150 | CZ | TYR | A | 415 | 65.549 | −0.188 | 70.376 | 1.00 | 12.14 | A |
| ATOM | 3151 | OH | TYR | A | 415 | 64.230 | −0.362 | 70.736 | 1.00 | 12.58 | A |
| ATOM | 3152 | C | TYR | A | 415 | 72.038 | 0.833 | 69.633 | 1.00 | 8.59 | A |
| ATOM | 3153 | O | TYR | A | 415 | 72.476 | −0.195 | 69.115 | 1.00 | 9.20 | A |
| ATOM | 3154 | N | PHE | A | 416 | 72.747 | 1.956 | 69.693 | 1.00 | 8.57 | A |
| ATOM | 3155 | CA | PHE | A | 416 | 74.071 | 2.030 | 69.073 | 1.00 | 8.50 | A |
| ATOM | 3156 | CB | PHE | A | 416 | 74.004 | 3.001 | 67.894 | 1.00 | 8.09 | A |
| ATOM | 3157 | CG | PHE | A | 416 | 73.016 | 2.585 | 66.842 | 1.00 | 7.52 | A |
| ATOM | 3158 | CD1 | PHE | A | 416 | 73.421 | 1.814 | 65.757 | 1.00 | 7.15 | A |
| ATOM | 3159 | CD2 | PHE | A | 416 | 71.668 | 2.914 | 66.965 | 1.00 | 8.25 | A |
| ATOM | 3160 | CE1 | PHE | A | 416 | 72.495 | 1.372 | 64.811 | 1.00 | 6.64 | A |
| ATOM | 3161 | CE2 | PHE | A | 416 | 70.734 | 2.478 | 66.027 | 1.00 | 8.11 | A |
| ATOM | 3162 | CZ | PHE | A | 416 | 71.148 | 1.704 | 64.944 | 1.00 | 7.54 | A |
| ATOM | 3163 | C | PHE | A | 416 | 75.190 | 2.406 | 70.037 | 1.00 | 8.52 | A |
| ATOM | 3164 | O | PHE | A | 416 | 75.044 | 3.304 | 70.864 | 1.00 | 9.20 | A |
| ATOM | 3165 | N | GLY | A | 417 | 76.316 | 1.711 | 69.899 | 1.00 | 7.63 | A |
| ATOM | 3166 | CA | GLY | A | 417 | 77.461 | 1.912 | 70.773 | 1.00 | 7.52 | A |
| ATOM | 3167 | C | GLY | A | 417 | 77.996 | 3.308 | 71.032 | 1.00 | 8.54 | A |
| ATOM | 3168 | O | GLY | A | 417 | 78.092 | 4.136 | 70.129 | 1.00 | 8.49 | A |
| ATOM | 3169 | N | GLY | A | 418 | 78.334 | 3.552 | 72.296 | 1.00 | 9.04 | A |
| ATOM | 3170 | CA | GLY | A | 418 | 78.915 | 4.813 | 72.719 | 1.00 | 8.60 | A |
| ATOM | 3171 | C | GLY | A | 418 | 78.116 | 6.102 | 72.679 | 1.00 | 10.22 | A |
| ATOM | 3172 | O | GLY | A | 418 | 77.930 | 6.743 | 73.713 | 1.00 | 12.32 | A |
| ATOM | 3173 | N | ILE | A | 419 | 77.645 | 6.486 | 71.497 | 1.00 | 9.61 | A |
| ATOM | 3174 | CA | ILE | A | 419 | 76.911 | 7.740 | 71.341 | 1.00 | 9.48 | A |
| ATOM | 3175 | CB | ILE | A | 419 | 77.377 | 8.485 | 70.060 | 1.00 | 9.33 | A |
| ATOM | 3176 | CG2 | ILE | A | 419 | 78.848 | 8.867 | 70.186 | 1.00 | 10.84 | A |
| ATOM | 3177 | CG1 | ILE | A | 419 | 77.154 | 7.601 | 68.826 | 1.00 | 10.51 | A |
| ATOM | 3178 | CD1 | ILE | A | 419 | 77.472 | 8.303 | 67.499 | 1.00 | 10.39 | A |
| ATOM | 3179 | C | ILE | A | 419 | 75.385 | 7.668 | 71.319 | 1.00 | 9.17 | A |
| ATOM | 3180 | O | ILE | A | 419 | 74.723 | 8.702 | 71.270 | 1.00 | 11.12 | A |
| ATOM | 3181 | N | ASP | A | 420 | 74.821 | 6.467 | 71.371 | 1.00 | 8.72 | A |
| ATOM | 3182 | CA | ASP | A | 420 | 73.367 | 6.316 | 71.328 | 1.00 | 8.03 | A |
| ATOM | 3183 | CB | ASP | A | 420 | 72.910 | 6.369 | 69.862 | 1.00 | 9.07 | A |
| ATOM | 3184 | CG | ASP | A | 420 | 71.414 | 6.262 | 69.714 | 1.00 | 8.63 | A |
| ATOM | 3185 | OD1 | ASP | A | 420 | 70.702 | 6.686 | 70.650 | 1.00 | 10.27 | A |
| ATOM | 3186 | OD2 | ASP | A | 420 | 70.942 | 5.777 | 68.659 | 1.00 | 8.69 | A |
| ATOM | 3187 | C | ASP | A | 420 | 73.040 | 4.969 | 71.956 | 1.00 | 9.79 | A |
| ATOM | 3188 | O | ASP | A | 420 | 72.251 | 4.186 | 71.425 | 1.00 | 8.86 | A |
| ATOM | 3189 | N | ASN | A | 421 | 73.650 | 4.736 | 73.115 | 1.00 | 10.20 | A |
| ATOM | 3190 | CA | ASN | A | 421 | 73.558 | 3.472 | 73.836 | 1.00 | 10.71 | A |
| ATOM | 3191 | CB | ASN | A | 421 | 74.977 | 3.047 | 74.216 | 1.00 | 11.44 | A |
| ATOM | 3192 | CG | ASN | A | 421 | 75.701 | 4.112 | 75.021 | 1.00 | 12.30 | A |
| ATOM | 3193 | OD1 | ASN | A | 421 | 75.127 | 5.151 | 75.351 | 1.00 | 14.44 | A |
| ATOM | 3194 | ND2 | ASN | A | 421 | 76.964 | 3.858 | 75.343 | 1.00 | 11.52 | A |
| ATOM | 3195 | C | ASN | A | 421 | 72.672 | 3.355 | 75.071 | 1.00 | 12.59 | A |
| ATOM | 3196 | O | ASN | A | 421 | 72.810 | 2.392 | 75.821 | 1.00 | 13.35 | A |
| ATOM | 3197 | N | SER | A | 422 | 71.774 | 4.304 | 75.302 | 1.00 | 12.19 | A |
| ATOM | 3198 | CA | SER | A | 422 | 70.900 | 4.213 | 76.470 | 1.00 | 13.76 | A |
| ATOM | 3199 | CB | SER | A | 422 | 71.524 | 4.932 | 77.671 | 1.00 | 14.58 | A |
| ATOM | 3200 | OG | SER | A | 422 | 71.672 | 6.320 | 77.418 | 1.00 | 16.38 | A |
| ATOM | 3201 | C | SER | A | 422 | 69.544 | 4.825 | 76.151 | 1.00 | 14.56 | A |
| ATOM | 3202 | O | SER | A | 422 | 69.408 | 5.562 | 75.178 | 1.00 | 14.19 | A |
| ATOM | 3203 | N | ALA | A | 423 | 68.539 | 4.509 | 76.961 | 1.00 | 16.37 | A |
| ATOM | 3204 | CA | ALA | A | 423 | 67.204 | 5.046 | 76.740 | 1.00 | 16.38 | A |
| ATOM | 3205 | CB | ALA | A | 423 | 66.241 | 4.508 | 77.787 | 1.00 | 17.06 | A |
| ATOM | 3206 | C | ALA | A | 423 | 67.276 | 6.565 | 76.822 | 1.00 | 17.06 | A |
| ATOM | 3207 | O | ALA | A | 423 | 66.497 | 7.270 | 76.177 | 1.00 | 16.41 | A |
| ATOM | 3208 | N | THR | A | 424 | 68.229 | 7.059 | 77.606 | 1.00 | 16.50 | A |
| ATOM | 3209 | CA | THR | A | 424 | 68.409 | 8.491 | 77.785 | 1.00 | 17.51 | A |
| ATOM | 3210 | CB | THR | A | 424 | 69.283 | 8.804 | 79.029 | 1.00 | 20.15 | A |
| ATOM | 3211 | OG1 | THR | A | 424 | 69.865 | 10.106 | 78.890 | 1.00 | 26.03 | A |
| ATOM | 3212 | CG2 | THR | A | 424 | 70.377 | 7.782 | 79.195 | 1.00 | 20.47 | A |
| ATOM | 3213 | C | THR | A | 424 | 68.991 | 9.218 | 76.575 | 1.00 | 15.36 | A |
| ATOM | 3214 | O | THR | A | 424 | 69.031 | 10.449 | 76.558 | 1.00 | 16.41 | A |
| ATOM | 3215 | N | THR | A | 425 | 69.447 | 8.480 | 75.564 | 1.00 | 13.05 | A |
| ATOM | 3216 | CA | THR | A | 425 | 69.980 | 9.150 | 74.383 | 1.00 | 11.24 | A |
| ATOM | 3217 | CB | THR | A | 425 | 71.156 | 8.362 | 73.723 | 1.00 | 10.50 | A |
| ATOM | 3218 | OG1 | THR | A | 425 | 70.722 | 7.059 | 73.312 | 1.00 | 12.21 | A |
| ATOM | 3219 | CG2 | THR | A | 425 | 72.325 | 8.231 | 74.710 | 1.00 | 11.98 | A |
| ATOM | 3220 | C | THR | A | 425 | 68.853 | 9.400 | 73.372 | 1.00 | 11.42 | A |
| ATOM | 3221 | O | THR | A | 425 | 69.071 | 9.983 | 72.306 | 1.00 | 11.63 | A |
| ATOM | 3222 | N | ARG | A | 426 | 67.644 | 8.968 | 73.731 | 1.00 | 10.56 | A |
| ATOM | 3223 | CA | ARG | A | 426 | 66.454 | 9.190 | 72.908 | 1.00 | 10.93 | A |
| ATOM | 3224 | CB | ARG | A | 426 | 65.364 | 8.167 | 73.244 | 1.00 | 12.08 | A |
| ATOM | 3225 | CG | ARG | A | 426 | 65.749 | 6.732 | 72.955 | 1.00 | 11.58 | A |
| ATOM | 3226 | CD | ARG | A | 426 | 64.756 | 5.744 | 73.552 | 1.00 | 12.88 | A |
| ATOM | 3227 | NE | ARG | A | 426 | 65.109 | 4.374 | 73.196 | 1.00 | 13.50 | A |
| ATOM | 3228 | CZ | ARG | A | 426 | 64.521 | 3.287 | 73.686 | 1.00 | 14.61 | A |
| ATOM | 3229 | NH1 | ARG | A | 426 | 63.538 | 3.395 | 74.569 | 1.00 | 15.27 | A |
| ATOM | 3230 | NH2 | ARG | A | 426 | 64.919 | 2.086 | 73.288 | 1.00 | 13.37 | A |
| ATOM | 3231 | C | ARG | A | 426 | 65.977 | 10.586 | 73.302 | 1.00 | 11.09 | A |
| ATOM | 3232 | O | ARG | A | 426 | 65.429 | 10.776 | 74.391 | 1.00 | 13.32 | A |
| ATOM | 3233 | N | LEU | A | 427 | 66.180 | 11.559 | 72.421 | 1.00 | 9.86 | A |
| ATOM | 3234 | CA | LEU | A | 427 | 65.803 | 12.934 | 72.717 | 1.00 | 10.69 | A |
| ATOM | 3235 | CB | LEU | A | 427 | 66.999 | 13.861 | 72.465 | 1.00 | 12.74 | A |
| ATOM | 3236 | CG | LEU | A | 427 | 68.282 | 13.555 | 73.249 | 1.00 | 14.12 | A |
| ATOM | 3237 | CD1 | LEU | A | 427 | 69.383 | 14.504 | 72.816 | 1.00 | 15.63 | A |
| ATOM | 3238 | CD2 | LEU | A | 427 | 68.019 | 13.683 | 74.745 | 1.00 | 15.41 | A |
| ATOM | 3239 | C | LEU | A | 427 | 64.592 | 13.430 | 71.937 | 1.00 | 11.83 | A |
| ATOM | 3240 | O | LEU | A | 427 | 64.524 | 13.285 | 70.713 | 1.00 | 11.52 | A |
| ATOM | 3241 | N | ALA | A | 428 | 63.642 | 14.023 | 72.656 | 1.00 | 12.30 | A |
| ATOM | 3242 | CA | ALA | A | 428 | 62.418 | 14.540 | 72.048 | 1.00 | 12.12 | A |
| ATOM | 3243 | CB | ALA | A | 428 | 61.272 | 14.465 | 73.057 | 1.00 | 14.69 | A |
| ATOM | 3244 | C | ALA | A | 428 | 62.561 | 15.967 | 71.523 | 1.00 | 11.50 | A |
| ATOM | 3245 | O | ALA | A | 428 | 61.729 | 16.426 | 70.742 | 1.00 | 12.16 | A |
| ATOM | 3246 | N | THR | A | 429 | 63.608 | 16.666 | 71.954 | 1.00 | 10.46 | A |
| ATOM | 3247 | CA | THR | A | 429 | 63.851 | 18.036 | 71.505 | 1.00 | 10.58 | A |
| ATOM | 3248 | CB | THR | A | 429 | 63.348 | 19.094 | 72.516 | 1.00 | 11.43 | A |
| ATOM | 3249 | OG1 | THR | A | 429 | 64.120 | 19.013 | 73.718 | 1.00 | 12.52 | A |
| ATOM | 3250 | CG2 | THR | A | 429 | 61.886 | 18.873 | 72.843 | 1.00 | 11.43 | A |
| ATOM | 3251 | C | THR | A | 429 | 65.337 | 18.284 | 71.294 | 1.00 | 10.09 | A |
| ATOM | 3252 | O | THR | A | 429 | 66.185 | 17.525 | 71.764 | 1.00 | 10.03 | A |
| ATOM | 3253 | N | GLY | A | 430 | 65.641 | 19.367 | 70.590 | 1.00 | 10.48 | A |
| ATOM | 3254 | CA | GLY | A | 430 | 67.021 | 19.722 | 70.328 | 1.00 | 9.17 | A |
| ATOM | 3255 | C | GLY | A | 430 | 67.097 | 20.709 | 69.186 | 1.00 | 8.68 | A |
| ATOM | 3256 | O | GLY | A | 430 | 66.195 | 21.533 | 69.013 | 1.00 | 8.02 | A |
| ATOM | 3257 | N | TYR | A | 431 | 68.165 | 20.621 | 68.402 | 1.00 | 8.30 | A |
| ATOM | 3258 | CA | TYR | A | 431 | 68.361 | 21.520 | 67.273 | 1.00 | 7.71 | A |
| ATOM | 3259 | CB | TYR | A | 431 | 69.449 | 22.550 | 67.595 | 1.00 | 8.19 | A |
| ATOM | 3260 | CG | TYR | A | 431 | 69.101 | 23.481 | 68.733 | 1.00 | 9.11 | A |
| ATOM | 3261 | CD1 | TYR | A | 431 | 69.238 | 23.078 | 70.060 | 1.00 | 11.58 | A |
| ATOM | 3262 | CE1 | TYR | A | 431 | 68.898 | 23.932 | 71.111 | 1.00 | 12.86 | A |
| ATOM | 3263 | CD2 | TYR | A | 431 | 68.617 | 24.763 | 68.479 | 1.00 | 11.07 | A |
| ATOM | 3264 | CE2 | TYR | A | 431 | 68.274 | 25.624 | 69.521 | 1.00 | 12.45 | A |
| ATOM | 3265 | CZ | TYR | A | 431 | 68.416 | 25.199 | 70.831 | 1.00 | 15.17 | A |
| ATOM | 3266 | OH | TYR | A | 431 | 68.059 | 26.042 | 71.858 | 1.00 | 17.41 | A |
| ATOM | 3267 | C | TYR | A | 431 | 68.766 | 20.759 | 66.018 | 1.00 | 8.24 | A |
| ATOM | 3268 | O | TYR | A | 431 | 69.484 | 19.766 | 66.083 | 1.00 | 7.75 | A |
| ATOM | 3269 | N | TYR | A | 432 | 68.280 | 21.223 | 64.876 | 1.00 | 7.11 | A |
| ATOM | 3270 | CA | TYR | A | 432 | 68.628 | 20.618 | 63.599 | 1.00 | 7.84 | A |
| ATOM | 3271 | CB | TYR | A | 432 | 67.375 | 20.199 | 62.818 | 1.00 | 8.14 | A |
| ATOM | 3272 | CG | TYR | A | 432 | 66.817 | 18.808 | 63.039 | 1.00 | 7.46 | A |
| ATOM | 3273 | CD1 | TYR | A | 432 | 67.357 | 17.929 | 63.980 | 1.00 | 8.46 | A |
| ATOM | 3274 | CE1 | TYR | A | 432 | 66.802 | 16.657 | 64.175 | 1.00 | 7.30 | A |
| ATOM | 3275 | CD2 | TYR | A | 432 | 65.710 | 18.383 | 62.297 | 1.00 | 8.38 | A |
| ATOM | 3276 | CE2 | TYR | A | 432 | 65.150 | 17.124 | 62.481 | 1.00 | 8.45 | A |
| ATOM | 3277 | CZ | TYR | A | 432 | 65.699 | 16.265 | 63.421 | 1.00 | 7.83 | A |
| ATOM | 3278 | OH | TYR | A | 432 | 65.154 | 15.012 | 63.608 | 1.00 | 8.20 | A |
| ATOM | 3279 | C | TYR | A | 432 | 69.333 | 21.677 | 62.758 | 1.00 | 8.13 | A |
| ATOM | 3280 | O | TYR | A | 432 | 69.150 | 22.879 | 62.968 | 1.00 | 8.45 | A |
| ATOM | 3281 | N | ARG | A | 433 | 70.136 | 21.216 | 61.808 | 1.00 | 6.78 | A |
| ATOM | 3282 | CA | ARG | A | 433 | 70.805 | 22.091 | 60.862 | 1.00 | 7.20 | A |
| ATOM | 3283 | CB | ARG | A | 433 | 72.292 | 22.273 | 61.164 | 1.00 | 7.79 | A |
| ATOM | 3284 | CG | ARG | A | 433 | 72.975 | 23.180 | 60.131 | 1.00 | 7.49 | A |
| ATOM | 3285 | CD | ARG | A | 433 | 74.434 | 23.434 | 60.452 | 1.00 | 9.12 | A |
| ATOM | 3286 | NE | ARG | A | 433 | 75.199 | 22.195 | 60.487 | 1.00 | 8.97 | A |
| ATOM | 3287 | CZ | ARG | A | 433 | 76.407 | 22.074 | 61.029 | 1.00 | 10.06 | A |
| ATOM | 3288 | NH1 | ARG | A | 433 | 77.001 | 23.121 | 61.585 | 1.00 | 9.95 | A |
| ATOM | 3289 | NH2 | ARG | A | 433 | 77.012 | 20.891 | 61.034 | 1.00 | 10.36 | A |
| ATOM | 3290 | C | ARG | A | 433 | 70.660 | 21.360 | 59.542 | 1.00 | 8.03 | A |
| ATOM | 3291 | O | ARG | A | 433 | 70.972 | 20.168 | 59.442 | 1.00 | 7.94 | A |
| ATOM | 3292 | N | VAL | A | 434 | 70.165 | 22.066 | 58.536 | 1.00 | 7.32 | A |
| ATOM | 3293 | CA | VAL | A | 434 | 69.979 | 21.488 | 57.216 | 1.00 | 7.15 | A |
| ATOM | 3294 | CB | VAL | A | 434 | 68.543 | 21.734 | 56.694 | 1.00 | 7.41 | A |
| ATOM | 3295 | CG1 | VAL | A | 434 | 68.420 | 21.246 | 55.250 | 1.00 | 8.34 | A |
| ATOM | 3296 | CG2 | VAL | A | 434 | 67.536 | 21.034 | 57.586 | 1.00 | 8.21 | A |
| ATOM | 3297 | C | VAL | A | 434 | 70.947 | 22.133 | 56.244 | 1.00 | 7.54 | A |
| ATOM | 3298 | O | VAL | A | 434 | 71.109 | 23.353 | 56.233 | 1.00 | 8.35 | A |
| ATOM | 3299 | N | ARG | A | 435 | 71.610 | 21.306 | 55.448 | 1.00 | 7.59 | A |
| ATOM | 3300 | CA | ARG | A | 435 | 72.522 | 21.807 | 54.439 | 1.00 | 7.66 | A |
| ATOM | 3301 | CB | ARG | A | 435 | 73.962 | 21.451 | 54.780 | 1.00 | 7.71 | A |
| ATOM | 3302 | CG | ARG | A | 435 | 74.532 | 22.381 | 55.823 | 1.00 | 9.67 | A |
| ATOM | 3303 | CD | ARG | A | 435 | 75.952 | 22.034 | 56.198 | 1.00 | 9.77 | A |
| ATOM | 3304 | NE | ARG | A | 435 | 76.558 | 23.145 | 56.930 | 1.00 | 10.54 | A |
| ATOM | 3305 | CZ | ARG | A | 435 | 77.559 | 23.018 | 57.794 | 1.00 | 11.98 | A |
| ATOM | 3306 | NH1 | ARG | A | 435 | 78.072 | 21.820 | 58.048 | 1.00 | 10.91 | A |
| ATOM | 3307 | NH2 | ARG | A | 435 | 78.053 | 24.094 | 58.390 | 1.00 | 10.66 | A |
| ATOM | 3308 | C | ARG | A | 435 | 72.109 | 21.179 | 53.134 | 1.00 | 7.28 | A |
| ATOM | 3309 | O | ARG | A | 435 | 71.746 | 20.003 | 53.089 | 1.00 | 7.76 | A |
| ATOM | 3310 | N | ALA | A | 436 | 72.131 | 21.973 | 52.073 | 1.00 | 7.02 | A |
| ATOM | 3311 | CA | ALA | A | 436 | 71.742 | 21.465 | 50.774 | 1.00 | 6.65 | A |
| ATOM | 3312 | CB | ALA | A | 436 | 70.318 | 21.920 | 50.439 | 1.00 | 7.58 | A |
| ATOM | 3313 | C | ALA | A | 436 | 72.715 | 21.914 | 49.695 | 1.00 | 7.29 | A |
| ATOM | 3314 | O | ALA | A | 436 | 73.246 | 23.023 | 49.727 | 1.00 | 7.54 | A |
| ATOM | 3315 | N | TRP | A | 437 | 72.959 | 21.013 | 48.753 | 1.00 | 5.95 | A |
| ATOM | 3316 | CA | TRP | A | 437 | 73.852 | 21.272 | 47.639 | 1.00 | 6.21 | A |
| ATOM | 3317 | CB | TRP | A | 437 | 75.053 | 20.322 | 47.668 | 1.00 | 5.98 | A |
| ATOM | 3318 | CG | TRP | A | 437 | 75.998 | 20.521 | 48.806 | 1.00 | 7.30 | A |
| ATOM | 3319 | CD2 | TRP | A | 437 | 75.881 | 19.966 | 50.121 | 1.00 | 6.72 | A |
| ATOM | 3320 | CE2 | TRP | A | 437 | 77.023 | 20.373 | 50.850 | 1.00 | 8.43 | A |
| ATOM | 3321 | CE3 | TRP | A | 437 | 74.916 | 19.172 | 50.760 | 1.00 | 7.33 | A |
| ATOM | 3322 | CD1 | TRP | A | 437 | 77.169 | 21.222 | 48.787 | 1.00 | 8.09 | A |
| ATOM | 3323 | NE1 | TRP | A | 437 | 77.794 | 21.136 | 50.011 | 1.00 | 9.55 | A |
| ATOM | 3324 | CZ2 | TRP | A | 437 | 77.239 | 19.998 | 52.181 | 1.00 | 6.64 | A |
| ATOM | 3325 | CZ3 | TRP | A | 437 | 75.129 | 18.801 | 52.088 | 1.00 | 7.27 | A |
| ATOM | 3326 | CH2 | TRP | A | 437 | 76.284 | 19.220 | 52.783 | 1.00 | 8.05 | A |
| ATOM | 3327 | C | TRP | A | 437 | 73.082 | 21.030 | 46.362 | 1.00 | 7.13 | A |
| ATOM | 3328 | O | TRP | A | 437 | 72.403 | 20.013 | 46.223 | 1.00 | 7.55 | A |
| ATOM | 3329 | N | ILE | A | 438 | 73.170 | 21.973 | 45.433 | 1.00 | 7.28 | A |
| ATOM | 3330 | CA | ILE | A | 438 | 72.494 | 21.805 | 44.155 | 1.00 | 8.68 | A |
| ATOM | 3331 | CB | ILE | A | 438 | 72.713 | 23.024 | 43.246 | 1.00 | 9.03 | A |
| ATOM | 3332 | CG2 | ILE | A | 438 | 72.167 | 22.741 | 41.840 | 1.00 | 8.38 | A |
| ATOM | 3333 | CG1 | ILE | A | 438 | 72.015 | 24.244 | 43.860 | 1.00 | 9.34 | A |
| ATOM | 3334 | CD1 | ILE | A | 438 | 72.348 | 25.559 | 43.164 | 1.00 | 10.41 | A |
| ATOM | 3335 | C | ILE | A | 438 | 73.105 | 20.561 | 43.511 | 1.00 | 10.41 | A |
| ATOM | 3336 | O | ILE | A | 438 | 72.334 | 19.713 | 43.019 | 1.00 | 12.97 | A |
| ATOM | 3337 | OXT | ILE | A | 438 | 74.352 | 20.461 | 43.517 | 1.00 | 11.63 | A |
| ATOM | 3338 | O | HOH | S | 1 | 78.887 | 30.418 | 52.400 | 1.00 | 8.17 | S |
| ATOM | 3339 | O | HOH | S | 2 | 81.058 | 18.434 | 40.151 | 1.00 | 7.63 | S |
| ATOM | 3340 | O | HOH | S | 3 | 75.652 | 33.462 | 45.538 | 1.00 | 10.32 | S |
| ATOM | 3341 | O | HOH | S | 4 | 77.879 | 26.501 | 46.646 | 1.00 | 8.28 | S |
| ATOM | 3342 | O | HOH | S | 5 | 90.143 | 27.507 | 49.065 | 1.00 | 7.73 | S |
| ATOM | 3343 | O | HOH | S | 6 | 71.527 | −2.802 | 69.227 | 1.00 | 9.28 | S |
| ATOM | 3344 | O | HOH | S | 7 | 88.756 | 21.620 | 49.989 | 1.00 | 8.34 | S |
| ATOM | 3345 | O | HOH | S | 8 | 107.021 | 45.889 | 60.497 | 1.00 | 9.11 | S |
| ATOM | 3346 | O | HOH | S | 9 | 62.887 | 28.357 | 47.427 | 1.00 | 10.41 | S |
| ATOM | 3347 | O | HOH | S | 10 | 111.538 | 38.477 | 60.666 | 1.00 | 9.96 | S |
| ATOM | 3348 | O | HOH | S | 11 | 88.626 | 24.835 | 56.146 | 1.00 | 8.12 | S |
| ATOM | 3349 | O | HOH | S | 12 | 70.802 | 13.489 | 56.799 | 1.00 | 9.70 | S |
| ATOM | 3350 | O | HOH | S | 13 | 100.724 | 52.678 | 55.449 | 1.00 | 11.33 | S |
| ATOM | 3351 | O | HOH | S | 14 | 99.364 | 39.633 | 54.622 | 1.00 | 8.43 | S |
| ATOM | 3352 | O | HOH | S | 15 | 93.746 | 25.971 | 49.325 | 1.00 | 9.97 | S |
| ATOM | 3353 | O | HOH | S | 16 | 70.556 | 28.923 | 43.285 | 1.00 | 11.46 | S |
| ATOM | 3354 | O | HOH | S | 17 | 76.361 | 26.032 | 61.109 | 1.00 | 12.39 | S |
| ATOM | 3355 | O | HOH | S | 18 | 92.050 | 49.550 | 53.925 | 1.00 | 11.12 | S |
| ATOM | 3356 | O | HOH | S | 19 | 100.328 | 21.980 | 46.608 | 1.00 | 9.96 | S |
| ATOM | 3357 | O | HOH | S | 20 | 94.849 | 41.474 | 52.182 | 1.00 | 10.32 | S |
| ATOM | 3358 | O | HOH | S | 21 | 70.672 | 13.661 | 49.835 | 1.00 | 9.74 | S |
| ATOM | 3359 | O | HOH | S | 22 | 87.766 | 40.149 | 42.565 | 1.00 | 12.15 | S |
| ATOM | 3360 | O | HOH | S | 23 | 82.479 | 35.400 | 38.409 | 1.00 | 10.23 | S |
| ATOM | 3361 | O | HOH | S | 24 | 100.464 | 26.411 | 57.083 | 1.00 | 10.38 | S |
| ATOM | 3362 | O | HOH | S | 25 | 79.248 | 17.900 | 59.688 | 1.00 | 9.53 | S |
| ATOM | 3363 | O | HOH | S | 26 | 86.844 | 45.646 | 64.249 | 1.00 | 11.15 | S |
| ATOM | 3364 | O | HOH | S | 27 | 83.616 | 21.583 | 38.678 | 1.00 | 10.26 | S |
| ATOM | 3365 | O | HOH | S | 28 | 77.129 | 24.342 | 43.920 | 1.00 | 10.59 | S |
| ATOM | 3366 | O | HOH | S | 29 | 105.863 | 26.381 | 42.966 | 1.00 | 12.30 | S |
| ATOM | 3367 | O | HOH | S | 30 | 117.495 | 51.124 | 62.843 | 1.00 | 10.55 | S |
| ATOM | 3368 | O | HOH | S | 31 | 54.404 | 20.526 | 62.733 | 1.00 | 11.08 | S |
| ATOM | 3369 | O | HOH | S | 32 | 75.471 | 51.494 | 45.429 | 1.00 | 11.69 | S |
| ATOM | 3370 | O | HOH | S | 33 | 80.829 | 19.809 | 51.495 | 1.00 | 11.21 | S |
| ATOM | 3371 | O | HOH | S | 34 | 96.194 | 37.786 | 69.597 | 1.00 | 10.67 | S |
| ATOM | 3372 | O | HOH | S | 35 | 98.313 | 43.962 | 55.858 | 1.00 | 10.55 | S |
| ATOM | 3373 | O | HOH | S | 36 | 94.463 | 39.704 | 49.835 | 1.00 | 11.24 | S |
| ATOM | 3374 | O | HOH | S | 37 | 68.779 | 14.259 | 48.004 | 1.00 | 11.02 | S |
| ATOM | 3375 | O | HOH | S | 38 | 56.135 | 23.328 | 58.621 | 1.00 | 13.34 | S |
| ATOM | 3376 | O | HOH | S | 39 | 77.104 | 19.231 | 57.285 | 1.00 | 9.15 | S |
| ATOM | 3377 | O | HOH | S | 40 | 103.181 | 28.335 | 58.020 | 1.00 | 10.26 | S |
| ATOM | 3378 | O | HOH | S | 41 | 79.454 | 25.874 | 55.646 | 1.00 | 10.59 | S |
| ATOM | 3379 | O | HOH | S | 42 | 81.328 | 30.606 | 64.813 | 1.00 | 13.57 | S |
| ATOM | 3380 | O | HOH | S | 43 | 93.756 | 39.535 | 59.646 | 1.00 | 11.25 | S |
| ATOM | 3381 | O | HOH | S | 44 | 59.094 | 22.247 | 47.290 | 1.00 | 13.18 | S |
| ATOM | 3382 | O | HOH | S | 45 | 73.606 | 9.030 | 62.728 | 1.00 | 13.68 | S |
| ATOM | 3383 | O | HOH | S | 46 | 56.589 | 13.978 | 50.896 | 1.00 | 12.24 | S |
| ATOM | 3384 | O | HOH | S | 47 | 68.293 | 8.673 | 65.595 | 1.00 | 10.37 | S |
| ATOM | 3385 | O | HOH | S | 48 | 70.007 | 20.837 | 36.441 | 1.00 | 11.13 | S |
| ATOM | 3386 | O | HOH | S | 49 | 78.068 | 30.960 | 63.289 | 1.00 | 11.63 | S |
| ATOM | 3387 | O | HOH | S | 50 | 89.483 | 36.920 | 45.417 | 1.00 | 11.34 | S |
| ATOM | 3388 | O | HOH | S | 51 | 90.741 | 43.212 | 41.846 | 1.00 | 13.68 | S |
| ATOM | 3389 | O | HOH | S | 52 | 79.813 | 20.714 | 60.027 | 1.00 | 13.94 | S |
| ATOM | 3390 | O | HOH | S | 53 | 96.362 | 41.199 | 42.441 | 1.00 | 13.46 | S |
| ATOM | 3391 | O | HOH | S | 54 | 59.033 | 25.192 | 59.643 | 1.00 | 12.14 | S |
| ATOM | 3392 | O | HOH | S | 55 | 79.150 | 22.991 | 46.668 | 1.00 | 10.89 | S |
| ATOM | 3393 | O | HOH | S | 56 | 82.730 | 24.862 | 47.007 | 1.00 | 14.93 | S |
| ATOM | 3394 | O | HOH | S | 57 | 64.032 | 25.794 | 42.248 | 1.00 | 13.58 | S |
| ATOM | 3395 | O | HOH | S | 58 | 81.613 | 43.374 | 32.205 | 1.00 | 12.25 | S |
| ATOM | 3396 | O | HOH | S | 59 | 98.547 | 46.318 | 54.177 | 1.00 | 11.72 | S |
| ATOM | 3397 | O | HOH | S | 60 | 87.622 | 43.624 | 39.271 | 1.00 | 13.12 | S |
| ATOM | 3398 | O | HOH | S | 61 | 100.570 | 21.471 | 38.512 | 1.00 | 14.54 | S |
| ATOM | 3399 | O | HOH | S | 62 | 93.225 | 43.622 | 52.360 | 1.00 | 12.97 | S |
| ATOM | 3400 | O | HOH | S | 63 | 69.632 | 8.809 | 63.023 | 1.00 | 12.84 | S |
| ATOM | 3401 | O | HOH | S | 64 | 96.530 | 50.003 | 59.772 | 1.00 | 13.21 | S |
| ATOM | 3402 | O | HOH | S | 65 | 73.860 | 20.281 | 58.597 | 1.00 | 11.01 | S |
| ATOM | 3403 | O | HOH | S | 66 | 75.267 | 32.572 | 53.262 | 1.00 | 12.15 | S |
| ATOM | 3404 | O | HOH | S | 67 | 54.545 | 22.621 | 54.808 | 1.00 | 12.37 | S |
| ATOM | 3405 | O | HOH | S | 68 | 59.233 | 20.246 | 49.145 | 1.00 | 12.82 | S |
| ATOM | 3406 | O | HOH | S | 69 | 97.890 | 25.513 | 55.905 | 1.00 | 16.42 | S |
| ATOM | 3407 | O | HOH | S | 70 | 72.080 | 38.835 | 54.367 | 1.00 | 12.38 | S |
| ATOM | 3408 | O | HOH | S | 71 | 76.348 | 31.142 | 61.163 | 1.00 | 12.33 | S |
| ATOM | 3409 | O | HOH | S | 72 | 109.506 | 27.961 | 55.827 | 1.00 | 17.00 | S |
| ATOM | 3410 | O | HOH | S | 73 | 89.257 | 52.642 | 52.465 | 1.00 | 13.75 | S |
| ATOM | 3411 | O | HOH | S | 74 | 74.953 | 50.170 | 47.925 | 1.00 | 15.51 | S |
| ATOM | 3412 | O | HOH | S | 75 | 80.455 | 45.762 | 34.566 | 1.00 | 14.53 | S |
| ATOM | 3413 | O | HOH | S | 76 | 104.706 | 22.743 | 37.433 | 1.00 | 13.49 | S |
| ATOM | 3414 | O | HOH | S | 77 | 96.218 | 30.556 | 69.691 | 1.00 | 15.12 | S |
| ATOM | 3415 | O | HOH | S | 78 | 76.897 | 21.709 | 44.596 | 1.00 | 13.84 | S |
| ATOM | 3416 | O | HOH | S | 79 | 63.686 | 28.592 | 44.810 | 1.00 | 14.34 | S |
| ATOM | 3417 | O | HOH | S | 80 | 99.656 | 21.916 | 49.418 | 1.00 | 12.14 | S |
| ATOM | 3418 | O | HOH | S | 81 | 84.954 | 34.540 | 70.823 | 1.00 | 16.21 | S |
| ATOM | 3419 | O | HOH | S | 82 | 66.996 | 12.400 | 57.477 | 1.00 | 17.30 | S |
| ATOM | 3420 | O | HOH | S | 83 | 72.166 | 29.579 | 53.068 | 1.00 | 15.24 | S |
| ATOM | 3421 | O | HOH | S | 84 | 98.883 | 35.887 | 72.822 | 1.00 | 15.24 | S |
| ATOM | 3422 | O | HOH | S | 85 | 75.434 | 27.911 | 62.897 | 1.00 | 16.28 | S |
| ATOM | 3423 | O | HOH | S | 86 | 83.017 | 44.309 | 40.793 | 1.00 | 12.54 | S |
| ATOM | 3424 | O | HOH | S | 87 | 81.307 | 38.060 | 64.604 | 1.00 | 16.19 | S |
| ATOM | 3425 | O | HOH | S | 88 | 77.898 | 25.110 | 64.401 | 1.00 | 17.11 | S |
| ATOM | 3426 | O | HOH | S | 89 | 91.770 | 36.855 | 47.001 | 1.00 | 13.96 | S |
| ATOM | 3427 | O | HOH | S | 90 | 82.922 | 46.303 | 38.650 | 1.00 | 13.87 | S |
| ATOM | 3428 | O | HOH | S | 91 | 102.741 | 42.421 | 71.578 | 1.00 | 20.12 | S |
| ATOM | 3429 | O | HOH | S | 92 | 95.880 | 38.670 | 45.527 | 1.00 | 15.72 | S |
| ATOM | 3430 | O | HOH | S | 93 | 91.065 | 32.151 | 40.723 | 1.00 | 15.04 | S |
| ATOM | 3431 | O | HOH | S | 94 | 73.321 | 12.973 | 70.785 | 1.00 | 16.96 | S |
| ATOM | 3432 | O | HOH | S | 95 | 56.405 | 22.759 | 63.006 | 1.00 | 15.18 | S |
| ATOM | 3433 | O | HOH | S | 96 | 94.047 | 30.249 | 38.734 | 1.00 | 17.17 | S |
| ATOM | 3434 | O | HOH | S | 97 | 59.081 | 23.128 | 66.868 | 1.00 | 19.10 | S |
| ATOM | 3435 | O | HOH | S | 98 | 62.165 | 25.956 | 64.912 | 1.00 | 19.39 | S |
| ATOM | 3436 | O | HOH | S | 99 | 80.739 | 39.187 | 68.004 | 1.00 | 16.08 | S |
| ATOM | 3437 | O | HOH | S | 100 | 88.648 | 29.396 | 68.804 | 1.00 | 17.64 | S |
| ATOM | 3438 | O | HOH | S | 101 | 72.654 | −0.361 | 72.840 | 1.00 | 15.35 | S |
| ATOM | 3439 | O | HOH | S | 102 | 59.099 | 17.004 | 71.640 | 1.00 | 17.72 | S |
| ATOM | 3440 | O | HOH | S | 103 | 102.196 | 45.753 | 66.353 | 1.00 | 16.27 | S |
| ATOM | 3441 | O | HOH | S | 104 | 76.876 | 28.286 | 52.642 | 1.00 | 14.09 | S |
| ATOM | 3442 | O | HOH | S | 105 | 84.381 | 45.740 | 67.038 | 1.00 | 17.43 | S |
| ATOM | 3443 | O | HOH | S | 106 | 98.352 | 27.490 | 64.051 | 1.00 | 15.67 | S |
| ATOM | 3444 | O | HOH | S | 107 | 81.673 | 27.498 | 54.735 | 1.00 | 15.10 | S |
| ATOM | 3445 | O | HOH | S | 108 | 95.164 | 43.575 | 42.733 | 1.00 | 17.20 | S |
| ATOM | 3446 | O | HOH | S | 109 | 95.783 | 43.059 | 55.187 | 1.00 | 20.43 | S |
| ATOM | 3447 | O | HOH | S | 110 | 78.029 | 27.599 | 59.480 | 1.00 | 14.41 | S |
| ATOM | 3448 | O | HOH | S | 111 | 95.041 | 44.928 | 69.282 | 1.00 | 16.99 | S |
| ATOM | 3449 | O | HOH | S | 112 | 70.154 | 29.454 | 49.416 | 1.00 | 16.44 | S |
| ATOM | 3450 | O | HOH | S | 113 | 75.591 | 11.156 | 71.736 | 1.00 | 14.60 | S |
| ATOM | 3451 | O | HOH | S | 114 | 71.498 | 26.718 | 72.424 | 1.00 | 19.73 | S |
| ATOM | 3452 | O | HOH | S | 115 | 112.035 | 48.205 | 71.932 | 1.00 | 19.02 | S |
| ATOM | 3453 | O | HOH | S | 116 | 104.550 | 39.046 | 71.261 | 1.00 | 15.78 | S |
| ATOM | 3454 | O | HOH | S | 117 | 74.689 | 26.648 | 58.940 | 1.00 | 17.74 | S |
| ATOM | 3455 | O | HOH | S | 118 | 71.968 | 44.167 | 56.624 | 1.00 | 16.21 | S |
| ATOM | 3456 | O | HOH | S | 119 | 90.842 | 51.852 | 54.531 | 1.00 | 15.62 | S |
| ATOM | 3457 | O | HOH | S | 120 | 91.031 | 45.275 | 49.347 | 1.00 | 17.56 | S |
| ATOM | 3458 | O | HOH | S | 121 | 98.474 | 61.784 | 52.767 | 1.00 | 16.75 | S |
| ATOM | 3459 | O | HOH | S | 122 | 115.589 | 42.510 | 65.453 | 1.00 | 17.53 | S |
| ATOM | 3460 | O | HOH | S | 123 | 72.702 | 29.277 | 46.431 | 1.00 | 14.07 | S |
| ATOM | 3461 | O | HOH | S | 124 | 65.526 | 28.026 | 66.927 | 1.00 | 16.86 | S |
| ATOM | 3462 | O | HOH | S | 125 | 105.747 | 30.495 | 34.621 | 1.00 | 21.51 | S |
| ATOM | 3463 | O | HOH | S | 126 | 87.128 | 45.249 | 36.938 | 1.00 | 16.28 | S |
| ATOM | 3464 | O | HOH | S | 127 | 106.420 | 43.495 | 37.632 | 1.00 | 17.23 | S |
| ATOM | 3465 | O | HOH | S | 128 | 99.192 | 48.657 | 61.598 | 1.00 | 17.21 | S |
| ATOM | 3466 | O | HOH | S | 129 | 73.957 | 30.107 | 69.457 | 1.00 | 17.05 | S |
| ATOM | 3467 | O | HOH | S | 130 | 104.355 | 44.248 | 70.000 | 1.00 | 18.67 | S |
| ATOM | 3468 | O | HOH | S | 131 | 114.266 | 42.827 | 69.707 | 1.00 | 17.71 | S |
| ATOM | 3469 | O | HOH | S | 132 | 63.779 | −0.739 | 73.384 | 1.00 | 17.54 | S |
| ATOM | 3470 | O | HOH | S | 133 | 74.221 | 32.892 | 59.503 | 1.00 | 17.43 | S |
| ATOM | 3471 | O | HOH | S | 134 | 62.030 | 28.916 | 55.296 | 1.00 | 19.72 | S |
| ATOM | 3472 | O | HOH | S | 135 | 68.101 | 17.025 | 36.703 | 1.00 | 15.49 | S |
| ATOM | 3473 | O | HOH | S | 136 | 94.883 | 48.762 | 35.869 | 1.00 | 18.05 | S |
| ATOM | 3474 | O | HOH | S | 137 | 55.541 | 20.282 | 49.348 | 1.00 | 19.38 | S |
| ATOM | 3475 | O | HOH | S | 138 | 71.841 | 45.343 | 51.960 | 1.00 | 21.04 | S |
| ATOM | 3476 | O | HOH | S | 139 | 76.951 | 15.001 | 69.407 | 1.00 | 16.31 | S |
| ATOM | 3477 | O | HOH | S | 140 | 99.626 | 45.528 | 65.443 | 1.00 | 17.13 | S |
| ATOM | 3478 | O | HOH | S | 141 | 75.868 | 46.423 | 39.902 | 1.00 | 15.37 | S |
| ATOM | 3479 | O | HOH | S | 142 | 64.533 | 28.242 | 38.624 | 1.00 | 15.71 | S |
| ATOM | 3480 | O | HOH | S | 143 | 55.060 | 6.578 | 66.584 | 1.00 | 15.72 | S |
| ATOM | 3481 | O | HOH | S | 144 | 62.533 | 1.275 | 76.308 | 1.00 | 21.43 | S |
| ATOM | 3482 | O | HOH | S | 145 | 80.489 | 41.793 | 63.367 | 1.00 | 21.36 | S |
| ATOM | 3483 | O | HOH | S | 146 | 98.122 | 29.363 | 25.763 | 1.00 | 22.01 | S |
| ATOM | 3484 | O | HOH | S | 147 | 82.420 | 28.300 | 57.858 | 1.00 | 19.65 | S |
| ATOM | 3485 | O | HOH | S | 148 | 93.625 | 36.188 | 44.122 | 1.00 | 17.55 | S |
| ATOM | 3486 | O | HOH | S | 149 | 111.922 | 31.079 | 54.134 | 1.00 | 16.66 | S |
| ATOM | 3487 | O | HOH | S | 150 | 73.039 | 35.125 | 60.379 | 1.00 | 17.15 | S |
| ATOM | 3488 | O | HOH | S | 151 | 51.663 | 18.969 | 54.454 | 1.00 | 21.84 | S |
| ATOM | 3489 | O | HOH | S | 152 | 68.475 | 18.048 | 73.325 | 1.00 | 19.63 | S |
| ATOM | 3490 | O | HOH | S | 153 | 85.872 | 39.866 | 74.524 | 1.00 | 23.21 | S |
| ATOM | 3491 | O | HOH | S | 154 | 74.927 | 30.661 | 56.331 | 1.00 | 17.67 | S |
| ATOM | 3492 | O | HOH | S | 155 | 64.890 | 22.692 | 71.149 | 1.00 | 21.65 | S |
| ATOM | 3493 | O | HOH | S | 156 | 104.445 | 40.073 | 37.588 | 1.00 | 17.32 | S |
| ATOM | 3494 | O | HOH | S | 157 | 78.034 | 28.074 | 64.263 | 1.00 | 23.28 | S |
| ATOM | 3495 | O | HOH | S | 158 | 97.580 | 20.219 | 53.364 | 1.00 | 19.21 | S |
| ATOM | 3496 | O | HOH | S | 159 | 106.528 | 62.197 | 53.357 | 1.00 | 23.46 | S |
| ATOM | 3497 | O | HOH | S | 160 | 70.591 | 39.144 | 46.923 | 1.00 | 20.46 | S |
| ATOM | 3498 | O | HOH | S | 161 | 116.720 | 43.333 | 55.143 | 1.00 | 16.71 | S |
| ATOM | 3499 | O | HOH | S | 162 | 112.486 | 36.852 | 69.543 | 1.00 | 21.68 | S |
| ATOM | 3500 | O | HOH | S | 163 | 74.558 | 45.734 | 62.711 | 1.00 | 25.46 | S |
| ATOM | 3501 | O | HOH | S | 164 | 67.019 | 1.970 | 65.031 | 1.00 | 20.52 | S |
| ATOM | 3502 | O | HOH | S | 165 | 103.635 | 63.023 | 43.611 | 1.00 | 25.79 | S |
| ATOM | 3503 | O | HOH | S | 166 | 113.761 | 43.907 | 66.926 | 1.00 | 18.65 | S |
| ATOM | 3504 | O | HOH | S | 167 | 85.777 | 49.270 | 68.431 | 1.00 | 21.32 | S |
| ATOM | 3505 | O | HOH | S | 168 | 106.209 | 23.945 | 50.173 | 1.00 | 14.75 | S |
| ATOM | 3506 | O | HOH | S | 169 | 68.599 | 20.700 | 73.596 | 1.00 | 22.53 | S |
| ATOM | 3507 | O | HOH | S | 170 | 88.589 | 45.469 | 34.630 | 1.00 | 19.68 | S |
| ATOM | 3508 | O | HOH | S | 171 | 95.172 | 51.484 | 35.969 | 1.00 | 20.30 | S |
| ATOM | 3509 | O | HOH | S | 172 | 57.864 | 12.043 | 71.902 | 1.00 | 22.04 | S |
| ATOM | 3510 | O | HOH | S | 173 | 111.558 | 28.443 | 54.056 | 1.00 | 17.63 | S |
| ATOM | 3511 | O | HOH | S | 174 | 111.964 | 57.303 | 61.527 | 1.00 | 17.73 | S |
| ATOM | 3512 | O | HOH | S | 175 | 73.961 | 53.477 | 54.439 | 1.00 | 21.04 | S |
| ATOM | 3513 | O | HOH | S | 176 | 89.936 | 56.242 | 59.767 | 1.00 | 21.34 | S |
| ATOM | 3514 | O | HOH | S | 177 | 112.842 | 54.118 | 56.111 | 1.00 | 20.11 | S |
| ATOM | 3515 | O | HOH | S | 178 | 64.964 | 29.255 | 54.623 | 1.00 | 21.73 | S |
| ATOM | 3516 | O | HOH | S | 179 | 118.957 | 43.011 | 56.353 | 1.00 | 19.16 | S |
| ATOM | 3517 | O | HOH | S | 180 | 80.176 | 24.029 | 60.304 | 1.00 | 17.28 | S |
| ATOM | 3518 | O | HOH | S | 181 | 62.530 | 5.651 | 76.155 | 1.00 | 19.07 | S |
| ATOM | 3519 | O | HOH | S | 182 | 118.350 | 49.291 | 56.438 | 1.00 | 20.17 | S |
| ATOM | 3520 | O | HOH | S | 183 | 72.763 | 45.447 | 47.638 | 1.00 | 19.81 | S |
| ATOM | 3521 | O | HOH | S | 184 | 112.735 | 24.678 | 50.604 | 1.00 | 21.79 | S |
| ATOM | 3522 | O | HOH | S | 185 | 86.058 | 35.926 | 74.262 | 1.00 | 22.01 | S |
| ATOM | 3523 | O | HOH | S | 186 | 114.834 | 49.889 | 42.771 | 1.00 | 23.31 | S |
| ATOM | 3524 | O | HOH | S | 187 | 75.602 | 21.157 | 72.044 | 1.00 | 22.14 | S |
| ATOM | 3525 | O | HOH | S | 188 | 112.672 | 32.786 | 56.690 | 1.00 | 26.39 | S |
| ATOM | 3526 | O | HOH | S | 189 | 70.424 | 31.689 | 66.892 | 1.00 | 30.22 | S |
| ATOM | 3527 | O | HOH | S | 190 | 86.180 | 52.387 | 35.161 | 1.00 | 22.09 | S |
| ATOM | 3528 | O | HOH | S | 191 | 116.177 | 46.004 | 55.671 | 1.00 | 23.58 | S |
| ATOM | 3529 | O | HOH | S | 192 | 83.139 | 23.136 | 61.992 | 1.00 | 26.02 | S |
| ATOM | 3530 | O | HOH | S | 193 | 79.533 | 22.863 | 62.806 | 1.00 | 23.58 | S |
| ATOM | 3531 | O | HOH | S | 194 | 109.505 | 57.366 | 67.879 | 1.00 | 23.69 | S |
| ATOM | 3532 | O | HOH | S | 195 | 91.401 | 42.959 | 50.697 | 1.00 | 20.05 | S |
| ATOM | 3533 | O | HOH | S | 196 | 107.856 | 39.841 | 74.251 | 1.00 | 24.41 | S |
| ATOM | 3534 | O | HOH | S | 197 | 110.663 | 42.166 | 72.833 | 1.00 | 25.27 | S |
| ATOM | 3535 | O | HOH | S | 198 | 101.532 | 66.507 | 53.191 | 1.00 | 23.70 | S |
| ATOM | 3536 | O | HOH | S | 199 | 106.601 | 37.539 | 36.443 | 1.00 | 21.26 | S |
| ATOM | 3537 | O | HOH | S | 200 | 118.404 | 37.746 | 60.351 | 1.00 | 20.07 | S |
| ATOM | 3538 | O | HOH | S | 201 | 77.352 | 56.839 | 49.247 | 1.00 | 22.97 | S |
| ATOM | 3539 | O | HOH | S | 202 | 101.121 | 59.914 | 60.079 | 1.00 | 24.47 | S |
| ATOM | 3540 | O | HOH | S | 203 | 79.363 | 16.613 | 43.206 | 1.00 | 19.29 | S |
| ATOM | 3541 | O | HOH | S | 204 | 95.479 | 40.285 | 33.289 | 1.00 | 21.93 | S |
| ATOM | 3542 | O | HOH | S | 205 | 61.574 | 30.762 | 47.670 | 1.00 | 28.96 | S |
| ATOM | 3543 | O | HOH | S | 206 | 86.028 | 28.705 | 67.206 | 1.00 | 25.71 | S |
| ATOM | 3544 | O | HOH | S | 207 | 114.925 | 32.587 | 47.892 | 1.00 | 23.87 | S |
| ATOM | 3545 | O | HOH | S | 208 | 84.545 | 52.471 | 37.319 | 1.00 | 20.62 | S |
| ATOM | 3546 | O | HOH | S | 209 | 99.929 | 48.662 | 34.518 | 1.00 | 21.50 | S |
| ATOM | 3547 | O | HOH | S | 210 | 79.506 | 58.124 | 55.197 | 1.00 | 29.73 | S |
| ATOM | 3548 | O | HOH | S | 211 | 55.219 | −2.463 | 71.023 | 1.00 | 24.32 | S |
| ATOM | 3549 | O | HOH | S | 212 | 55.933 | 17.599 | 69.830 | 1.00 | 23.08 | S |
| ATOM | 3550 | O | HOH | S | 213 | 95.320 | 57.844 | 36.544 | 1.00 | 22.50 | S |
| ATOM | 3551 | O | HOH | S | 214 | 83.537 | 48.307 | 35.916 | 1.00 | 21.82 | S |
| ATOM | 3552 | O | HOH | S | 215 | 78.218 | 53.116 | 60.484 | 1.00 | 27.09 | S |
| ATOM | 3553 | O | HOH | S | 216 | 93.360 | 46.860 | 69.662 | 1.00 | 22.12 | S |
| ATOM | 3554 | O | HOH | S | 217 | 117.615 | 42.729 | 52.472 | 1.00 | 23.44 | S |
| ATOM | 3555 | O | HOH | S | 218 | 112.109 | 56.288 | 41.991 | 1.00 | 23.46 | S |
| ATOM | 3556 | O | HOH | S | 219 | 56.051 | 22.673 | 50.382 | 1.00 | 27.49 | S |
| ATOM | 3557 | O | HOH | S | 220 | 63.814 | 22.334 | 40.191 | 1.00 | 25.82 | S |
| ATOM | 3558 | O | HOH | S | 221 | 106.268 | 27.738 | 60.070 | 1.00 | 30.69 | S |
| ATOM | 3559 | O | HOH | S | 222 | 80.342 | 48.282 | 64.542 | 1.00 | 19.35 | S |
| ATOM | 3560 | O | HOH | S | 223 | 93.855 | 64.305 | 52.328 | 1.00 | 22.91 | S |
| ATOM | 3561 | O | HOH | S | 224 | 110.496 | 48.556 | 37.495 | 1.00 | 23.02 | S |
| ATOM | 3562 | O | HOH | S | 225 | 88.449 | 28.551 | 37.582 | 1.00 | 20.48 | S |
| ATOM | 3563 | O | HOH | S | 226 | 83.799 | 21.051 | 66.947 | 1.00 | 34.14 | S |
| ATOM | 3564 | O | HOH | S | 227 | 89.485 | 62.780 | 57.516 | 1.00 | 25.20 | S |
| ATOM | 3565 | O | HOH | S | 228 | 63.910 | 7.689 | 77.322 | 1.00 | 27.69 | S |
| ATOM | 3566 | O | HOH | S | 229 | 79.129 | 36.247 | 70.166 | 1.00 | 26.35 | S |
| ATOM | 3567 | O | HOH | S | 230 | 117.677 | 46.182 | 47.671 | 1.00 | 18.71 | S |
| ATOM | 3568 | O | HOH | S | 231 | 92.493 | 42.317 | 44.329 | 1.00 | 21.96 | S |
| ATOM | 3569 | O | HOH | S | 232 | 79.799 | 50.887 | 64.071 | 1.00 | 27.84 | S |
| ATOM | 3570 | O | HOH | S | 233 | 86.890 | 57.927 | 40.633 | 1.00 | 27.81 | S |
| ATOM | 3571 | O | HOH | S | 234 | 76.083 | 8.213 | 75.663 | 1.00 | 28.30 | S |
| ATOM | 3572 | O | HOH | S | 235 | 106.484 | 32.473 | 32.869 | 1.00 | 30.35 | S |
| ATOM | 3573 | O | HOH | S | 236 | 60.245 | 6.928 | 75.254 | 1.00 | 20.43 | S |
| ATOM | 3574 | O | HOH | S | 237 | 70.929 | 40.961 | 57.089 | 1.00 | 25.53 | S |
| ATOM | 3575 | O | HOH | S | 238 | 87.119 | 32.698 | 37.059 | 1.00 | 26.57 | S |
| ATOM | 3576 | O | HOH | S | 239 | 90.604 | 41.169 | 74.679 | 1.00 | 27.18 | S |
| ATOM | 3577 | O | HOH | S | 240 | 98.426 | 27.245 | 39.721 | 1.00 | 22.26 | S |
| ATOM | 3578 | O | HOH | S | 241 | 59.767 | 28.520 | 49.379 | 1.00 | 30.99 | S |
| ATOM | 3579 | O | HOH | S | 242 | 106.785 | 23.682 | 54.767 | 1.00 | 26.85 | S |
| ATOM | 3580 | O | HOH | S | 243 | 105.845 | 56.259 | 68.858 | 1.00 | 25.31 | S |
| ATOM | 3581 | O | HOH | S | 244 | 77.138 | 32.119 | 70.331 | 1.00 | 29.02 | S |
| ATOM | 3582 | O | HOH | S | 245 | 96.511 | 46.735 | 64.963 | 1.00 | 23.85 | S |
| ATOM | 3583 | O | HOH | S | 246 | 74.193 | 6.873 | 77.617 | 1.00 | 27.74 | S |
| ATOM | 3584 | O | HOH | S | 247 | 97.163 | 38.630 | 41.100 | 1.00 | 27.28 | S |
| ATOM | 3585 | O | HOH | S | 248 | 71.467 | 11.480 | 72.272 | 1.00 | 19.45 | S |
| ATOM | 3586 | O | HOH | S | 249 | 115.428 | 25.486 | 47.708 | 1.00 | 29.48 | S |
| ATOM | 3587 | O | HOH | S | 250 | 64.227 | 31.447 | 50.431 | 1.00 | 31.46 | S |
| ATOM | 3588 | O | HOH | S | 251 | 58.302 | 27.955 | 52.629 | 1.00 | 30.23 | S |
| ATOM | 3589 | O | HOH | S | 252 | 97.011 | 46.975 | 34.756 | 1.00 | 22.79 | S |
| ATOM | 3590 | O | HOH | S | 253 | 84.236 | 60.734 | 66.398 | 1.00 | 28.58 | S |
| ATOM | 3591 | O | HOH | S | 254 | 102.852 | 50.198 | 67.839 | 1.00 | 32.58 | S |
| ATOM | 3592 | O | HOH | S | 255 | 73.211 | 13.188 | 52.786 | 1.00 | 22.55 | S |
| ATOM | 3593 | O | HOH | S | 256 | 75.331 | 18.311 | 70.464 | 1.00 | 21.00 | S |
| ATOM | 3594 | O | HOH | S | 257 | 107.712 | 62.611 | 47.795 | 1.00 | 32.03 | S |
| ATOM | 3595 | O | HOH | S | 258 | 62.092 | 22.277 | 70.778 | 1.00 | 26.88 | S |
| ATOM | 3596 | O | HOH | S | 259 | 111.132 | 57.360 | 48.385 | 1.00 | 29.50 | S |
| ATOM | 3597 | O | HOH | S | 260 | 79.507 | 42.776 | 68.342 | 1.00 | 28.97 | S |
| ATOM | 3598 | O | HOH | S | 261 | 67.421 | 12.007 | 78.097 | 1.00 | 28.73 | S |
| ATOM | 3599 | O | HOH | S | 262 | 77.485 | 37.442 | 64.584 | 1.00 | 25.39 | S |
| ATOM | 3600 | O | HOH | S | 263 | 107.530 | 41.946 | 33.095 | 1.00 | 33.97 | S |
| ATOM | 3601 | O | HOH | S | 264 | 100.746 | 27.883 | 65.152 | 1.00 | 27.51 | S |
| ATOM | 3602 | O | HOH | S | 265 | 102.853 | 40.039 | 32.809 | 1.00 | 26.58 | S |
| ATOM | 3603 | O | HOH | S | 266 | 72.337 | 30.470 | 57.341 | 1.00 | 29.49 | S |
| ATOM | 3604 | O | HOH | S | 267 | 63.882 | 14.218 | 75.483 | 1.00 | 30.81 | S |
| ATOM | 3605 | O | HOH | S | 268 | 66.563 | 30.287 | 51.517 | 1.00 | 28.01 | S |
| ATOM | 3606 | O | HOH | S | 269 | 78.712 | 60.400 | 61.627 | 1.00 | 30.01 | S |
| ATOM | 3607 | O | HOH | S | 270 | 94.892 | 35.853 | 76.677 | 1.00 | 29.68 | S |
| ATOM | 3608 | O | HOH | S | 271 | 101.118 | 29.764 | 40.201 | 1.00 | 20.33 | S |
| ATOM | 3609 | O | HOH | S | 272 | 117.176 | 39.526 | 55.413 | 1.00 | 31.58 | S |
| ATOM | 3610 | O | HOH | S | 273 | 77.043 | 34.847 | 69.146 | 1.00 | 28.47 | S |
| ATOM | 3611 | O | HOH | S | 274 | 80.802 | 21.552 | 36.801 | 1.00 | 21.40 | S |
| ATOM | 3612 | O | HOH | S | 275 | 88.362 | 33.429 | 42.333 | 1.00 | 22.15 | S |
| ATOM | 3613 | O | HOH | S | 276 | 68.049 | 24.948 | 74.294 | 1.00 | 34.45 | S |
| ATOM | 3614 | O | HOH | S | 277 | 102.418 | 22.053 | 33.756 | 1.00 | 34.29 | S |
| ATOM | 3615 | O | HOH | S | 278 | 114.067 | 35.288 | 67.215 | 1.00 | 30.00 | S |
| ATOM | 3616 | O | HOH | S | 279 | 94.356 | 32.826 | 39.830 | 1.00 | 29.88 | S |
| ATOM | 3617 | O | HOH | S | 280 | 110.533 | 60.123 | 63.684 | 1.00 | 25.44 | S |
| ATOM | 3618 | O | HOH | S | 281 | 74.401 | 15.967 | 68.989 | 1.00 | 26.47 | S |
| ATOM | 3619 | O | HOH | S | 282 | 93.397 | 43.931 | 55.242 | 1.00 | 20.92 | S |
| ATOM | 3620 | O | HOH | S | 283 | 112.712 | 58.724 | 64.627 | 1.00 | 28.77 | S |
| ATOM | 3621 | O | HOH | S | 284 | 102.548 | 66.246 | 55.932 | 1.00 | 25.27 | S |
| ATOM | 3622 | O | HOH | S | 285 | 104.326 | 45.932 | 33.192 | 1.00 | 35.84 | S |
| ATOM | 3623 | O | HOH | S | 286 | 102.364 | 43.791 | 30.462 | 1.00 | 34.15 | S |
| ATOM | 3624 | O | HOH | S | 287 | 96.453 | 40.095 | 71.886 | 1.00 | 27.93 | S |
| ATOM | 3625 | O | HOH | S | 288 | 99.702 | 28.233 | 37.259 | 1.00 | 26.57 | S |
| ATOM | 3626 | O | HOH | S | 289 | 107.236 | 30.421 | 39.055 | 1.00 | 26.78 | S |
| ATOM | 3627 | O | HOH | S | 290 | 83.901 | 36.644 | 75.730 | 1.00 | 28.98 | S |
| ATOM | 3628 | O | HOH | S | 291 | 69.013 | 30.041 | 55.709 | 1.00 | 31.77 | S |
| ATOM | 3629 | O | HOH | S | 292 | 94.347 | 41.789 | 56.645 | 1.00 | 27.51 | S |
| ATOM | 3630 | O | HOH | S | 293 | 91.830 | 56.247 | 61.826 | 1.00 | 24.02 | S |
| ATOM | 3631 | O | HOH | S | 294 | 96.814 | 26.388 | 59.145 | 1.00 | 35.77 | S |
| ATOM | 3632 | O | HOH | S | 295 | 67.964 | 43.323 | 51.455 | 1.00 | 32.32 | S |
| ATOM | 3633 | O | HOH | S | 296 | 115.587 | 44.348 | 35.337 | 1.00 | 43.10 | S |
| ATOM | 3634 | O | HOH | S | 297 | 112.406 | 53.745 | 45.763 | 1.00 | 37.43 | S |
| ATOM | 3635 | O | HOH | S | 298 | 113.918 | 41.435 | 43.781 | 1.00 | 25.92 | S |
| ATOM | 3636 | O | HOH | S | 299 | 119.456 | 49.232 | 53.292 | 1.00 | 24.52 | S |
| ATOM | 3637 | O | HOH | S | 300 | 112.818 | 40.545 | 36.421 | 1.00 | 31.15 | S |
| ATOM | 3638 | O | HOH | S | 301 | 114.646 | 39.974 | 46.708 | 1.00 | 33.94 | S |
| ATOM | 3639 | O | HOH | S | 302 | 92.750 | 68.122 | 52.517 | 1.00 | 34.98 | S |
| ATOM | 3640 | O | HOH | S | 303 | 61.613 | 28.055 | 57.711 | 1.00 | 29.71 | S |
| ATOM | 3641 | O | HOH | S | 304 | 90.449 | 41.333 | 48.565 | 1.00 | 25.05 | S |
| ATOM | 3642 | O | HOH | S | 305 | 73.874 | 53.930 | 41.718 | 1.00 | 33.37 | S |
| ATOM | 3643 | O | HOH | S | 306 | 116.205 | 34.495 | 62.782 | 1.00 | 26.60 | S |
| ATOM | 3644 | O | HOH | S | 307 | 79.550 | 38.432 | 74.675 | 1.00 | 41.00 | S |
| ATOM | 3645 | O | HOH | S | 308 | 106.114 | 31.157 | 65.602 | 1.00 | 30.44 | S |
| ATOM | 3646 | O | HOH | S | 309 | 67.172 | 32.820 | 46.565 | 1.00 | 31.09 | S |
| ATOM | 3647 | O | HOH | S | 310 | 109.806 | 24.960 | 54.952 | 1.00 | 32.53 | S |
| ATOM | 3648 | O | HOH | S | 311 | 99.200 | 65.141 | 46.682 | 1.00 | 30.74 | S |
| ATOM | 3649 | O | HOH | S | 312 | 119.964 | 40.281 | 58.266 | 1.00 | 35.96 | S |
| ATOM | 3650 | O | HOH | S | 313 | 92.398 | 45.756 | 53.729 | 1.00 | 28.56 | S |
| ATOM | 3651 | O | HOH | S | 314 | 77.302 | 56.736 | 41.434 | 1.00 | 28.67 | S |
| ATOM | 3652 | O | HOH | S | 315 | 117.870 | 35.329 | 48.578 | 1.00 | 28.32 | S |
| ATOM | 3653 | O | HOH | S | 316 | 118.250 | 52.296 | 50.849 | 1.00 | 31.04 | S |
| ATOM | 3654 | O | HOH | S | 317 | 85.718 | 32.135 | 72.137 | 1.00 | 28.42 | S |
| ATOM | 3655 | O | HOH | S | 318 | 65.192 | 9.912 | 76.910 | 1.00 | 31.76 | S |
| ATOM | 3656 | O | HOH | S | 319 | 109.255 | 31.199 | 63.481 | 1.00 | 32.35 | S |
| ATOM | 3657 | O | HOH | S | 320 | 107.951 | 48.654 | 72.521 | 1.00 | 34.23 | S |
| ATOM | 3658 | O | HOH | S | 321 | 78.567 | 56.734 | 61.178 | 1.00 | 44.27 | S |
| ATOM | 3659 | O | HOH | S | 322 | 116.740 | 33.836 | 52.185 | 1.00 | 31.57 | S |
| ATOM | 3660 | O | HOH | S | 323 | 52.064 | 16.874 | 52.491 | 1.00 | 34.09 | S |
| ATOM | 3661 | O | HOH | S | 324 | 101.785 | 50.511 | 60.866 | 1.00 | 30.60 | S |
| ATOM | 3662 | O | HOH | S | 325 | 61.024 | 23.809 | 68.812 | 1.00 | 31.02 | S |
| ATOM | 3663 | O | HOH | S | 326 | 81.314 | 54.868 | 39.824 | 1.00 | 29.23 | S |
| ATOM | 3664 | O | HOH | S | 327 | 72.887 | 54.170 | 46.373 | 1.00 | 39.81 | S |
| ATOM | 3665 | O | HOH | S | 328 | 112.146 | 31.972 | 38.743 | 1.00 | 42.21 | S |
| ATOM | 3666 | O | HOH | S | 329 | 57.609 | 21.812 | 71.254 | 1.00 | 31.35 | S |
| ATOM | 3667 | O | HOH | S | 330 | 92.571 | 37.445 | 75.657 | 1.00 | 29.19 | S |
| ATOM | 3668 | O | HOH | S | 331 | 114.461 | 52.724 | 40.120 | 1.00 | 35.30 | S |
| ATOM | 3669 | O | HOH | S | 332 | 107.384 | 64.230 | 50.085 | 1.00 | 34.17 | S |
| ATOM | 3670 | O | HOH | S | 333 | 101.852 | 18.724 | 54.674 | 1.00 | 30.29 | S |
| ATOM | 3671 | O | HOH | S | 334 | 97.454 | 22.994 | 39.318 | 1.00 | 24.31 | S |
| ATOM | 3672 | O | HOH | S | 335 | 114.456 | 38.019 | 44.843 | 1.00 | 39.25 | S |
| ATOM | 3673 | O | HOH | S | 336 | 97.203 | 27.162 | 33.793 | 1.00 | 32.56 | S |
| ATOM | 3674 | O | HOH | S | 337 | 75.416 | 43.577 | 65.602 | 1.00 | 36.58 | S |
| ATOM | 3675 | O | HOH | S | 338 | 80.899 | 24.641 | 71.265 | 1.00 | 43.27 | S |
| ATOM | 3676 | O | HOH | S | 339 | 109.927 | 33.467 | 38.972 | 1.00 | 40.54 | S |
| ATOM | 3677 | O | HOH | S | 340 | 82.259 | 57.574 | 46.159 | 1.00 | 33.03 | S |
| ATOM | 3678 | O | HOH | S | 341 | 91.746 | 45.276 | 56.612 | 1.00 | 30.62 | S |
| ATOM | 3679 | O | HOH | S | 342 | 58.371 | 27.379 | 57.748 | 1.00 | 32.69 | S |
| ATOM | 3680 | O | HOH | S | 343 | 64.555 | 16.646 | 75.166 | 1.00 | 39.40 | S |
| ATOM | 3681 | O | HOH | S | 344 | 89.187 | 50.046 | 72.586 | 1.00 | 33.04 | S |
| ATOM | 3682 | O | HOH | S | 345 | 87.191 | 43.989 | 73.680 | 1.00 | 37.70 | S |
| ATOM | 3683 | O | HOH | S | 346 | 62.209 | 10.849 | 73.949 | 1.00 | 36.93 | S |
| ATOM | 3684 | O | HOH | S | 347 | 79.001 | 25.483 | 66.851 | 1.00 | 29.03 | S |
| ATOM | 3685 | O | HOH | S | 348 | 110.046 | 37.177 | 37.661 | 1.00 | 41.31 | S |
| ATOM | 3686 | O | HOH | S | 349 | 75.621 | 36.405 | 66.678 | 1.00 | 33.41 | S |
| ATOM | 3687 | O | HOH | S | 350 | 94.615 | 63.380 | 44.736 | 1.00 | 31.19 | S |
| ATOM | 3688 | O | HOH | S | 351 | 65.672 | 21.575 | 73.606 | 1.00 | 41.89 | S |
| ATOM | 3689 | O | HOH | S | 352 | 76.681 | 25.436 | 73.525 | 1.00 | 34.56 | S |
| ATOM | 3690 | O | HOH | S | 353 | 109.732 | 31.367 | 42.570 | 1.00 | 31.53 | S |
| ATOM | 3691 | O | HOH | S | 354 | 113.831 | 36.638 | 53.595 | 1.00 | 28.21 | S |
| ATOM | 3692 | O | HOH | S | 355 | 103.662 | 27.741 | 62.967 | 1.00 | 40.44 | S |
| ATOM | 3693 | O | HOH | S | 356 | 78.499 | 20.878 | 72.331 | 1.00 | 34.68 | S |
| ATOM | 3694 | O | HOH | S | 357 | 79.135 | 55.114 | 53.724 | 1.00 | 41.80 | S |
| ATOM | 3695 | O | HOH | S | 358 | 102.549 | 65.216 | 48.531 | 1.00 | 83.53 | S |
| ATOM | 3696 | O | HOH | S | 359 | 80.250 | 45.352 | 71.373 | 1.00 | 33.37 | S |
| ATOM | 3697 | O | HOH | S | 360 | 100.906 | 49.837 | 63.424 | 1.00 | 36.66 | S |
| ATOM | 3698 | O | HOH | S | 361 | 69.598 | 29.519 | 69.434 | 1.00 | 33.28 | S |
| ATOM | 3699 | O | HOH | S | 362 | 96.613 | 30.275 | 32.572 | 1.00 | 33.11 | S |
| ATOM | 3700 | O | HOH | S | 363 | 88.034 | 52.008 | 69.212 | 1.00 | 34.04 | S |
| ATOM | 3701 | O | HOH | S | 364 | 88.006 | 60.343 | 48.244 | 1.00 | 32.15 | S |
| ATOM | 3702 | O | HOH | S | 365 | 94.632 | 59.066 | 34.154 | 1.00 | 42.33 | S |
| ATOM | 3703 | O | HOH | S | 366 | 96.021 | 25.095 | 35.406 | 1.00 | 34.86 | S |
| ATOM | 3704 | O | HOH | S | 367 | 78.422 | 59.901 | 58.347 | 1.00 | 39.90 | S |
| ATOM | 3705 | O | HOH | S | 368 | 90.589 | 57.022 | 38.655 | 1.00 | 36.89 | S |
| ATOM | 3706 | O | HOH | S | 369 | 81.899 | 31.605 | 67.233 | 1.00 | 27.07 | S |
| ATOM | 3707 | O | HOH | S | 370 | 99.774 | 61.489 | 39.856 | 1.00 | 38.31 | S |
| ATOM | 3708 | O | HOH | S | 371 | 107.262 | 20.225 | 54.593 | 1.00 | 39.36 | S |
| ATOM | 3709 | O | HOH | S | 372 | 79.973 | 57.526 | 50.419 | 1.00 | 38.11 | S |
| ATOM | 3710 | O | HOH | S | 373 | 54.795 | 9.851 | 69.325 | 1.00 | 30.76 | S |
| ATOM | 3711 | O | HOH | S | 374 | 112.940 | 39.820 | 71.258 | 1.00 | 36.73 | S |
| ATOM | 3712 | O | HOH | S | 375 | 111.889 | 38.749 | 40.735 | 1.00 | 35.23 | S |
| ATOM | 3713 | O | HOH | S | 376 | 85.331 | 63.188 | 49.990 | 1.00 | 45.55 | S |
| ATOM | 3714 | O | HOH | S | 377 | 108.143 | 36.000 | 29.772 | 1.00 | 53.98 | S |
| ATOM | 3715 | O | HOH | S | 378 | 96.775 | 54.035 | 62.235 | 1.00 | 44.92 | S |
| ATOM | 3716 | O | HOH | S | 379 | 93.408 | 58.812 | 38.760 | 1.00 | 38.56 | S |
| ATOM | 3717 | O | HOH | S | 380 | 117.514 | 36.042 | 53.398 | 1.00 | 37.89 | S |
| ATOM | 3718 | O | HOH | S | 381 | 100.891 | 64.783 | 44.669 | 1.00 | 48.32 | S |
| ATOM | 3719 | O | HOH | S | 382 | 100.881 | 29.677 | 33.169 | 1.00 | 46.51 | S |
| ATOM | 3720 | O | HOH | S | 383 | 92.275 | 31.013 | 72.776 | 1.00 | 29.67 | S |
| ATOM | 3721 | O | HOH | S | 384 | 105.144 | 32.860 | 67.811 | 1.00 | 43.13 | S |
| ATOM | 3722 | O | HOH | S | 385 | 118.817 | 37.912 | 57.760 | 1.00 | 46.58 | S |
| ATOM | 3723 | O | HOH | S | 386 | 65.839 | 2.070 | 62.113 | 1.00 | 72.88 | S |
| ATOM | 3724 | O | HOH | S | 387 | 94.528 | 27.907 | 29.484 | 1.00 | 33.68 | S |
| ATOM | 3725 | O | HOH | S | 388 | 71.086 | 52.157 | 49.836 | 1.00 | 44.75 | S |
| ATOM | 3726 | O | HOH | S | 389 | 109.926 | 62.610 | 56.745 | 1.00 | 64.25 | S |
| ATOM | 3727 | O | HOH | S | 390 | 82.548 | 63.708 | 60.959 | 1.00 | 35.43 | S |
| ATOM | 3728 | O | HOH | S | 391 | 89.200 | 55.060 | 34.476 | 1.00 | 93.59 | S |
| ATOM | 3729 | O | HOH | S | 392 | 78.936 | 18.115 | 72.214 | 1.00 | 33.02 | S |
| ATOM | 3730 | O | HOH | S | 393 | 108.361 | 53.463 | 35.359 | 1.00 | 31.47 | S |
| ATOM | 3731 | O | HOH | S | 394 | 67.325 | 28.508 | 71.112 | 1.00 | 32.98 | S |
| ATOM | 3732 | O | HOH | S | 395 | 71.105 | 12.429 | 76.836 | 1.00 | 47.23 | S |
| ATOM | 3733 | O | HOH | S | 396 | 109.036 | 35.778 | 73.249 | 1.00 | 38.51 | S |
| ATOM | 3734 | O | HOH | S | 397 | 99.857 | 68.565 | 53.134 | 1.00 | 37.71 | S |
| ATOM | 3735 | O | HOH | S | 398 | 83.368 | 42.565 | 77.189 | 1.00 | 37.60 | S |
| ATOM | 3736 | O | HOH | S | 399 | 98.907 | 47.637 | 64.237 | 1.00 | 46.79 | S |
| ATOM | 3737 | O | HOH | S | 400 | 104.264 | 29.555 | 67.449 | 1.00 | 38.04 | S |
| ATOM | 3738 | O | HOH | S | 401 | 113.919 | 33.963 | 54.418 | 1.00 | 31.70 | S |
| ATOM | 3739 | O | HOH | S | 402 | 110.657 | 60.739 | 48.609 | 1.00 | 57.38 | S |
| ATOM | 3740 | O | HOH | S | 403 | 74.688 | 53.874 | 58.942 | 1.00 | 32.02 | S |
| ATOM | 3741 | O | HOH | S | 404 | 89.167 | 62.827 | 48.639 | 1.00 | 48.19 | S |
| ATOM | 3742 | O | HOH | S | 405 | 66.909 | 17.631 | 76.093 | 1.00 | 39.77 | S |
| ATOM | 3743 | O | HOH | S | 406 | 108.814 | 50.711 | 37.189 | 1.00 | 36.91 | S |
| ATOM | 3744 | O | HOH | S | 407 | 95.671 | 47.723 | 67.836 | 1.00 | 34.93 | S |
| ATOM | 3745 | O | HOH | S | 408 | 109.535 | 40.024 | 35.097 | 1.00 | 35.31 | S |
| ATOM | 3746 | O | HOH | S | 409 | 107.805 | 35.828 | 47.555 | 1.00 | 83.21 | S |
| ATOM | 3747 | O | HOH | S | 410 | 114.323 | 50.353 | 45.264 | 1.00 | 50.43 | S |
| ATOM | 3748 | O | HOH | S | 411 | 109.567 | 58.976 | 47.080 | 1.00 | 75.66 | S |
| ATOM | 3749 | O | HOH | S | 412 | 103.225 | 56.942 | 30.707 | 1.00 | 48.11 | S |
| ATOM | 3750 | O | HOH | S | 413 | 102.001 | 60.142 | 39.362 | 1.00 | 54.06 | S |
| ATOM | 3751 | O | HOH | S | 414 | 116.256 | 29.417 | 46.763 | 1.00 | 46.06 | S |
| ATOM | 3752 | O | HOH | S | 415 | 65.067 | 12.442 | 76.948 | 1.00 | 49.65 | S |
| ATOM | 3753 | O | HOH | S | 416 | 83.780 | 27.564 | 65.292 | 1.00 | 35.03 | S |
| ATOM | 3754 | O | HOH | S | 417 | 93.322 | 59.295 | 64.490 | 1.00 | 38.67 | S |
| ATOM | 3755 | O | HOH | S | 418 | 70.805 | 23.912 | 74.701 | 1.00 | 35.57 | S |
| ATOM | 3756 | O | HOH | S | 419 | 108.378 | 57.665 | 36.677 | 1.00 | 41.25 | S |
| ATOM | 3757 | O | HOH | S | 420 | 64.253 | 9.606 | 61.343 | 1.00 | 78.58 | S |
| ATOM | 3758 | O | HOH | S | 421 | 96.093 | 31.063 | 45.028 | 1.00 | 76.42 | S |
| ATOM | 3759 | O | HOH | S | 422 | 80.258 | 42.586 | 42.164 | 1.00 | 86.41 | S |
| ATOM | 3760 | O | HOH | S | 423 | 93.411 | 57.649 | 31.959 | 1.00 | 39.88 | S |
| ATOM | 3761 | O | HOH | S | 424 | 104.731 | 66.714 | 49.606 | 1.00 | 50.91 | S |
| ATOM | 3762 | O | HOH | S | 425 | 96.024 | 37.034 | 42.957 | 1.00 | 46.20 | S |
| ATOM | 3763 | O | HOH | S | 426 | 113.455 | 39.196 | 48.771 | 1.00 | 88.39 | S |
| ATOM | 3764 | O | HOH | S | 427 | 68.066 | 28.784 | 66.907 | 1.00 | 31.70 | S |
| ATOM | 3765 | O | HOH | S | 428 | 116.968 | 43.938 | 43.274 | 1.00 | 37.48 | S |
| ATOM | 3766 | O | HOH | S | 429 | 79.943 | 27.724 | 67.550 | 1.00 | 32.89 | S |
| ATOM | 3767 | O | HOH | S | 430 | 85.480 | 56.708 | 38.273 | 1.00 | 50.50 | S |
| ATOM | 3768 | O | HOH | S | 431 | 101.326 | 45.512 | 50.418 | 1.00 | 86.19 | S |
| ATOM | 3769 | O | HOH | S | 432 | 94.561 | 27.649 | 23.781 | 1.00 | 34.27 | S |
| ATOM | 3770 | O | HOH | S | 433 | 53.481 | −4.935 | 67.898 | 1.00 | 37.86 | S |
| ATOM | 3771 | O | HOH | S | 434 | 109.211 | 25.771 | 57.434 | 1.00 | 63.68 | S |
| ATOM | 3772 | O | HOH | S | 435 | 107.137 | 33.142 | 70.638 | 1.00 | 37.06 | S |
| ATOM | 3773 | O | HOH | S | 436 | 80.450 | 27.250 | 71.876 | 1.00 | 48.04 | S |
| ATOM | 3774 | O | HOH | S | 437 | 96.707 | 63.561 | 41.896 | 1.00 | 39.66 | S |
| ATOM | 3775 | O | HOH | S | 438 | 70.107 | 30.942 | 46.853 | 1.00 | 49.21 | S |
| ATOM | 3776 | O | HOH | S | 439 | 97.898 | 24.956 | 41.221 | 1.00 | 12.14 | S |
| ATOM | 3777 | O | HOH | S | 440 | 115.533 | 40.484 | 69.783 | 1.00 | 14.54 | S |
| ATOM | 3778 | O | HOH | S | 441 | 84.833 | 43.093 | 39.160 | 1.00 | 14.89 | S |
| ATOM | 3779 | O | HOH | S | 442 | 98.609 | 32.138 | 69.821 | 1.00 | 15.07 | S |
| ATOM | 3780 | O | HOH | S | 443 | 53.318 | 23.074 | 59.045 | 1.00 | 17.58 | S |
| ATOM | 3781 | O | HOH | S | 444 | 74.959 | 29.913 | 53.576 | 1.00 | 16.02 | S |
| ATOM | 3782 | O | HOH | S | 445 | 89.685 | 39.441 | 44.394 | 1.00 | 15.55 | S |
| ATOM | 3783 | O | HOH | S | 446 | 102.257 | 48.566 | 65.564 | 1.00 | 18.69 | S |
| ATOM | 3784 | O | HOH | S | 447 | 72.833 | 48.214 | 48.177 | 1.00 | 19.02 | S |
| ATOM | 3785 | O | HOH | S | 448 | 98.839 | 33.068 | 72.366 | 1.00 | 17.73 | S |
| ATOM | 3786 | O | HOH | S | 449 | 82.386 | 40.225 | 63.618 | 1.00 | 24.87 | S |
| ATOM | 3787 | O | HOH | S | 450 | 102.303 | 46.127 | 69.076 | 1.00 | 21.68 | S |
| ATOM | 3788 | O | HOH | S | 451 | 54.134 | 23.592 | 52.256 | 1.00 | 21.58 | S |
| ATOM | 3789 | O | HOH | S | 452 | 89.462 | 35.608 | 42.998 | 1.00 | 21.30 | S |
| ATOM | 3790 | O | HOH | S | 453 | 119.192 | 44.951 | 52.060 | 1.00 | 20.18 | S |
| ATOM | 3791 | O | HOH | S | 454 | 97.042 | 28.002 | 69.055 | 1.00 | 20.21 | S |
| ATOM | 3792 | O | HOH | S | 455 | 91.338 | 44.916 | 34.168 | 1.00 | 23.25 | S |
| ATOM | 3793 | O | HOH | S | 456 | 87.893 | 33.134 | 34.654 | 1.00 | 22.01 | S |
| ATOM | 3794 | O | HOH | S | 457 | 70.463 | 12.270 | 52.254 | 1.00 | 27.20 | S |
| ATOM | 3795 | O | HOH | S | 458 | 98.735 | 19.571 | 51.161 | 1.00 | 20.51 | S |
| ATOM | 3796 | O | HOH | S | 459 | 75.733 | 29.152 | 58.455 | 1.00 | 18.07 | S |
| ATOM | 3797 | O | HOH | S | 460 | 92.524 | 39.217 | 47.839 | 1.00 | 25.05 | S |
| ATOM | 3798 | O | HOH | S | 461 | 100.017 | 42.765 | 72.028 | 1.00 | 26.03 | S |
| ATOM | 3799 | O | HOH | S | 462 | 106.833 | 29.006 | 42.326 | 1.00 | 24.61 | S |
| ATOM | 3800 | O | HOH | S | 463 | 97.367 | 28.862 | 35.912 | 1.00 | 26.97 | S |
| ATOM | 3801 | O | HOH | S | 464 | 73.532 | 32.049 | 44.757 | 1.00 | 23.33 | S |
| ATOM | 3802 | O | HOH | S | 465 | 68.858 | 13.491 | 55.126 | 1.00 | 25.48 | S |
| ATOM | 3803 | O | HOH | S | 466 | 55.457 | 16.452 | 72.215 | 1.00 | 23.11 | S |
| ATOM | 3804 | O | HOH | S | 467 | 97.479 | 23.115 | 56.740 | 1.00 | 26.60 | S |
| ATOM | 3805 | O | HOH | S | 469 | 104.987 | 34.965 | 32.970 | 1.00 | 29.36 | S |
| ATOM | 3806 | O | HOH | S | 470 | 96.745 | 26.534 | 66.537 | 1.00 | 31.31 | S |
| ATOM | 3807 | O | HOH | S | 471 | 84.407 | 45.760 | 36.407 | 1.00 | 27.94 | S |
| ATOM | 3808 | O | HOH | S | 472 | 84.592 | 18.978 | 68.011 | 1.00 | 31.08 | S |
| ATOM | 3809 | O | HOH | S | 473 | 78.926 | 39.145 | 65.789 | 1.00 | 25.99 | S |
| ATOM | 3810 | O | HOH | S | 474 | 73.349 | 51.764 | 43.907 | 1.00 | 30.21 | S |
| ATOM | 3811 | O | HOH | S | 475 | 103.472 | 29.517 | 33.705 | 1.00 | 25.39 | S |
| ATOM | 3812 | O | HOH | S | 476 | 73.103 | 29.205 | 43.944 | 1.00 | 26.58 | S |
| ATOM | 3813 | O | HOH | S | 477 | 95.533 | 43.499 | 71.633 | 1.00 | 32.71 | S |
| ATOM | 3814 | O | HOH | S | 478 | 72.304 | 35.535 | 62.955 | 1.00 | 26.06 | S |
| ATOM | 3815 | O | HOH | S | 479 | 105.352 | 39.868 | 32.144 | 1.00 | 35.06 | S |
| ATOM | 3816 | O | HOH | S | 480 | 108.140 | 59.781 | 68.111 | 1.00 | 27.66 | S |
| ATOM | 3817 | O | HOH | S | 481 | 93.789 | 40.083 | 44.762 | 1.00 | 29.96 | S |
| ATOM | 3818 | O | HOH | S | 482 | 113.387 | 26.737 | 54.869 | 1.00 | 28.59 | S |
| ATOM | 3819 | O | HOH | S | 483 | 100.427 | 25.651 | 59.702 | 1.00 | 34.87 | S |
| ATOM | 3820 | O | HOH | S | 484 | 58.952 | 7.314 | 72.304 | 1.00 | 97.06 | S |
| ATOM | 3821 | O | HOH | S | 485 | 118.952 | 40.605 | 53.204 | 1.00 | 30.89 | S |
| ATOM | 3822 | O | HOH | S | 486 | 78.919 | 41.873 | 65.737 | 1.00 | 28.65 | S |
| ATOM | 3823 | O | HOH | S | 487 | 71.456 | 47.400 | 50.357 | 1.00 | 33.12 | S |
| ATOM | 3824 | O | HOH | S | 488 | 70.570 | 34.392 | 59.121 | 1.00 | 28.93 | S |
| ATOM | 3825 | O | HOH | S | 489 | 81.969 | 47.800 | 67.011 | 1.00 | 30.23 | S |
| ATOM | 3826 | O | HOH | S | 490 | 95.453 | 30.120 | 72.201 | 1.00 | 30.00 | S |
| ATOM | 3827 | O | HOH | S | 491 | 73.393 | 13.055 | 49.642 | 1.00 | 29.23 | S |
| ATOM | 3828 | O | HOH | S | 492 | 111.878 | 29.866 | 42.055 | 1.00 | 31.00 | S |
| ATOM | 3829 | O | HOH | S | 493 | 104.046 | 36.379 | 71.321 | 1.00 | 34.58 | S |
| ATOM | 3830 | O | HOH | S | 494 | 106.428 | 37.281 | 33.776 | 1.00 | 31.61 | S |
| ATOM | 3831 | O | HOH | S | 495 | 68.265 | 31.409 | 53.321 | 1.00 | 29.37 | S |
| ATOM | 3832 | O | HOH | S | 496 | 57.845 | 25.065 | 62.398 | 1.00 | 32.65 | S |
| ATOM | 3833 | O | HOH | S | 497 | 59.512 | 25.978 | 64.535 | 1.00 | 28.76 | S |
| ATOM | 3834 | O | HOH | S | 498 | 66.778 | 28.843 | 56.714 | 1.00 | 27.00 | S |
| ATOM | 3835 | O | HOH | S | 499 | 55.985 | 9.922 | 72.041 | 1.00 | 33.82 | S |
| ATOM | 3836 | O | HOH | S | 500 | 62.905 | 25.840 | 39.847 | 1.00 | 29.08 | S |
| ATOM | 3837 | O | HOH | S | 501 | 82.621 | 62.329 | 67.899 | 1.00 | 35.68 | S |
| ATOM | 3838 | O | HOH | S | 502 | 69.501 | 31.360 | 43.725 | 1.00 | 31.90 | S |
| ATOM | 3839 | O | HOH | S | 503 | 114.337 | 58.166 | 62.653 | 1.00 | 34.19 | S |
| ATOM | 3840 | O | HOH | S | 504 | 101.758 | 35.800 | 73.365 | 1.00 | 30.71 | S |
| ATOM | 3841 | O | HOH | S | 505 | 74.097 | 29.854 | 72.245 | 1.00 | 52.08 | S |
| ATOM | 3842 | O | HOH | S | 506 | 97.806 | 31.364 | 24.069 | 1.00 | 32.02 | S |
| ATOM | 3843 | O | HOH | S | 507 | 60.914 | 8.782 | 73.469 | 1.00 | 50.53 | S |
| ATOM | 3844 | O | HOH | S | 508 | 58.302 | 9.692 | 73.225 | 1.00 | 34.43 | S |
| ATOM | 3845 | O | HOH | S | 509 | 69.546 | 41.602 | 47.366 | 1.00 | 34.16 | S |
| ATOM | 3846 | O | HOH | S | 510 | 94.190 | 51.582 | 62.814 | 1.00 | 38.97 | S |
| ATOM | 3847 | O | HOH | S | 511 | 92.150 | 35.831 | 41.936 | 1.00 | 32.57 | S |
| ATOM | 3848 | O | HOH | S | 512 | 73.982 | 53.102 | 39.101 | 1.00 | 32.90 | S |
| ATOM | 3849 | O | HOH | S | 513 | 71.395 | 46.008 | 54.523 | 1.00 | 34.30 | S |
| ATOM | 3850 | O | HOH | S | 514 | 92.514 | 47.099 | 72.302 | 1.00 | 31.25 | S |
| ATOM | 3851 | O | HOH | S | 515 | 68.897 | 37.064 | 47.677 | 1.00 | 39.28 | S |
| ATOM | 3852 | O | HOH | S | 516 | 103.933 | 40.091 | 73.762 | 1.00 | 34.38 | S |
| ATOM | 3853 | O | HOH | S | 517 | 55.329 | 26.841 | 50.360 | 1.00 | 45.55 | S |
| ATOM | 3854 | O | HOH | S | 518 | 74.036 | 15.310 | 71.559 | 1.00 | 63.16 | S |
| ATOM | 3855 | O | HOH | S | 519 | 61.730 | 28.426 | 42.882 | 1.00 | 30.62 | S |
| ATOM | 3856 | O | HOH | S | 520 | 58.656 | 18.392 | 74.377 | 1.00 | 49.38 | S |
| ATOM | 3857 | O | HOH | S | 521 | 57.385 | 24.189 | 64.934 | 1.00 | 30.98 | S |
| ATOM | 3858 | O | HOH | S | 522 | 98.254 | 46.912 | 67.872 | 1.00 | 32.21 | S |
| ATOM | 3859 | O | HOH | S | 523 | 108.531 | 21.984 | 52.717 | 1.00 | 33.90 | S |
| ATOM | 3860 | O | HOH | S | 524 | 72.606 | 12.340 | 74.631 | 1.00 | 32.66 | S |
| ATOM | 3861 | O | HOH | S | 525 | 75.105 | 21.035 | 74.725 | 1.00 | 36.11 | S |
| ATOM | 3862 | O | HOH | S | 526 | 108.578 | 40.720 | 31.237 | 1.00 | 37.78 | S |
| ATOM | 3863 | O | HOH | S | 527 | 111.254 | 46.931 | 74.195 | 1.00 | 38.53 | S |
| ATOM | 3864 | O | HOH | S | 528 | 74.542 | 27.358 | 72.929 | 1.00 | 53.24 | S |
| ATOM | 3865 | O | HOH | S | 529 | 89.427 | 43.633 | 74.800 | 1.00 | 31.72 | S |
| ATOM | 3866 | O | HOH | S | 530 | 116.286 | 47.543 | 43.182 | 1.00 | 38.99 | S |
| ATOM | 3867 | O | HOH | S | 531 | 69.356 | 38.980 | 54.585 | 1.00 | 37.44 | S |
| ATOM | 3868 | O | HOH | S | 532 | 113.871 | 39.130 | 42.603 | 1.00 | 41.76 | S |
| ATOM | 3869 | O | HOH | S | 533 | 84.683 | 51.633 | 32.897 | 1.00 | 36.25 | S |
| ATOM | 3870 | O | HOH | S | 534 | 111.339 | 41.771 | 33.324 | 1.00 | 41.88 | S |
| ATOM | 3871 | O | HOH | S | 535 | 97.678 | 42.339 | 70.764 | 1.00 | 38.41 | S |
| ATOM | 3872 | O | HOH | S | 536 | 99.843 | 49.683 | 31.874 | 1.00 | 37.26 | S |
| ATOM | 3873 | O | HOH | S | 537 | 90.286 | 30.261 | 36.959 | 1.00 | 41.32 | S |
| ATOM | 3874 | O | HOH | S | 538 | 99.148 | 31.358 | 32.137 | 1.00 | 36.54 | S |
| ATOM | 3875 | O | HOH | S | 539 | 94.902 | 68.199 | 60.005 | 1.00 | 43.55 | S |
| ATOM | 3876 | O | HOH | S | 540 | 110.369 | 32.045 | 66.000 | 1.00 | 36.29 | S |
| ATOM | 3877 | O | HOH | S | 541 | 112.153 | 25.143 | 53.103 | 1.00 | 36.40 | S |
| ATOM | 3878 | O | HOH | S | 542 | 52.351 | 10.762 | 68.574 | 1.00 | 43.03 | S |
| ATOM | 3879 | O | HOH | S | 543 | 95.088 | 24.446 | 59.011 | 1.00 | 31.37 | S |
| ATOM | 3880 | O | HOH | S | 544 | 70.158 | 32.073 | 57.533 | 1.00 | 32.00 | S |
| ATOM | 3881 | O | HOH | S | 545 | 117.475 | 25.180 | 49.812 | 1.00 | 36.63 | S |
| ATOM | 3882 | O | HOH | S | 546 | 118.329 | 45.081 | 45.249 | 1.00 | 37.26 | S |
| ATOM | 3883 | O | HOH | S | 547 | 88.930 | 29.834 | 71.420 | 1.00 | 38.44 | S |
| ATOM | 3884 | O | HOH | S | 548 | 99.244 | 58.990 | 31.862 | 1.00 | 41.38 | S |
| ATOM | 3885 | O | HOH | S | 549 | 106.791 | 46.331 | 72.657 | 1.00 | 38.60 | S |
| ATOM | 3886 | O | HOH | S | 550 | 80.606 | 51.988 | 66.436 | 1.00 | 34.95 | S |
| ATOM | 3887 | O | HOH | S | 551 | 118.874 | 38.112 | 54.133 | 1.00 | 71.68 | S |
| ATOM | 3888 | O | HOH | S | 552 | 107.216 | 24.299 | 58.929 | 1.00 | 38.61 | S |
| ATOM | 3889 | O | HOH | S | 553 | 72.733 | 12.236 | 55.287 | 1.00 | 32.78 | S |
| ATOM | 3890 | O | HOH | S | 554 | 117.506 | 32.629 | 49.596 | 1.00 | 32.08 | S |
| ATOM | 3891 | O | HOH | S | 555 | 64.234 | 31.262 | 44.768 | 1.00 | 33.38 | S |
| ATOM | 3892 | O | HOH | S | 556 | 67.256 | 41.705 | 49.177 | 1.00 | 38.96 | S |
| ATOM | 3893 | O | HOH | S | 557 | 90.353 | 42.130 | 46.089 | 1.00 | 35.65 | S |
| ATOM | 3894 | O | HOH | S | 558 | 70.417 | 31.163 | 51.588 | 1.00 | 35.99 | S |
| ATOM | 3895 | O | HOH | S | 559 | 108.643 | 60.970 | 65.420 | 1.00 | 37.08 | S |
| ATOM | 3896 | O | HOH | S | 560 | 106.888 | 53.175 | 33.245 | 1.00 | 42.10 | S |
| ATOM | 3897 | O | HOH | S | 561 | 93.327 | 51.275 | 33.022 | 1.00 | 37.99 | S |
| ATOM | 3898 | O | HOH | S | 562 | 53.694 | 16.168 | 49.310 | 1.00 | 32.59 | S |
| ATOM | 3899 | O | HOH | S | 563 | 92.962 | 62.288 | 64.295 | 1.00 | 40.02 | S |
| ATOM | 3900 | O | HOH | S | 564 | 99.036 | 69.995 | 55.241 | 1.00 | 36.89 | S |
| ATOM | 3901 | O | HOH | S | 565 | 81.792 | 29.855 | 69.180 | 1.00 | 37.87 | S |
| ATOM | 3902 | O | HOH | S | 566 | 93.641 | 65.239 | 46.496 | 1.00 | 72.71 | S |
| ATOM | 3903 | O | HOH | S | 567 | 88.377 | 57.211 | 36.919 | 1.00 | 41.60 | S |
| ATOM | 3904 | O | HOH | S | 568 | 116.937 | 41.243 | 42.702 | 1.00 | 38.27 | S |
| ATOM | 3905 | O | HOH | S | 569 | 61.146 | 22.869 | 73.062 | 1.00 | 37.31 | S |
| ATOM | 3906 | O | HOH | S | 570 | 114.088 | 54.266 | 48.511 | 1.00 | 33.10 | S |
| ATOM | 3907 | O | HOH | S | 571 | 72.287 | 33.892 | 67.244 | 1.00 | 38.56 | S |
| ATOM | 3908 | O | HOH | S | 572 | 77.843 | 38.575 | 68.442 | 1.00 | 42.96 | S |
| ATOM | 3909 | O | HOH | S | 573 | 104.458 | 34.055 | 70.041 | 1.00 | 32.50 | S |
| ATOM | 3910 | O | HOH | S | 574 | 109.580 | 43.221 | 31.836 | 1.00 | 41.74 | S |
| ATOM | 3911 | O | HOH | S | 575 | 78.352 | 28.016 | 73.341 | 1.00 | 43.01 | S |
| ATOM | 3912 | O | HOH | S | 576 | 78.044 | 48.028 | 66.608 | 1.00 | 37.32 | S |
| ATOM | 3913 | O | HOH | S | 577 | 52.904 | 19.663 | 49.427 | 1.00 | 39.39 | S |
| ATOM | 3914 | O | HOH | S | 578 | 72.456 | −3.062 | 73.726 | 1.00 | 40.43 | S |
| ATOM | 3915 | O | HOH | S | 579 | 71.483 | 42.012 | 59.449 | 1.00 | 46.92 | S |
| ATOM | 3916 | O | HOH | S | 580 | 60.532 | −0.586 | 76.593 | 1.00 | 38.24 | S |
| ATOM | 3917 | O | HOH | S | 581 | 76.680 | 47.475 | 52.463 | 1.00 | 92.24 | S |
| ATOM | 3918 | O | HOH | S | 582 | 74.586 | 41.157 | 64.727 | 1.00 | 40.99 | S |
| ATOM | 3919 | O | HOH | S | 583 | 110.478 | 58.439 | 40.085 | 1.00 | 40.27 | S |
| ATOM | 3920 | O | HOH | S | 584 | 56.321 | 26.034 | 59.936 | 1.00 | 45.21 | S |
| ATOM | 3921 | O | HOH | S | 585 | 77.172 | 56.213 | 52.289 | 1.00 | 48.52 | S |
| ATOM | 3922 | O | HOH | S | 586 | 70.758 | 44.878 | 45.950 | 1.00 | 35.82 | S |
| ATOM | 3923 | O | HOH | S | 587 | 52.644 | −1.852 | 70.441 | 1.00 | 43.73 | S |
| ATOM | 3924 | O | HOH | S | 588 | 87.481 | 39.926 | 76.844 | 1.00 | 42.18 | S |
| ATOM | 3925 | O | HOH | S | 589 | 63.253 | 28.371 | 65.580 | 1.00 | 41.33 | S |
| ATOM | 3926 | O | HOH | S | 590 | 115.422 | 41.630 | 35.893 | 1.00 | 42.35 | S |
| ATOM | 3927 | O | HOH | S | 591 | 61.587 | 3.655 | 77.956 | 1.00 | 42.24 | S |
| ATOM | 3928 | O | HOH | S | 592 | 117.442 | 55.227 | 51.881 | 1.00 | 39.44 | S |
| ATOM | 3929 | O | HOH | S | 593 | 119.113 | 48.636 | 48.271 | 1.00 | 43.70 | S |
| ATOM | 3930 | O | HOH | S | 594 | 82.341 | 33.779 | 70.964 | 1.00 | 42.06 | S |
| ATOM | 3931 | O | HOH | S | 595 | 88.296 | 27.516 | 66.696 | 1.00 | 49.81 | S |
| ATOM | 3932 | O | HOH | S | 596 | 117.012 | 31.902 | 54.050 | 1.00 | 39.61 | S |
| ATOM | 3933 | O | HOH | S | 597 | 114.815 | 30.315 | 48.504 | 1.00 | 83.02 | S |
| ATOM | 3934 | O | HOH | S | 598 | 109.668 | 63.164 | 50.855 | 1.00 | 48.87 | S |
| ATOM | 3935 | O | HOH | S | 599 | 75.109 | 48.828 | 64.841 | 1.00 | 35.06 | S |
| ATOM | 3936 | O | HOH | S | 600 | 85.165 | 28.815 | 69.854 | 1.00 | 45.15 | S |
| ATOM | 3937 | O | HOH | S | 601 | 87.737 | 64.196 | 51.323 | 1.00 | 44.88 | S |
| ATOM | 3938 | O | HOH | S | 602 | 83.217 | 55.953 | 41.485 | 1.00 | 38.92 | S |
| ATOM | 3939 | O | HOH | S | 603 | 105.753 | 58.988 | 69.189 | 1.00 | 41.13 | S |
| ATOM | 3940 | O | HOH | S | 604 | 62.472 | 29.198 | 40.185 | 1.00 | 40.57 | S |
| ATOM | 3941 | O | HOH | S | 605 | 72.770 | 44.256 | 59.995 | 1.00 | 38.47 | S |
| ATOM | 3942 | O | HOH | S | 606 | 107.412 | 44.046 | 39.790 | 1.00 | 84.44 | S |
| ATOM | 3943 | O | HOH | S | 607 | 90.199 | 66.564 | 56.046 | 1.00 | 43.68 | S |
| ATOM | 3944 | O | HOH | S | 608 | 105.471 | 41.446 | 34.679 | 1.00 | 43.13 | S |
| ATOM | 3945 | O | HOH | S | 609 | 65.085 | 28.542 | 69.615 | 1.00 | 42.09 | S |
| ATOM | 3946 | O | HOH | S | 610 | 75.130 | 26.428 | 75.442 | 1.00 | 49.87 | S |
| ATOM | 3947 | O | HOH | S | 611 | 63.740 | −2.928 | 74.893 | 1.00 | 45.49 | S |
| ATOM | 3948 | O | HOH | S | 612 | 98.505 | 69.485 | 51.067 | 1.00 | 48.32 | S |
| ATOM | 3949 | O | HOH | S | 613 | 83.552 | 60.026 | 46.232 | 1.00 | 46.79 | S |
| ATOM | 3950 | O | HOH | S | 614 | 109.111 | 32.543 | 28.365 | 1.00 | 58.63 | S |
| ATOM | 3951 | O | HOH | S | 615 | 73.199 | 36.309 | 65.337 | 1.00 | 39.11 | S |
| ATOM | 3952 | O | HOH | S | 616 | 112.021 | 48.174 | 34.823 | 1.00 | 47.54 | S |
| ATOM | 3953 | O | HOH | S | 617 | 93.619 | 40.294 | 74.590 | 1.00 | 44.08 | S |
| ATOM | 3954 | O | HOH | S | 618 | 94.336 | 49.501 | 69.276 | 1.00 | 39.85 | S |
| ATOM | 3955 | O | HOH | S | 619 | 109.925 | 34.285 | 35.188 | 1.00 | 40.15 | S |
| ATOM | 3956 | O | HOH | S | 620 | 57.418 | 27.295 | 48.945 | 1.00 | 81.24 | S |
| ATOM | 3957 | O | HOH | S | 621 | 70.442 | 0.738 | 75.534 | 1.00 | 40.83 | S |
| ATOM | 3958 | O | HOH | S | 622 | 99.174 | 19.583 | 55.584 | 1.00 | 58.65 | S |
| ATOM | 3959 | O | HOH | S | 623 | 73.819 | 54.971 | 56.707 | 1.00 | 43.07 | S |
| ATOM | 3960 | O | HOH | S | 624 | 115.667 | 26.710 | 45.100 | 1.00 | 39.67 | S |
| ATOM | 3961 | O | HOH | S | 625 | 92.685 | 54.340 | 63.523 | 1.00 | 40.74 | S |
| ATOM | 3962 | O | HOH | S | 626 | 106.609 | 64.388 | 62.582 | 1.00 | 39.34 | S |
| ATOM | 3963 | O | HOH | S | 627 | 116.850 | 50.827 | 40.937 | 1.00 | 47.36 | S |
| ATOM | 3964 | O | HOH | S | 628 | 104.125 | 25.579 | 60.763 | 1.00 | 41.89 | S |
| ATOM | 3965 | O | HOH | S | 629 | 93.640 | 67.445 | 64.096 | 1.00 | 47.10 | S |
| ATOM | 3966 | O | HOH | S | 630 | 100.512 | 62.597 | 60.677 | 1.00 | 42.91 | S |
| ATOM | 3967 | O | HOH | S | 631 | 89.048 | 55.274 | 31.827 | 1.00 | 48.66 | S |
| ATOM | 3968 | O | HOH | S | 632 | 113.614 | 38.271 | 38.578 | 1.00 | 37.96 | S |
| ATOM | 3969 | O | HOH | S | 633 | 50.507 | 20.933 | 52.873 | 1.00 | 45.04 | S |
| ATOM | 3970 | O | HOH | S | 634 | 72.079 | 55.675 | 42.939 | 1.00 | 53.38 | S |
| ATOM | 3971 | O | HOH | S | 635 | 106.401 | 61.539 | 42.887 | 1.00 | 49.81 | S |
| ATOM | 3972 | O | HOH | S | 636 | 71.352 | 48.771 | 55.468 | 1.00 | 38.80 | S |
| ATOM | 3973 | O | HOH | S | 637 | 78.749 | 40.132 | 72.827 | 1.00 | 41.08 | S |
| ATOM | 3974 | O | HOH | S | 638 | 85.569 | 59.479 | 42.360 | 1.00 | 53.91 | S |
| ATOM | 3975 | O | HOH | S | 639 | 94.735 | 55.793 | 30.201 | 1.00 | 42.25 | S |
| ATOM | 3976 | O | HOH | S | 640 | 76.916 | 53.521 | 63.015 | 1.00 | 41.37 | S |
| ATOM | 3977 | O | HOH | S | 641 | 111.683 | 62.552 | 62.654 | 1.00 | 37.53 | S |
| ATOM | 3978 | O | HOH | S | 642 | 91.556 | 54.362 | 31.631 | 1.00 | 44.12 | S |
| ATOM | 3979 | O | HOH | S | 643 | 109.180 | 62.922 | 53.604 | 1.00 | 41.11 | S |
| ATOM | 3980 | O | HOH | S | 644 | 56.780 | 23.251 | 46.220 | 1.00 | 51.73 | S |
| ATOM | 3981 | O | HOH | S | 645 | 98.168 | 36.070 | 75.483 | 1.00 | 44.97 | S |
| ATOM | 3982 | O | HOH | S | 646 | 80.183 | 57.655 | 47.765 | 1.00 | 56.75 | S |
| ATOM | 3983 | O | HOH | S | 647 | 100.580 | 57.101 | 30.431 | 1.00 | 42.26 | S |
| ATOM | 3984 | O | HOH | S | 648 | 77.521 | 57.646 | 57.357 | 1.00 | 47.65 | S |
| ATOM | 3985 | O | HOH | S | 649 | 98.730 | 51.205 | 62.185 | 1.00 | 42.42 | S |
| ATOM | 3986 | O | HOH | S | 650 | 65.285 | 30.706 | 40.008 | 1.00 | 40.02 | S |
| ATOM | 3987 | O | HOH | S | 651 | 106.145 | 56.237 | 46.484 | 1.00 | 81.50 | S |
| ATOM | 3988 | O | HOH | S | 652 | 111.155 | 29.979 | 61.798 | 1.00 | 52.61 | S |
| ATOM | 3989 | O | HOH | S | 653 | 100.391 | 52.207 | 64.763 | 1.00 | 49.80 | S |
| ATOM | 3990 | O | HOH | S | 654 | 111.546 | 30.165 | 57.582 | 1.00 | 45.61 | S |
| ATOM | 3991 | O | HOH | S | 655 | 81.276 | 64.020 | 56.845 | 1.00 | 43.54 | S |
| ATOM | 3992 | O | HOH | S | 656 | 114.731 | 50.561 | 37.556 | 1.00 | 71.63 | S |
| ATOM | 3993 | O | HOH | S | 657 | 58.556 | 3.430 | 78.153 | 1.00 | 45.44 | S |
| ATOM | 3994 | O | HOH | S | 658 | 102.366 | 44.165 | 45.213 | 1.00 | 93.72 | S |
| ATOM | 3995 | O | HOH | S | 659 | 62.063 | 12.603 | 76.791 | 1.00 | 42.87 | S |
| ATOM | 3996 | O | HOH | S | 660 | 106.457 | 54.974 | 71.489 | 1.00 | 37.97 | S |
| ATOM | 3997 | O | HOH | S | 661 | 114.061 | 33.688 | 37.361 | 1.00 | 43.75 | S |
| ATOM | 3998 | O | HOH | S | 662 | 107.928 | 28.933 | 64.188 | 1.00 | 43.88 | S |
| ATOM | 3999 | O | HOH | S | 663 | 95.706 | 63.114 | 55.053 | 1.00 | 104.80 | S |
| ATOM | 4000 | O | HOH | S | 664 | 96.529 | 61.733 | 35.051 | 1.00 | 46.52 | S |
| ATOM | 4001 | O | HOH | S | 665 | 92.033 | 70.582 | 57.868 | 1.00 | 40.24 | S |
| ATOM | 4002 | O | HOH | S | 666 | 104.496 | 58.731 | 33.996 | 1.00 | 51.50 | S |
| ATOM | 4003 | O | HOH | S | 667 | 74.993 | 10.057 | 77.796 | 1.00 | 50.22 | S |
| ATOM | 4004 | O | HOH | S | 668 | 83.392 | 58.064 | 43.085 | 1.00 | 51.79 | S |
| ATOM | 4005 | O | HOH | S | 669 | 96.273 | 18.435 | 55.190 | 1.00 | 43.38 | S |
| ATOM | 4006 | O | HOH | S | 670 | 91.592 | 58.562 | 66.427 | 1.00 | 47.90 | S |
| ATOM | 4007 | O | HOH | S | 671 | 113.092 | 31.218 | 59.994 | 1.00 | 41.28 | S |
| ATOM | 4008 | O | HOH | S | 672 | 111.566 | 58.006 | 50.890 | 1.00 | 46.74 | S |
| ATOM | 4009 | O | HOH | S | 673 | 77.568 | 54.829 | 55.835 | 1.00 | 64.31 | S |
| ATOM | 4010 | O | HOH | S | 674 | 61.169 | 26.335 | 61.940 | 1.00 | 63.83 | S |
| ATOM | 4011 | O | HOH | S | 675 | 92.663 | 48.820 | 33.306 | 1.00 | 49.91 | S |
| ATOM | 4012 | O | HOH | S | 676 | 83.468 | 18.457 | 72.335 | 1.00 | 48.75 | S |
| ATOM | 4013 | O | HOH | S | 677 | 91.275 | 28.198 | 72.686 | 1.00 | 46.00 | S |
| ATOM | 4014 | O | HOH | S | 678 | 50.983 | 16.418 | 55.152 | 1.00 | 45.44 | S |
| ATOM | 4015 | O | HOH | S | 679 | 70.036 | −2.020 | 73.686 | 1.00 | 38.11 | S |
| ATOM | 4016 | O | HOH | S | 680 | 76.383 | 17.460 | 72.840 | 1.00 | 38.28 | S |
| ATOM | 4017 | O | HOH | S | 681 | 74.669 | 28.415 | 51.438 | 1.00 | 46.34 | S |
| ATOM | 4018 | O | HOH | S | 682 | 102.565 | 48.864 | 32.669 | 1.00 | 49.65 | S |
| ATOM | 4019 | O | HOH | S | 683 | 115.774 | 62.569 | 57.199 | 1.00 | 49.54 | S |
| ATOM | 4020 | O | HOH | S | 684 | 112.092 | 39.145 | 73.725 | 1.00 | 48.53 | S |
| ATOM | 4021 | O | HOH | S | 685 | 58.847 | 19.913 | 72.399 | 1.00 | 65.61 | S |
| ATOM | 4022 | O | HOH | S | 686 | 68.776 | 24.453 | 56.059 | 1.00 | 68.49 | S |
| ATOM | 4023 | O | HOH | S | 687 | 73.851 | 55.058 | 52.125 | 1.00 | 43.81 | S |
| ATOM | 4024 | O | HOH | S | 688 | 56.985 | 22.906 | 42.636 | 1.00 | 46.74 | S |
| ATOM | 4025 | O | HOH | S | 689 | 79.072 | 35.800 | 73.222 | 1.00 | 49.41 | S |
| ATOM | 4026 | O | HOH | S | 690 | 97.783 | 39.056 | 74.034 | 1.00 | 46.89 | S |
| ATOM | 4027 | O | HOH | S | 691 | 88.318 | 34.733 | 75.404 | 1.00 | 51.54 | S |
| ATOM | 4028 | O | HOH | S | 692 | 86.539 | 55.035 | 34.586 | 1.00 | 50.54 | S |
| ATOM | 4029 | O | HOH | S | 693 | 96.378 | 38.321 | 76.445 | 1.00 | 47.25 | S |
| ATOM | 4030 | O | HOH | S | 694 | 67.790 | 29.675 | 64.051 | 1.00 | 44.69 | S |
| ATOM | 4031 | O | HOH | S | 695 | 87.305 | 67.532 | 61.456 | 1.00 | 46.22 | S |
| ATOM | 4032 | O | HOH | S | 696 | 92.671 | 37.006 | 59.125 | 1.00 | 85.28 | S |
| ATOM | 4033 | O | HOH | S | 697 | 94.315 | 28.875 | 32.064 | 1.00 | 53.63 | S |
| ATOM | 4034 | O | HOH | S | 698 | 80.992 | 27.904 | 65.164 | 1.00 | 68.05 | S |
| ATOM | 4035 | O | HOH | S | 699 | 98.939 | 65.597 | 61.504 | 1.00 | 45.60 | S |
| ATOM | 4036 | O | HOH | S | 700 | 118.902 | 35.114 | 62.083 | 1.00 | 41.64 | S |
| ATOM | 4037 | O | HOH | S | 701 | 77.404 | 51.724 | 65.052 | 1.00 | 46.57 | S |
| ATOM | 4038 | O | HOH | S | 702 | 87.334 | 62.419 | 45.461 | 1.00 | 64.03 | S |
| ATOM | 4039 | O | HOH | S | 703 | 63.195 | 32.775 | 52.642 | 1.00 | 50.12 | S |
| ATOM | 4040 | O | HOH | S | 704 | 68.994 | 42.042 | 54.981 | 1.00 | 48.80 | S |
| ATOM | 4041 | O | HOH | S | 705 | 90.496 | 27.069 | 68.037 | 1.00 | 43.01 | S |
| ATOM | 4042 | O | HOH | S | 706 | 64.792 | 25.511 | 72.203 | 1.00 | 40.11 | S |
| ATOM | 4043 | O | HOH | S | 707 | 59.461 | 29.407 | 59.121 | 1.00 | 55.49 | S |
| ATOM | 4044 | O | HOH | S | 708 | 96.584 | 65.688 | 48.486 | 1.00 | 45.30 | S |
| ATOM | 4045 | O | HOH | S | 709 | 82.718 | 54.542 | 37.256 | 1.00 | 43.40 | S |
| ATOM | 4046 | O | HOH | S | 710 | 72.898 | 24.783 | 76.113 | 1.00 | 51.23 | S |
| ATOM | 4047 | O | HOH | S | 711 | 75.024 | 11.142 | 74.369 | 1.00 | 72.49 | S |
| ATOM | 4048 | O | HOH | S | 712 | 109.968 | 49.900 | 34.897 | 1.00 | 97.24 | S |
| ATOM | 4049 | O | HOH | S | 713 | 114.187 | 35.278 | 41.022 | 1.00 | 43.77 | S |
| ATOM | 4050 | O | HOH | S | 714 | 112.092 | 44.139 | 74.615 | 1.00 | 43.72 | S |
| ATOM | 4051 | O | HOH | S | 715 | 77.458 | 10.343 | 73.699 | 1.00 | 76.53 | S |
| ATOM | 4052 | O | HOH | S | 716 | 54.937 | 21.381 | 46.947 | 1.00 | 49.55 | S |
| ATOM | 4053 | O | HOH | S | 717 | 83.899 | 63.831 | 52.215 | 1.00 | 46.65 | S |
| ATOM | 4054 | O | HOH | S | 718 | 102.989 | 68.880 | 49.748 | 1.00 | 43.40 | S |
| ATOM | 4055 | O | HOH | S | 719 | 58.927 | 8.177 | 77.312 | 1.00 | 43.86 | S |
| ATOM | 4056 | O | HOH | S | 720 | 99.748 | 54.248 | 30.734 | 1.00 | 43.48 | S |
| ATOM | 4057 | O | HOH | S | 721 | 113.810 | 44.886 | 32.848 | 1.00 | 56.94 | S |
| ATOM | 4058 | O | HOH | S | 722 | 97.484 | 64.952 | 44.562 | 1.00 | 53.88 | S |
| ATOM | 4059 | O | HOH | S | 723 | 115.681 | 36.565 | 42.867 | 1.00 | 68.32 | S |
| ATOM | 4060 | O | HOH | S | 724 | 114.250 | 53.235 | 37.338 | 1.00 | 51.97 | S |
| ATOM | 4061 | O | HOH | S | 725 | 74.683 | 21.882 | 51.999 | 1.00 | 93.79 | S |
| ATOM | 4062 | O | HOH | S | 726 | 113.454 | 55.540 | 39.786 | 1.00 | 57.91 | S |

TABLE 3
Amino acid residues corresponding to the putative active
site/neuraminidase inhibitor-binding site (residues within 10 Å).

| | | | | | | | |
|---|---|---|---|---|---|---|---|
| Tyr21 | Val46 | Val80 | Ala130 | Tyr180 | Gly200 | Val237 | Ser295 |
| His23 | Gly47 | Thr95 | His 131 | Asn181 | Ser201 | Ala238 | Gly296 |
| Phe24 | Asp76 | Tyr97 | Tyr146 | Glu182 | Gly202 | Thr258 | Tyr297 |
| Glu44 | Arg78 | Tyr127 | Tyr153 | Arg198 | Ile235 | Arg260 | Phe313 |
| His45 | Asp79 | Phe129 | Pro179 | Val199 | Leu236 | Ala294 | Glu315 |

### Example 14

In silico screening. To demonstrate the feasibility of identifying potential molecule inhibitors, in silico, computational modeling software was utilized in conjunction with high-resolution crystal structure results to screen databases for existing compounds that would bind to the active site of the Pseudomonas neuraminidase (see Sherman et al., (2006) Chem Biol Drug Des 67(1): 83-4). This limited search carried out by Schrodinger (Schrodinger, LLC, New York) yielded candidate neuraminidase inhibitors that are predicted to bind the active site (see Table 4 below). Visualization, structural refinement, and docking were performed using Maestro 7.0, MacroModel 9.0, Prime 1.2, Glide 3.5, and IFD script from Schrodinger, LLC (New York).

TABLE 4
Candidate neuraminidase inhibitors predicted
to bind to the active site of Pseudomonas neuraminidase.

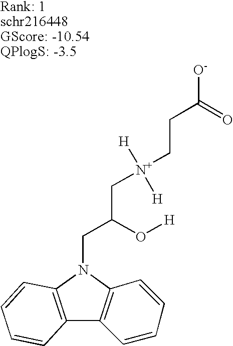

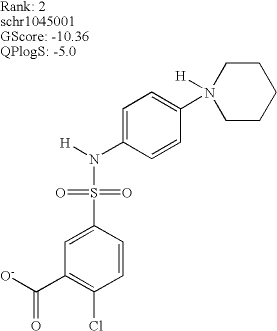

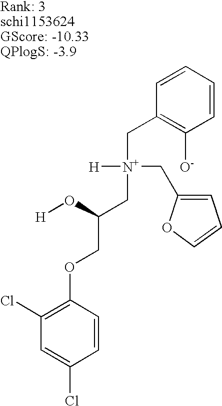

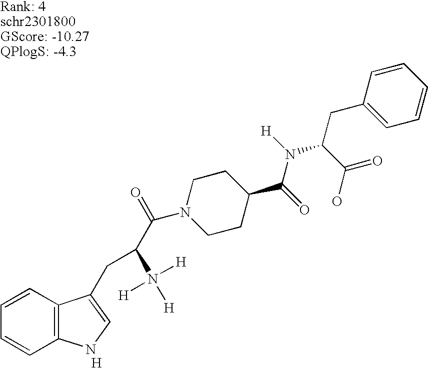

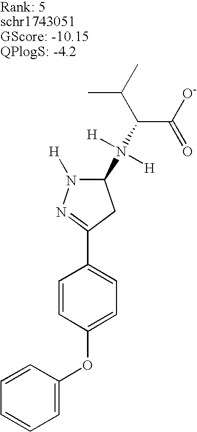

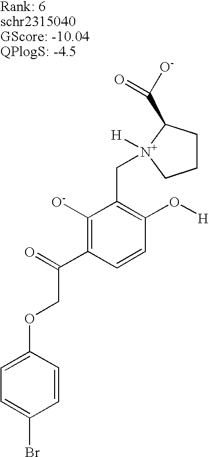

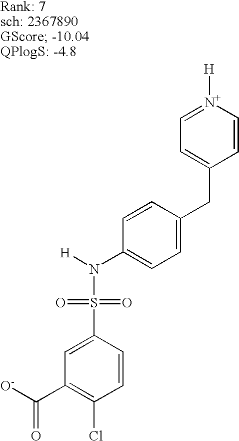

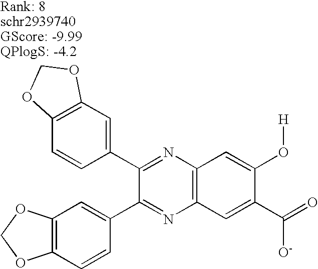

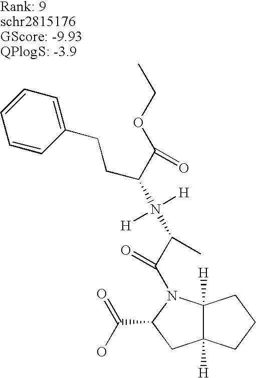

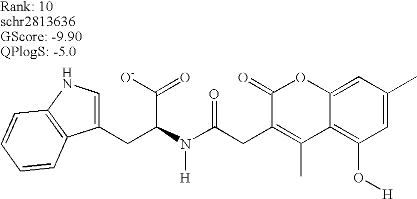

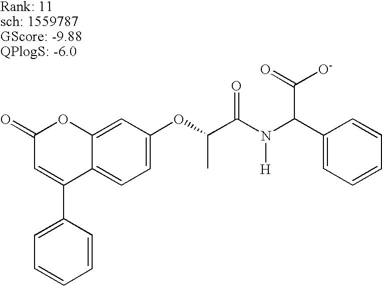

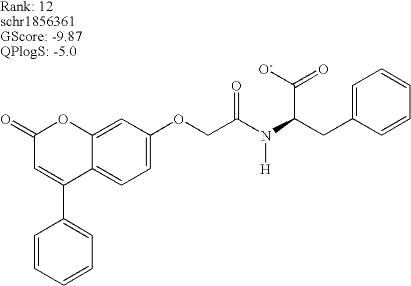

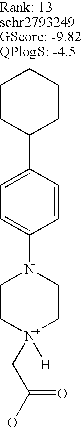

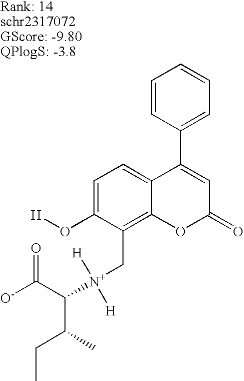

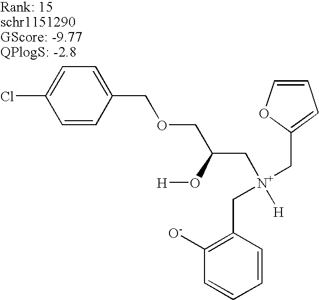

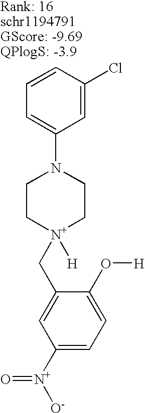

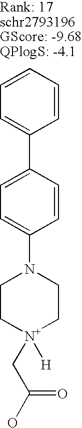

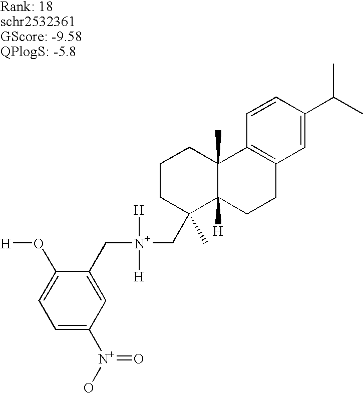

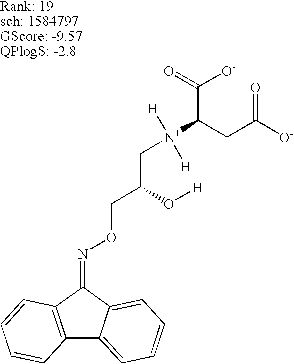

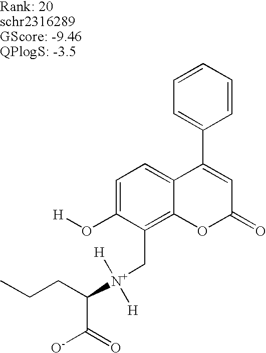

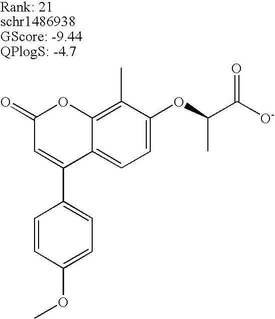

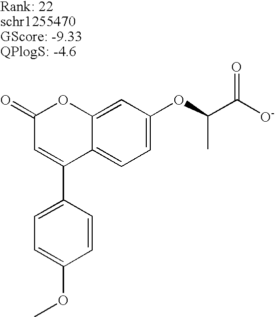

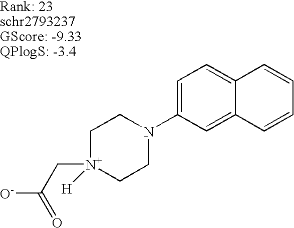

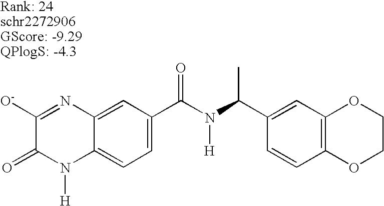

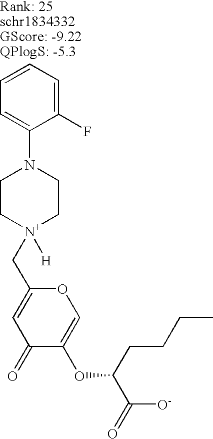

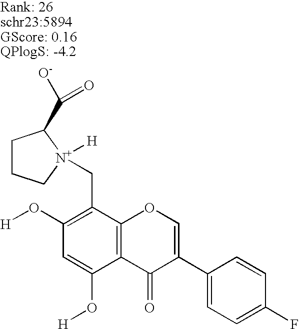

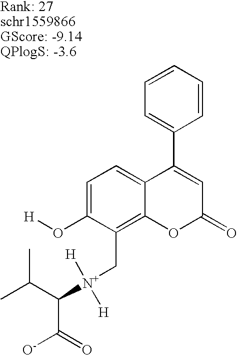

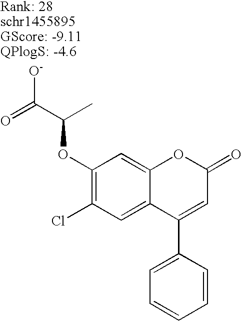

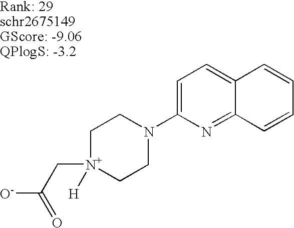

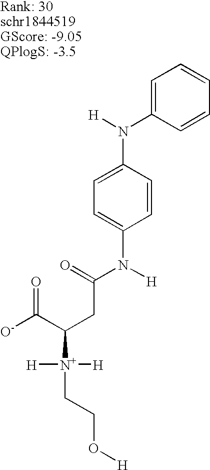

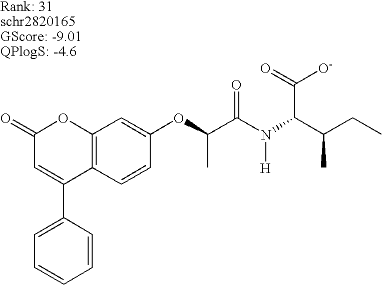

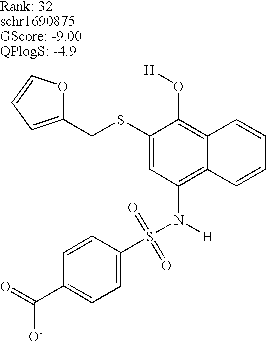

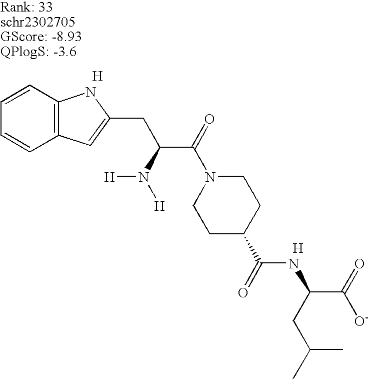

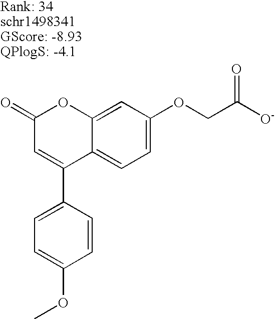

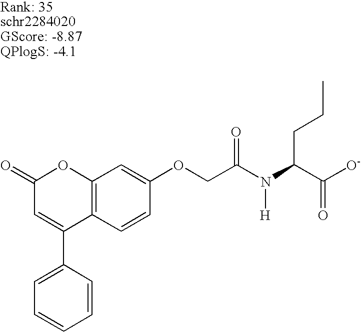

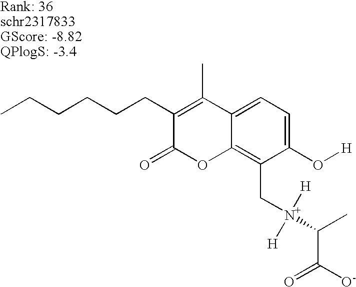

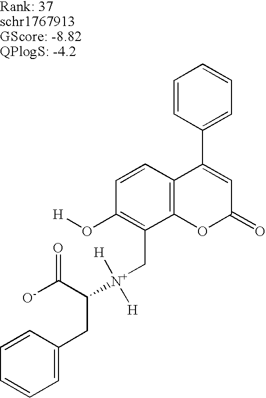

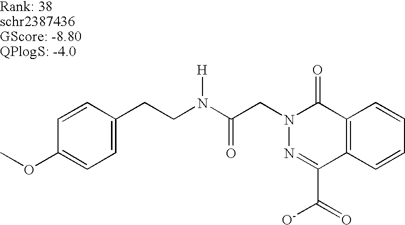

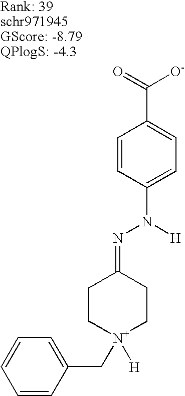

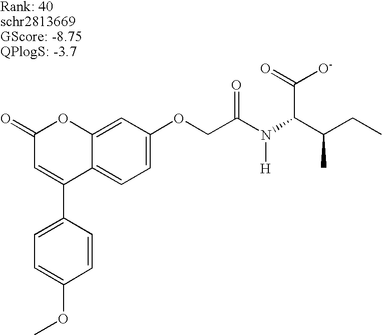

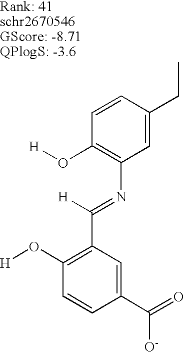

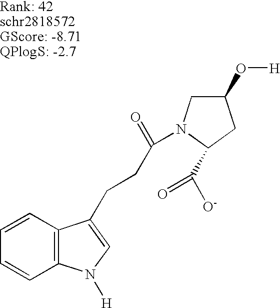

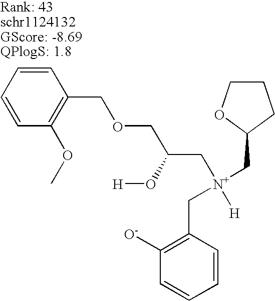

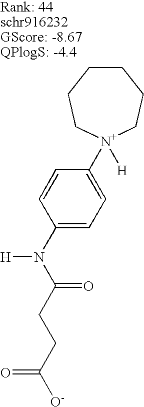

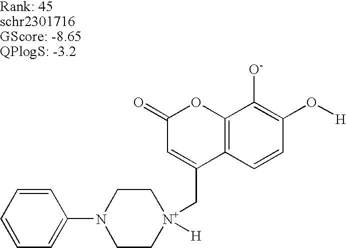

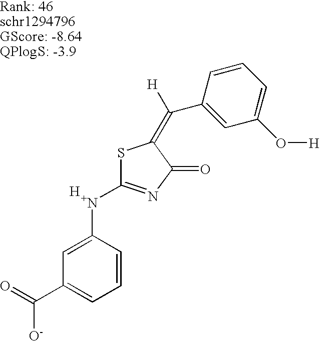

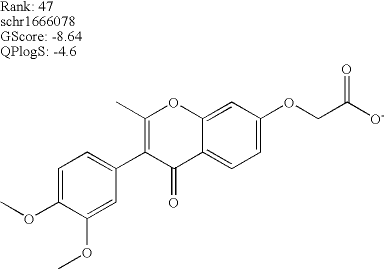

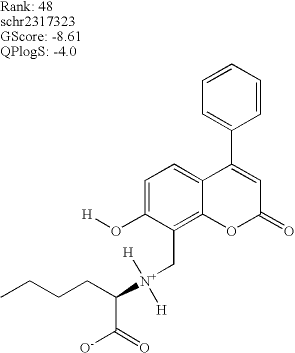

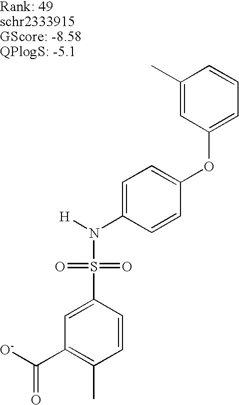

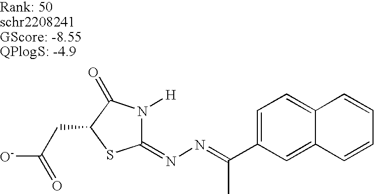

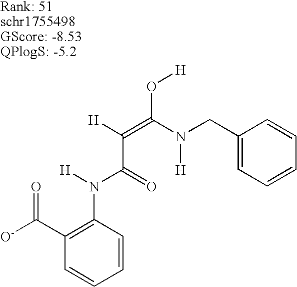

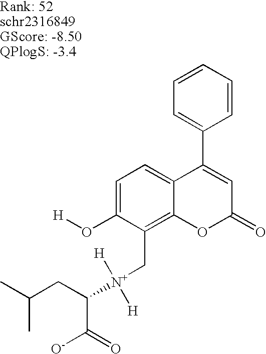

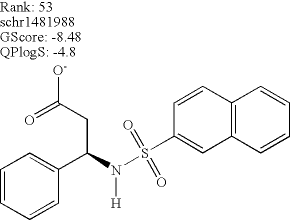

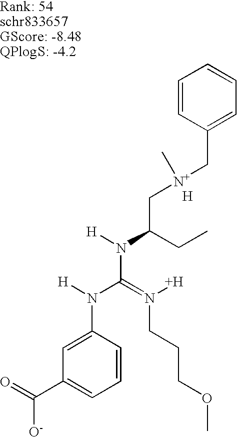

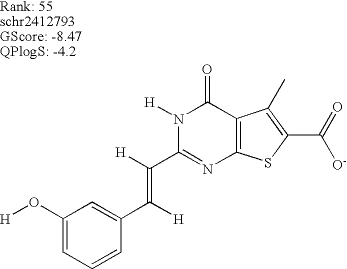

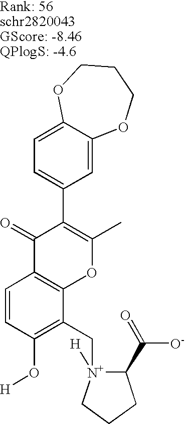

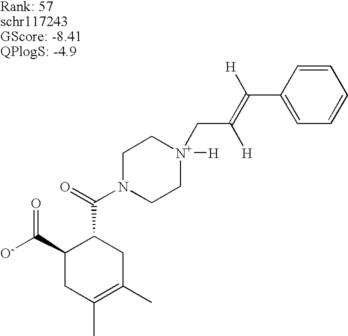

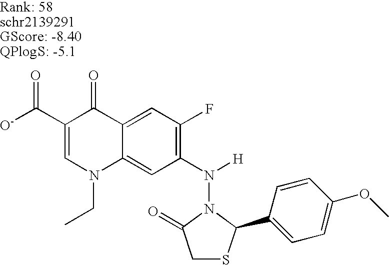

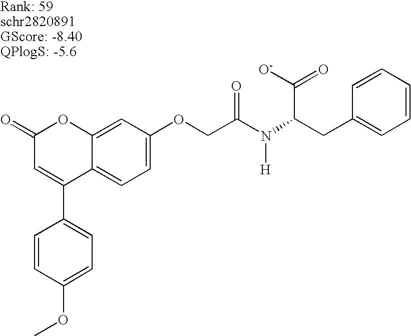

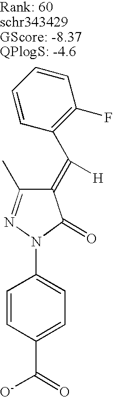

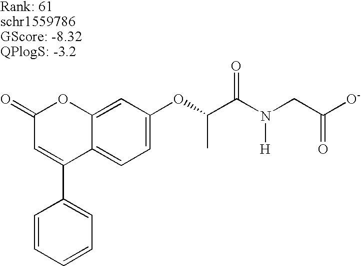

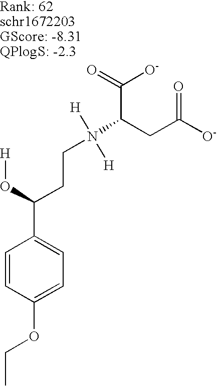

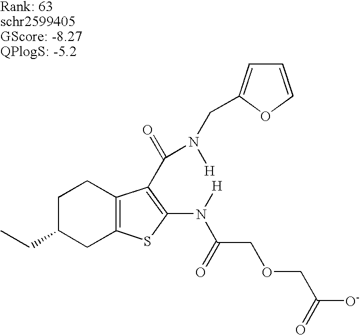

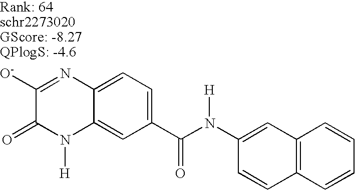

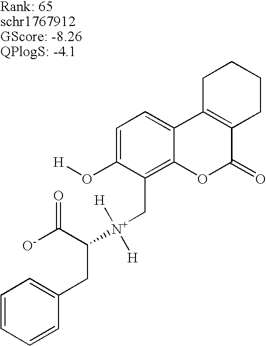

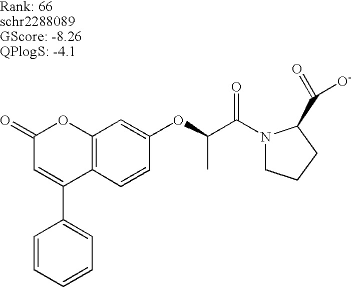

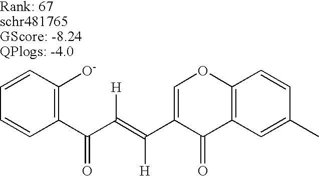

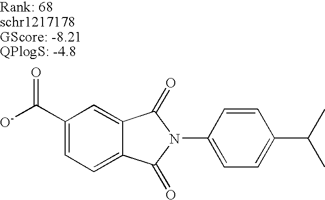

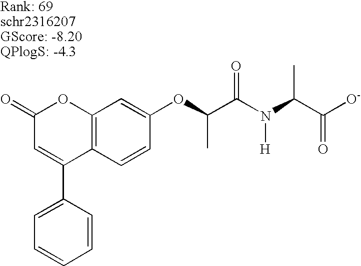

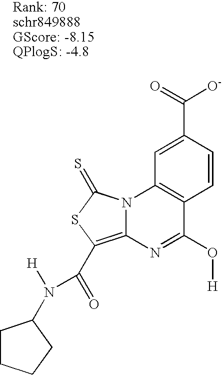

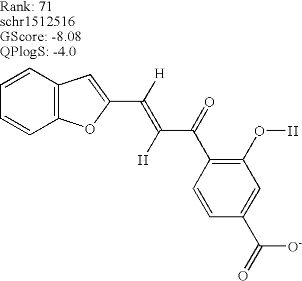

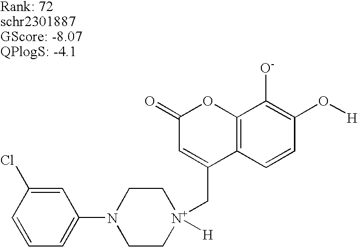

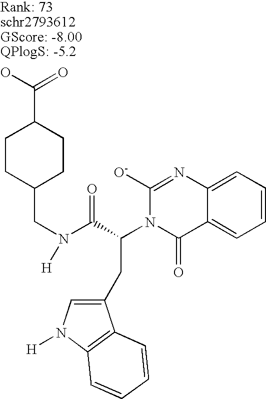

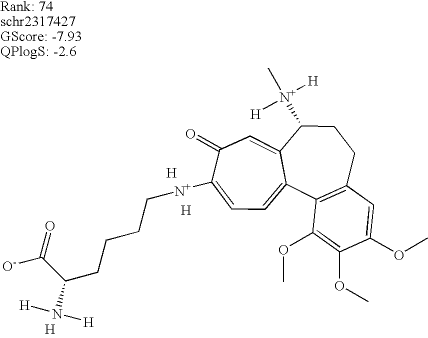

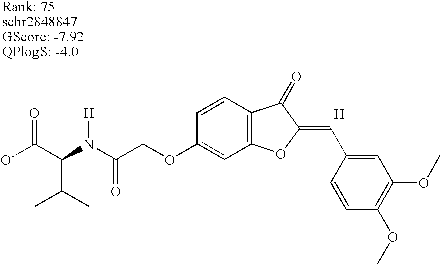

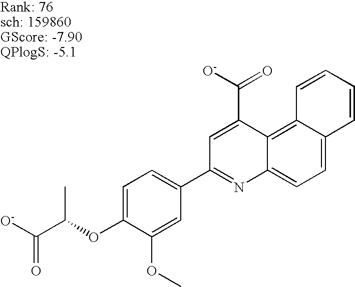

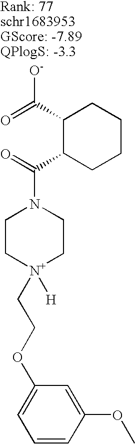

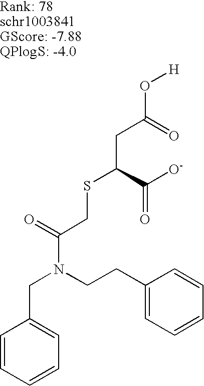

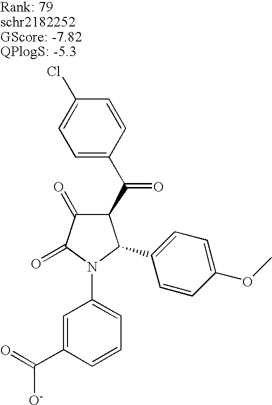

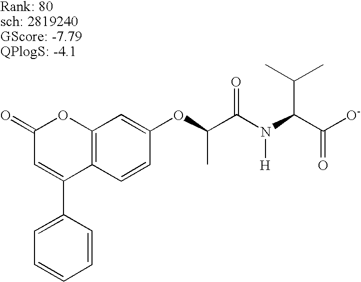

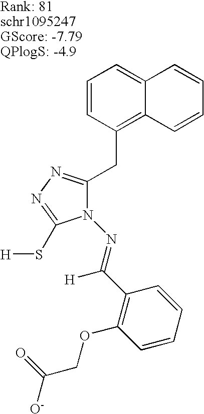

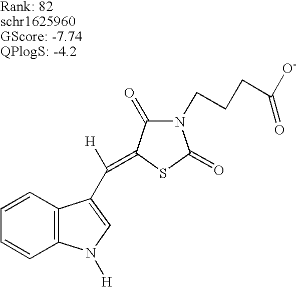

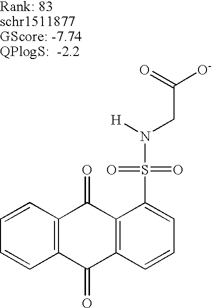

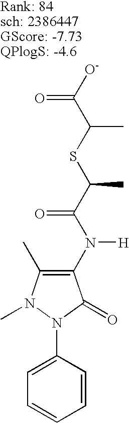

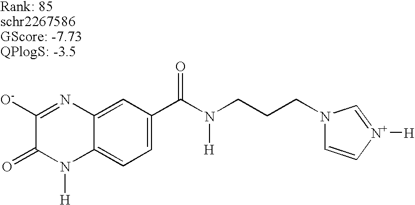

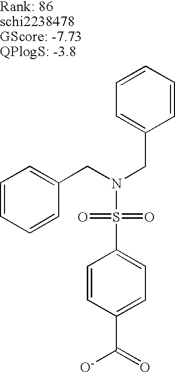

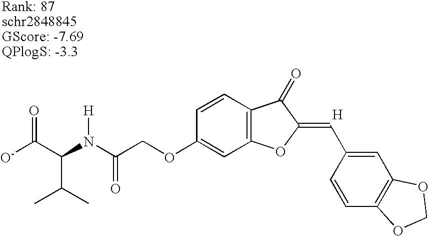

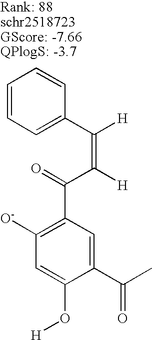

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method for reducing or inhibiting biofilm formation, the method comprising contacting a surface with a neuraminidase inhibitor for a sufficient time so as to modulate neuraminidase activity, thereby reducing or inhibiting formation of the biofilm.

2. The method of claim 1, wherein the surface comprises a biofilm.

3. The method of claim 1, wherein the biofilm is produced by a bacterium, a virus, a protozoan, a fungus, or any combination thereof.

4. The method of claim 1, wherein the neuraminidase is a bacterial neuraminidase or a viral neuraminidase.

5. The method of claim 1, wherein the neuraminidase inhibitor comprises one or more compounds having a structure depicted in Table 4.

6. The method of claim 1, wherein the neuraminidase inhibitor comprises oseltamivir, peramivir, zanamivir, or a variant thereof.

7. The method of claim 1, wherein the neuraminidase inhibitor comprises Formula I: 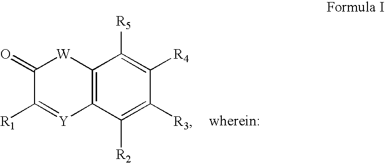
wherein:
W is —O—, or —NH—;
Y is N, or CR6;
R1 is —H, —OH, —R7, or —C1-C6 alkyl;
R2 is —H, or —OH;
R3 is —H, -halogen, or —C(O)—NH—CR8R8R9;
R4 is —H, -methyl, —C(O)—NH-naphthyl, or —OR7;
R5 is —H, —OH, or —CH2-R10;
R6 is -methyl, -phenyl, or —CH2-R11, or R1 and R6 can combine to form a carbocycle;
R7 is —H, or —CR8R8-C(O)—R10, wherein —CR8R8- can be achiral, an R or S enantiomer or a mixture of both enantiomers;
each R8 is independently —H, or -methyl;
R9 is —H, -phenyl, -2-(imidazol-1-yl)ethyl, or -2,3-dihydrobenzo[b][1,4]dioxin-6-yl;
R10 is —OH, or an amino acid linked through the a-nitrogen of the amino acid;
R11 is -4-phenyl-piperazin-1-yl;
wherein the amino acid can be a natural or unnatural amino acid including alanine, arginine, aspartic acid, cystine, glutamic acid, glycine, histidine, isoleucine, leucine, lysine, methionine, phenylalanine, proline, serine, threonine, tryptophan, tyrosine, valine, phenylglycine, norleucine, homoproline, or norvaline, and the amino acid (except glycine) can be the D- or L-isomer or the compound of the above Formula can be a mixture of both amino acid isomers; and, wherein each phenyl or naphthyl group is unsubstituted or substituted with one or more of C1-6 alkoxy including methoxy, a halogen, or any combination thereof; or a pharmaceutically acceptable salt, hydrate, cation, or anion thereof.

8. The method of claim 7, wherein C1-C6 alkyl is methyl, ethyl, propyl, butyl, pentyl, or hexyl.

9. The method of claim 1, wherein the neuraminidase inhibitor comprises Formula
II: 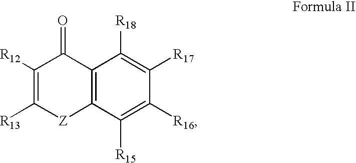
wherein: Z is —O—, —NEt-, or —CR14-;
R12 is -phenyl, —CO2H, -3,4-dihydro-2H-benzo[b][1,4]dioxepine-7-yl, or -(1-phenyl-but-2-en-1-one)-4-yl;
R13 is —H or -methyl;
R14 is —CO2H;
R15 is —H or —CH2-R19
R16 is —H, —OH, —O—CH2CO2H, or —NH-(2-phenyl-thiazolidin-4-one)-3-yl;
R17 is —H, -methyl, —C1-6-alkyl, or -halogen;
R18 is —H or —OH;
R19 is —OH, or an amino acid linked through the a-nitrogen of the amino acid;
wherein the amino acid can be a natural or unnatural amino acid including alanine, arginine, aspartic acid, cystine, glutamic acid, glycine, histidine, isoleucine, leucine, lysine, methionine, phenylalanine, proline, serine, threonine, tryptophan, tyrosine, valine, phenylglycine, norleucine, homoproline, or norvaline, and the amino acid (except glycine) can be the D- or L-isomer, or the compound of the above Formula can be a mixture of both amino acid isomers; and, wherein, each phenyl group is unsubstituted or substituted with one or more of C1-6-alkoxy including methoxy, a halogen, or any combination thereof; or a pharmaceutically acceptable salt, hydrate, cation, or anion thereof.

10. The method of claim 1, wherein the neuraminidase inhibitor comprises Formula
III: 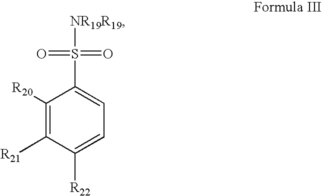
wherein: each R19 is independently —H, -benzyl, -phenyl, -naphthyl, —O-phenyl, or R23;
R20 is —H or —OH;
R21 is —H or —CO2H, or R20 and R21 combine to form a saturated or aromatic carbocyclic ring;
R22 is —H, —CO2H, —C1-6-alkyl, -methyl, or -halogen;
R23 is: 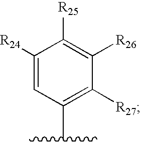
R24 is —H or —S—(CH₂)ₙ-furanyl;
R25 is —H, —OH, N-piperidinyl, or pyridinylmethyl;
R26 is —H; and,
R27 is —H, or R26 and R27 combine to form a saturated or aromatic carbocyclic ring, n is 1-6,
wherein each phenyl or naphthyl is unsubstituted or substituted with one or more of C1-6-alkyl including methyl, C1-6-alkoxy including methoxy, or halogen, or any combination thereof; or a pharmaceutically acceptable salt, hydrate, cation, or anion thereof.

11. The method of claim 1, wherein the neuraminidase inhibitor comprises Formula
IV: 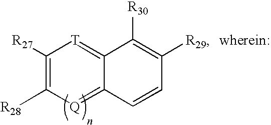
wherein:
T is CR31 or N;
Q is CR31 or N;
n is 0, 1
R27 is —H, phenyl, or benzo-3,4-dioxolane;
R28 is -phenyl, 4-carboxymethyl-piperazin-1-yl, benzo-3,4-dioxolane, 4-([1-carboxyethoxy]-3-methoxy)-phenyl, or 2-(5-(carboxymethyl)-4-oxothiazolidin-2-ylidene)hydrazono-ethyl;
R29 is —H, —OH, or halogen;
R30 is —H, —OH, -halogen, —CO2H, or R31 and R32 combine to form an unsubstituted or substituted aromatic or saturated carbocyclic ring; and
R31 is —H, —CO2H, or (4-(2-(carboxymethoxy)benzylideneamino)-5-mercapto-4H-1,2,4-triazol-3-yl)methyl, wherein each saturated or aromatic carbocyclic ring, including phenyl, are unsubstituted or substituted with one or more of —OH, α-halogen, a C1-6-alkyl group, or a C1-6-alkoxy group; or a pharmaceutically acceptable salt, hydrate, cation, or anion thereof.

12. The method of claim 1, wherein the neuraminidase inhibitor comprises Formula
V: 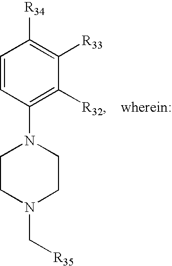
wherein:
R32 is —H or -halogen;
R33 is —H or -halogen;
R34 is —H, phenyl, or cyclohexyl, or R33 and R34 combine to form an aromatic carbocyclic ring;
R35 is —CO2H, -phenyl, 2-hydroxy-5-nitrophenyl, or 5-(1-carboxypentyloxy)-4-oxo-4H-pyran-2-yl; wherein each phenyl or cyclohexyl group is unsubstituted or substituted with one or more of —OH, a C1-6-alkyl including methyl, a C1-6-alkoxy including methoxy, a halogen, a nitro group, or any combination thereof; or a pharmaceutically acceptable salt, hydrate, cation, or anion thereof.

13. The method of claim 3, wherein the bacterium is a Gram-negative bacterium.

14. The method of claim 3, wherein the bacterium is Pseudomonas, Haemophilus, Vibrio, Pseudomonas aeruginosa, Haemophilus influenzae, or Vibrio cholerae.

15. The method of claim 1, wherein the surface comprises a cellular surface of a subject, an in vitro surface, or an oral surface of a subject.

16. The method of claim 1, wherein the contacting comprises administering the neuraminidase inhibitor to a subject via subcutaneous, intra-muscular, intra-peritoneal, or intravenous injection; infusion; oral, nasal, or topical delivery; or a combination thereof.

17. The method of claim 15 or 16, wherein the subject is a human, mouse, rat, bird, dog, cat, cow, horse, or pig.

18. The method of claim 1, wherein the surface comprises a prosthetic graft, a catheter, a wound dressing, a wound site, a medical device, a contact lens, an implanted device, an oral device, a pipe, or industrial equipment.

19. The method of claim 1, wherein the contacting comprises applying the neuraminidase inhibitor to a surface of a prosthetic graft, a catheter, a wound dressing, a wound site, or a medical device, and further comprises administering the neuraminidase inhibitor to the subject prior to or during or after the implantation of the prosthetic graft, the implantation of the catheter, the application to the wound site, the application of the wound dressing, or the implantation or insertion of the medical device.

20. The method of claim 18, wherein industrial equipment is found in a GMP facility.

21. The method of claim 18, wherein industrial equipment comprises a plumbing system.

22. The method of claim 1, further comprising administering an effective amount of a therapeutic composition to the subject, the therapeutic composition being different than the neuraminidase inhibitor.

23. The method of claim 22, wherein administering occurs sequentially or simultaneously.

24. The method of claim 22, wherein the therapeutic composition comprises an antibiotic.

25. The method of claim 24, wherein the antibiotic comprises a cephalosporin, a macrolide, a penicillin, a quinolone, a sulfonamide, a tetracycline, or any combination thereof.

26. The method of claim 1, wherein the neuraminidase inhibitor is in a formulation of a paste, a liquid, a powder, a gel, or a tablet.

27. The method of claim 26, wherein the paste formulation further comprises an abrasive.

28. The method of claim 27, wherein the paste formulation is toothpaste.

29. The method of claim 26, wherein the liquid formulation is a mouthwash.

30. A method for treating a biofilm production-related disorder in a subject in need thereof, the method comprising administering to the subject an effective amount of a neuraminidase inhibitor, thereby treating the biofilm production-related disorder.

31. The method of claim 30, wherein the subject is a human, mouse, rat, bird, dog, cat, cow, horse, or pig.

32. The method of claim 30, wherein the disorder affects an epithelial surface, a mucosal surface, or a combination thereof.

33. The method of claim 32, wherein the surface is a lung surface.

34. The method of claim 30, wherein the biofilm production-related disorder is caused by a bacterium.

35. The method of claim 30, wherein the disorder is cystic fibrosis (CF), otitis media, or chronic obstructive pulmonary disease (COPD).

36. The method of claim 30, wherein the disorder is a medical device-related bacterial infection, the device being implanted or inserted into the subject.

37. The method of claim 34, wherein the bacterium comprises a Gram-negative bacterium.

38. The method of claim 34, wherein the bacterium comprises Pseudomonas, Haemophilus, Vibrio, Pseudomonas aeruginosa, Haemophilus influenzae, Vibrio cholerae, or a combination thereof.

39. A method for preventing biofilm formation in the airway of an asymptomatic subject afflicted with cystic fibrosis and who is free of bacterial infection in his/her airway, the method comprising administering to the subject an effective amount of a neuraminidase inhibitor, thereby preventing formation of the biofilm by a bacterium.

40. The method of claim 39, wherein the subject is about 5 years of age or less.

41. The method of claim 39, wherein the bacterium is a Gram-negative bacterium.

42. The method of claim 39, wherein the bacterium is Pseudomonas aeruginosa.

43. The method of claim 39, wherein the neuraminidase inhibitor is administered by subcutaneous, intramuscular, intra-peritoneal, or intravenous injection; infusion; by oral, nasal, or topical delivery; or a combination thereof.

44. A method for identifying a compound that modulates neuraminidase activity, the method comprising:
a) providing an electronic library of test compounds stored on a computer;
b) providing atomic coordinates for at least twenty amino acid residues of Pseudomonas neuraminidase listed in Table 2, or coordinates having a root mean square deviation therefrom, with respect to at least 50% of the Cα atoms, not more than about 2 Å, in a computer readable format;
c) converting the atomic coordinates into electrical signals readable by a computer processor to generate a three dimensional model of the neuraminidase;
d) performing a data processing method, wherein electronic test compounds from the library are superimposed upon the three dimensional model of the neuraminidase; and
e) determining which test compound fits into the binding pocket of the three dimensional model of the neuraminidase,
thereby identifying which compound would modulate the activity of the neuraminidase.

45. A method for identifying a compound that modulates neuraminidase activity, the method comprising:
a) providing an electronic library of test compounds stored on a computer;
b) providing atomic coordinates listed in Table 2 in a computer readable format for at least 10, 15, 20, 25, 30, 35, or 40 amino acid residues located within about 10 Å of the neuraminidase active site, wherein the residues comprise 10 or more of the following residues: Tyr21, His23, Phe24, Glu44, His45, Val46, Gly47, Asp76, Arg78, Asp79, Val80, Thr95, Tyr97, Tyr127, Phe129, Ala130, His 131, Tyr146, Tyr153, Pro179, Tyr180, Asn181, Glu182, Arg198, Val199, Gly200, Ser201, Gly202, Ile235, Leu236, Val237, Ala238, Thr258, Arg260, Ala294, Ser295, Gly296, Tyr297, Phe313, or Glu315;
c) converting the atomic coordinates into electrical signals readable by a computer processor to generate a three dimensional model of the neuraminidase active site;
d) performing a data processing method, wherein electronic test compounds from the library are superimposed upon the three dimensional model of the neuraminidase active site; and
e) determining which test compound fits into the binding pocket of the three dimensional model of the neuraminidase active site,
thereby identifying which compound would modulate the activity of the neuraminidase.

46. The method of claim 44 or 45, further comprising:
f) obtaining or synthesizing the compound determined to be a potential modulator of the neuraminidase activity;
g) contacting a bacterium with the compound in vitro; and
h) determining whether the compound modulates neuraminidase activity using a biological assay.

47. The method of claim 46, wherein the bacterium is a Gram-negative bacterium.

48. The method of claim 46, wherein the bacterium is Pseudomonas, Haemophilus, Vibrio, Pseuidomonas aeruginosa, Haemophilus influenzae, or Vibrio cholerae.

49. The method of claim 46, wherein the biological assay comprises a biofilm assay, an adherence assay, or a combination thereof.

50. A compound identified by the method of claim 44 or 45, wherein the compound binds to the neuraminidase active site, and comes within 10 Å of amino acid residues listed in Table 3.

51. The compound of claim 50, wherein the compound inhibits or reduces biofilm formation.

52. The compound of claim 50, wherein the compound is a peptide that binds to a neuraminidase.

53. The compound of claim 52, wherein the peptide is an anti-neuraminidase antibody or a binding fragment thereof.

54. The compound of claim 52, wherein the peptide interacts with a protein having the amino acid sequence of SEQ ID NO: 2.

55. The compound of claim 50, wherein the compound interacts with a protein having the amino acid sequence of SEQ ID NO: 2.

56. A mutant P. aeruginosa strain having a deletion in a gene encoding a neuraminidase protein.

57. The mutant of claim 56, wherein the deletion is in the PΔ2794 gene having a nucleic acid sequence of SEQ ID NO:1.
